(19) United States
(12) Patent Application Publication
(10) Pub. No.: US 2010/0121056 A1
(43) Pub. Date: May 13, 2010

(54) PSEUDONUCLEOTIDE COMPRISING AN INTERCALATOR

(75) Inventors: Ulf Bech Christensen, Vissenbjerg (DK); Erik Bjerregaard Pedersen, Odense SV (DK)

Correspondence Address: KNOBBE MARTENS OLSON & BEAR LLP, 2040 MAIN STREET, FOURTEENTH FLOOR, IRVINE, CA, 92614, US

(73) Assignee: Human Genetic Signatures Pty. Ltd., North Ryde (AU)

(21) Appl. No.: 12/534,743

(22) Filed: Aug. 3, 2009

Related U.S. Application Data
(63) Continuation of application No. 10/499,479, filed on Jun. 18, 2004, now abandoned.
(60) Provisional application No. 60/365,545, filed on Mar. 20, 2002.

(30) Foreign Application Priority Data
Dec. 18, 2001 (DK) PA 2001 01897
Dec. 18, 2001 (DK) PA 2001 01898
Dec. 18, 2001 (DK) PA 2001 01899
Dec. 18, 2001 (DK) PA 2001 01900
Oct. 14, 2002 (DK) PA 2002 01575
Oct. 14, 2002 (DK) PA 2002 01576
Oct. 14, 2002 (DK) PA 2002 01577
Oct. 14, 2002 (DK) PA 2002 01578

(51) Int. Cl. C07D 471/14; C07F 9/02; C07D 471/04

(52) U.S. Cl. 544/251; 564/12; 544/250; 546/88

(57) ABSTRACT

The present invention relates to intercalator pseudonucleotides. Intercalator pseudonucleotides according to the invention are capable of being incorporated into the backbone of a nucleic acid or nucleic acid analogue and they comprise an intercalator comprising a flat conjugated system capable of co-stacking with nucleobases of DNA. The invention also relates to oligonucleotides or oligonucleotide analogues comprising at least one intercalator pseudo nucleotide. The invention furthermore relates to methods of synthesising intercalator pseudo nucleotides and methods of synthesising oligonucleotides or oligonucleotide analogues comprising at least one intercalator pseudonucleotide. In addition, the invention describes methods of separating sequence specific DNA(s) from a mixture comprising nucleic acids, methods of detecting a sequence specific DNA (target DNA) in a mixture comprising nucleic acids and/or nucleic acid analogues and methods of detecting a sequence specific RNA in a mixture comprising nucleic acids and/or nucleic acid analogues. In particular said methods may involve the use of oligonucleotides comprising intercalator pseudo nucleotides. The invention furthermore relates to pairs of oligonucleotides or oligonucleotide analogues capable of hybridising to one another, wherein said pairs comprise at least one intercalator pseudonucleotide. Methods for inhibiting a DNAse and/or a RNAse and methods of modulating transcription of one or more specific genes are also described.

### FIELD OF INVENTION

The present invention relates to the field of synthetic nucleotide like molecules, which may be incorporated into the backbone of a nucleic acid or nucleic acid analogue. In particular the present invention relates to such synthetic nucleotide like molecules comprising an intercalator, herein designated intercalator pseudonucleotide.

The invention also relates to nucleic acid analogues comprising intercalator pseudonucleotides and to methods of preparing intercalator monomer units.

Furthermore, the invention relates to methods of separating or targeting sequence specific DNA from a nucleic acid mixture as well as methods of decreasing the self-hybridisation of a nucleic acid analogue, methods of increasing the specificity of hybridisation events and methods of levelling melting temperature differences between different hybridisation events in parallel assays optionally being carried out in the same reaction vessel.

### BACKGROUND OF INVENTION

Nucleic acids, such as DNA and RNA as well as a number of nucleic acid analogues such as PNA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, 2′-OR-RNA, α-L-RNA, α-D-RNA, β-D-RNA and others are capable of specifically hybridising to their complementary strands. This specific recognition may be utilised to detect the presence of specific nucleic acid sequences for example for diagnostic purposes.

Certain synthetic nucleic acids have an increased affinity for nucleic acids in general. High affinity towards target nucleic acids may greatly facilitate detection assays and furthermore synthetic nucleic acids with high affinity towards target nucleic acids may be useful for a number of other purposes, such as gene targeting and purification of nucleic acids.

Unfortunately, many of the presently available synthetic nucleic acids also have a very high affinity for complementary synthetic nucleic acids of the same kind. For many purposes this is very undesirable. For example, certain synthetic nucleic acid probes have a tendency to form hairpin loops, which impairs binding to another complementary nucleic acid.

Furthermore, most nucleic acids as well as most synthetic nucleic acid analogues do not discriminate rigidly between different kinds of nucleic acids, i.e. they bind roughly equally well to complementary DNA and complementary RNA.

Although it has been known for some time that there are relatively large differences in the three-dimensional structure of DNA/DNA duplexes and DNA/RNA hybrids and that some enzymes like RNase H is able to recognize one from the other, chemically modified oligonucleotides in general are not able to differentiate between ssRNA and ssDNA.

Some synthetic nucleic acids such as HNA and LNA which comprise modifications in the sugar ring have an increased affinity towards ssDNA and ssRNA in general. These modifications preferentially stabilize hybridization to ssRNA (ΔTₘ+3 to +5° C. and +4 to +8° C. for HNA and LNA respectively) over ssDNA (ΔTₘ+1 to +3° C. and +3 to +5° C. for HNA and LNA respectively). Some modifications are reported to be totally RNA selective, meaning that these oligonucleotide analogues will hybridize only with RNA and not with DNA, but these duplexes have a lower melting temperature than the comparable non-modified hybrids. On the other hand there are only a few reports on modified oligonucleotides that are DNA selective.

Nucleoside analogues with fluorescent labels have attracted interest for the last couple of decades in connection with the development of new methods for distinguishing and detecting specific nucleic acid sequences. Many different fluorescent probes have been used, and pyrene, which is a polycyclic excimer-forming aromate, is one of the most commonly used. Several acyclic nucleoside analogues comprising pyrene have been described.

In the prior art synthetic nucleotide like molecules comprising intercalators are described:

U.S. Pat. No. 5,446,578 describes synthetic nucleotide like molecules comprising fluorescent molecules, which shows a change in spectra with concentration, for example pyrene. In particular, the document describes nucleic acids derivatised with such fluorescent molecule on the phosphate of a nucleic acid backbone or nucleic acids comprising an acyclic backbone monomer unit consisting of 5 atoms between two phosphates of the nucleic acid backbone, coupled to such a fluorescent molecule. The document states that the fluorescent molecules should be positioned at the exterior of a nucleic acid helix so that they are not capable of intercalating with nucleobases of a nucleic acid. Furthermore, it is explained that the fluorescence of the fluorescent molecule increases upon hybridisation and that a cationic surfactant must be present to achieve this effect. The document does neither disclose stabilisation of nucleic acid duplexes nor discrimination between RNA and DNA.

Yamana et al., 1999, describes an oligonucleotide containing a 2′-O-(1-pyrenylmethyl)uridine at the center position. Said oligonucleotide has higher affinity for DNA and lower affinity for RNA compared to an unmodified oligognucleotide. Upon hybridisation monomer and exciplex fluorescence is enhanced.

Yamana et al., 1997, describes a phosphoramidit coupled to pyrene, which may be incorporated into a nucleic acid at any desired position. In particular said phosphoramidit may be incorporated into a nucleic acid, as an acyclic backbone monomer consisting of 5 atoms between two phosphates of the nucleic acid backbone. Upon hybridisation, excimer fluorescence is greatly enhanced and nucleic acids into which said phosphoramidites have been incorporated retain normal binding affinity for DNA.

Korshun et al., 1999, describes a phosphoramidit coupled to a pyrene, which may be incorporated into a nucleic acid at any desired position. In particular said phoshoramidit may be incorporated into a nucleic acid, as an acyclic backbone monomer consisting of 5 atoms between two phosphates of the nucleic acid backbone. Furthermore the document describes oligonucleotides into which said phosphoramidits have been incorporated and it is described that the oligonucleotides have higher affinity for DNA, than an unmodified oligonucleotide. It is mentioned that close coplanar mutual approach of two pyrene residues located in the neighboring positions of a modified oligonucleotide chain is strongly inhibited because of the small length of the linker. Excimer fluorescence increases upon hybridisation, however oligonucleotides comprising 5 such pyrene pseudonucleotides at the end exhibit high excimer fluorescence when unhybridised as well.

U.S. Pat. No. 5,414,077 describes pseudonucleotides, which may comprise an intercalator such as acridines or anthraquinones. The pseudonucleotide comprises an achiral or a single enantiomer organic backbone, such as diethanolamine. The pseudonucleotides may be incorporated at any desired position within an oligonucleotide. Such oligonucleotides in general have higher affinity for complementary nucleotides, in particular when the pseudonucleotides are inserted at the end. The document does not describe fluorescence data.

U.S. Pat. No. 6,031,091 describes pseudonucleotides which may be incorporated at any position in an oligonucleotide. In particular the document describes acyclic phosphor-containing backbones and it is mentioned that the pseudonucleotides may comprise an intercalator. Specific pseudonucleotides described in the document comprises very long linkers connecting polyaromates to the nucleic acid backbone.

EP 0 916 737 A2 describes polynucleotides derivatised with for example intercalating compounds. The intercalating compounds should preferably be positioned with approx. 10 nucleotides in between. The polynucleotide may be derivatised on the phosphate, the sugar or the nucleobase moiety. In particular, they may be derivatised on the nucleobase by a 7 or a 11 atoms long linker coupled to a polyaromate in a manner that does not interfere with Watson-Crick base pairing. It is stated that fluorescence intensity is enhanced by intercalation.

Strässler et al., 1999, describes pseudonucleoside comprising a fluorescent molecule for example pyrene instead of a nucleobase.

Ebata et al., 1995, describes incorporation of a pyrene-modified nucleotide in the 5′ end of a DNA oligonucleotide and a pyrene-modified nucleotide into the 3′ end of another. By hybridising to a target sequence in a way that the pyrene moieties from the two strands come into close proximity of each other, an excimer band at 490 nm was generated.

Paris et al., 1998, described a system similar to the one disclosed by Ebata et al. wherein the system may be utilised to detect mismatches. However the ability of the system to differentiate between a fully complementary sequence (wt) and a single point mutant (mut) is due to the ability of one of the probes to hybridise in one case but not in the other. This means that the phenomena is temperature controlled and limits the length of the probe and hence the selectivity and sets high requirements to the temperature control.

### SUMMARY OF INVENTION

This application claims benefit under §119(e) to U.S. provisional patent application Ser. No. 60/365,545 filed 20 Mar. 2002, which is hereby incorporated by reference in its entirety.

All patent and non-patent references cited in the application, or in the present application, are also hereby incorporated by reference in their entirety.

The present invention relates to pseudonucleotides or polynucleotide analogues comprising intercalators and having one or more of the following characteristics:

Being able to

1. Intercalate into the double helix at a predetermined position; and/or

2. Substantially increase the affinity for DNA; and/or

3. Inhibit or decrease self and cross hybridisation; and/or

4. Discriminate between different nucleic acids, such as RNA and DNA; and/or

5. Substantially increase the specificity of hybridisation; and/or

6. Increase nuclease stability; and/or

7. Enhance strand invasion significantly; and/or

8. Show a change in fluorescence intensity upon hybridisation

Hence there exists an unmet need for inexpensive pseudonucleotides, that are capable of altering the properties of An oligonucleotide according to the above mentioned criteria.

It is an aspect of the present invention to provide an intercalator pseudonucleotide of the general structure

X—Y-Q

wherein
X is a backbone monomer unit capable of being incorporated into the backbone of a nucleic acid or nucleic acid analogue,
Q is an intercalator comprising at least one essentially flat conjugated system, which is capable of co-stacking with nucleobases of DNA; and
Y is a linker moiety linking said backbone monomer unit and said intercalator.

More preferably the invention relates to an intercalator pseudonucleotide of the general structure

X—Y-Q

wherein
X is a backbone monomer unit capable of being incorporated into the backbone
of a nucleic acid or nucleic acid analogue of the general formula,

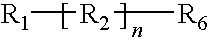

Wherein n=1 to 6
R₁ is a trivalent or pentavalent substituted phosphoratom,
R₂ is individually selected from an atom capable of forming at least two bonds, R₂ optionally being individually substituted, and
R₆ is a protecting group.
Q is an intercalator comprising at least one essentially flat conjugated system, which is capable of co-stacking with nucleobases of DNA; and
Y is a linker moiety linking any of R₂ of said backbone monomer unit and said intercalator; and
wherein the total length of Q and Y is in the range from 7 å to 20 å,
with the proviso that when the intercalator is pyrene the total length of Q and Y is in the range from 9 A to 13 Å, preferably from 9 Å to 11 Å.

By the term “incorporated into the backbone of a nucleic acid or nucleic acid analogue” is meant that the intercalator pseudonucleotide may be inserted into a sequence of nucleic acids and/or nucleic acid analogues.

By the term “flat conjugated system” is meant that all atoms included in the conjugated system are located in one plane. By the term “essentially flat conjugated system” is meant that at most 20% of all atoms included in the conjugated system are not located in said plane at any time.

By the term “conjugated system ” is meant a structural unit containing chemical bonds with overlap of atomic p orbitals of three or more adjacent atoms (Gold et al., 1987. Compendium of Chemical Terminology, Blackwell Scientific Publications, Oxford, UK).

Co-stacking according to the present invention is used as short for coaxial stacking. Co-axial stacking is an energetically favorable structure where flat molecules align on top of each other (flat side against flat side) along a common axis in a stack-like structure. Co-stacking according to the present invention requires interaction between two pi-electron clouds of individual molecules. In the case of intercalator pseudonucleotides co-stacking with nucleobases in a duplex, preferably there is an interaction with a pi electron system on an opposite strand, more preferably there is interaction with pi electron systems on both strands. Co-stacking interactions are found both inter- and intra-molecularly. For example nucleic acids adopt a duplex structure to allow nucleobase co-stacking.

It is a second aspect of the present invention to provide a method of synthesising such an intercalator pseudonucleotide, wherein synthesis may comprise the steps of
a1) providing a compound containing an intercalator comprising at least one essentially flat conjugated system, which is capable of co-stacking with nucleobases of a nucleic acid and optionally a linker part coupled to a reactive group; and
b1) providing a linker precursor molecule comprising at least two reactive groups, said two reactive groups may optionally be individually protected; and
c1) reacting said intercalator with said linker precursor and thereby obtaining an intercalator-linker; and
d1) providing a backbone monomer precursor unit comprising at least two reactive groups, said two reactive groups may optionally be individually protected and/or masked) and optionally comprising a linker part; and
e1) reacting said intercalator-linker with said backbone monomer precursor and obtaining an intercalator-linker-backbone monomer precursor;

or
a2) providing a backbone monomer precursor unit comprising at least two reactive groups, said two reactive groups may optionally be individually protected and/or masked) and optionally comprising a linker part; and
b2) providing a linker precursor molecule comprising at least two reactive groups, said two reactive groups may optionally be individually protected; and
c2) reacting said monomer precursor unit with said linker precursor and thereby obtaining a backbone-linker; and
d2) providing a compound containing an intercalator comprising at least one essentially flat conjugated system, which is capable of co-stacking with nucleobases of a nucleic acid and optionally a linker part coupled to a reactive group; and
e2) reacting said intercalator with said backbone-linker and obtaining an intercalator-linker-backbone monomer precursor;

or
a3) providing a compound containing an intercalator comprising at least one essentially flat conjugated system, which is capable of co-stacking with nucleobases of a nucleic acid and a linker part coupled to a reactive group; and
b3) providing a backbone monomer precursor unit comprising at least two reactive groups, said two reactive groups may optionally be individually protected and/or masked), and a linker part; and
c3) reacting said intercalator-linker part with said backbone monomer precursor-linker and obtaining an intercalator-linker-backbone monomer precursor;

and
f) optionally protecting and/or de-protecting said intercalator-linker-backbone monomer precursor; and
g) providing a phosphor containing compound capable of linking two pseudonucleotides, nucleotides and/or nucleotide analogues together; and
h) reacting said phosphorous containing compound with said intercalator-linker-backbone monomer precursor; and
i) obtaining an intercalator pseudonucleotide
It is a third aspect of the present invention to provide oligonucleotides or oligonucleotide analogues comprising at least one of the intercalator pseudonucleotides according to the present invention, such as An oligonucleotide or oligonucleotide analogue comprising at least one intercalator pseudo nucleotide of the general structure

X—Y-Q

wherein
X is a backbone monomer unit capable of being incorporated into the backbone of a nucleic acid or nucleic acid analogue,
Q is an intercalator comprising at least one essentially flat conjugated system, which is capable of co-stacking with nucleobases of DNA; and
Y is a linker moiety linking said backbone monomer unit and said intercalator.

It is furthermore an aspect of the present invention to provide methods of synthesising oligonucleotides or oligonucleotide analogues comprising at least one intercalator pseudonucleotide, wherein said methods comprise the steps of
a) bringing an intercalator pseudonucleotide according to the present invention into contact with a growing chain of a support-bound nucleotide, oligonucleotide, nucleotide analogue and/or oligonucleotide analogue; and
b) reacting said intercalator pseudonucleotide with said support-bound nucleotide, oligonucleotide, nucleotide analogue or oligonucleotide analogue; and
c) optionally further elongating said oligonucleotide and/or oligonucleotide analogue by adding one or more nucleotides, nucleotide analogues or intercalator pseudonucleotides to the oligonucleotide and/or oligonucleotide analogue in a desired sequence; and
d) cleaving said oligonucleotide and/or oligonucleotide analogue from said solid support; and
e) thereby obtaining said oligonucleotide and/or oligonucleotide analogue comprising at least one intercalator pseudonucleotide.

Furthermore it is an aspect of the present invention to provide oligonucleotides or oligonucleotide analogues comprising at least one intercalator pseudonucleotide according to the present invention, wherein the melting temperature of a duplex consisting of said oligonucleotide or oligonucleotide analogue and a complementary DNA (DNA hybrid), is significantly higher than the melting temperature of a hybrid consisting of said oligonucleotide analogue comprising at least one intercalator and a complementary RNA (RNA hybrid).

Also, the incorporation of at least one intercalator pseudonucleotide according to the invention into an oligonucleotide or oligonucleotide analogue leads to an increase in the melting temperature of a duplex of said oligonucleotide with pseudonucleotide and a complementary oligonucleotide or oligonucleotide analogue as compared to the melting temperature of a duplex of said oligonucleotide without pseudonucleotide.

It is also an aspect of the present invention to provide oligonucleotides and/or oligonucleotide analogues comprising at least one intercalator pseudonucleotide wherein the melting temperature of a hybrid consisting of said oligonucleotide and/or oligonucleotide analogue and a complementary DNA sequence (DNA hybrid) is significantly higher than the melting temperature of a hybrid between said oligonucleotide and/or oligonucleotide analogue lacking the intercalator pseudonucleotide(s) consisting of the same nucleotide sequence as said oligonucleotide and/or oligonucleotide analogue and said complementary DNA.

Additionally it is an aspect of the present invention to provide oligonucleotides and/or oligonucleotide analogues comprising at least one intercalator pseudonucleotide wherein the melting temperature of a hybrid consisting of said oligonucleotide analogue and a complementary RNA (RNA hybrid sequence) is significantly higher than the melting temperature of a hybrid between said oligonucleotide analogue lacking the intercalator pseudonucleotide(s) consisting of the same nucleotide sequence as said oligonucleotide analogue and said complementary RNA.

The intercalator pseudonucleotide(s) may be positioned at any suitable position in the oligonucleotide, with respect to RNA sequences the intercalator pseudonucleotide(s) is(are) preferentially positioned at either or both ends of the oligonucleotide analogue.

It is another aspect of the present invention to provide methods of separating sequence specific DNA(s) from a mixture comprising nucleic acids comprising the steps of
a) providing a mixture comprising nucleic acids; and
b) providing one or more different oligonucleotides or oligonucleotide analogues comprising at least one intercalator pseudonucleotide, wherein the melting temperature of a hybrid consisting of said oligonucleotide or oligonucleotide analogue comprising at least one intercalator pseudonucleotide and a homologously complementary DNA sequence (DNA hybrid), is significantly higher than the melting temperature of a hybrid consisting of said oligonucleotide or oligonucleotide analogue comprising at least one intercalator pseudonucleotide and a homologously complementary RNA (RNA hybrid), and wherein said oligonucleotides or oligonucleotide analogues comprising at least one intercalator pseudonucleotide are capable of hybridising with said sequence specific DNA; and
c) incubating said mixture with said oligonucleotide or oligonucleotide analogue under conditions that allow for hybridisation between said oligonucleotide or oligonucleotide analogue and said sequence specific DNA (DNA hybrid); and
d) separating the oligonucleotides or oligonucleotide analogues together with nucleic acids hybridised to said oligonucleotides from the mixture; and
thereby obtaining separated sequence specific DNA(s) and a separated remaining mixture comprising nucleic acids.

It is a still further aspect of the present invention to provide methods of detecting a sequence specific RNA in a mixture comprising nucleic acids and/or nucleic acid analogues comprising the steps of
a) providing a mixture of nucleic acids; and
b) providing one or more different oligonucleotides or oligonucleotide analogues comprising at least one intercalator pseudonucleotide, wherein the melting temperature of a hybrid consisting of said oligonucleotide and/or oligonucleotide analogue comprising at least one intercalator pseudonucleotide and a homologously complementary DNA (DNA hybrid), is significantly higher than the melting temperature of a hybrid consisting of said oligonucleotide and/or oligonucleotide analogue comprising at least one intercalator pseudonucleotide and a homologously complementary RNA (RNA hybrid), and wherein said oligonucleotides and/or oligonucleotide analogues comprising at least one intercalator pseudonucleotide are substantially complementary to said sequence specific RNA; and
c) providing a probe comprising a detectable label and a nucleic acid sequence capable of hybridising with said sequence specific RNA; and
d) incubating said mixture with said oligonucleotide or oligonucleotide analogue under conditions that allow for hybridisation; and
e) incubating said mixture with said probe under conditions that allow for hybridisation; and
f) detecting said detectable label; and
thereby detecting said sequence specific RNA.

In addition it is an aspect of the present invention to provide oligonucleotides or oligonucleotide analogues comprising at least one intercalator pseudo nucleotide according to the present invention, wherein the melting temperature of a self-hybrid consisting of said oligonucleotide or oligonucleotide analogue is significantly lower than the melting temperature of a hybrid consisting of said oligonucleotide or oligonucleotide analogue comprising at least one intercalator pseudonucleotide and a homologously complementary DNA (DNA hybrid).

Further it is an aspect of the present invention to provide methods of increasing the specificity of hybridisation between oligonucleotides or oligonucleotide analogues comprising at least one intercalator pseudonucleotide and a complementary nucleic acid target or nucleic acid analogue target, wherein the hybrid of said oligonucleotides or oligonucleotide analogues and said target has a significantly higher melting temperature than the hybrid of said oligonucleotide analogue and a nucleic acid and/or nucleic acid analogue not identical to said target.

It is also an aspect of the present invention to provide methods for leveling the melting temperature in multiplex hybridisation assays between different oligonucleotides and/or oligonucleotide analogue sequences, where at least two oligonucleotides or oligo nucleotide analogues comprising at least one intercalator pseudonucleotide, and their complementary and optionally their homologously complementary nucleic acid and/or nucleic acid analogue targets, wherein the melting temperatures of the hybrid between said oligonucleotides and/or oligonucleotide analogues and said targets are significantly more homogeneous than the melting temperatures of said oligonucleotides and/or oligonucleotide analogues of the same sequences with no intercalator pseudonucleotide(s) and said targets.

Furthermore it is an aspect of the present invention to provide oligonucleotides or oligonucleotide analogues comprising at least one intercalating pseudonucleotide that are significantly more nuclease stable than a corresponding oligonucleotide acid comprising no intercalator pseudonucleotides.

It is a still further aspect of the present invention to provide oligonucleotides and/or oligonucleotide analogues comprising at least one fluorescent intercalating pseudonucleotide, wherein said oligonucleotides or oligonucleotide analogues are capable of hybridising to a complementary DNA, and wherein said hybridisation results in a decrease in fluorescence of said oligonucleotides and/or oligonucleotide analogues. Accordingly, the fluorescence properties can be used for detecting hybridisation between said oligonucleotides and/or oligonucleotide analogues comprising at least one intercalator pseudonucleotide and said complementary DNA.

Furthermore, it is an aspect to provide a pair of oligonucleotides or oligonucleotide analogues comprising a first sequence, which is an oligonucleotide and/or oligonucleotide analogue comprising at least one intercalator pseudonucleotide and a second sequence capable of hybridising to said first sequence, wherein the oligonucleotides or oligonucleotide analogues comprising a first sequence is as defined above.

Also, it is an aspect to provide a method for inhibiting a DNAse and/or a RNAse comprising the delivery of at least one oligonucleotide and/or oligonucleotide analogue comprising at least one intercalator pseudonucleotide to the RNAses and/or DNAses thereby inhibiting said DNAse and/or a RNAse.

In a still further aspect the invention relates to a method of modulating transcription of, one or more specific genes, comprising the steps of
a) providing a transcription system;
b) providing at least one oligonucleotide analogue as defined above,
   and wherein said oligonucleotide and/or oligonucleotide analogue is capable of hybridizing with said gene and/or regulatory sequences thereof or the complementary strand of said gene and/or regulatory sequences thereof; and
c) introducing said oligonucleotide and/or oligonucleotide analogue into the transcription system; and
d) allowing hybridization of oligonucleotide and/or oligonucleotide analogue with said one or more genes and/or regulatory sequences hereof or the complementary strand of the gene and/or regulatory sequences hereof; and
thereby modulating transcription of said gene.

Finally, it is an aspect of the present invention to combine two or more, preferentially most or all, of the properties and methods described herein above in new methods to obtain methods and products of advantageous functional and hybridisation-related characteristics.

### LEGENDS OF FIGURES

FIG. 1 illustrates the synthesis of an intercalator pseudonucleotide, a phosphoramidite as depited in 5.

FIG. 2 illustrates a structural calculation of the self-complementary DNA duplex with the sequence 5′-XCGCGCG-3′ done in “MacroModel”, X=the pyrene module. The pyrene moiety is co-axial stacked with the underlying base pair.

FIG. 3 illustrates a slice of the calculated structure of the duplex 5′-AGCTTGCCTTGAG-3′+5′-CTCAAGXCAACCT-3′, X=5. The pyrene makes co-axial stacking with both the upper and lower neighboring nucleobases of the opposite strand.

FIG. 4 illustrates the calculated structure of a 12/13-mer duplex with the sequence 5′-AGCTTGCTTGAG-3′+5′-CTCAAGXCAACCT-3′, X=5 (FIG. 1). The pyrene moiety is able to interact with both the upper and lower neighboring nucleobases of the opposite strand. The distance between the nucleobases and the pyrene moiety is shown to the right.

FIG. 5 illustrates fluorescent measurements of a 13-mer, mono pyrene inserted ssDNA ( [symbol] ); its duplex with complementary, 12-mer RNA ( [symbol] ) and its duplex with complementary, 12-mer DNA (♦). The sequences are the same as those shown in Table 3.

FIG. 6 illustrates fluorescent measurements of a 14-mer ssDNA with two pyrene insertions separated by one nucleotide ( [symbol] ); its duplex with complementary, 12-mer RNA ( [symbol] ) and its duplex with complementary, 12-mer DNA (♦).The sequences are the same as those shown in Table 3.

FIG. 7 illustrates a procedure to prepare a sample for RT-PCR

FIG. 8 illustrates a procedure to prepare a sample for RT-PCR

FIG. 9 illustrates a procedure to prepare a sample for RT-PCR

FIG. 10 illustrates a procedure to prepare sequence specific DNA

FIG. 11 illustrates a procedure to prepare a sequence specific DNA

FIG. 12 illustrates a method to detect sequence specific DNA using a chip

FIG. 13 illustrates different kinds of oligonucleotides that may be useful as probes on a chip

FIG. 14 illustrates PCR quantification.

FIG. 15 illustrates transcription blockage using a pair of oligonucleotides according to the invention indicated as A and B, respectively.

FIG. 16: Nuclease resistance of two oligonucleotides whereof one comprises intercalating pseudonucleotides (INA oligo) and the duplex of said two oligonucleotides.

FIG. 17: Secondary structure of the hairpin forming probe I. In this conformation the monomer and excimer fluorescence is quenched.

FIG. 18: Secondary structure of probe I when hybridised to at target sequence. When hybridized to a target sequence, the excimer complex is free to be formed and hence excimer fluorescence can be observed. The monomer fluorescence is also increased.

FIG. 19 SYBR green II stained INA oligos, visualized on an ArrayWorx scanner.

FIG. 20: illustrates a test of oligo binding on Asper SAL slides.

FIG. 21: Exciplex fluorescence between molecules X and Y when placed as next-nearest neighbours (Sequence I)

FIG. 22: Exciplex fluorescence between molecules X and Y when placed as neighbours (Sequence II)

FIG. 23: Exciplex fluorescence between molecules Y and Z when placed as neighbours

FIG. 24: illustrates EtBr staining

FIG. 25: Sequence of the employed double-stranded target oligo, the attacking IOs and the complimentary pairing IOs. Y denote intercalating units.

FIG. 26: IOs spontaneously bind target DNA.

Reactions where carried out in 20 μl volumes containing 126 nM IOs with or without 20 nM target DNA, for 1 h at 37° C. Binding was assayed by electrophoresis in a 10% polyacrylamide/½× TBE gel and visualized by phosphorimaging.

FIG. 27: IO-DNA complex formation requires sequence complimentarity.

Reactions were carried out in 15 μl volumes containing the indicated concentrations of IOs with or without 20nM target DNA (single or double stranded), for 2 h at 37° C. Binding was assayed by electrophoresis in a 10% polyacrylamide/½× TBE gel and visualized by phosphorimaging.

FIG. 28: IO pairing in spontaneous target binding.

Reactions were carried out in 15 μl volumes containing 20 nM target DNA and the indicated concentrations of pre-annealed P32-labelled IOs for 2 h at 37° C. Binding was assayed by electrophoresis in a 10% polyacrylamide/½× TBE gel and visualized by phosphorimaging.

FIG. 29: Pairing does not affect the efficiency of spontaneous binding.

Reactions were carried out in 15 μl volumes containing 20nM target DNA and increasing amounts of IOs (40-80-160 nM) as indicated for 4 h at 37° C. Binding was assayed by electrophoresis in a 10% polyacrylamide/½× TBE gel and visualized by phosphorimaging. Band intensities are relative numbers representing intensities of the band areas.

FIG. 30: IO-DNA complex formation in nuclear extracts

Reactions were carried out in 15 μl volumes containing pre-annealed 180 nM IOs and 20 nM target DNA where indicated, nuclear extracts (NE) were added to the reactions as indicated. Reactions were incubated at 37° C. for 10 min, and then another 60 min upon addition of 1.125 μl 10% SDS and 37.5 μg Proteinase K. Binding was assayed by electrophoresis in a 7% polyacrylamide/½× TBE gel and visualized by phosphorimaging.

FIG. 31: Nuclear factors favour IO-DNA complex formation by IO pairs

Reactions were carried out in 15 82 l volumes containing 180 nM IOs and 20 nM target DNA. 10 μg HeLa nuclear extract were added to the reactions. Reactions were incubated at 37° C. for 10 min, and then another 60 min upon addition of 1.125 μl 10% SDS and 37.5 μg Proteinase K. Binding was assayed by electrophoresis in a 10% polyacrylamide/½× TBE gel and visualized by phosphorimaging

FIG. 32: Chemical structures of LNA and INA P nucleotide monomers. B=nucleobase.

FIG. 33: Melting temperature data of INAs with different insertion patterns when hybridised to the complementary structure and LNA targets. P=INA monomer P. T^{L} and ^{Me}C^{L} are locked nucleotides of thymine and 5-methylcytosine, respectively.

FIG. 34: Transition temperatures, Tₘ (° C.) for hairpin probes with ssDNA targets. T^{L} and ^{Me}C^{L} are locked nucleotides of thymine and 5-methylcytosine, respectively.

FIG. 35: A) transition curves of the non-intercalating pseudonucleotide comprising probes B) Two LNA probes comprising one intercalating pseudonucleotide together with the unmodified reference duplex. C) LNA probes comprising one or two intercalating pseudonucleotide together with the unmodified reference duplex. D) A non-intercalating pseudonucleotide comprising LNA probes and two probes comprising one intercalating pseudonucleotide together with corresponding DNA probe all hybridized to a target sequence comprising one intercalating pseudonucleotide.

FIG. 36: Scheme 1. Schematic presentation of the conformations formed by T₄-LNA oligonucleotides at transition temperature.

FIG. 37: Synthesis of 1′-aza pyrenmethyl pseudonucleotide

FIG. 38: Sequences and hybridisation data of synthesized ODNs in DNA/DNA(RNA) duplexes

FIG. 39: Hybridisation data for DNA Three-Way Junction

FIG. 40: illustrates a beacon primer

FIG. 41: —illustrates a PCR quantification strategy using beacon primers

FIG. 42: illustrates complete complementarity and mismatch/excimer formation

### DETAILED DESCRIPTION OF THE INVENTION

Nucleic Acids

The term “nucleic acid” covers the naturally occurring nucleic acids, DNA and RNA, including naturally occurring derivatives of DNA and RNA such as but not limited to methylated DNA, DNA containing adducts and RNA covalently bound to proteins. The term “nucleic acid analogues” covers synthetic derivatives and analogues of the naturally occurring nucleic acids, DNA and RNA. Synthetic analogues comprise one or more nucleotide analogues. The term “nucleotide analogue” comprises all nucleotide analogues capable of being incorporated into a nucleic acid backbone and capable of specific base-pairing (see herein below), essentially like naturally occurring nucleotides.

Hence the terms “nucleic acids” or “nucleic acid analogues” designates any molecule, which essentially consists of a plurality of nucleotides and/or nucleotide analogues and/or intercalator pseudonucleotides. Intercalator pseudonucleotides are described in detail herein below. Nucleic acids or nucleic acid analogues according to the present invention may comprise more different nucleotides and nucleotide analogues with different backbone monomer units (see herein below).

Preferably, single strands of nucleic acids or nucleic acid analogues according to the present invention are capable of hybridising with a substantially complementary single stranded nucleic acid and/or nucleic acid analogue to form a double stranded nucleic acid or nucleic acid analogue. More preferably such a double stranded analogue is capable of forming a double helix. Preferably, the double helix is formed due to hydrogen bonding, more preferably, the double helix is a double helix selected from the group consisting of double helices of A form, B form, Z form and intermediates thereof.

Hence, nucleic acids and nucleic acid analogues according to the present invention includes, but is not limited to the kind of nucleic acids and/or nucleic acid analogues selected from DNA, RNA, PNA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, 2′-OR-RNA, α-L-RNA, α-D-RNA, β-D-RNA and mixtures thereof and hybrids thereof, as well as phosphorous atom modifications thereof, such as but not limited to phosphorothioates, methyl phospholates, phosphoramidiates, phosphorodithiates, phosphoroselenoates, phosphotriesters and phosphoboranoates. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides according to the present invention.

Within this context “mixture” is meant to cover a nucleic acid or nucleic acid analogue strand comprising different kinds of nucleotides or nucleotide analogues. Furthermore, within this context, “hybrid” is meant to cover nucleic acids or nucleic acid analogues comprising one strand which comprises nucleotides or nucleotide analogues with one or more kinds of backbone and another strand which comprises nucleotides or nucleotide analogues with different kinds of backbone. By the term “duplex” is meant the hybridisation product of two strands of nucleic acids and/or nucleic acid analogues, wherein the strands preferably are of the same kind of nucleic acids and/or nucleic acid analogues.

By HNA is meant nucleic acids as for example described by Van Aetschot et al., 1995. By MNA is meant nucleic acids as described by Hossain et al, 1998. ANA refers to nucleic acids described by Allert et al, 1999. LNA may be any LNA molecule as described in WO 99/14226 (Exiqon), preferably, LNA is selected from the molecules depicted in the abstract of WO 99/14226. More preferably LNA is a nucleic acid as described in Singh et al, 1998, Koshkin et al, 1998 or Obika et al., 1997. PNA refers to peptide nucleic acids as for example described by Nielsen et al., 1991.

The term nucleotide designates the building blocks of nucleic acids or nucleic acid analogues and the term nucleotide covers naturally occurring nucleotides and derivatives thereof as well as nucleotides capable of performing essentially the same functions as naturally occurring nucleotides and derivatives thereof. Naturally occurring nucleotides comprise deoxyribonucleotides comprising one of the four nucleobases adenine (A), thymine (T), guanine (G) or cytosine (C), and ribonucleotides comprising on of the four nucleobases adenine (A), uracil (U), guanine (G) or cytosine (C).

Nucleotide analogues may be any nucleotide like molecule that is capable of being incorporated into a nucleic acid backbone and capable of specific base-pairing.

Non-naturally occurring nucleotides according to the present invention includes, but is not limited to the nucleotides selected from PNA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, 2′-OR-RNA, α-L-RNA, and α-D-RNA, β-D-RNA

The function of nucleotides and nucleotide analogues according to the present invention is to be able to interact specifically with complementary nucleotides via hydrogen bonding of the nucleobases of said complementary nucleotides as well as to be able to be incorporated into a nucleic acid or nucleic acid analogue. Naturally occuring nucleotides, as well as some nucleotide analogues are capable of being enzymatically incorporated into a nucleic acid or nucleic acid analogue, for example by RNA or DNA polymerases, however nucleotides or nucleotide analogues may also be chemically incorporated into a nucleic acid or nucleic acid analogue.

Furthermore nucleic acids or nucleic acid analogues may be prepared by coupling two smaller nucleic acids or nucleic acid analogues to another, for example this may be done enzymatically by ligases or it may be done chemically.

Nucleotides or nucleotide analogues comprise a backbone monomer unit and a nucleobase. The nucleobase may be a naturally occuring nucleobase or a derivative thereof or an analogue thereof capable of performing essentially the same function. The function of a nucleobase is to be capable of associating specifically with one or more other nucleobases via hydrogen bonds. Thus it is an important feature of a nucleobase that it can only form stable hydrogen bonds with one or a few other nucleobases, but that it can not form stable hydrogen bonds with most other nucleobases usually including itself. The specific interaction of one nucleobase with another nucleobase is generally termed “base-pairing”.

Base pairing results in a specific hybridisation between predetermined and complementary nucleotides. Complementary nucleotides according to the present invention are nucleotides that comprise nucleobases that are capable of base-pairing.

Of the naturally occurring nucleobases adenine (A) pairs with thymine (T) or uracil (U); and guanine (G) pairs with cytosine (C). Accordingly, e.g. a nucleotide comprising A is complementary to a nucleotide comprising either T or U, and a nucleotide comprising G is complementary to a nucleotide comprising C.

Nucleotides according to the present invention may further be derivatised to comprise an appended molecular entity. The nucleotides can be derivatised on the nucleobases or on the backbone monomer unit. Preferred sites of derivatisation on the bases include the 8-position of adenine, the 5-position of uracil, the 5- or 6-position of cytosine, and the 7-position of guanine. Especially the methylation of position 5 in cytosine is relevant, and hence it is a preferred embodiment of this invention to be able to discriminate between partially or full methylated sequences and non-methylated sequences. The heterocyclic modifications can be grouped into three structural classes: Enhanced, base stacking, additional hydrogen bonding and the combination of these. Modifications that enhance base stacking by expanding the π-electron cloud of planar systems are represented by conjugated, lipophilic modifications in the 5-position of pyrimidines and the 7-position of 7-deaza-purines. Substitutions in the 5-position of pyrimidines modifications include propynes, hexynes, thiazoles and simply a methyl group; and substituents in the 7-position of 7-deaza purines include iodo, propynyl, and cyano groups. It is also possible to modify the 5-position of cytosine from propynes to five-membered heterocycles and to tricyclic fused systems, which emanate from the 4- and 5-position (cytosine clamps). A second type of heterocycle modification is represented by the 2-amino-adenine where the additional amino group provides another hydrogen bond in the A-T base pair,-analogous to the three hydrogen bonds in a G-C base pair. Heterocycle modifications providing a combination of effects are represented by 2-amino-7-deaza-7-modified andenine and the tricyclic cytosine analog having an ethoxyamino functional group of heteroduplexes. Furthermore, N2-modified 2-amino adenine modified oligonucleotides are among commonly modifications. Preferred sites of derivatisation on ribose or deoxyribose moieties are modifications of nonconnecting carbon positions C-2′ and C-4′, modifications of connecting carbons C-1′, C-3′ and C-5′, replacement of sugar oxygen, O-4′, Anhydro sugar modifications (conformational restricted), cyclosugar modifications (conformational restricted), ribofuranosyl ring size change, connection sites—sugar to sugar, (C-3′ to C-5′/C-2′ to C-5′), hetero-atom ring—modified sugars and combinations of above modifications. However, other sites may be derivatised, as long as the overall base pairing specificity of a nucleic acid or nucleic acid analogue is not disrupted. Finally, when the backbone monomer unit comprises a phosohate group, the phosphates of some backbone monomer units may be derivatised.

Oligonucleotide or oligonucleotide analogue as used herein are molecules essentially consisting of a sequence of nucleotides and/or nucleotide analogues and/or intercalator pseudo-nucleotides. Preferably oligonucleotide or oligonucleotide analogue comprises 3-200, 5-100, 10-50 individual nucleotides and/or nucleotide analogues and/or intercalator pseudo-nucleotides, as defined above.

Target Nucleic Acids

A target nucleic acid or target nucleic acid analogue sequence refers to a nucleotide or nucleotide analogue sequence which comprise one or more sites/sequences for hybridisation of one or more oligonucleotide(s) and/or oligonucleotide analogue(s), for example primers or probes. Target sequences may be found in any nucleic acid or nucleic acid analogue including, but not limited too, other probes, RNA, genomic DNA, plasmid DNA, cDNA and can for example comprise a wild-type or mutant gene sequence or a regulatory sequence thereof or an amplified nucleic acid sequence, for example as when amplified by PCR. A target sequence may be of any length. The site addressed may or may not be one contiguous sequence. For example said site may be composed of two or more contiguous subsequences separated by any number of nucleotides and/or nucleotide analogues. Preferentially the total length of the site addressed, composed by all subsequences on that particular target nucleic acid or target nucleic acid analogue, by said oligonucleotide and/or oligonucleotide analogue, typically is less than 100 nucleotides and/or nucleotide analogues and/or intercalator pseudonucleotides.

Homologous Nucleic Acids

Nucleic acids, nucleic acid analogues, oligonucleotides or oligonucleotide analogues are said to be homologously complementary, when they are capable of hybridising. Preferably homologously complementary nucleic acids, nucleic acid analogues, oligonucleotides or oligonucleotide analogues are capable of hybridising under low stringency conditions, more preferably homologously complementary nucleic acids, nucleic acid analogues, oligonucleotides or oligonucleotide analogues are capable of hybridising under medium stringency conditions, more preferably homologously complementary nucleic acids, nucleic acid analogues, oligonucleotides or oligonucleotide analogues are capable of hybridising under high stringency conditions.

High stringency conditions as used herein shall denote stringency as in comparison to, or at least as stringent as, what is normally applied in connection with Southern blotting and hybridisation as described e.g. by Southern E. M., 1975, J. Mol. Biol. 98:503-517. For such purposes it is routine practice to include steps of prehybridization and hybridization. Such steps are normally performed using solutions containing 6×SSPE, 5% Denhardt's, 0.5% SDS, 50% formamide, 100 μg/ml denaturated salmon testis DNA (incubation for 18 hrs at 42° C.), followed by washings with 2×SSC and 0.5% SDS (at room temperature and at 37° C.), and washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

Medium stringency conditions as used herein shall denote hybridisation in a buffer containing 1 mM EDTA, 10 mM Na₂HPO₄.H₂0, 140 mM NaCl, at pH 7.0, or a buffer similar to this having approximately the same impact on hybridization stringency. Preferably, around 1.5 μM of each nucleic acid or nucleic acid analogue strand is provided. Alternatively medium stringency may denote hybridisation in a buffer containing 50 mM KCl, 10 mM TRIS-HCl (pH 9.0), 0.1% Triton X-100, 2 mM MgCl2.

Low stringency conditions according to the present invention denote hybridisation in a buffer constituting 1 M NaCl, 10 mM Na₃PO₄ at pH 7.0, or a buffer similar to this having approximately the same impact on hybridization stringency.

Alternatively, homologously complementary nucleic acids, nucleic acid analogues, oligonucleotides or oligonucleotide analogues are nucleic acids, nucleic acid analogues, oligonucleotides or oligonucleotide analogues substantially complementary to each other over a given sequence, such as more than 70% complementary, for example more than 75% complementary, such as more than 80% complementary, for example more than 85% complementary, such as more than 90% complementary, for example more than 92% complementary, such as more than 94% complementary, for example more than 95% complementary, such as more than 96% complementary, for example more than 97% complementary.

Preferably said given sequence is at least 4 nucleotides long, for example at least 10 nucleotides, such as at least 15 nucleotides, for example at least 20 nucleotides,, such as at least 25 nucleotides, for example at least 30 nucleotides, such as between 10 and 500 nucleotides, for example between 4 and 100 nucleotides long, such as between 10 and 50 nucleotides long. More preferably homologously complementary oligonucleotides or oligonucleotide analogues are substantially homologously complementary over their entire length.

Specificity of Hybridisation

The specificity of hybridisation of nucleic acids and/or nucleic acid analogues and/or oligonucleotides and/or oligonucleotide analogues refers to the ability of which said hybridisation event distinguishes between homologously complementary hybridisation partners according to their sequence differences under given stringency conditions. Often it is the intention to target only one particular sequence (the target sequence) in a mixture of nucleic acids and/or nucleic acid analogues and/or oligonucleotides and/or oligonucleotide analogues and to avoid hybridization to other sequences even though they have strong similarity to said target sequence. Sometimes only one or few nucleotides differ among target and non-target sequences in the sequence-region used for hybridization.

High specificity in hybridisation as used herein denotes hybridisation under high stringency conditions at which an oligonucleotide or oligonucleotide analogue will hybridise with a homologous target sequence significantly better than to a nearly identical sequence differing only from said target sequence by one or few base-substitutions.

Discrimination

Discrimination refers to the ability of oligonucleotides and/or oligonucleotide analogues, in a sequence-independent manner, to hybridise preferentially with either RNA or DNA. Accordingly, the melting temperature of a hybrid consisting of oligonucleotide and/or oligonucleotide analogue and a homologously complementary RNA (RNA hybrid) is either significantly higher or lower than the melting temperature of a hybrid between said oligonucleotide and/or oligonucleotide analogue and a homologously complementary DNA (DNA hybrid).

RNA-like and DNA-like

RNA-like refers to nucleic acid analogues or oligonucleotide analogues behaving like RNA with respect to hybridisation to homologously complementary oligonucleotides and/or oligonucleotide analogues comprising at least one internal pseudonucleotide. Accordingly, RNA-like nucleic acid analogues or oligonucleotide analogues can be functionally categorized on the basis of their ability to hybridise with oligonucleotides and/or oligonucleotide analogues able to discriminate between RNA and DNA. Preferentially, said oligonucleotide analogues able to discriminate between RNA and DNA comprises one or more internally positioned pseudonucleotide intercalators and consequently, said oligonucleotide analogue comprising pseudonucleotide intercalators will preferentially not hybridise to said RNA-like nucleic acid analogues or oligonucleotide analogues.

Examples of RNA-like molecules are RNA, 2′-O-methyl RNA, LNA, α-L-Ribo-LNA, α-L-Xylo-LNA, β-D-Xylo-LNA, α-D-Ribo-LNA, [3.2.1]-LNA, 2′-R-RNA, 2′-OR-RNA, and mixtures thereof.

Likewise, DNA-like refers to nucleic acid analogues or oligonucleotide analogues behaving like DNA with respect to hybridisation to homologously complementary nucleic acids and/or nucleic acid analogues. Accordingly, DNA-like nucleic acids or nucleic acid analogues can be functionally categorized on the basis of their ability to hybridise with oligonucleotides or oligonucleotide analogues able to discriminate between RNA and DNA. Preferentially, said oligonucleotides or oligonucleotide analogues able to discriminate between RNA and DNA comprises one or more internally positioned pseudonucleotide intercalators, and consequently, said oligonucleotide analogue comprising pseudonucleotide intercalators will preferentially hybridise to said DNA-like nucleic acid analogues or oligonucleotide analogues.

Examples of DNA-like molecules is DNA and INA (Christensen, 2002. Intercalating nucleic acids containing insertions of 1-O-(1-pyrenylmethyl)glycerol: stabilisation of dsDNA and discrimination of DNA over RNA. Nucl. Acids. Res. 2002 30: 4918-4925).

Cross-Hybridisation

The term cross-hybridisation covers unattended hybridisation between at least two nucleic acids and/or nucleic acid analogues, i.e. cross-hybridisation may also be denoted intermolecular hybridisation. Hence the term cross-hybridization may be used to describe the hybridisation of for example a nucleic acid probe or nucleic acid analogue probe sequence to other nucleic acid sequences and/or nucleic acid analogue sequences than its intended target sequence.

Often cross-hybridization occurs between a probe and one or more homologously complementary non-target sequences, even though these have a lower degree of complementarity than the probe and its complementary target sequence. This unwanted effect could be due to a large excess of probe over target and/or fast annealing kinetics. Cross-hybridization also occurs by hydrogen bonding between few nucleobase pairs, e.g. between primers in a PCR reaction, resulting in primer dimer formation and/or formation of unspecific PCR products.

Especially nucleic acids comprising one or more nucleotide analogues with high affinity for nucleotide analogues of the same type tend to form dimer or higher order complexes based on base pairing. Especially probes comprising nucleotide analogues such as, but not limited to, DNA, RNA, 2′-O-methyl RNA, PNA, HNA, MNA, ANA, LNA, α-L-Ribo-LNA, α-L-Xylo-LNA, β-D-Xylo-LNA, α-D-Ribo-LNA, [3.2.1]-LNA, 2′-R-RNA, 2′-OR-RNA, and mixtures thereof generally have a high affinity for hybridising to other oligonucleotide analogues comprising backbone monomer units of the same type. Hence even though individual probe molecules only have a low degree of complementarity, they tend to hybridise.

Self-Hybridisation

The term self-hybridisation covers the process wherein a nucleic acid or nucleic acid analogue molecule anneals to itself by folding back on itself, generating a secondary structure like for example a hairpin structure, i.e. self-hybridisation may also be defined as intramolecular hybridisation. In most applications it is of importance to avoid self-hybridization. The generation of said secondary structures may inhibit hybridisation with desired nucleic acid target sequences. This is undesired in most assays for example when the nucleic acid or nucleic acid analogue is used as primer in PCR reactions or as fluorophore/quencher labeled probe for exonuclease assays. In both assays self-hybridisation will inhibit hybridization to the target nucleic acid and additionally the degree of fluorophore quenching in the exonuclease assay is lowered.

Especially nucleic acids comprising one or more nucleotide analogues with high affinity for nucleotide analogues of the same type tend to self-hybridise. Especially probes comprising nucleotide analogues such as, but not limited to, DNA, RNA, 2′-O-methyl RNA, PNA, HNA, MNA, ANA, LNA, α-L-Ribo-LNA, α-L-Xylo-LNA, β-D-Xylo-LNA, α-D-Ribo-LNA, [3.2.1]-LNA, 2′-R-RNA, 2′-OR-RNA generally have a high affinity for self-hybridising. Hence even though individual probe molecules only have a low degree of self-complementarity they tend to self-hybridise.

Melting Temperature

Melting of nucleic acids refer to thermal separation of the two strands of a double-stranded nucleic acid molecule.

The melting temperature (Tₘ) denotes the temperature in degrees centigrade at which 50% helical (hybridised) versus coil (unhybridised) forms are present.

A high melting temperature is indicative of a stable complex and accordingly of a high affinity between the individual strands. Vice versa a low melting temperature is indicative of a relatively low affinity between the individual strands. Accordingly, usually strong hydrogen bonding between the two strands results in a high melting temperature.

Furthermore, as disclosed by the present invention, intercalation of an intercalator between nucleobases of a double stranded nucleic acid may also stabilise double stranded nucleic acids and accordingly result in a higher melting temperature.

In addition the melting temperature is dependent on the physical/chemical state of the surroundings. For example the melting temperature is dependent on salt concentration and pH.

The melting temperature may be determined by a number of assays, for example it may be determined by using the UV spectrum to determine the formation and breakdown (melting) of hybridisation.

Backbone Monomer Unit

The backbone monomer unit of a nucleotide or a nucleotide analogue or an intercalator pseudonucleotide according to the present invention is the part of the nucleotide, which is involved in incorporation of the nucleotide or nucleotide analogue or intercalator pseudonucleotide into the backbone of a nucleic acid or a nucleic acid analogue. Any suitable backbone monomer unit may be employed with the present invention.

In particular the backbone monomer unit of intercalator pseudonucleotides according to the present invention may be selected from the backbone monomer units mentioned herein below.

The backbone monomer unit comprises the part of a nucleotide or nucleotide analogue or intercalator pseudonucleotide that may be incorporated into the backbone of an oligonucleotide or an oligonucleotide analogue. In addition, the backbone monomer unit may comprise one or more leaving groups, protecting groups and/or reactive groups, which may be removed or changed in any way during synthesis or subsequent to synthesis of an oligonucleotide or oligonucleotide analogue comprising said backbone monomer unit.

It is important to note that the term backbone monomer unit according to the present invention only includes the backbone monomer unit per se and it does not include for example a linker connecting a backbone monomer unit to an intercalator. Hence, the intercalator as well as the linker is not part of the backbone monomer unit.

Accordingly, backbone monomer units only include atoms, wherein the monomer is incorporated into a sequence, are selected from the group consisting of
a) atoms which are capable of forming a linkage to the backbone monomer unit of a neighboring nucleotide; or
b) atoms which at least at two sites are connected to other atoms of the backbone monomer unit; or
c) atoms which at one site is connected to the backbone monomer unit and otherwise is not connected with other atoms

More preferably, backbone monomer unit atoms are thus defined as the atoms involved in the direct linkage (shortest path) between the backbone Phosphor-atoms of neighbouring nucleotides, when the monomer is incorporated into a sequence, wherein the neighbouring nucleotides are naturally occurring nucleotides,.

The backbone monomer unit may be any suitable backbone monomer unit. In one embodiment of the present invention, the backbone monomer unit may for example be selected from the group consisting of the backbone monomer units of DNA, RNA, PNA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA.

Below is depicted a range of different backbone monomer units of nucleotides and nucleotide analogues, and how they are connected to the nucleobases via linkers that are attached at one or two positions of the backbone monomer unit:

Examples of oligomers of DNA, RNA & PNA

| | | |
|---|---|---|
| DNA | 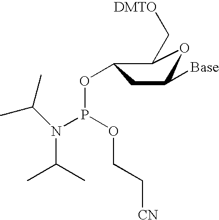 | 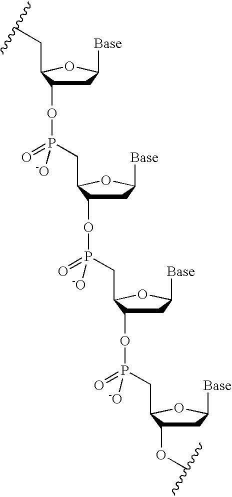 |
| RNA | 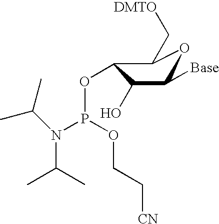 | 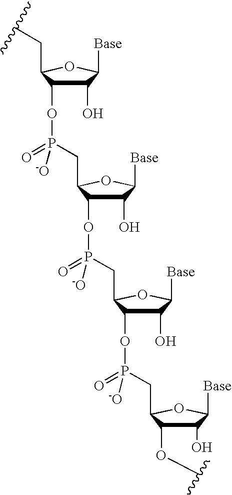 |
| PNA | 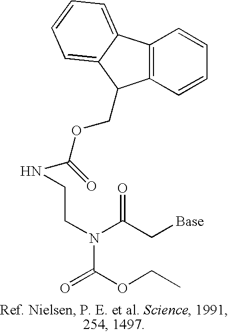 | 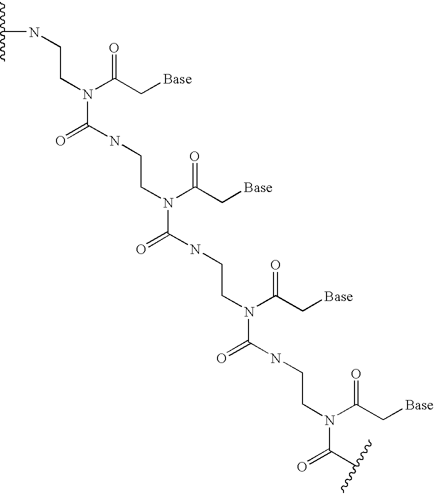 |

Examples of oligomers of some analogues

| | | |
|---|---|---|
| HNA | 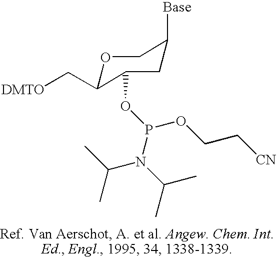 | 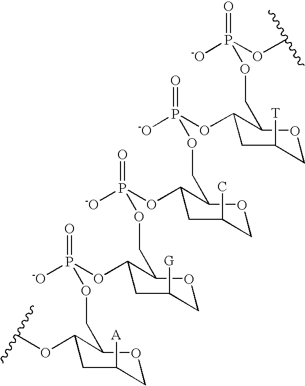 |
| MNA | 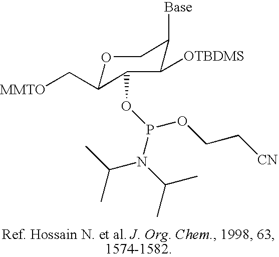 | 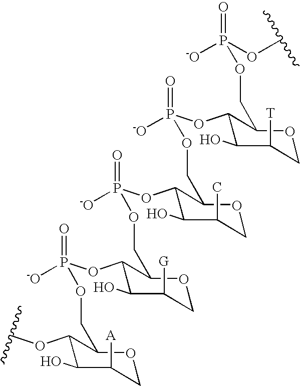 |
| ANA | 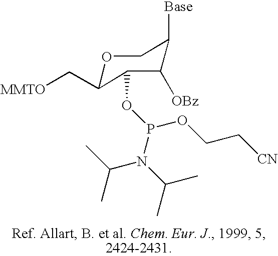 | 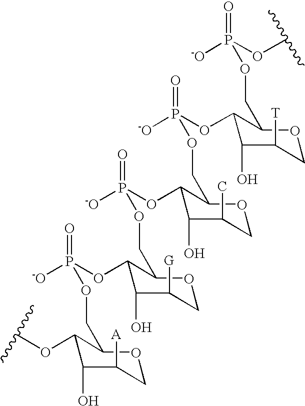 |
| LNA | 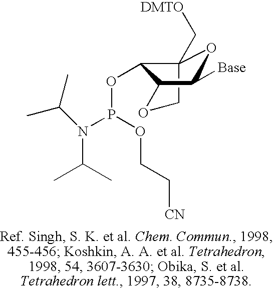 | 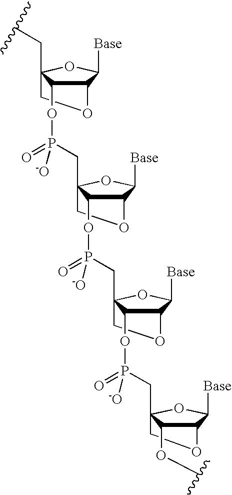 |

| | |
|---|---|
| 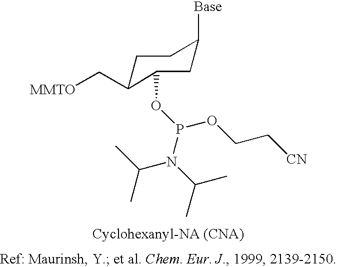 | 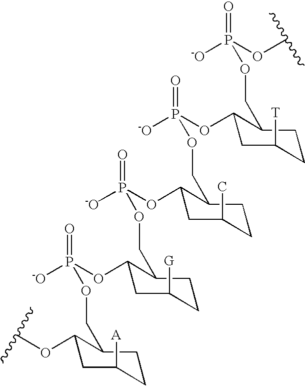 |
| 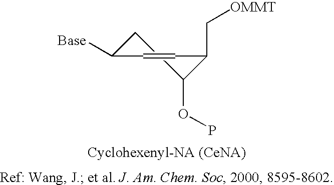 | 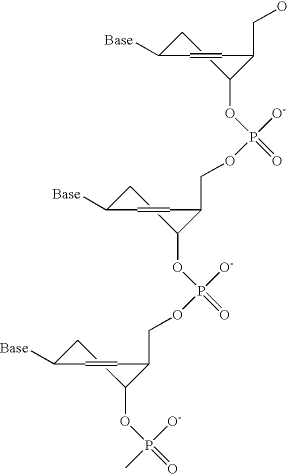 |
| 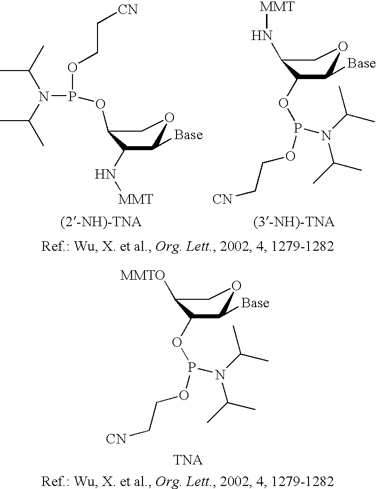 | 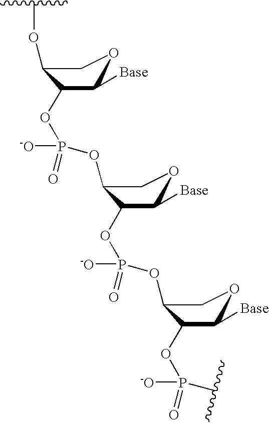 |

Section of a Nucleic acid of the respective analogues

| | |
|---|---|
| 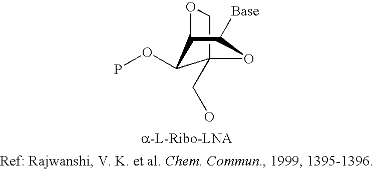 | 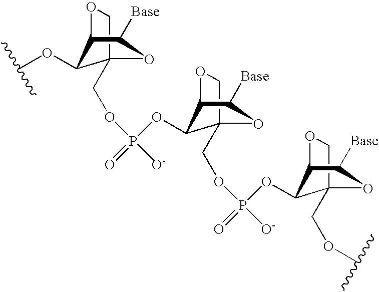 |
| 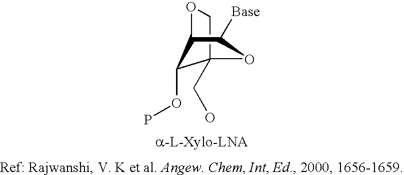 | 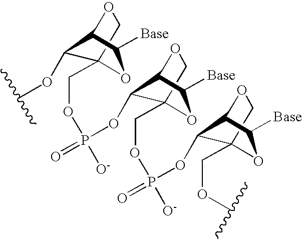 |
| 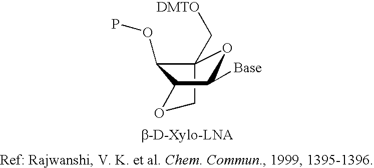 | 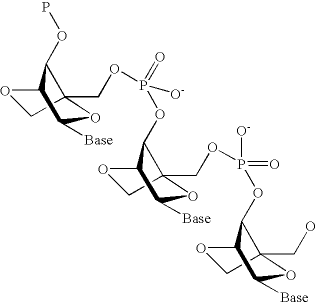 |
| 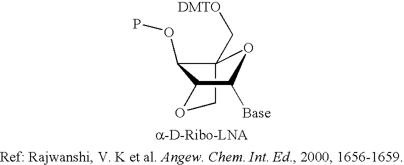 | 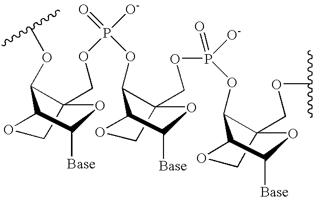 |
| 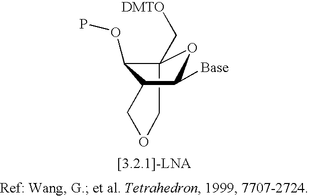 | 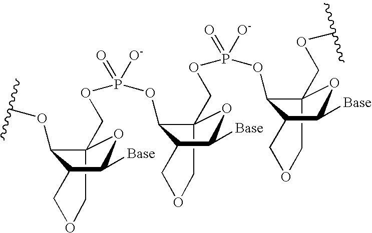 |

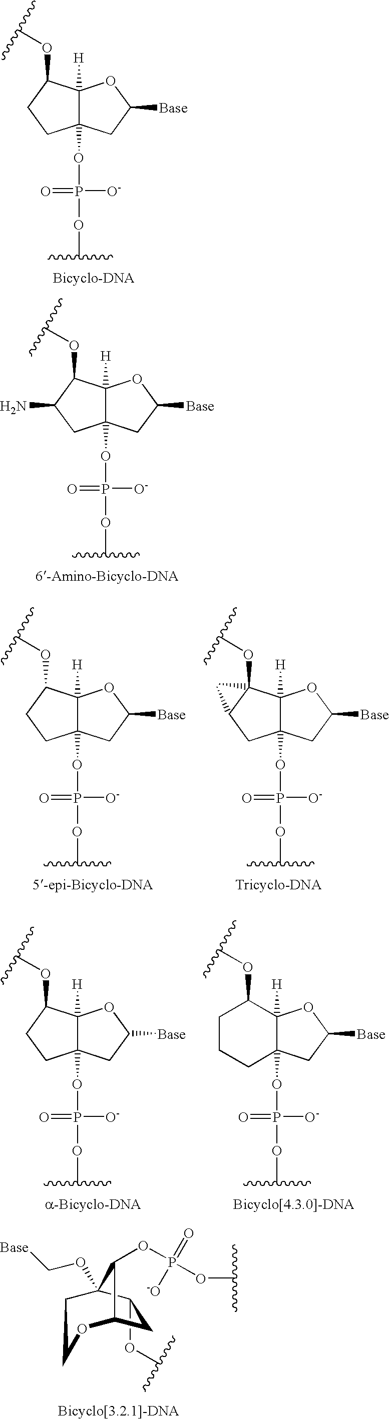

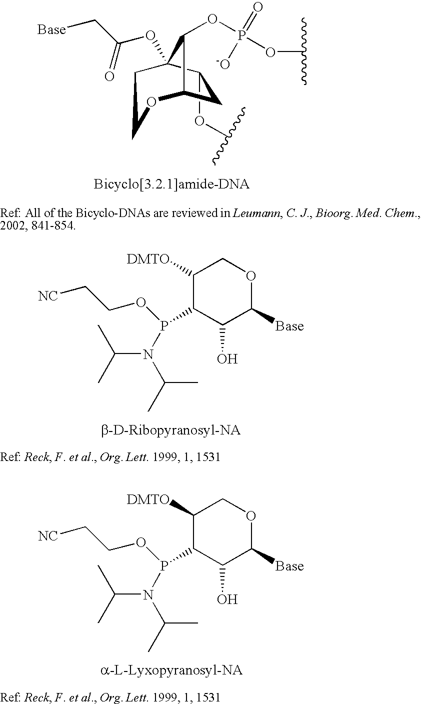

General structure of 2′-modified oligomers

| | |
|---|---|
| 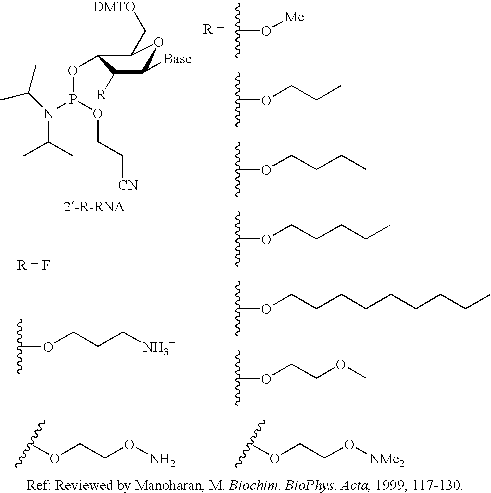 | 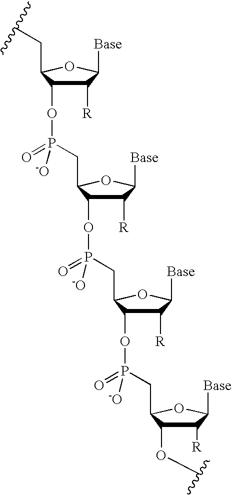 |
| 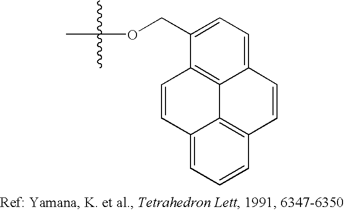 | 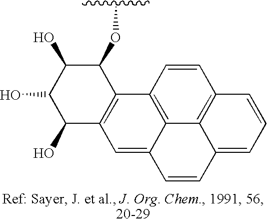 |

Examples of modifications that, to our knowledge, are not synthesised or published yet:

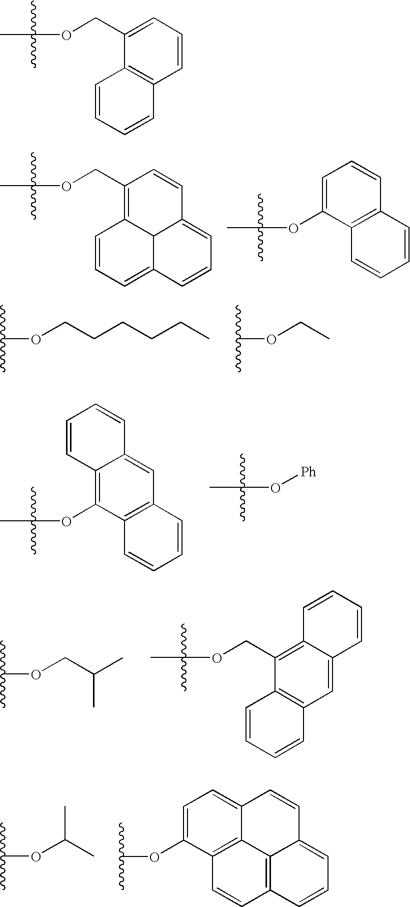

The backbone monomer unit of LNA (locked nucleic acid) is a sterically restricted DNA backbone monomer unit, which comprises an intramolecular bridge that restricts the usual conformational freedom of a DNA backbone monomer unit. LNA may be any LNA molecule as described in WO 99/14226 (Exiqon), preferably, LNA is selected from the molecules depicted in the abstract of WO 99/14226. Preferred LNA according to the present invention comprises a methyl linker connecting the 2′-O position to the 4′-C position, however other LNA's such as LNA's wherein the 2′ oxy atom is replaced by either nitrogen or sulphur are also comprised within the present invention.

The backbone monomer unit of intercalator pseudonucleotides according to present invention preferably have the general structure before being incorporated into an oligonucleotide and/or nucleotide analogue:

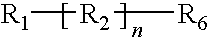

wherein

n=1 to 6, preferably n=2 to 6, more preferably n=3 to 6, more preferably n=2 to 5, more preferably n=3 to 5, more preferably n=3 to 4.

R₁ is a trivalent or pentavalent substituted phosphoratom, preferably R₁ is

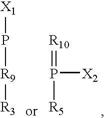

wherein

R₂ may individually be selected from an atom capable of forming at least two bonds, said atom optionally being individually substituted, preferably R₂ is individually selected from O, S, N, C, P, optionally individually substituted. By the term “individually” is meant that R₂ can represent one, two or more different groups in the same molecule. The bonds between two R₂ may be saturated or unsaturated or a part of a ring system or a combination thereof

Each R₂ may individually be substituted with any suitable substituent, such as a substituent selected from H, lower alkyl, C2-6 alkenyl, C6-10 aryl, C7-11 arylmethyl, C2-7 acyloxymethyl, C3-8 alkoxycarbonyloxymethyl, C7-11 aryloyloxymethyl, C3-8 S-acyl-2-thioethyl;

An “alkyl” group refers to an optionally substituted saturated aliphatic hydrocarbon, including straight-chain, branched-chain, and cyclic alkyl groups. Preferably, the alkyl group has 1 to 25 carbons and contains no more than 20 heteroatoms. More preferably, it is a lower alkyl of from 1 to 12 carbons, more preferably 1 to 6 carbons, more preferably 1 to 4 carbons. Heteroatoms are preferably selected from the group consisting of nitrogen, sulfur, phosphorus, and oxygen.

An “alkenyl” group refers to an optionally substituted hydrocarbon containing at least one double bond, including straight-chain, branched-chain, and cyclic alkenyl groups, all of which may be optionally substituted. Preferably, the alkenyl group has 2 to 25 carbons and contains no more than 20 heteroatoms. More preferably, it is a lower alkenyl of from 2 to 12 carbons, more preferably 2 to 4 carbons. Heteroatoms are preferably selected from the group consisting of nitrogen, sulfur, phosphorus, and oxygen.

An “alkynyl” group refers to an optionally substituted unsaturated hydrocarbon containing at least one triple bond, including straight-chain, branched-chain, and cyclic alkynyl groups, all of which may be optionally substituted. Preferably, the alkynyl group has 2 to 25 carbons and contains no more than 20 heteroatoms. More preferably, it is a lower alkynyl of from 2 to 12 carbons, more preferably 2 to 4 carbons. Heteroatoms are preferably selected from the group consisting of nitrogen, sulfur, phosphorus, and oxygen.

An “aryl” refers to an optionally substituted aromatic group having at least one ring with a conjugated pi electron system and includes carbocyclic aryl, heterocyclic aryl, biaryl, and triaryl groups. Examples of aryl substitution substituents include alkyl, alkenyl, alkynyl, aryl, amino, substituted amino, carboxy, hydroxy, alkoxy, nitro, sulfonyl, halogen, thiol and aryloxy.

A “carbocyclic aryl” refers to an aryl where all the atoms on the aromatic ring are carbon atoms. The carbon atoms are optionally substituted as described above for an aryl. Preferably, the carbocyclic aryl is an optionally substituted phenyl.

A “heterocyclic aryl” refers to an aryl having 1 to 3 heteroatoms as ring atoms in the aromatic ring and the remainder of the ring atoms are carbon atoms. Suitable heteroatoms include oxygen, sulfur, and nitrogen. Examples of heterocyclic aryls include furanyl, thienyl, pyridyl, pyrrolyl, N-lower alkyl pyrrolo, pyrimidyl, pyrazinyl, and imidazolyl. The heterocyclic aryl is optionally substituted as described above for an aryl.

The substituents on two or more R₂ may alternatively join to form a ring system, such as any of the ring systems as defined above.

Preferably R₂ is substituted with an atom or a group selected from H, methyl, R₄, hydroxyl, halogen, and amino, more preferably R₂ is substituted with an atom or a group selected from H, methyl, R₄.

More preferably R₂ is individually selected from O, S, NH, N(Me), N(R₄), C(R₄)₂, CH(R₄) or CH₂, wherein R₄ is as defined below,

R₃=methyl, beta-cyanoethyl, p-nitrophenetyl, o-chlorophenyl, or p-chlorophenyl.

R₄=lower alkyl, preferably lower alkyl such as methyl, ethyl, or isopropyl, or heterocyclic, such as morpholino, pyrrolidino, or 2,2,6,6-tetramethylpyrrolidino, wherein lower alkyl is defined as C₁-C₆, such as C₁-C₄.

R₅ =alkyl, alkoxy, aryl or H, with the proviso that R₅ is H when X₂═O⁻, preferably R₅ is selected from lower alkyl, lower alkoxy, aryloxy. In a preferred embodiment aryloxy is selected from phenyl, naphtyl or pyridine.

R₆ is a protecting group, selected from any suitable protecting groups. Preferably R₆ is selected from the group consisting of trityl, monomethoxytrityl, 2-chlorotrityl, 1,1,1,2-tetrachloro-2,2-bis(p-methoxyphenyI)-ethan (DATE), 9-phenylxanthine-9-yl (pixyl) and 9-(p-methoxyphenyl) xanthine-9-yl (MOX) or other protecting groups mentioned in “Current Protocols In Nucleic Acid Chemistry” volume 1, Beaucage et al. Wiley. More preferably the protecting group may be selected from the group consisting of monomethoxytrityl and dimethoxytrityl. Most preferably, the protecting group may be 4,4′-dimethoxytrityl (DMT).

R₉ is selected from O, S, N optionally substituted, preferably R₉ is selected from O, S, NH, N(Me).

R₁₀ is selected from O, S, N, C, optionally substituted.

X₁═Cl, Br, I, or N(R₄)₂

X₂═Cl, Br, I, N(R₄)₂, or O⁻

As described above with respect to the substituents the backbone monomer unit can be acyclic or part of a ring system.

In one preferred embodiment of the present invention the backbone monomer unit of an intercalator pseudonucleotide is selected from the group consisting of acyclic backbone monomer units. Acyclic is meant to cover any backbone monomer unit, which does not comprise a ringstructure, for example the backbone monomer unit preferably does not comprise a ribose or a deoxyribose group.

In particular, it is preferred that the backbone monomer unit of an intercalator pseudonucleotide is an acyclic backbone monomer unit, which is capable of stabilising a bulge insertion (see herein below).

In another preferred embodiment the backbone monomer unit of an intercalator pseudonucleotide according to the present invention may be selected from the group consisting of backbone monomer units comprising at least one chemical group selected from the group consisting of trivalent and pentavalent phosphorous atom such as a pentavalent phosphorous atom. More preferably the phosphate atom of the backbone monomer unit of an intercalator pseudonucleotide according to the present invention may be selected from the group consisting of backbone monomer units comprising at least one chemical group selected from the group consisting of, phosphoester, phosphodiester, phosphoramidate and phosphoramidit groups.

In particular it is preferred that the backbone monomer unit of an intercalator pseudonucleotide according to the present invention is selected from the group consisting of acyclic backbone monomer units comprising at least one chemical group selected from the group consisting of phosphate, phosphoester, phosphodiester, phosphoramidate and phosphoramidit groups.

Preferred backbone monomer units comprising at least one chemical group selected from the group consisting of phosphate, phosphoester, phosphodiester, phosphoramidate and phosphoramidit groups are backbone monomer units, wherein the distance from at least one phosphor atom to at least one phosphor atom of a neighbouring nucleotide, not including the phosphor atoms, is at the most 6 atoms long, for example 2, such as 3, for example 4, such as 5, for example 6 atoms long, when the backbone monomer unit is incorporated into a nucleic acid backbone.

The distance is measured as the direct linkage (i.e. the shortest path) as discussed above.

Preferably the backbone monomer unit is capable of being incorporated into a phosphate backbone of a nucleic acid or nucleic acid analogue in a manner so that at the most 5 atoms are separating the phosphor atom of the intercalator pseudonucleotide backbone monomer unit and the nearest neighbouring phosphor atom, more preferably 5 atoms are separating the phosphor atom of the intercalator pseudonucleotide backbone monomer unit and the nearest neighbouring phosphor atom, in both cases not including the phosphor atoms themselves.

Preferably the backbone monomer unit is capable of being incorporated into a phosphate backbone of a nucleic acid or nucleic acid analogue in a manner so that at the most 4 atoms are separating the phosphor atom of the intercalator pseudonucleotide backbone monomer unit and the nearest neighbouring phosphor atom, more preferably 4 atoms are separating the phosphor atom of the intercalator pseudonucleotide backbone monomer unit and the nearest neighbouring phosphor atom, in both cases not including the phosphor atoms themselves.

In a particularly preferred embodiment of the present invention the intercalator pseudonucleotide comprises a backbone monomer unit that comprises a phosphoramidit and more preferably the backbone monomer unit comprises a trivalent phosphoramidit.

Suitable trivalent phosphoramidits are trivalent phosphoramidits that may be incorporated into the backbone of a nucleic acid and/or a nucleic acid analogue. Usually, the amidit group per se may not be incorporated into the backbone of a nucleic acid, but rather the amidit group or part of the amidit group may serve as a leaving group and/or protecting group. However, it is preferred that the backbone monomer unit comprises a phosphoramidit group, because such a group may facilitate the incorporation of the backbone monomer unit into a nucleic acid backbone.

Preferably the acyclic backbone monomers may be selected from one of the general structures depicted below:

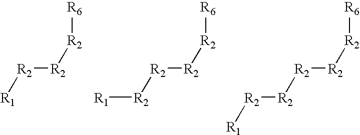

wherein R₁, R₂ and R₆ are as defined above.

More preferably, the acyclic backbone monomer unit may be selected from the group depicted below:

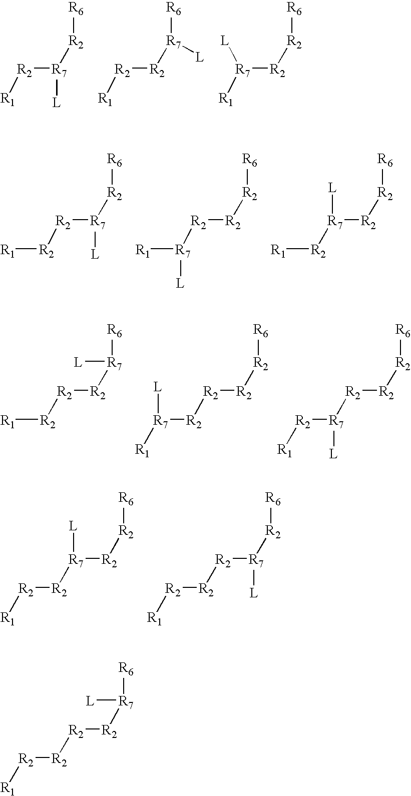

wherein R₁, R₂ and R₆ are as defined above, and R₇═N, or CH.

Below are specific examples of backbone monomer units numbered I) to, wherein R₁ and R₆ are as defined above, and R₈ may be R₄ or H, optionally substituted.

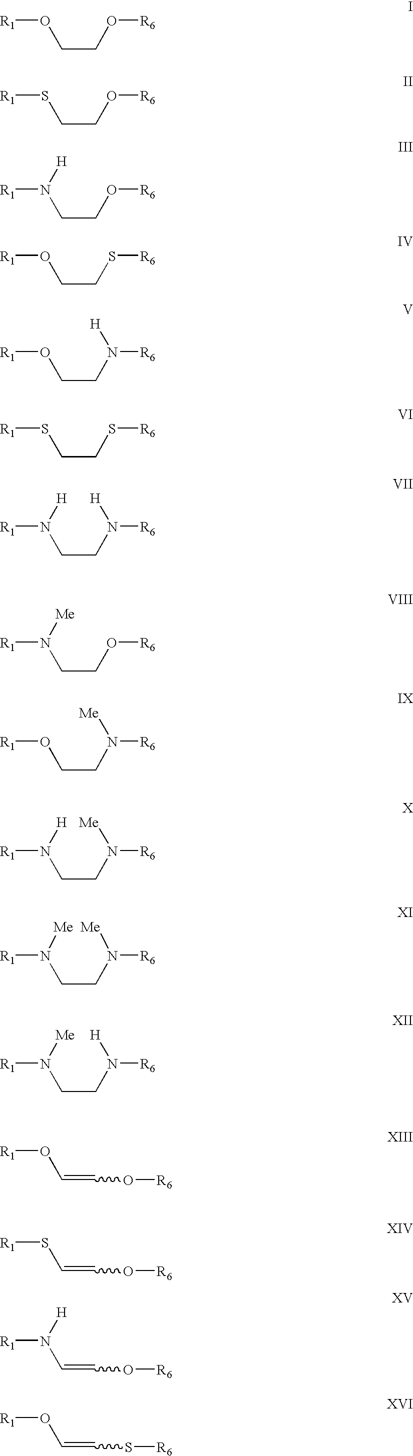

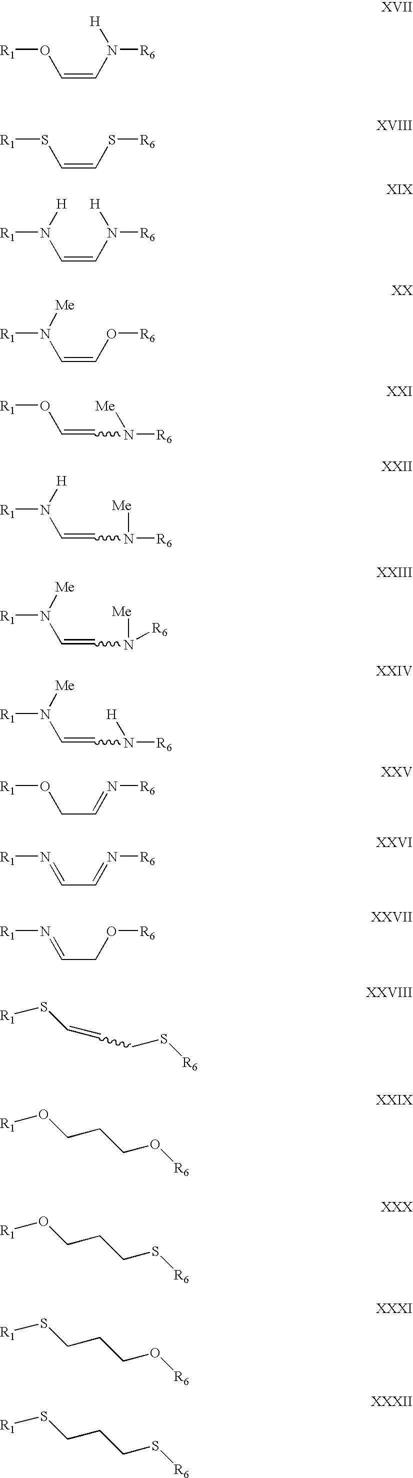

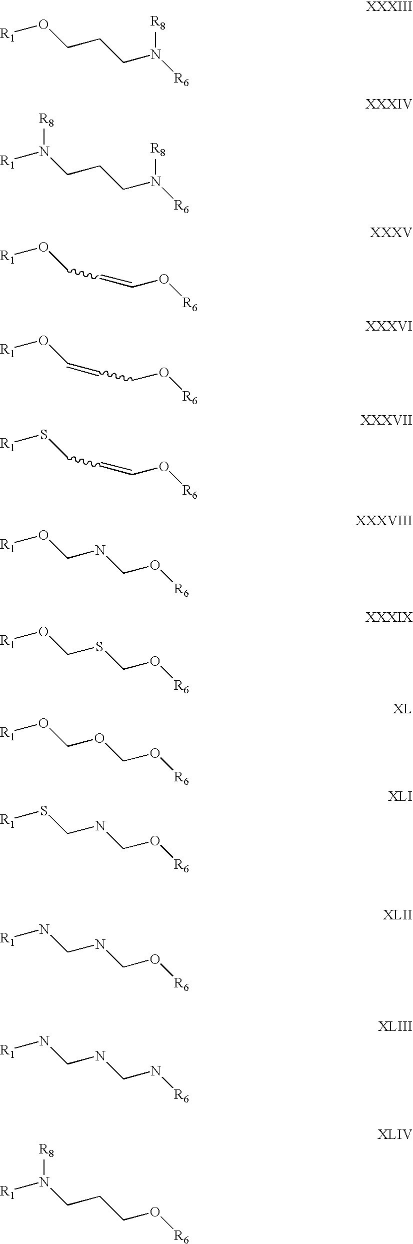

Me denotes methyl

Even more preferable the backbone monomer unit including optional protecting groups may be selected from the group consisting of the structures I) to XLIV) as indicated herein below:

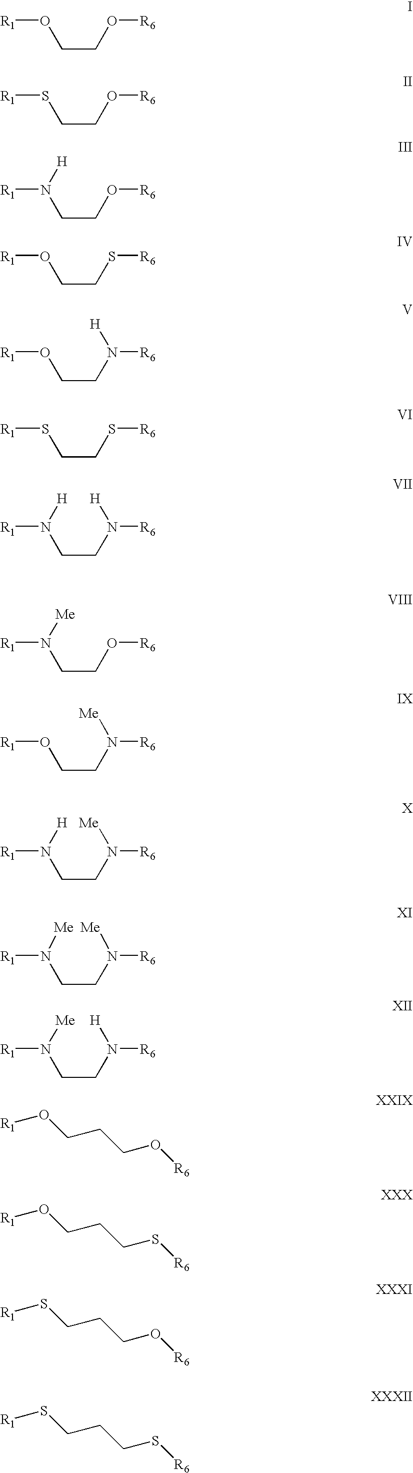

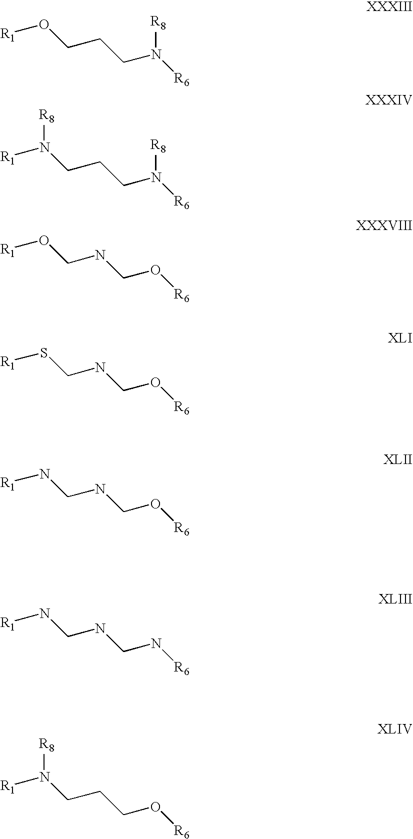

Most preferred are the backbone monomer units selected from the group consisting of:

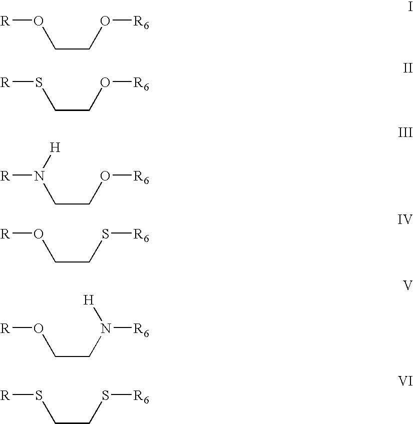

Preferably, the acyclic backbone monomer unit may be selected from the group consisting of the structures a) to g) as indicated below:

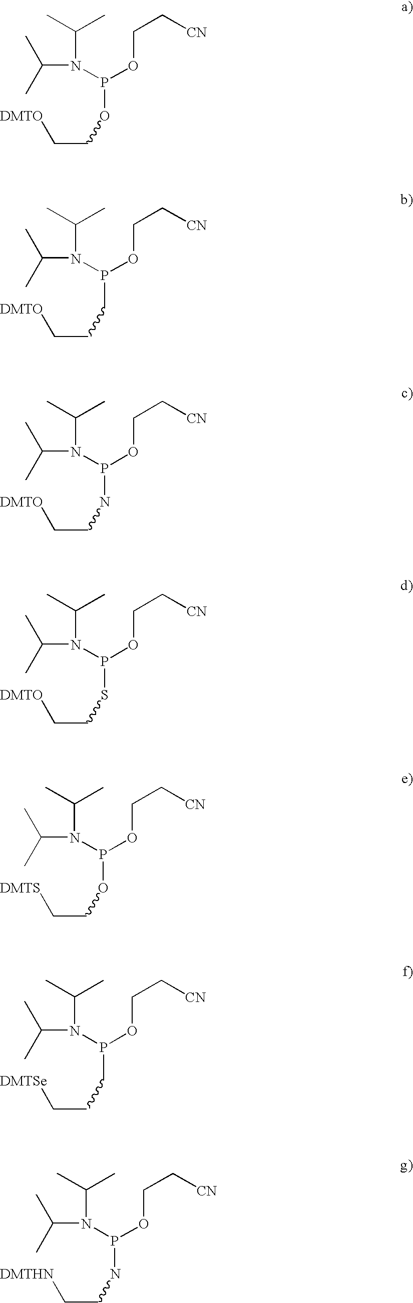

The backbone monomer unit of an intercalator pseudonucleotide which is inserted into an oligonucleotide or oligonucleotide analogue, according to the present invention may comprise a phosphodiester bond. Additionally, the backbone monomer unit of an intercalator pseudonucleotide according to the present invention may comprise a pentavalent phosphoramidate. Preferably, the backbone monomer unit of an intercalator pseudonucleotide according to the present invention is an acyclic backbone monomer unit that may comprise a pentavalent phosphoramidate.

Leaving Group

The backbone monomer unit according to the present invention may comprise one or more leaving groups. Leaving groups are chemical groups, which are part of the backbone monomer unit when the intercalator pseudonucleotide or the nucleotide is a monomer, but which are no longer present in the molecule once the intercalator pseudonucleotide or the nucleotide has been incorporated into an oligonucleotide or oligonucleotide analogue.

The nature of a leaving group depends of the backbone monomer unit. For example, when the backbone monomer unit is a phosphor amidit, the leaving group, may for example be an diisopropylamine group. In general, when the backbone monomer unit is a phosphor amidit, a leaving group is attached to the phosphor atom for example in the form of diisopropylamine and said leaving group is removed upon coupling of the phosphor atom to a nucleophilic group, whereas the rest of the phosphate group or part of the rest, may become part of the nucleic acid or nucleic acid analogue backbone.

Reactive Group

The backbone monomer units according to the present invention may furthermore comprise a reactive group which is capable of performing a chemical reaction with another nucleotide or oligonucleotide or nucleic acid or nucleic acid analogue to form a nucleic acid or nucleic acid analogue, which is one nucleotide longer than before the reaction.

Accordingly, when nucleotides are in their free form, i.e. not incorporated into a nucleic acid, they may comprise a reactive group capable of reacting with another nucleotide or a nucleic acid or nucleic acid analogue.

In preferred embodiments of the present invention said reactive group may be protected by a protecting group. Prior to said chemical reaction, said protection group may be removed. The protection group will thus not be a part of the newly formed nucleic acid or nucleic acid analogue.

Examples of reactive groups are nucleophiles such as the 5′-hydroxy group of DNA or RNA backbone monomer units.

Protecting Group

The backbone monomer unit according to the present invention may also comprise a protecting group, which can be removed, and wherein removal of the protecting group allows for a chemical reaction between the intercalator pseudonucleotide and a nucleotide or nucleotide analogue or another intercalator pseudonucleotide.

In particular, a nucleotide monomer or nucleotide analogue monomer or intercalator pseudonucleotide monomer may comprise a protecting group, which is no longer present in the molecule once the nucleotide or nucleotide analogue or intercalator pseudonucleotide has been incorporated into a nucleic acid or nucleic acid analogue.

Furthermore, backbone monomer units may comprise protecting groups which may be present in the oligonucleotide or oligonucleotide analogue subsequent to incorporation of the nucleotide or nucleotide analogue or intercalator pseudonucleotide, but which may no longer be present after introduction of an additional nucleotide or nucleotide analogue to the oligonucleotide or oligonucleotide analogue or which may be removed after the synthesis of the entire oligonucleotide or oligonucleotide analogue.

The protecting group may be removed by a number of suitable techniques known to the person skilled in the art, however preferably, the protecting group may be removed by a treatment selected from the group consisting of acid treatment, thiophenol treatment and alkali treatment.

Preferred protecting groups according to the present invention, which may be used to protect the 5′ end or the 5′ end analogue of a backbone monomer unit may be selected from the group consisting of trityl, monomethoxytrityl, 2-chlorotrityl, 1,1,1,2-tetrachloro-2,2-bis(p-methoxyphenyl)-ethan (DATE), 9-phenylxanthine-9-yl (pixyl) and 9-(p-methoxyphenyl) xanthine-9-yl (MOX) or other protecting groups mentioned in “Current Protocols In Nucleic Acid Chemistry” volume 1, Beaucage et al. Wiley. More preferably the protecting group may be selected from the group consisting of monomethoxytrityl and dimethoxytrityl. Most preferably, the protecting group may be 4,4′-dimethoxytrityl(DMT).

4,4′-dimethoxytrityl(DMT) groups may be removed by acid treatment, for example by brief incubation (30 to 60 seconds sufficient) in 3% trichloroacetic acid or in 3% dichlororacetic acid in CH₂Cl₂.

Preferred protecting groups which may protect a phosphate or phosphoramidit group of a backbone monomer unit may for example be selected from the group consisting of methyl and 2-cyanoethyl. Methyl protecting groups may for example be removed by treatment with thiophenol or disodium 2-carbamoyl 2-cyanoethylene-1,1-dithiolate. 2-cyanoethyl-groups may be removed by alkali treatment, for example treatment with concentrated aqueous ammonia, a 1:1 mixture of aqauos methylamine and concentrated aqueous ammonia or with ammonia gas.

Intercalator

The term intercalator according to the present invention covers any molecular moiety comprising at least one essentially flat conjugated system, which is capable of co-stacking with nucleobases of a nucleic acid. Preferably an intercalator according to the present invention essentially consists of at least one essentially flat conjugated system, which is capable of co-stacking with nucleobases of a nucleic acid or nucleic acid analogue.

Preferably, the intercalator comprises a chemical group selected from the group consisting of polyaromates and heteropolyaromates an even more preferably the intercalator essentially consists of a polyaromate or a heteropolyaromate. Most preferably the intercalator is selected from the group consisting of polyaromates and heteropolyaromates.

Polyaromates or heteropolyaromates according to the present invention may consist of any suitable number of rings, such as 1, for example 2, such as 3, for example 4, such as 5, for example 6, such as 7, for example 8, such as more than 8. Furthermore polyaromates or heteropolyaromates may be substituted with one or more selected from the group consisting of hydroxyl, bromo, fluoro, chloro, iodo, mercapto, thio, cyano, alkylthio, heterocycle, aryl, heteroaryl, carboxyl, carboalkoyl, alkyl, alkenyl, alkynyl, nitro, amino, alkoxyl and amido.

In one preferred embodiment of the present invention the intercalator may be selected from the group consisting of polyaromates and heteropolyaromates that are capable of fluorescing.

In another more preferred embodiment of the present invention the intercalator may be selected from the group consisting of polyaromates and heteropolyaromates that are capable of forming excimers, exciplexes, fluorescence resonance energy transfer (FRET) or charged transfer complexes.

Accordingly, the intercalator may preferably be selected from the group consisting of phenanthroline, phenazine, phenanthridine, anthraquinone, pyrene, anthracene, napthene, phenanthrene, picene, chrysene, naphtacene, acridones, benzanthracenes, stilbenes, oxalo-pyridocarbazoles, azidobenzenes, porphyrins, psoralens and any of the aforementioned intercalators substituted with one or more selected from the group consisting of hydroxyl, bromo, fluoro, chloro, iodo, mercapto, thio, cyano, alkylthio, heterocycle, aryl, heteroaryl, carboxyl, carboalkoyl, alkyl, alkenyl, alkynyl, nitro, amino, alkoxyl and/or amido.

Preferably, the intercalator is selected from the group consisting of phenanthroline, phenazine, phenanthridine, anthraquinone, pyrene, anthracene, napthene, phenanthrene, picene, chrysene, naphtacene, acridones, benzanthracenes, stilbenes, oxalo-pyridocarbazoles, azidobenzenes, porphyrins and psoralens.

More preferably the intercalator may be selected from the group of intercalators comprising one of the structures as indicated herein below:

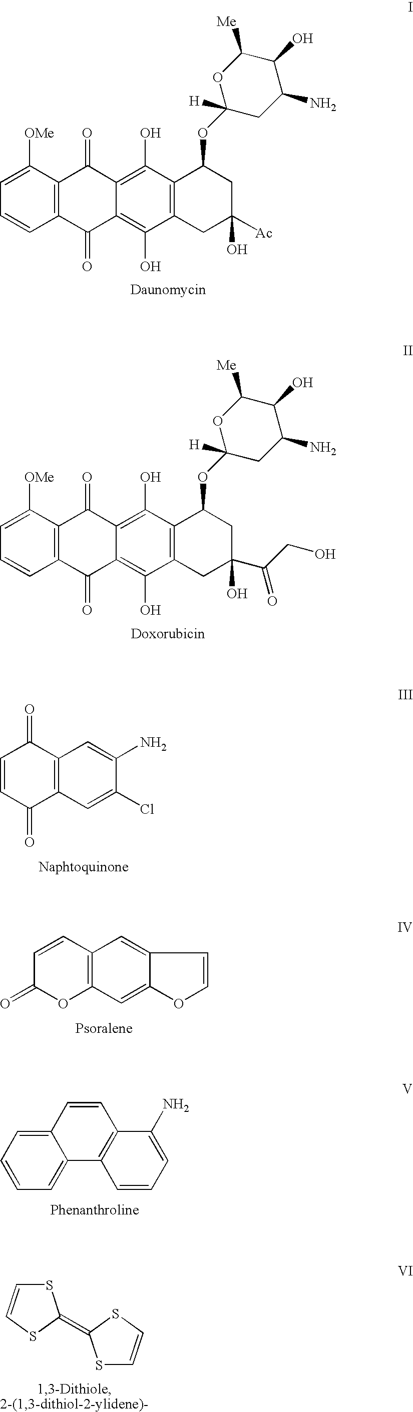

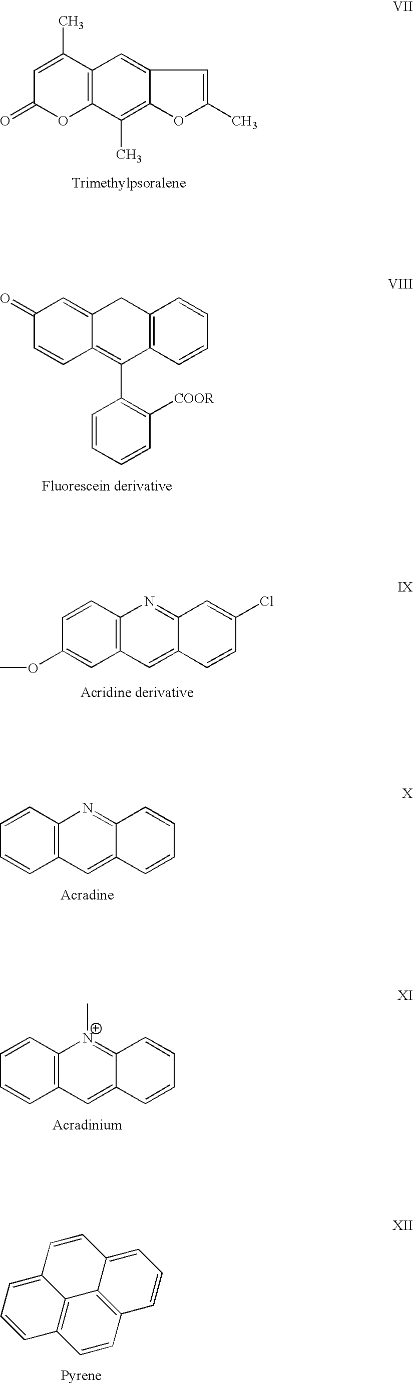

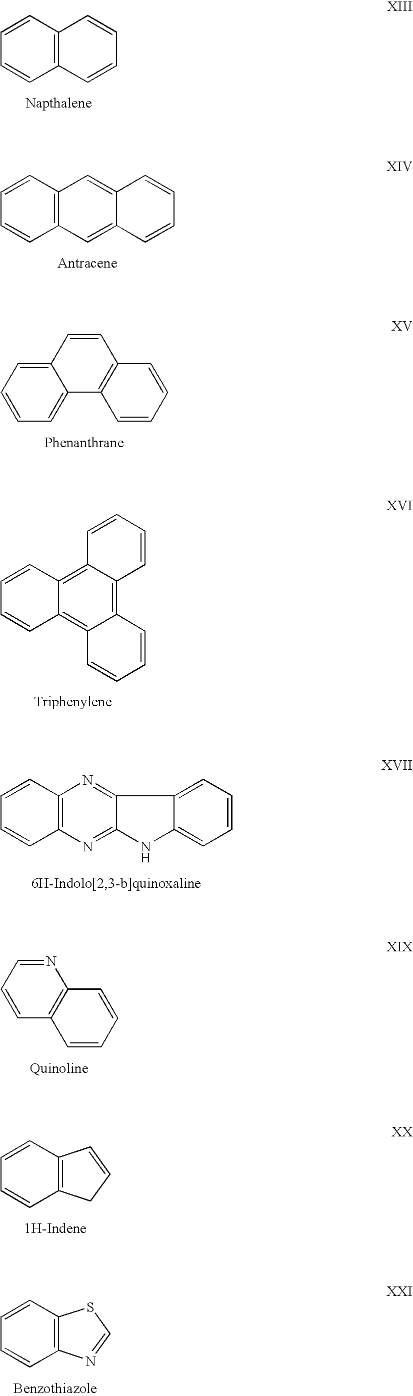

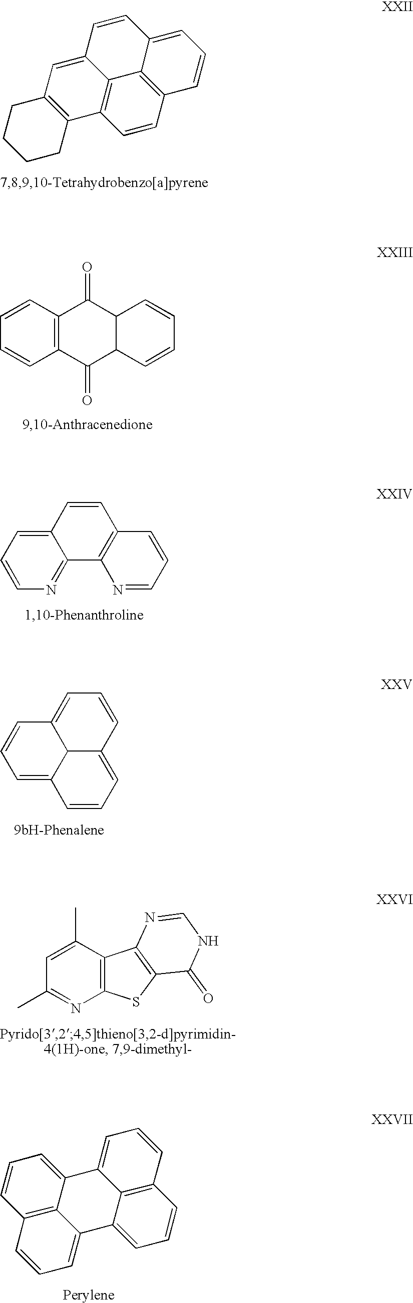

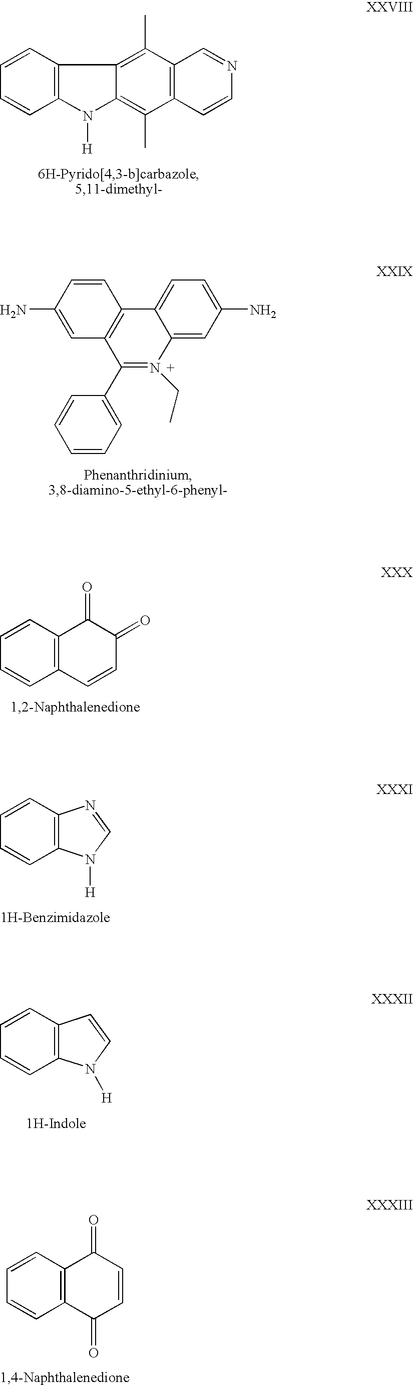

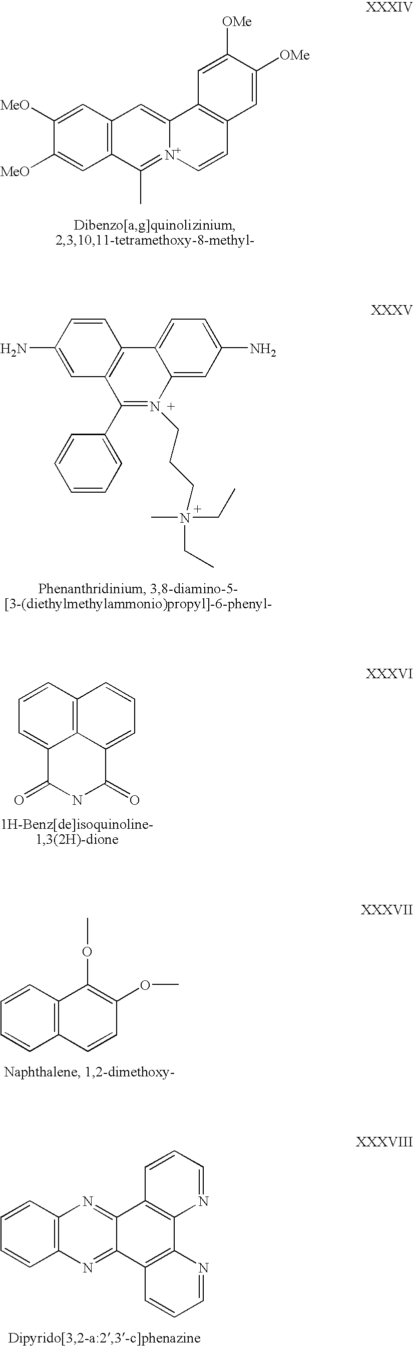

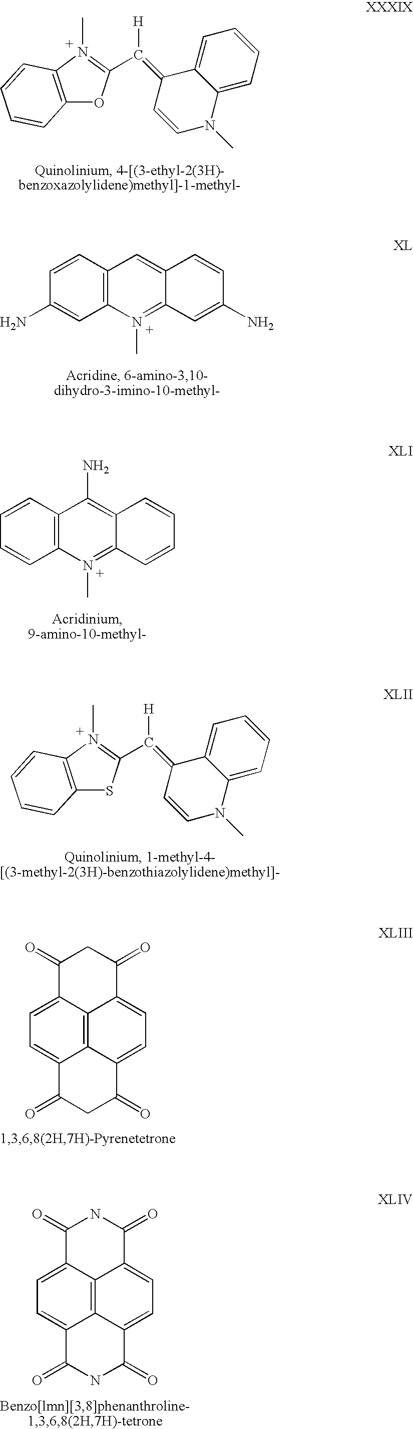

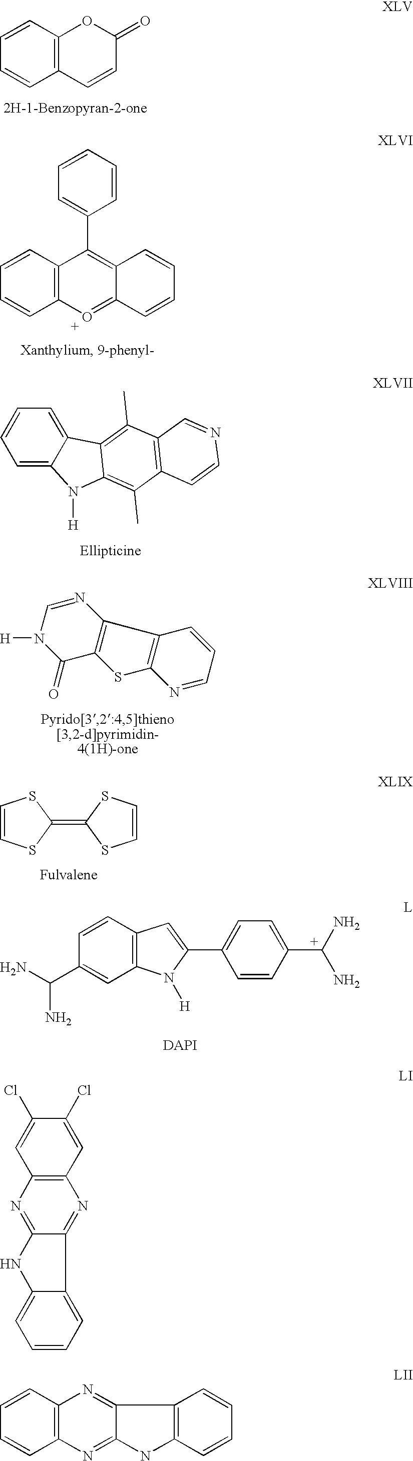

as well as derivatives thereof.

Even more preferably the intercalator may be selected from the group of intercalators comprising one of the intercalator structures above numbered V, XII, XIV, XV, XVII, XXIII, XXVI, XXVIII, XLVII, LI and LII as well as derivatives thereof.

Most preferably the interacalator is selected from the group of intercalator structures above numbered XII, XIV, XVII, XXIII, LI.

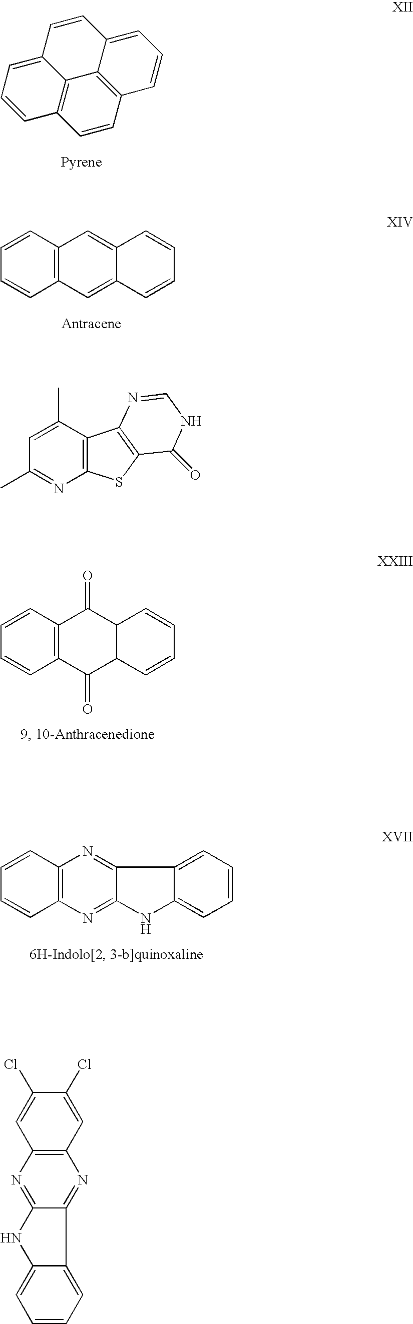

as well as derivatives thereof

The above list of examples is not to be understood as limiting in any way, but only as to provide examples of possible structures for use as intercalators. In addition, the substitution of one or more chemical groups on each intercalator to obtain modified structures is also included in the present invention.

The intercalator moiety of the intercalator pseudonucleotide is linked to the backbone unit by the linker. When going from the backbone along the linker to the intercalating moiety, the linker and intercalator connection is defined as the bond between a linker atom and the first atom being part of a conjugated system that is able to co-stack with nucleobases of a strand of a oligonucleotide or oligonucleotide analogue when said oligonucleotide or oligonucleotide analogue is hybridised to an oligonucleotide analogue comprising said intercalator pseudonucleotide.

In one embodiment of the present invention, the linker may comprise a conjugated system and the intercalator may comprise another conjugated system. In this case the linker conjugated system is not capable of costacking with nucleobases of said opposite oligonucleotide or oligonucleotide analogue strand.

Linker

The linker of a intercalator pseudonucleotide according to the present invention is a moiety connecting the intercalator and the backbone monomer of said intercalator pseudonucleotide. The linker may comprise one or more atom(s) or bond(s) between atoms.

By the definitions of backbone and intercalating moieties defined herein above, the linker is the shortest path linking the backbone and the intercalator. If the intercalator is linked directly to the backbone, the linker is a bond.

The linker usually consists of a chain of atoms or a branched chain of atoms. Chains can be saturated as well as unsaturated. The linker may also be a ring structure with or without conjugated bonds.

For example the linker may comprise a chain of m atoms selected from the group consisting of C, O, S, N. P, Se, Si, Ge, Sn and Pb, wherein one end of the chain is connected to the intercalator and the other end of the chain is connected to the backbone monomer unit.

In some embodiments the total length of the linker and the intercalator of the intercalator pseudonucleotides according to the present invention preferably is between 8 and 13 Å (see herein below). Accordingly, m should be selected dependent on the size of the intercalator of the specific intercalator pseudonucleotide.

I.e. m should be relevatively large, when the intercalator is small and m should be relatively small when the intercalator is large. For most purposes however m will be an integer from 1 to 7, such as from 1-6, such as from 1-5, such as from 1-4. As described above the linker may be an unsaturated chain or another system involving conjugated bonds. For example the linker may comprise cyclic conjugated structures. Preferably, m is from 1 to 4 when the linker is an saturated chain.

When the intercalator is pyrene, m is preferably an integer from 1 to 7, such as from 1-6, such as from 1-5, such as from 1-4, more preferably from 1 to 4, even more preferably from 1 to 3, most preferably m is 2 or 3.

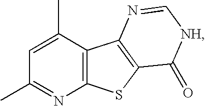

When the intercalator has the structure m is preferably from 2 to 6, more preferably 2.

The chain of the linker may be substituted with one or more atoms selected from the group consisting of C, H, O, S, N, P, Se, Si, Ge, Sn and Pb.

In one embodiment the linker is an azaalkyl, oxaalkyl, thiaalkyl or alkyl chain. For example the linker may be an alkyl chain substituted with one or more selected from the group consisting C, H, O, S, N, P, Se, Si, Ge, Sn and Pb. In a preferred embodiment the linker consists of an unbranched alkyl chain, wherein one end of the chain is connected to the intercalator and the other end of the chain is connected to the backbone monomer unit and wherein each C is substituted with 2 H. More preferably_{;} said unbranched alkyl chain is from 1 to 5 atoms long, such as from 1 to 4 atoms long, such as from 1 to 3 atoms long, such as from 2 to 3 atoms long.

In another embodiment of the invention the linker is a ring structure comprising atoms selected from the group consisting of C, O, S, N, P, Se, Si, Ge, Sn and Pb. For example the linker may be such a ring structure substituted with one or more selected from the group consisting of C, H, O, S, N, P, Se, Si, Ge, Sn and Pb.

In another embodiment the linker consists of from 1-6 C atoms, from 0-3 of each of the following atoms O, S, N. More preferably the linker consists of from 1-6 C atoms and from 0-1 of each of the atoms O, S, N.

In a preferred embodiment the linker consists of a chain of C, O, S and N atoms, optionally substituted. Preferably said chain should consist of at the most 3 atoms, thus comprising from 0 to 3 atoms selected individually from C, O, S, N, optionally substituted.

In a preferred embodiment the linker consists of a chain of C, N, S and O atoms, wherein one end of the chain is connected to the intercalator and the other end of the chain is connected to the backbone monomer unit.

Preferably such a chain comprise one of the linkers shown below, most preferably the linker consist of one of the molecule shown below:

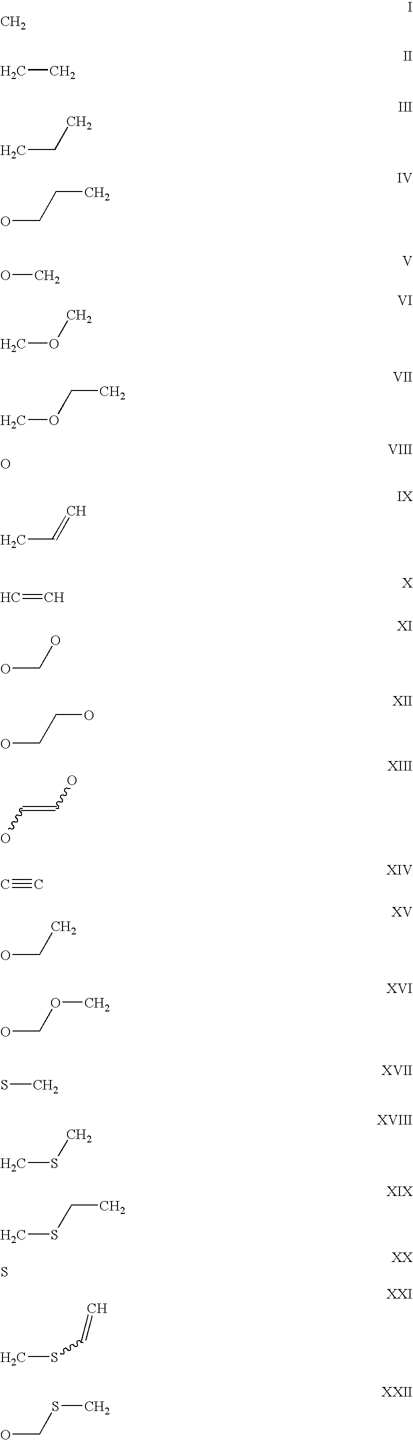

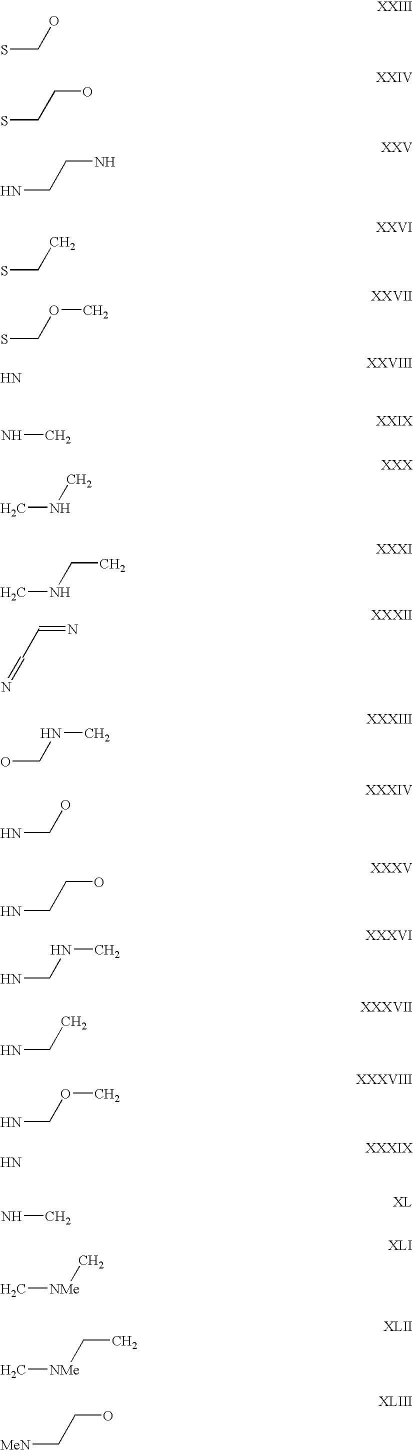

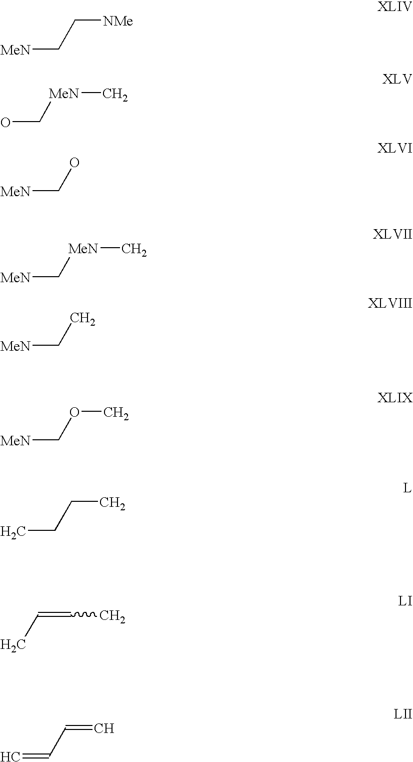

In a preferred embodiment the chain comprise one of the linkers shown below, more preferably the linker consist of one of the molecule shown below:

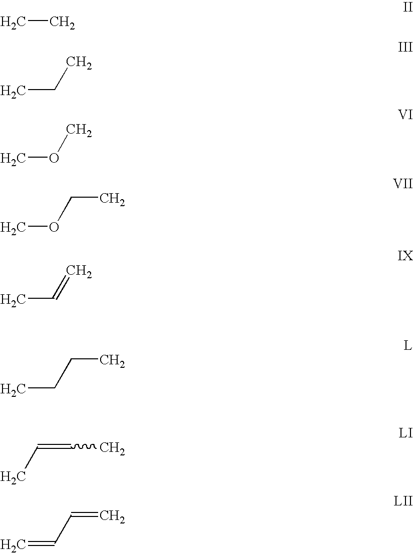

In a more preferred embodiment the chain comprise one of the linkers shown below, more preferably the linker consist of one of the molecule shown below:

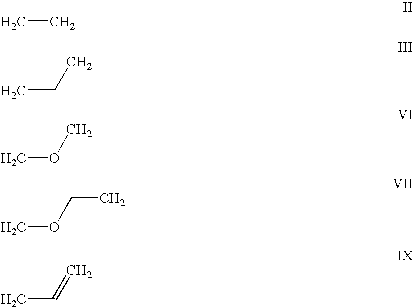

The linker constitutes Y in the formula for the intercalator pseudonucleotide X—Y-Q, as defined above, and hence X and Q are not part of the linker.

Intercalator Pseudonucleotides

Intercalator pseudonucleotides according to the present invention preferably have the general structure

X—Y-Q

wherein
X is a backbone monomer unit capable of being incorporated into the backbone of a nucleic acid or nucleic acid analogue,
Q is an intercalator comprising at least one essentially flat conjugated system, which is capable of co-stacking with nucleobases of a nucleic acid ; and
Y is a linker moiety linking said backbone monomer unit and said intercalator; and
wherein the total length of Q and Y is in the range from 7 Å to 20 Å,
with the proviso that when the intercalator is pyrene the total length of Q and Y is in the range from 9 Å to 13 Å.

Furthermore, in a preferred embodiment of the present invention the intercalator pseudonucleotide comprises a backbone monomer unit, wherein said backbone monomer unit is capable of being incorporated into the phosphate backbone of a nucleic acid or nucleic acid analogue in a manner so that at the most 4 atoms are separating the two phosphor atoms of the backbone that are closest to the intercalator.

The intercalator pseudonucleotides preferably do not comprise a nucleobase capable of forming Watson-Crick hydrogen bonding. Hence intercalator pseudonucleotides according to the invention are preferably not capable of Watson-Crick base pairing.

Preferably, the total length of Q and Y is in the range from 7 Å to 20 Å, more preferably, from 8 A to 15 Å, even more preferably from 8 Å to 13 Å, even more preferably from 8.4 Å to 12 Å, most preferably from 8.59 Å to 10 Å or from 8.4 Å to 10.5 Å.

When the intercalator is pyrene the total length of Q and Y is preferably in the range of 8 Å to 13 Å, such as from 9 Å to 13 A, more preferably from 9.05 Å to 11 Å, such as from 9.0 Å to 11 Å, even more preferably from 9.05 to 10 Å, such as from 9,0 to 10 Å, most preferably about 9.8 Å.

The total length of the linker (Y) and the intercalator (Q) should be determined by determining the distance from the center of the non-hydrogen atom of the linker which is furthest away from the intercalator to the center of the non-hydrogen atom of the essentially flat, conjugated system of the intercalator that is furthest away from the backbone monomer unit. Preferably, the distance should be the maximal distance in which bonding angles and normal chemical laws are not broken or distorted in any way.

The distance should preferably be determined by calculating the structure of the free intercalating pseudonucleotide with the lowest conformational energy level, and then determining the maximum distance that is possible from the center of the non-hydrogen atom of the linker which is furthest away from the intercalator to the center of the non-hydrogen atom of the essentially flat, conjugated system of the intercalator that is furthest away from the backbone monomer unit without bending, stretching or otherwise distorting the structure more than simple rotation of bonds that are free to rotate (e.g. not double bonds or bonds participating in a ring structure).

Preferably the energetically favorable structure is found by ab initio or forcefields calculations.

Even more preferably the distance should be determined by a method consisting of the following steps:
a) the structure of the intercalator pseudonucleotide of interest is drawn by computer using the programme ChemWindow® 6.0 (BioRad); and
b) the structure is transferred to the computer programme SymApps™ (BioRad); and
c) the 3-dimensional structure comprising calculated lengths of bonds and bonding angles of the intercalator pseudonucleotide is calculated using the computer programme SymApps™ (BioRad); and
d) the 3 dimensional structure is transferred to the computer programme RasWin Molecular Graphics Ver. 2.6-ucb; and
e) the bonds are rotated using RasWin Molecular Graphics Ver. 2.6-ucb to obtain the maximal distance (the distance as defined herein above); and
f) the distance is determined.

For example when the intercalator pseudonucleotide has the following structure:

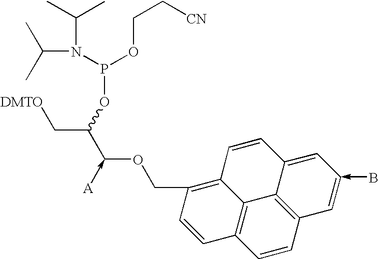

the total length of Q and Y is determined by measuring the linear distance from the center of the atom at A to the center of the atom at B, which in the above example is 9,79 Å.

In another example the intercalator pseudonucleotide has the following structure:

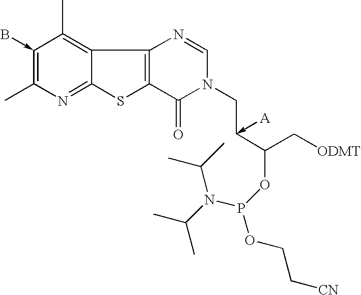

The total length of Q and Y, which is measured in a straight line from the center of the atom at A to the center of the atom at B is 8.71 Å.

Below here a measure for the length measured in a straight line for a preferred series of intercalator pseudonucleotides is disclosed:

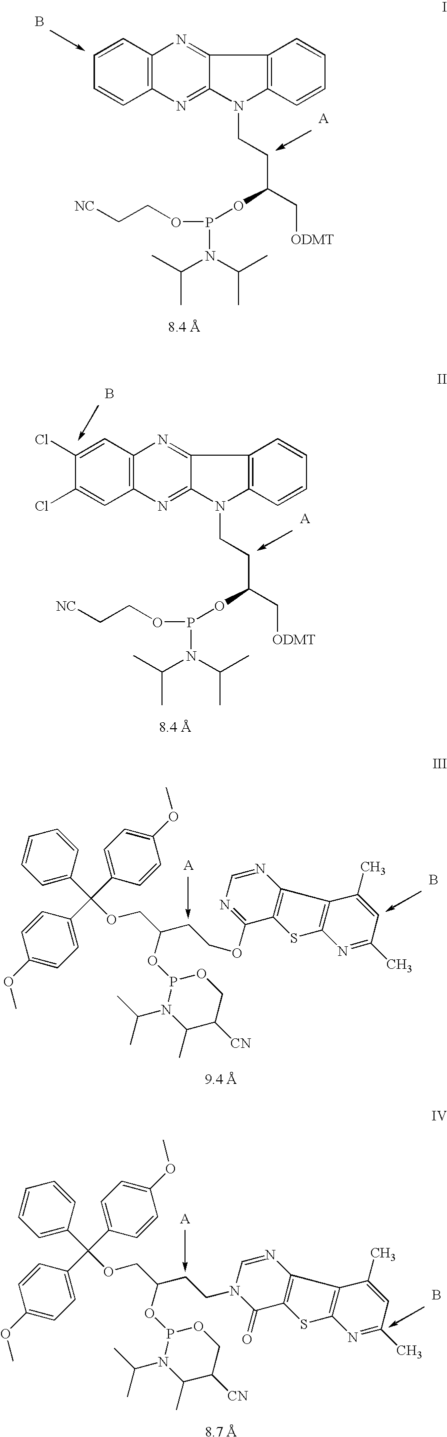

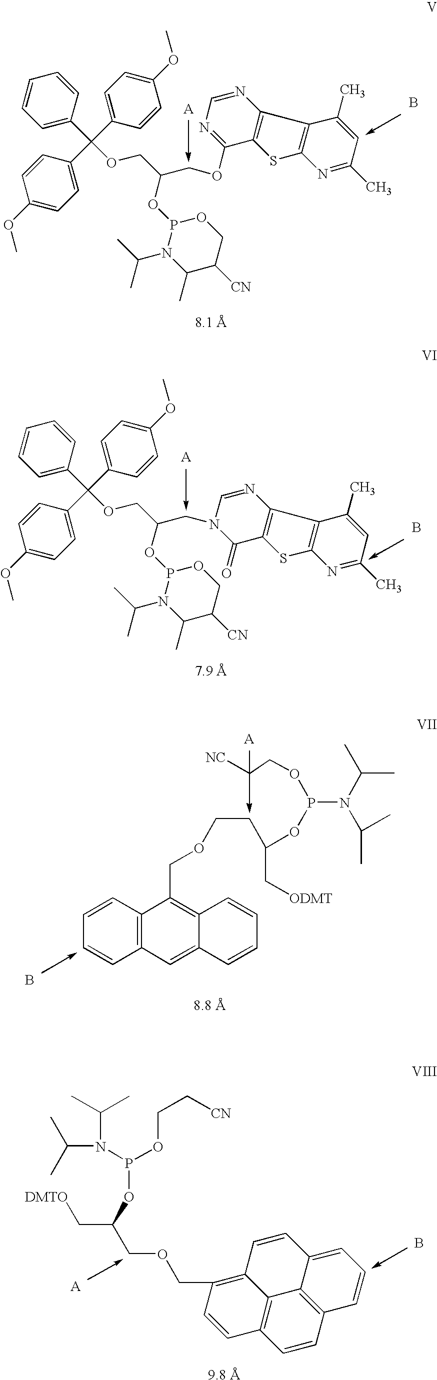

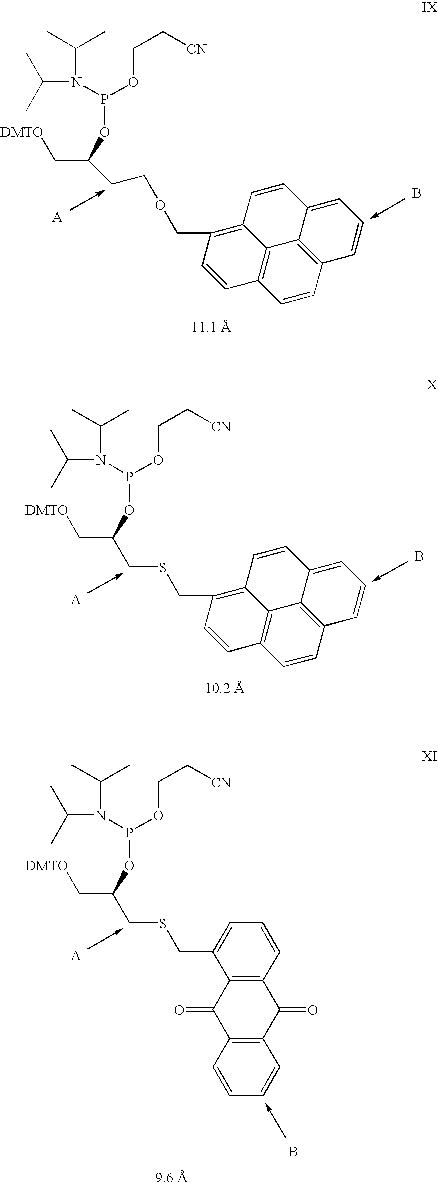

Intercalator pseudonucleotides according to the present invention may be any combination of the above mentioned backbone monomer units, linkers and intercalators.

In one embodiment of the invention the intercalator pseudonucleotide is selected from the group consisting of intercalator pseudonucleotides with the structures 1) to 9 as indicated herein below:

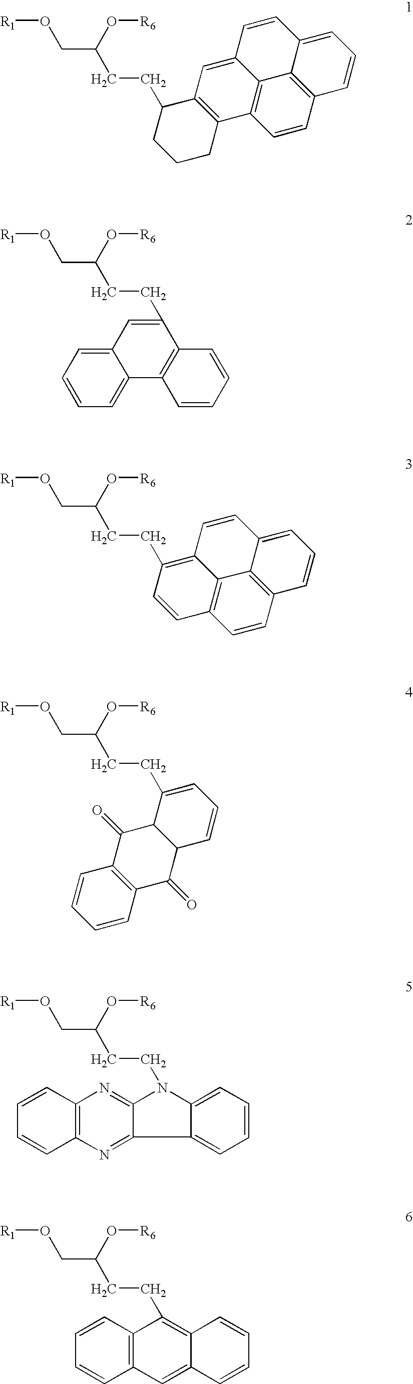

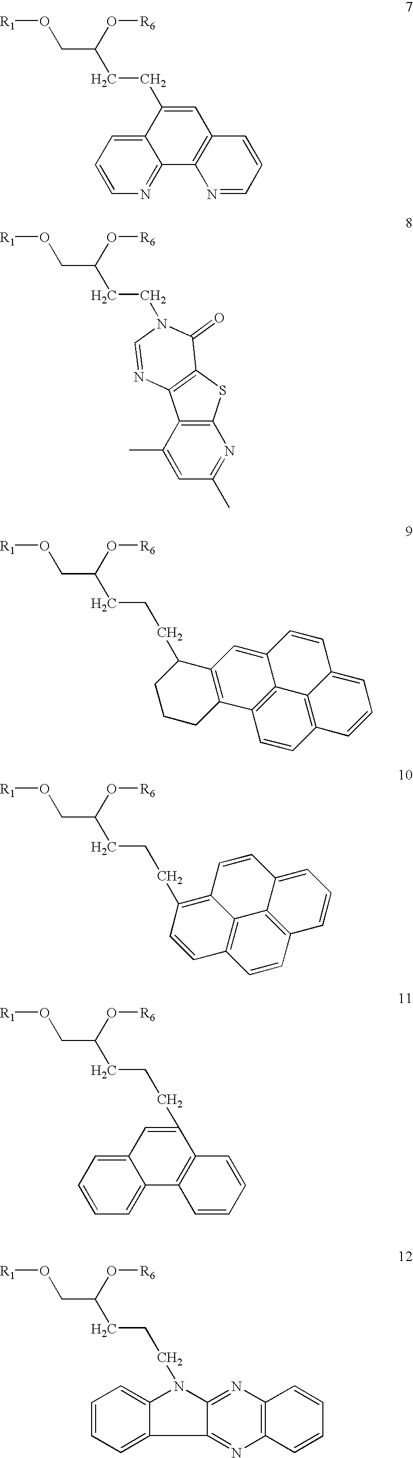

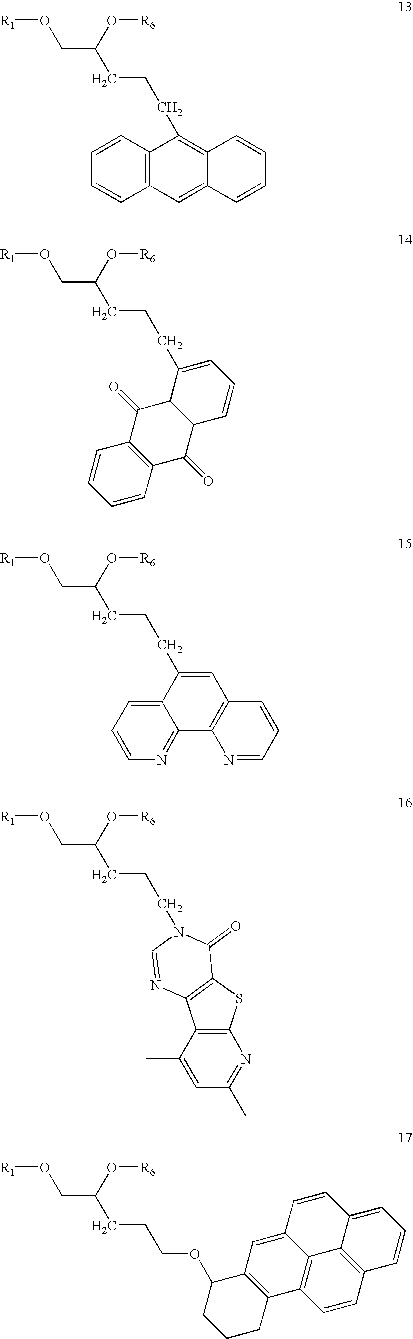

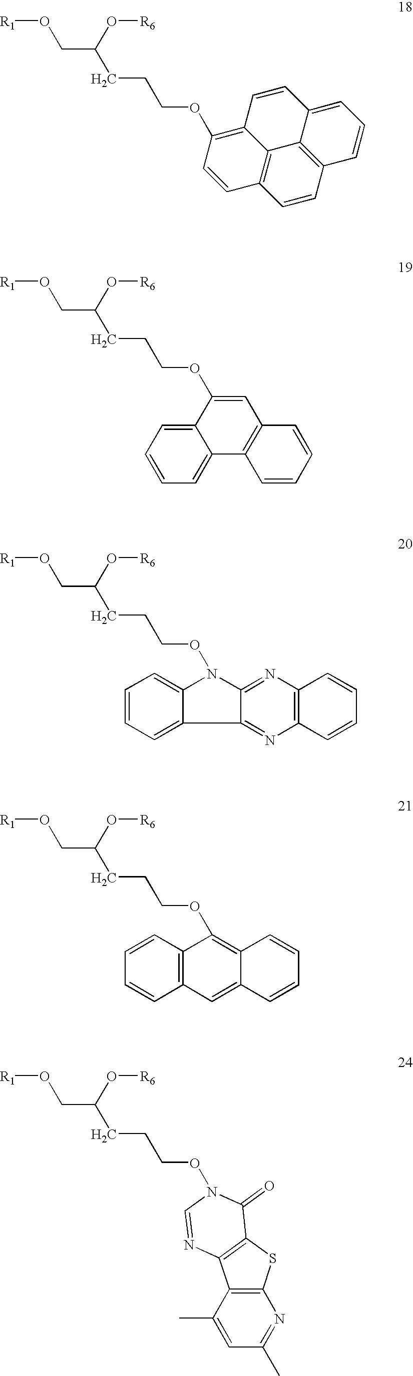

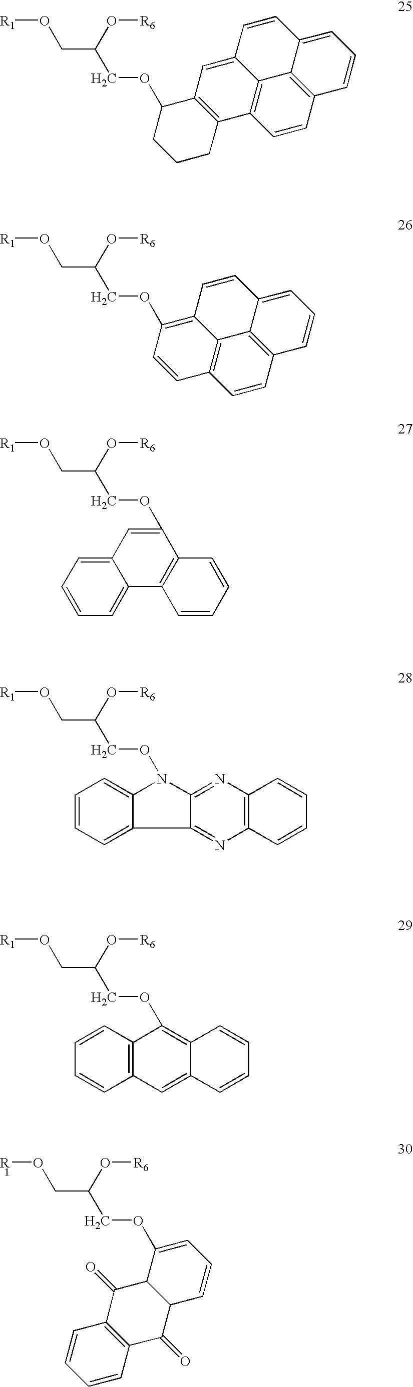

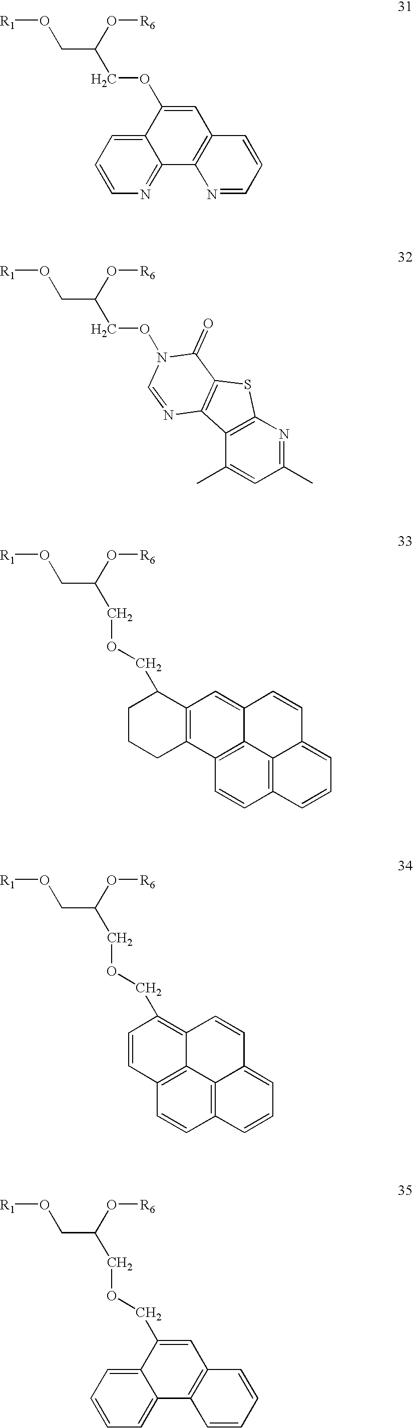

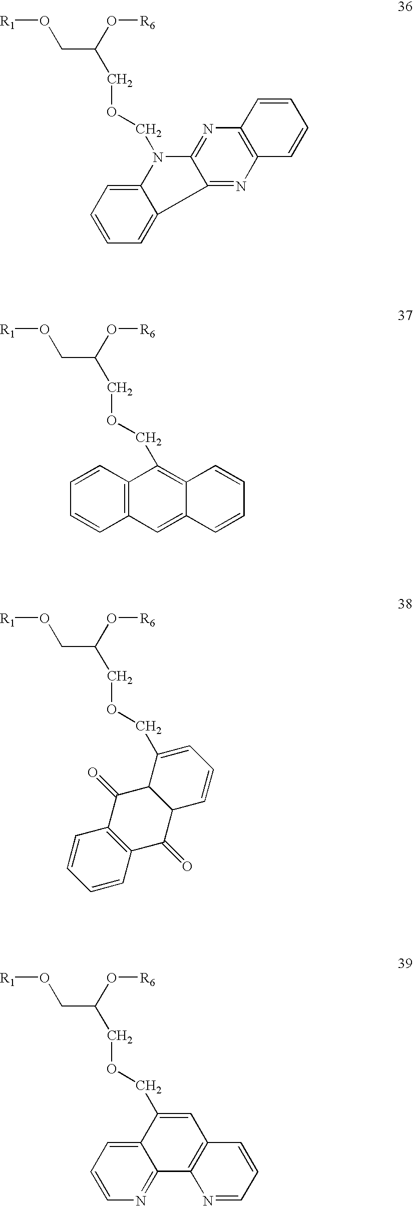

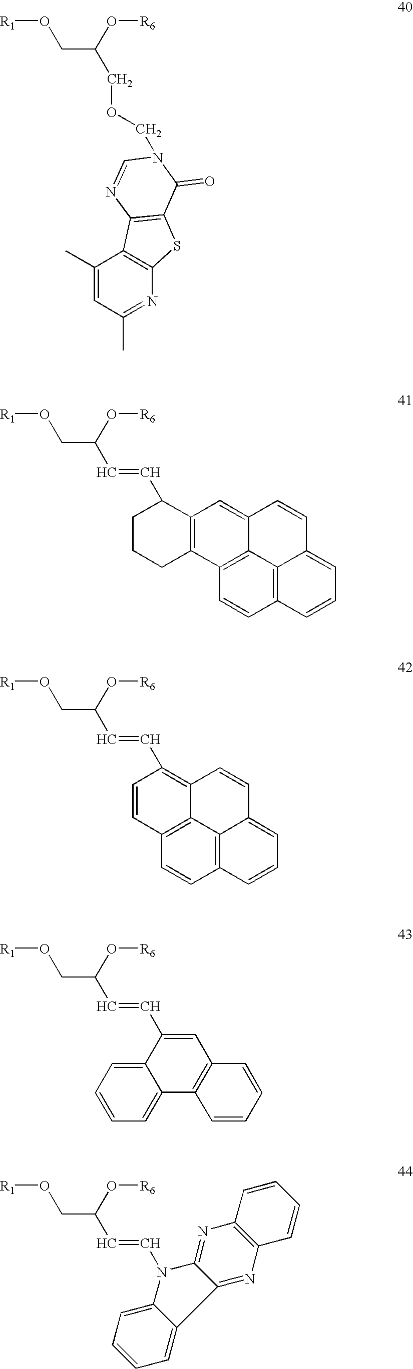

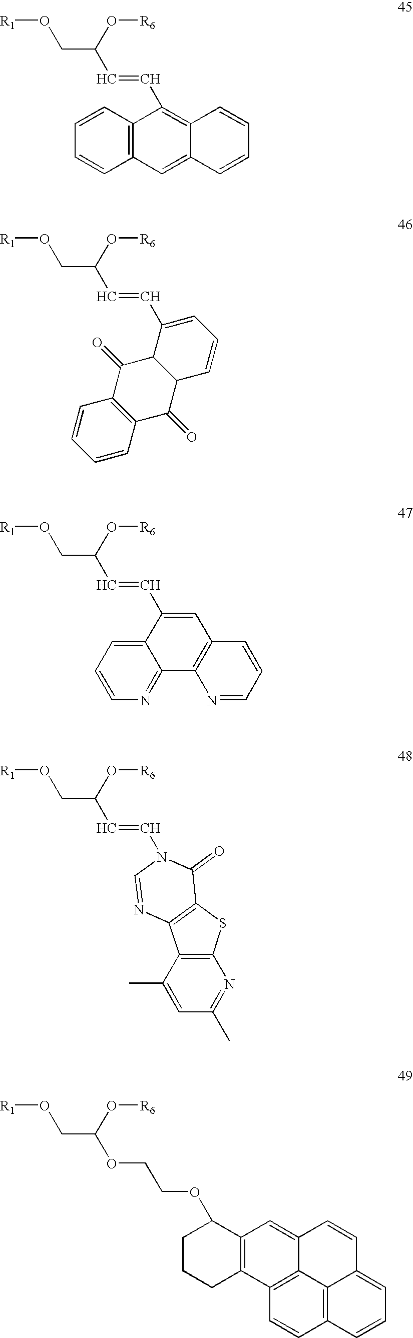

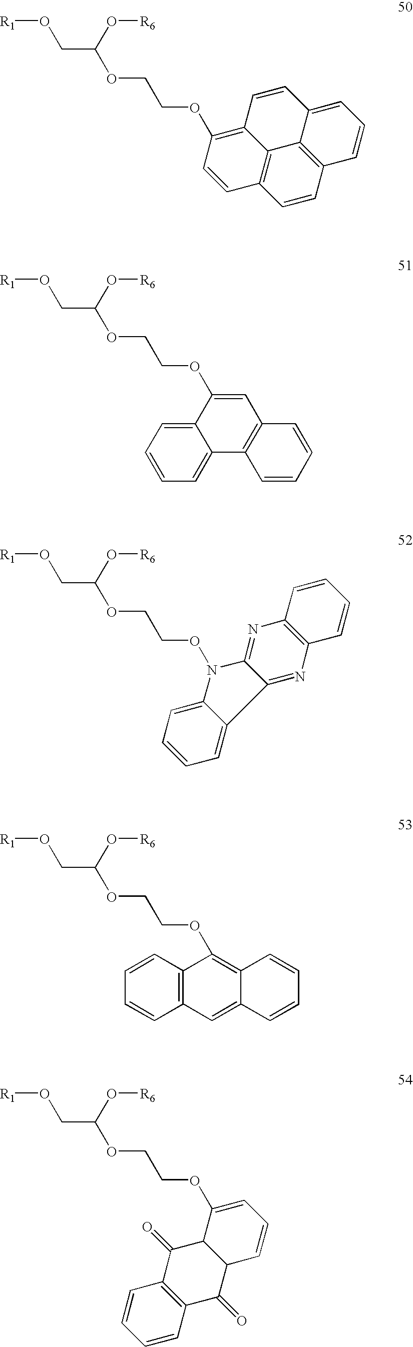

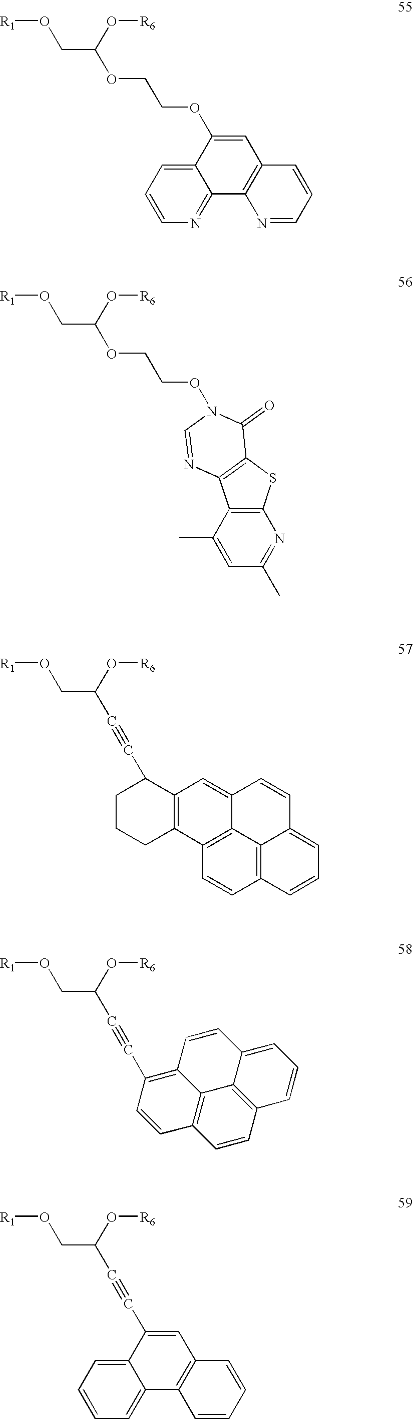

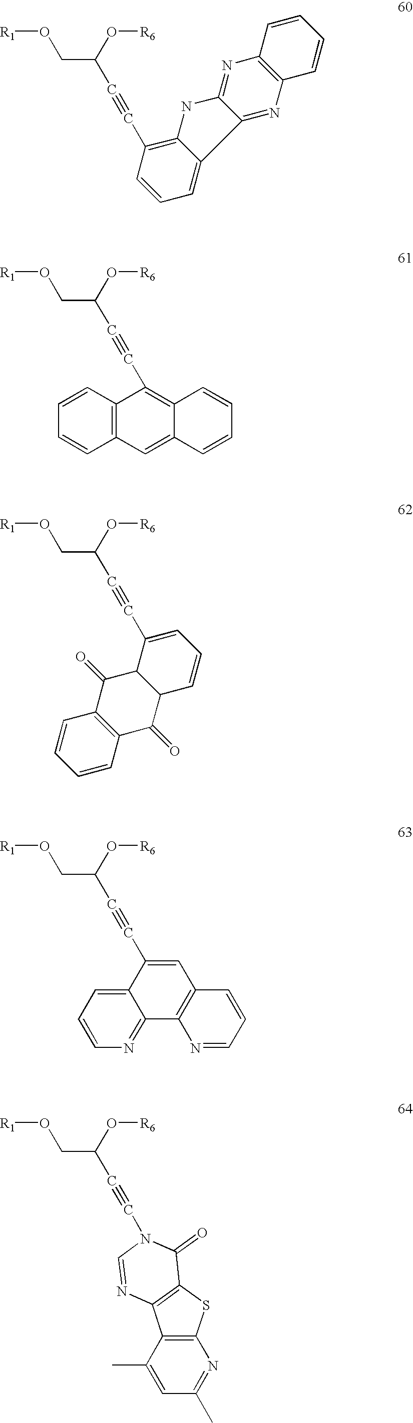

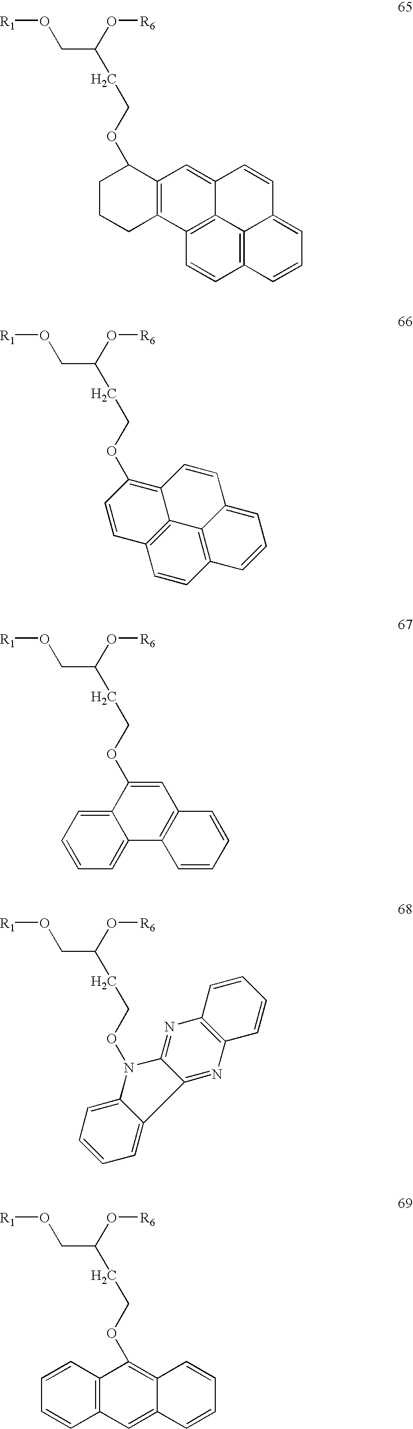

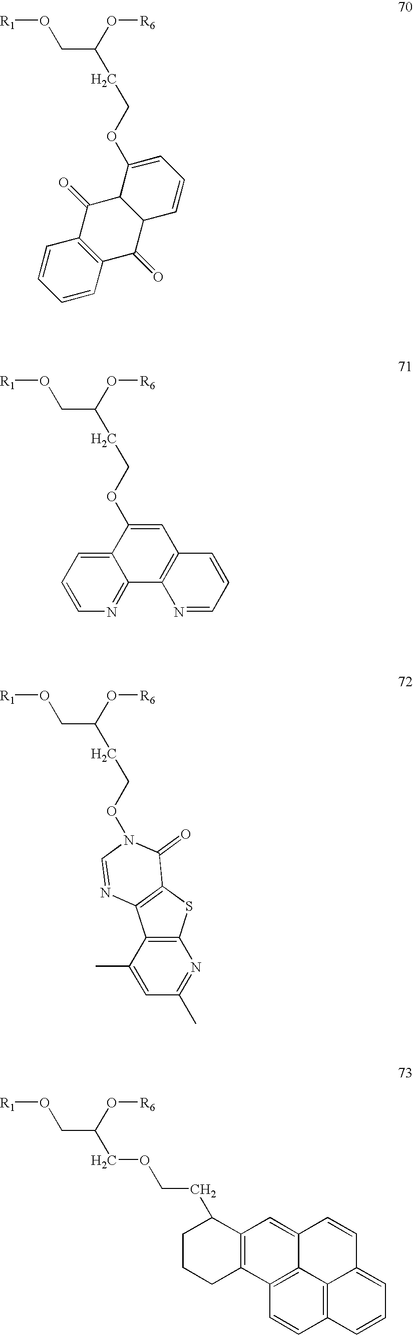

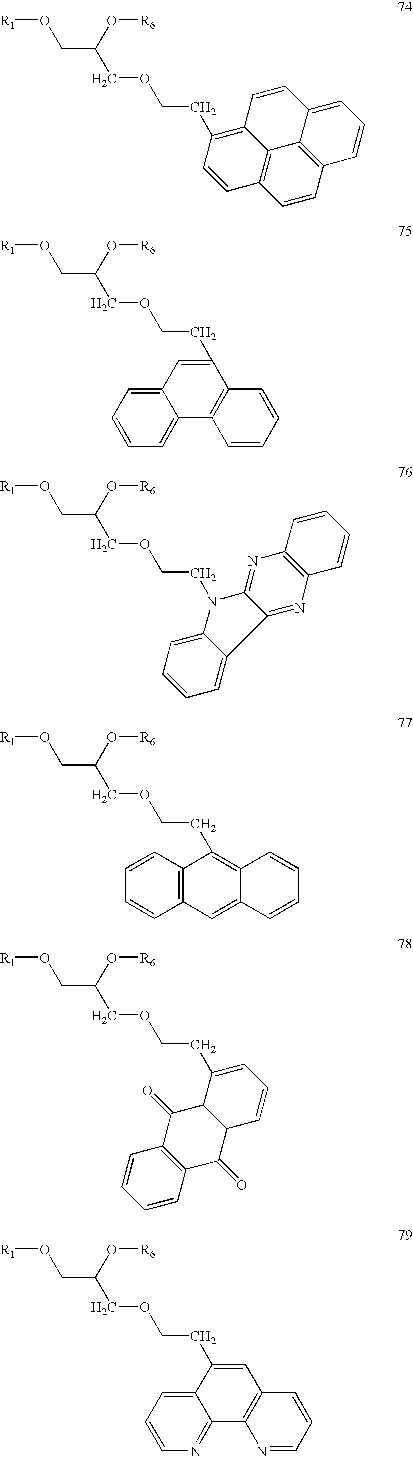

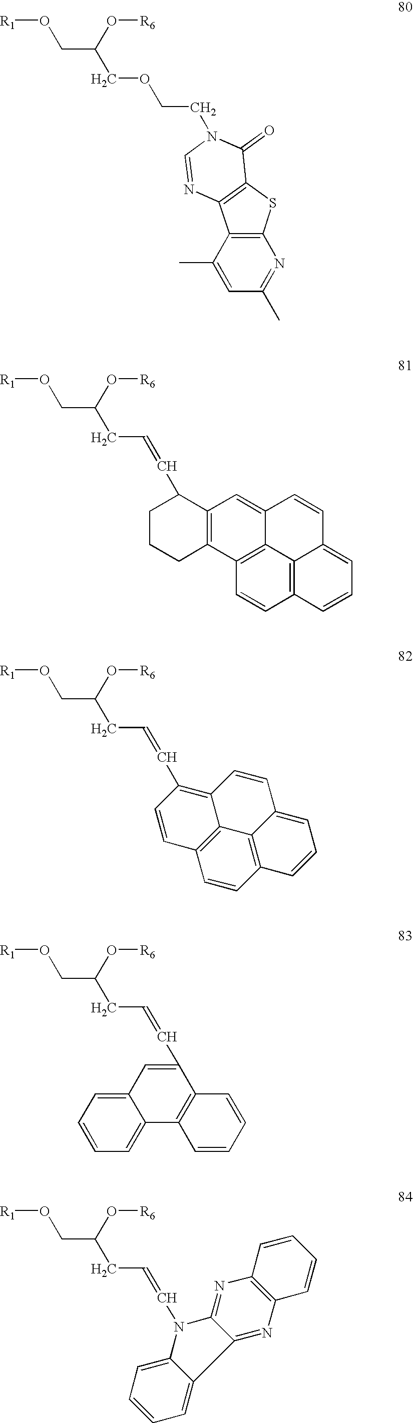

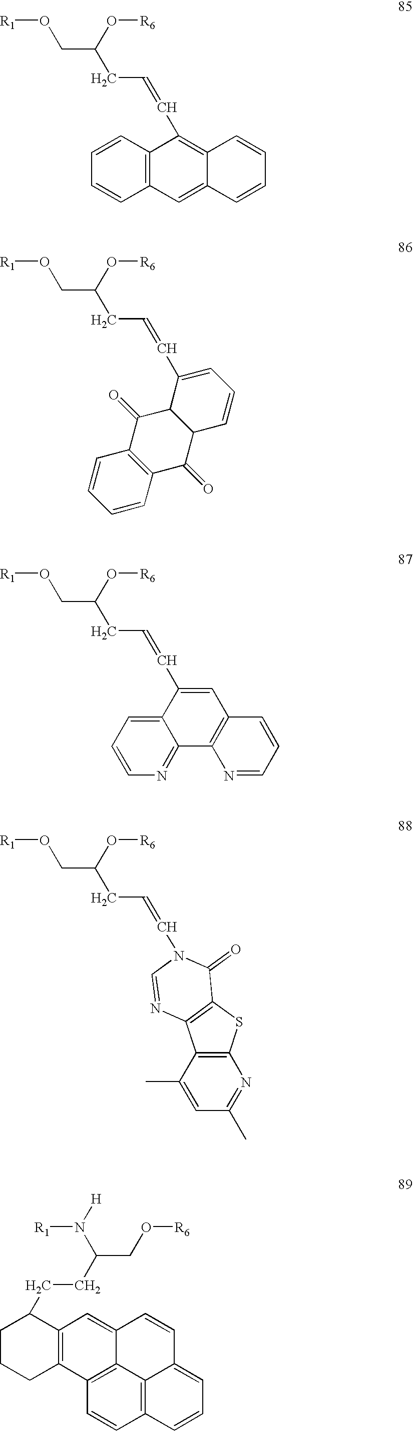

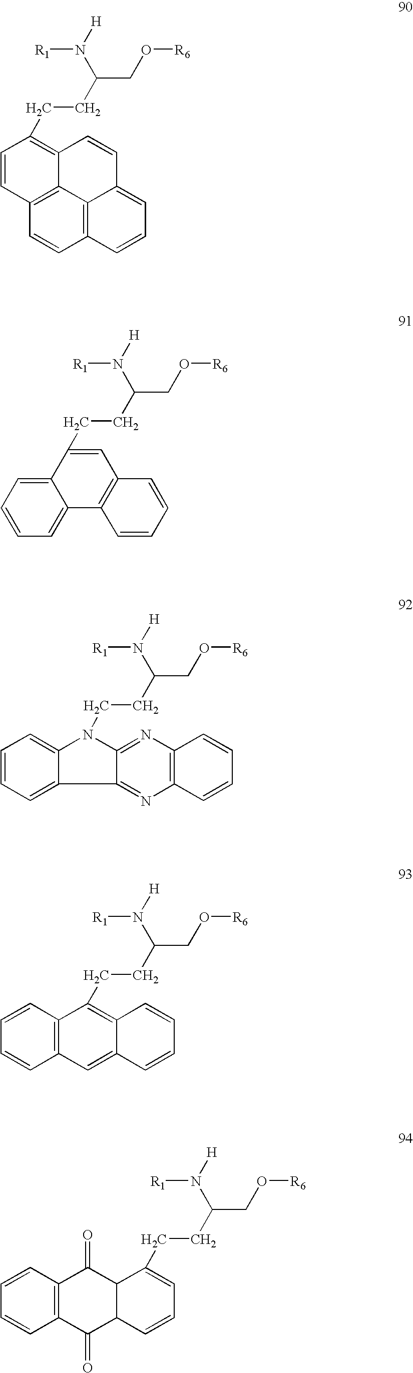

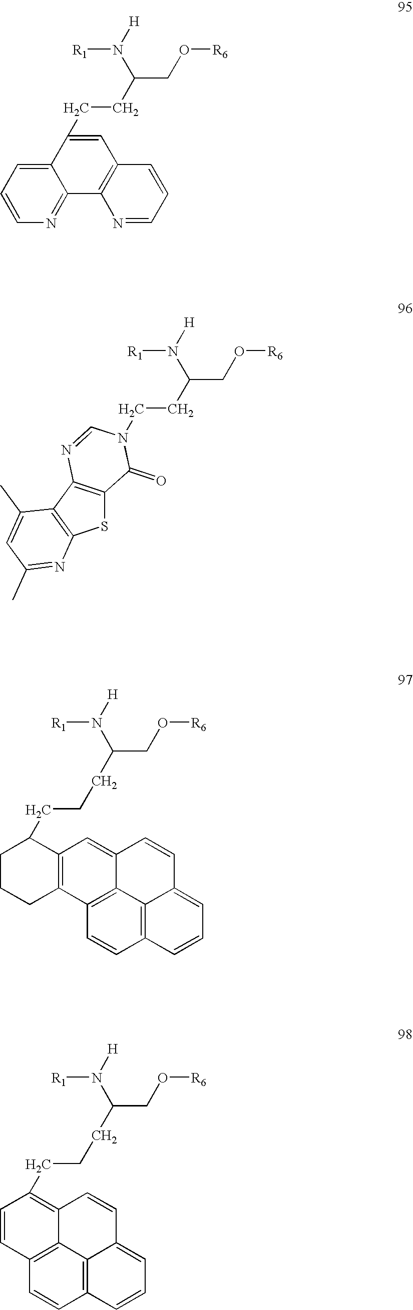

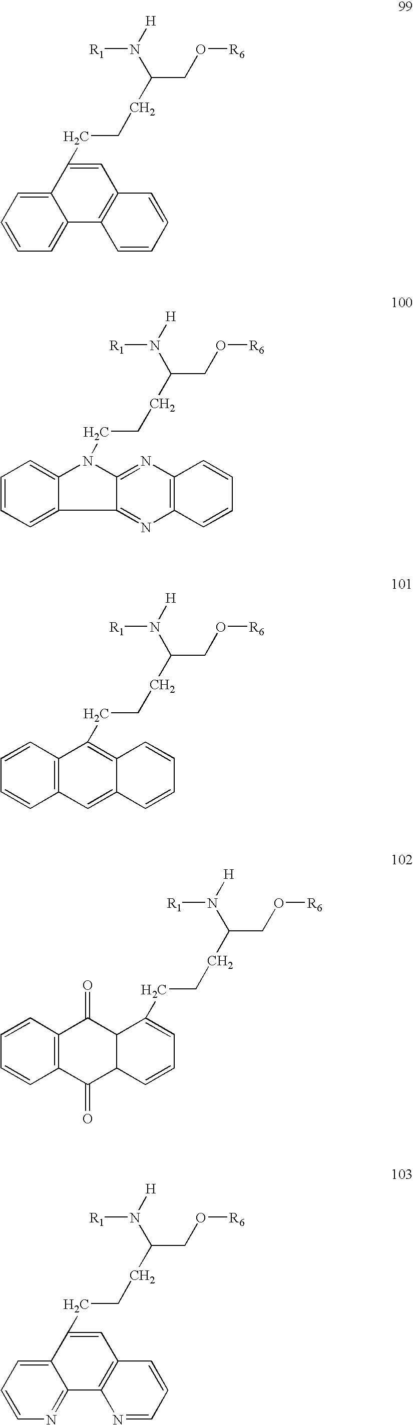

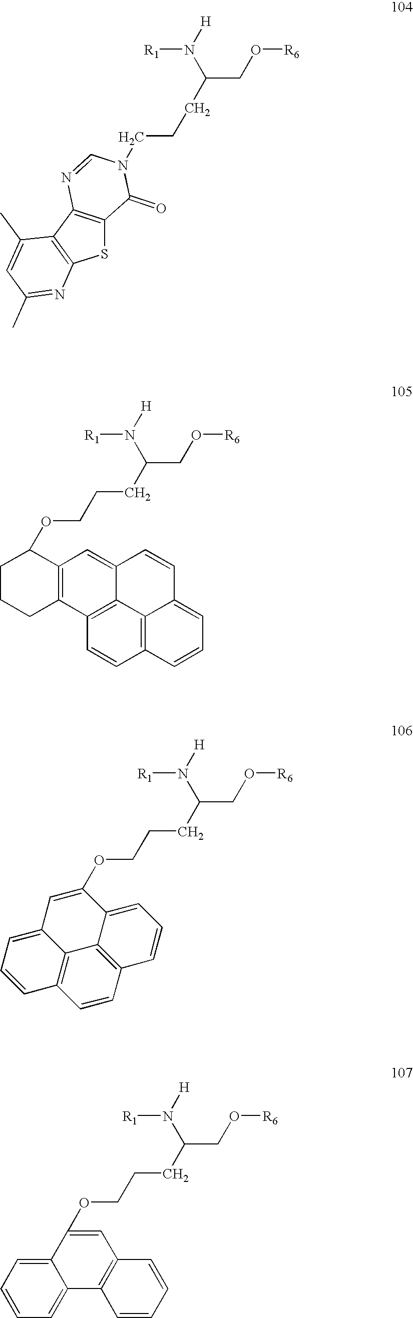

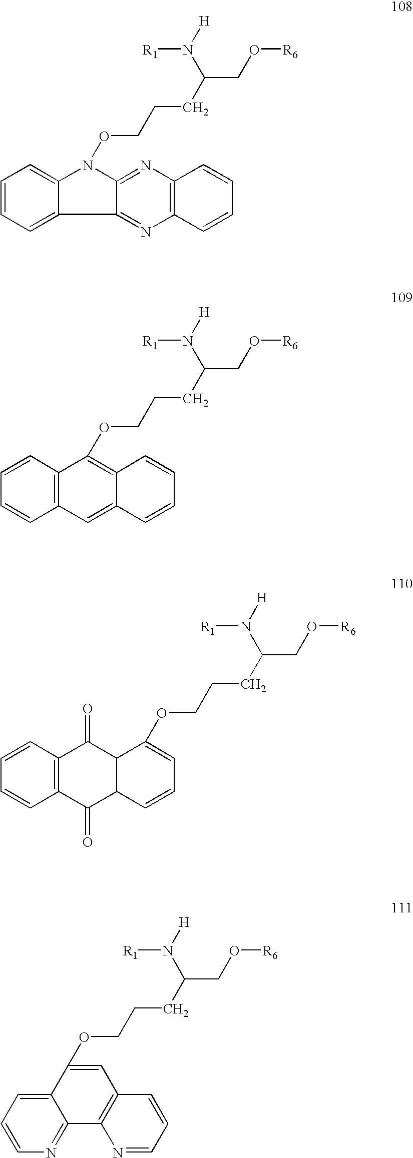

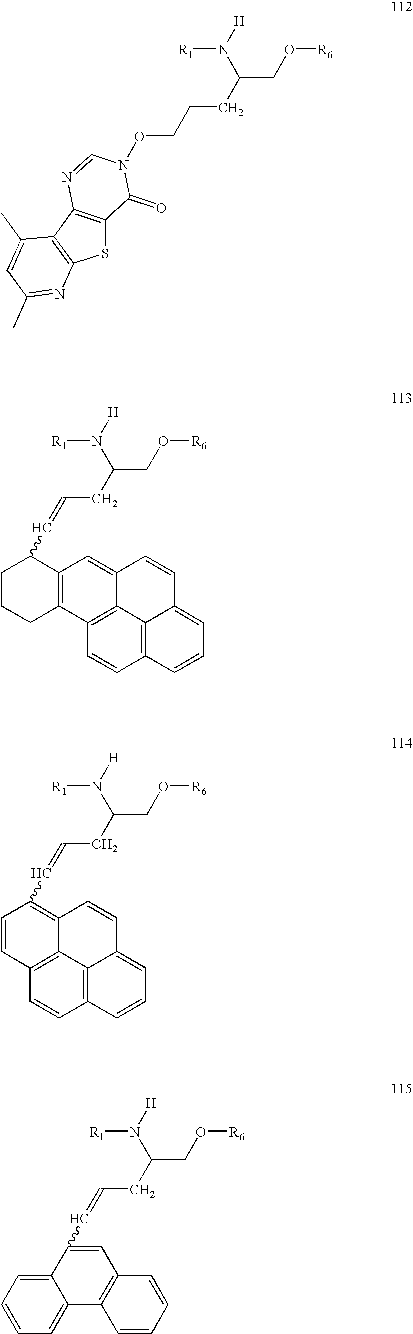

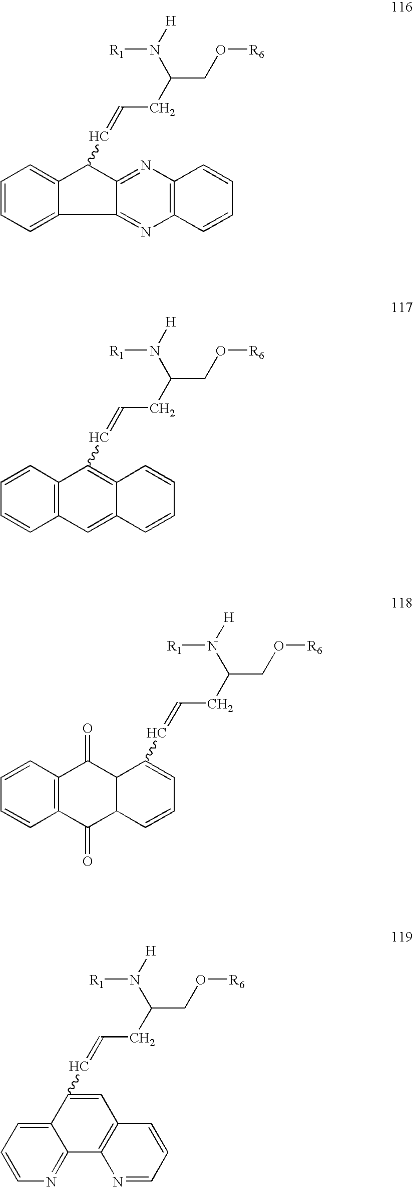

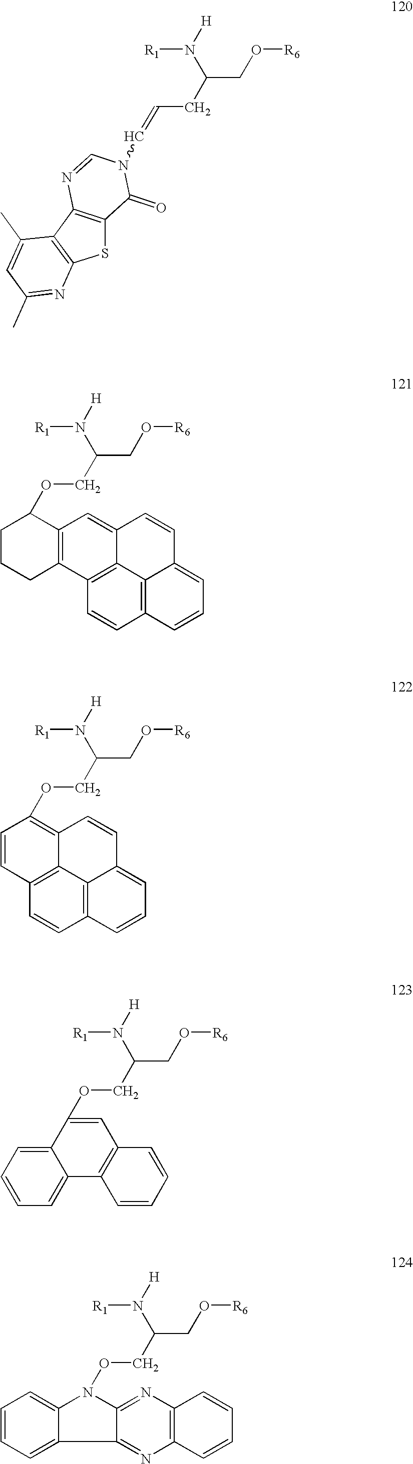

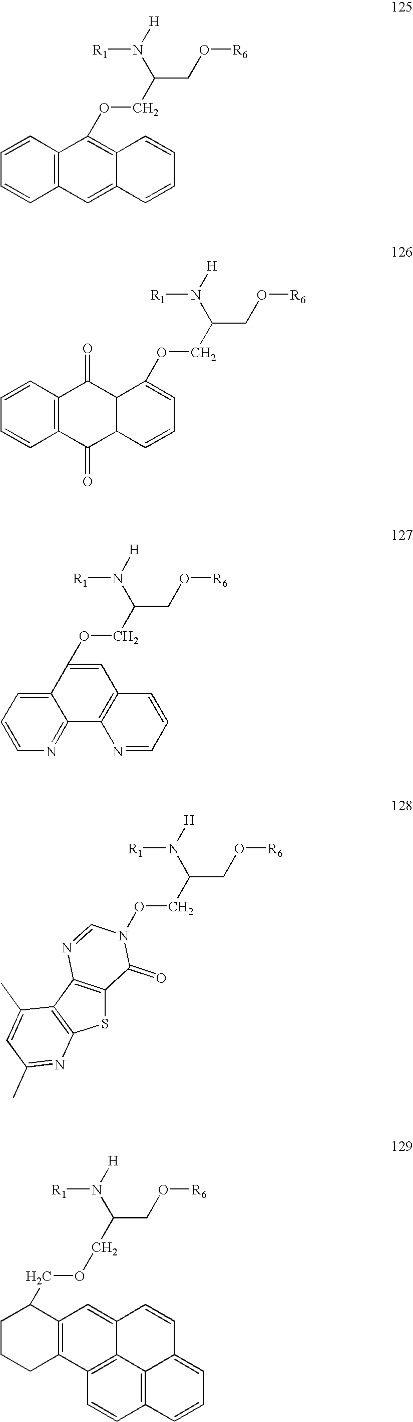

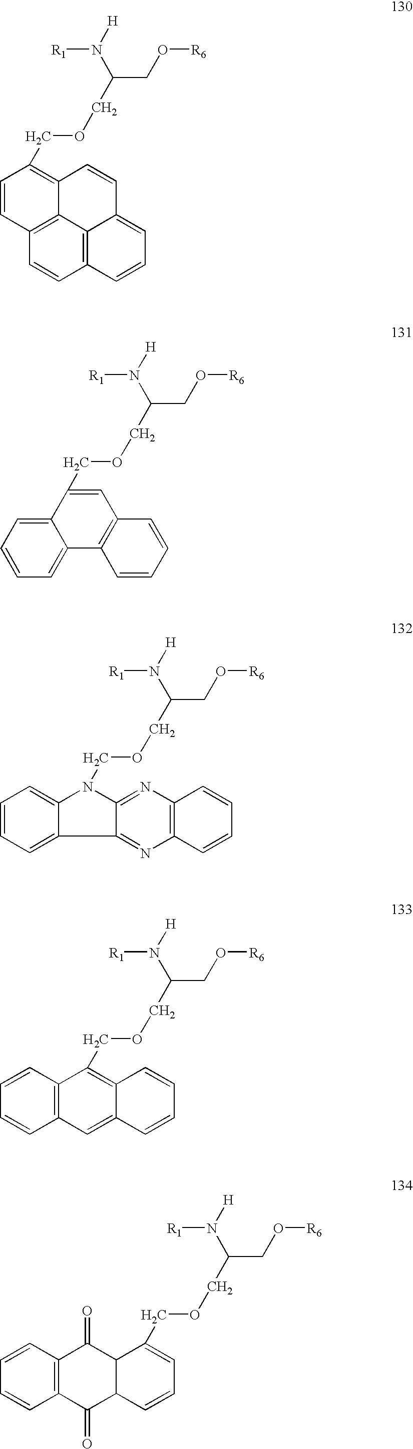

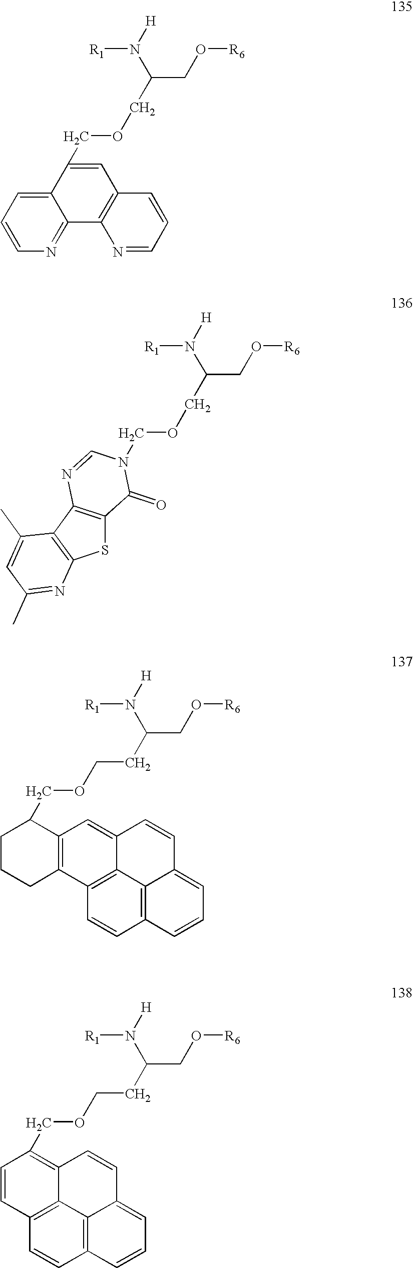

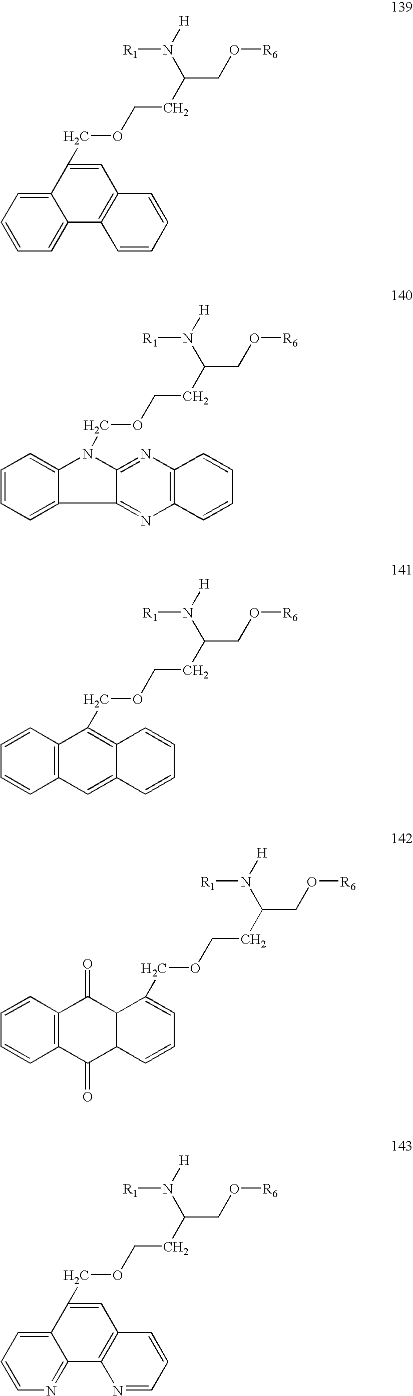

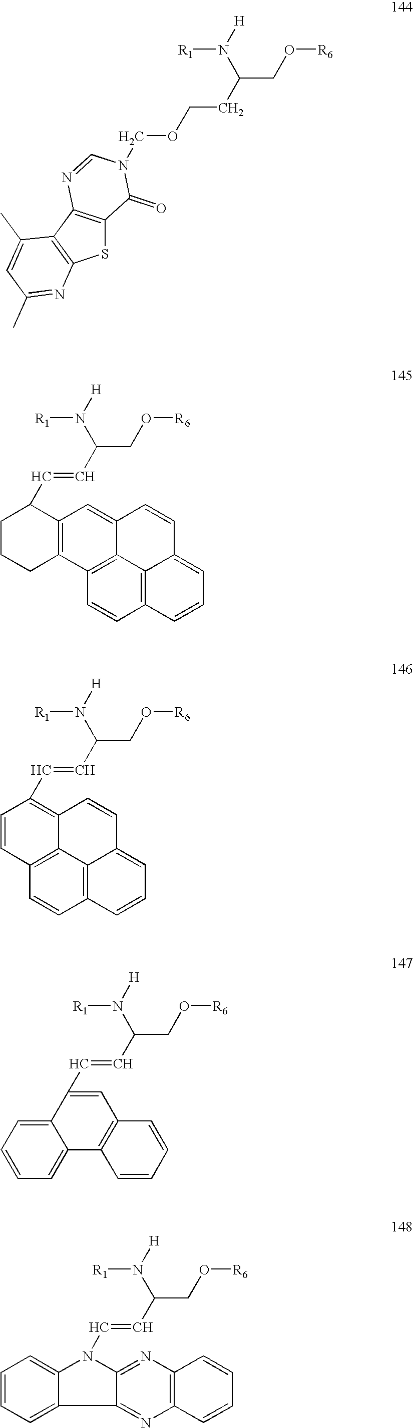

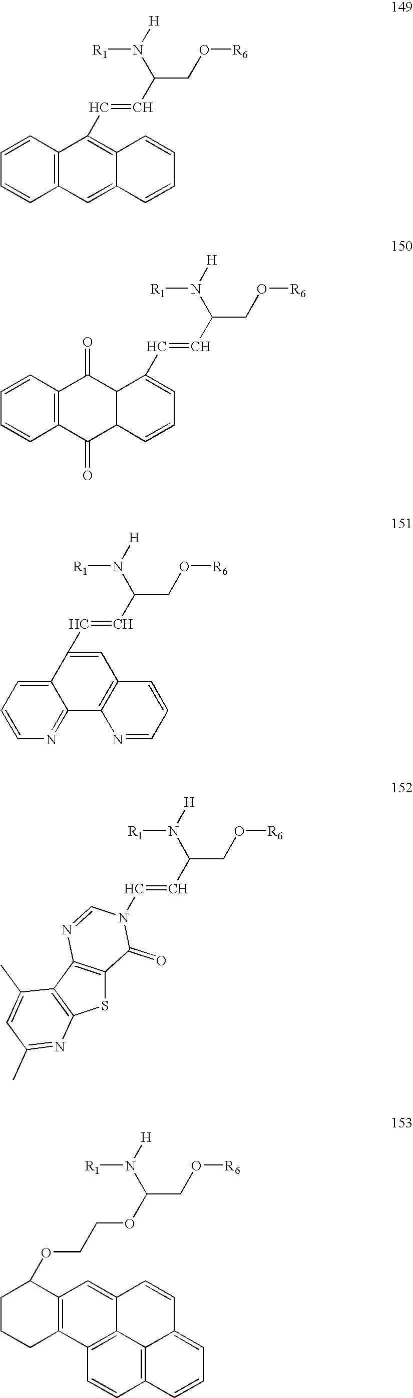

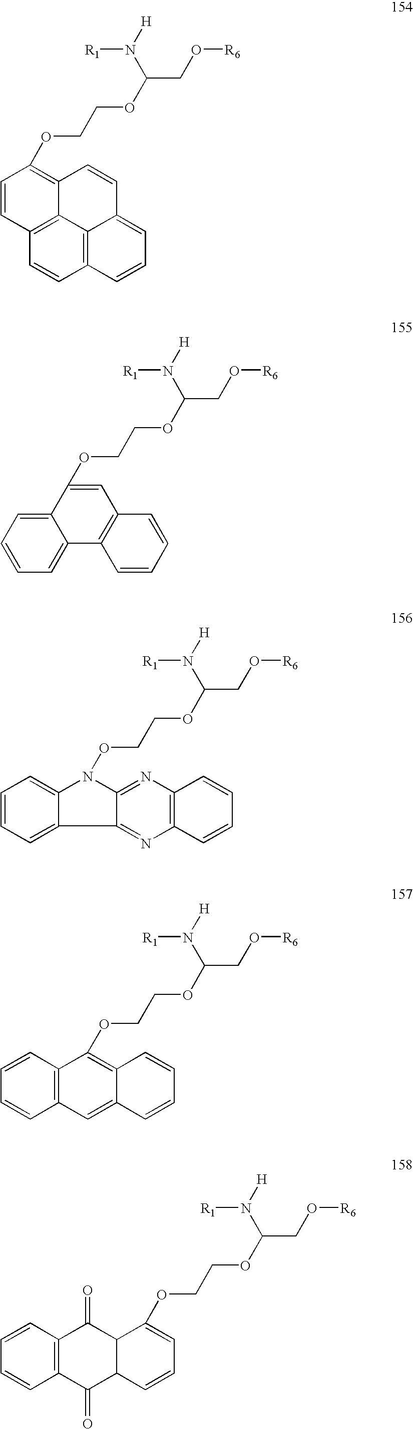

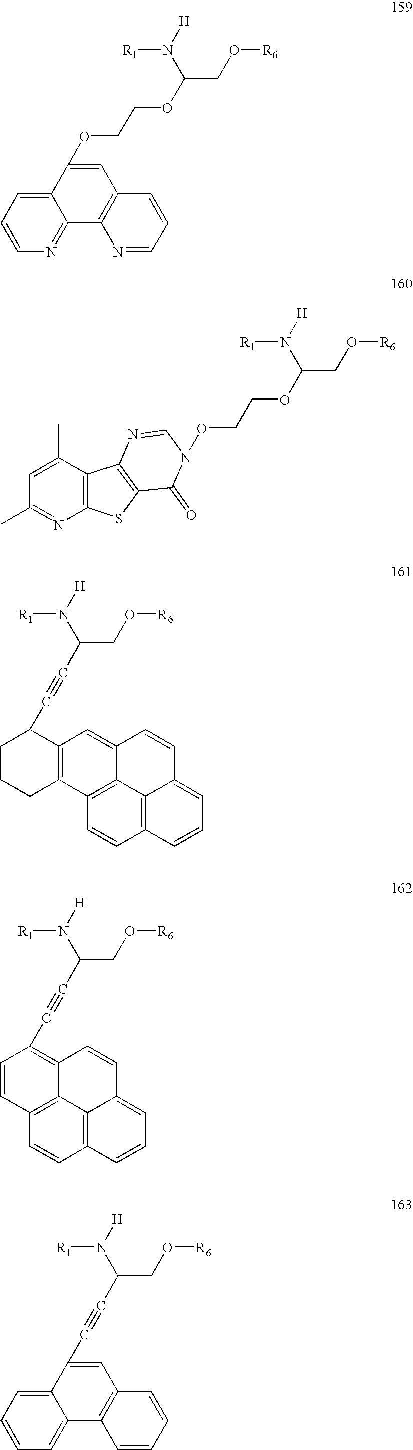

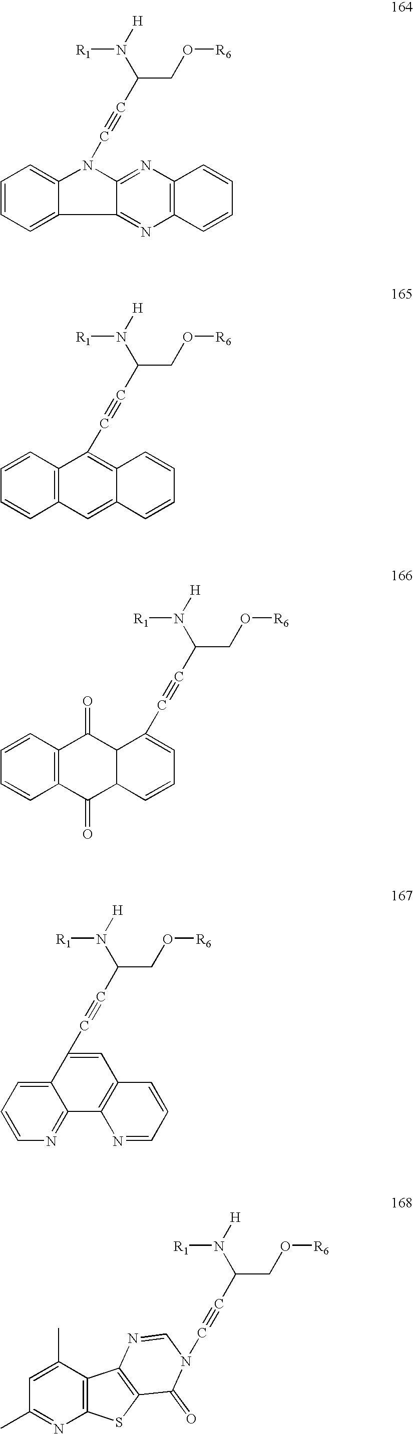

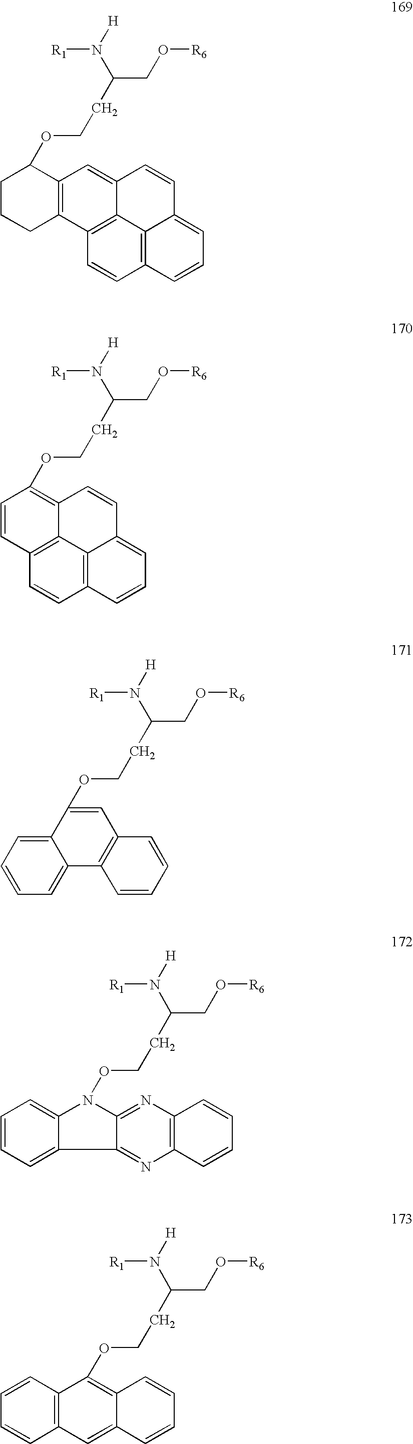

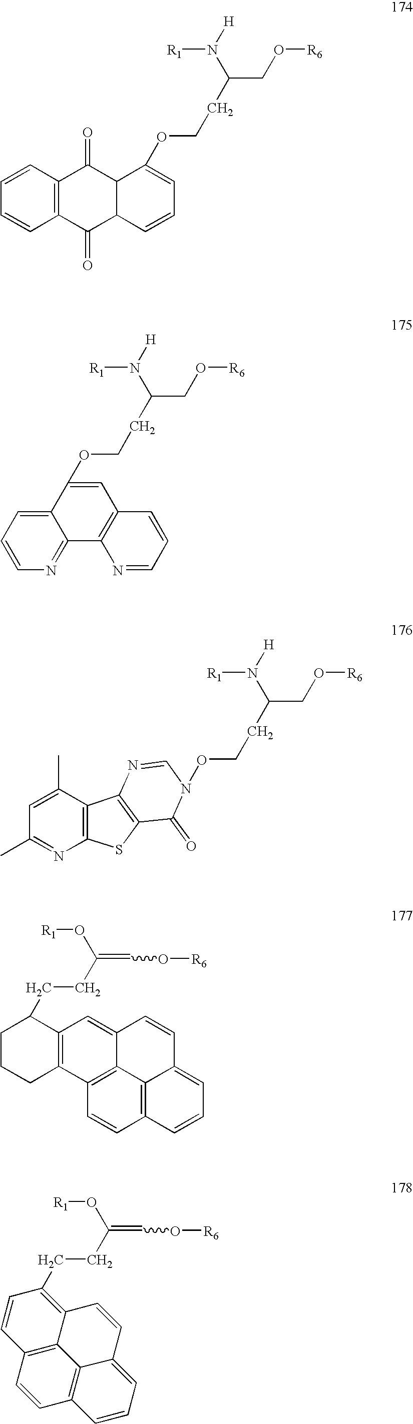

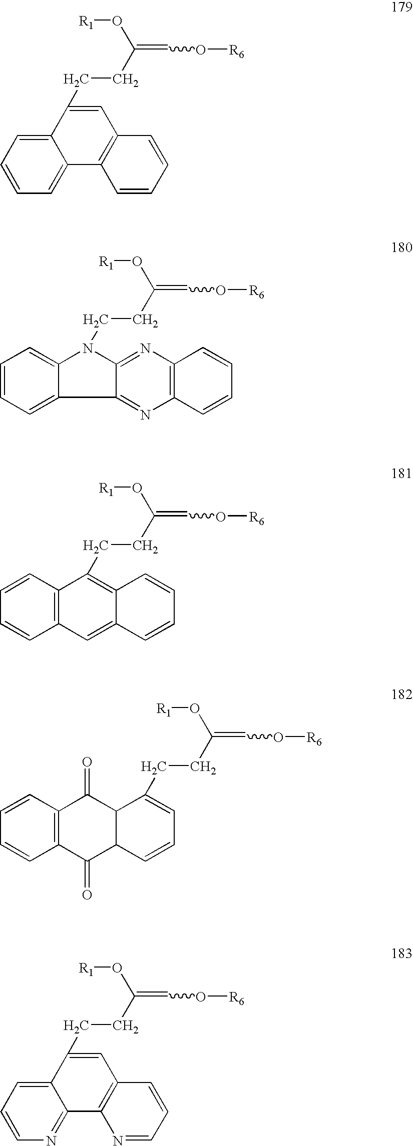

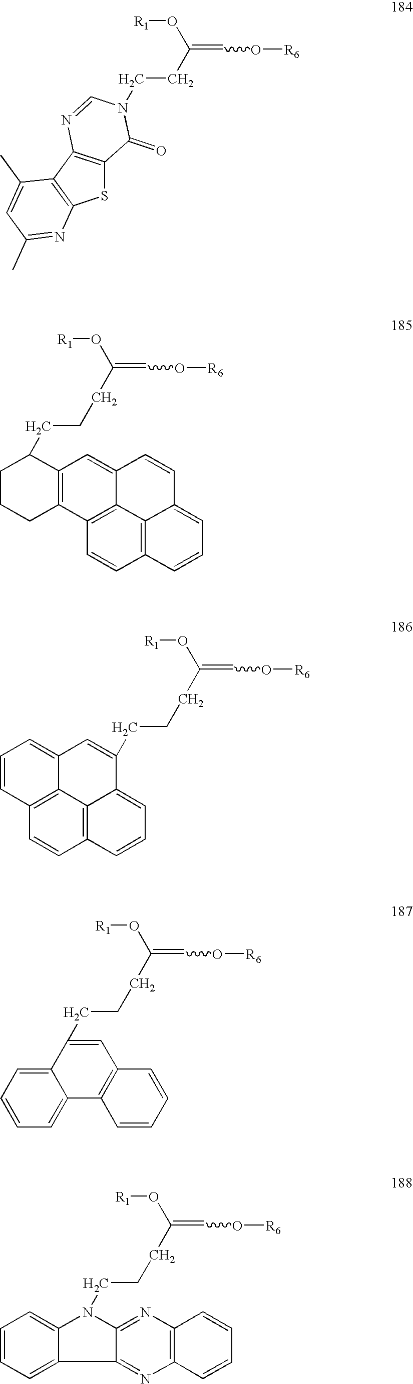

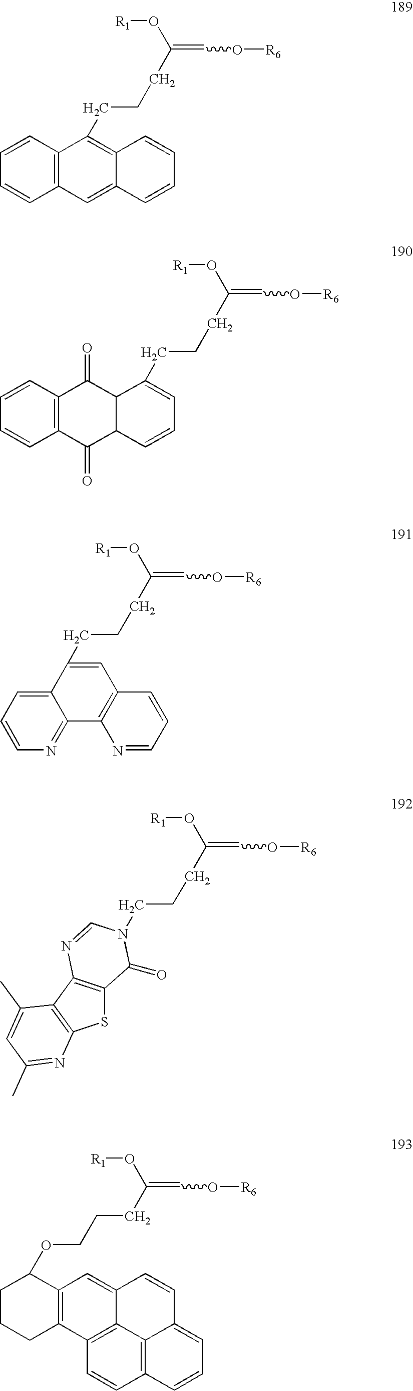

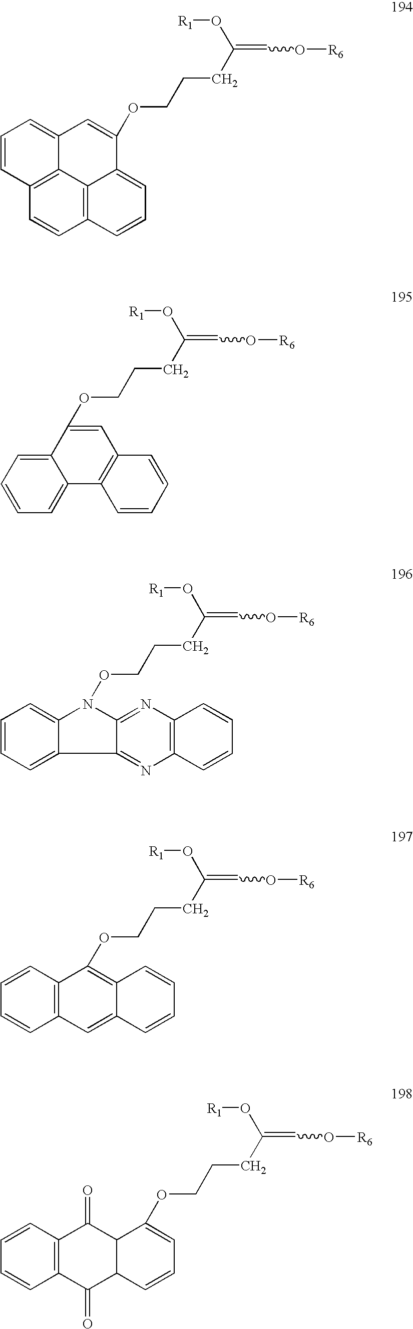

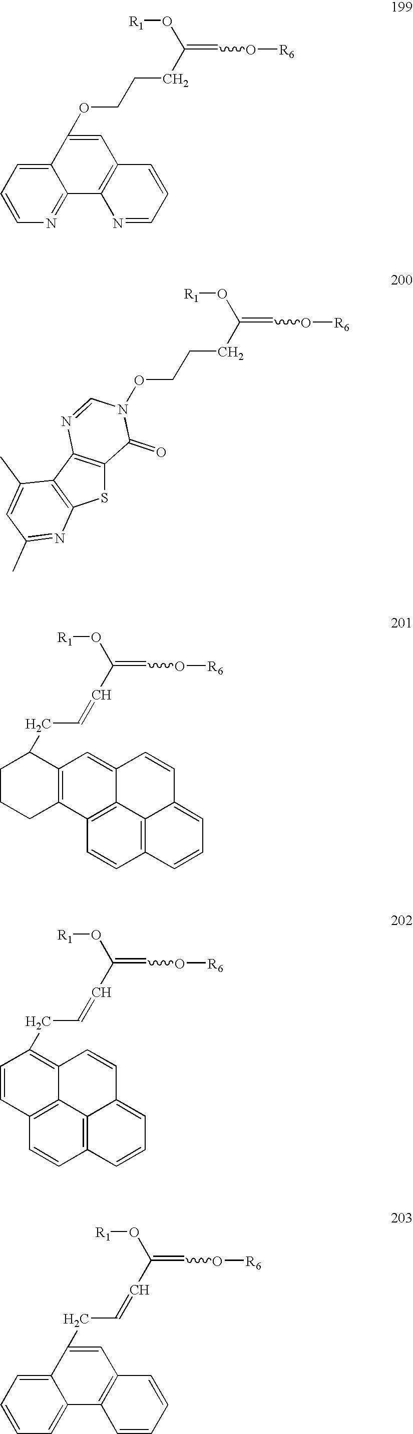

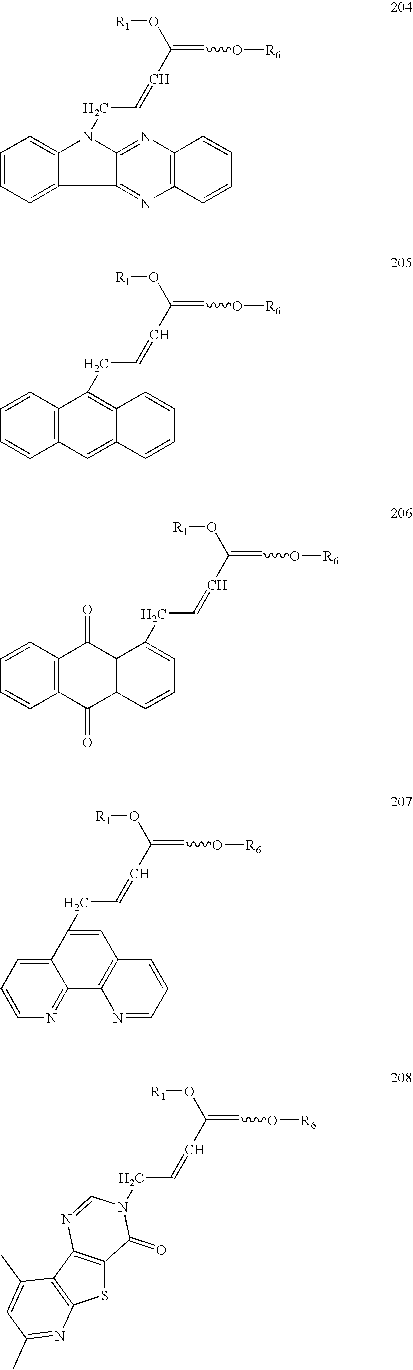

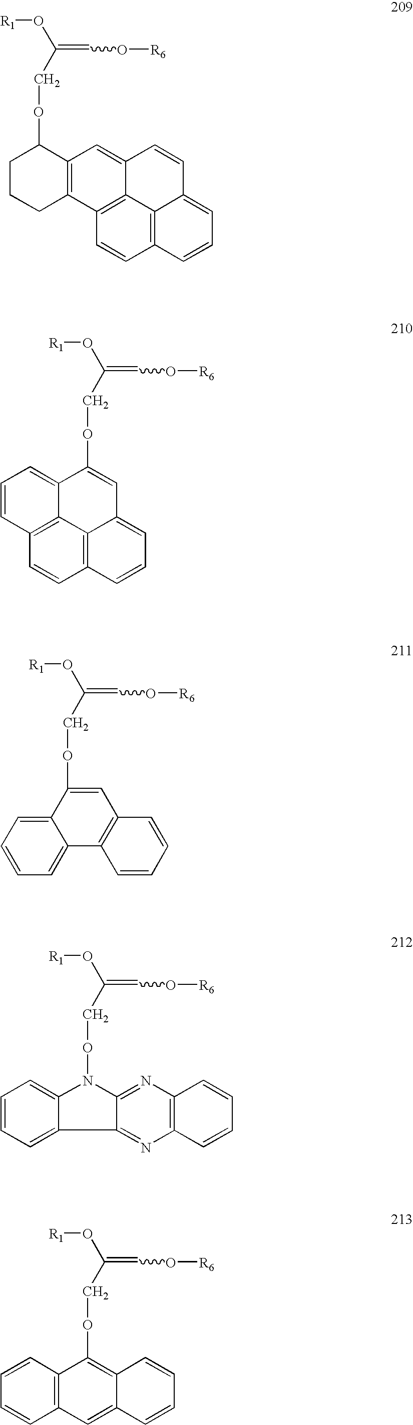

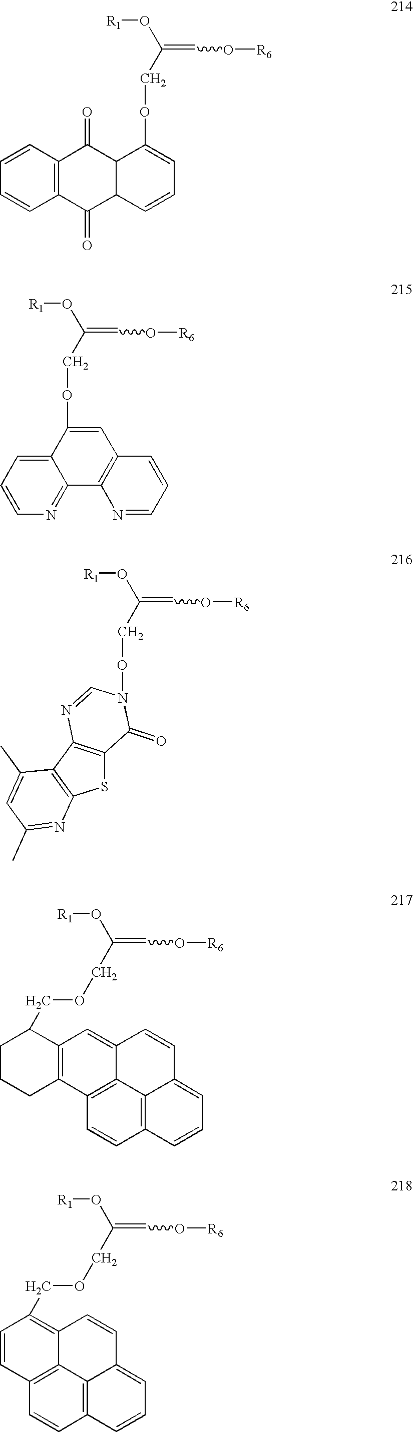

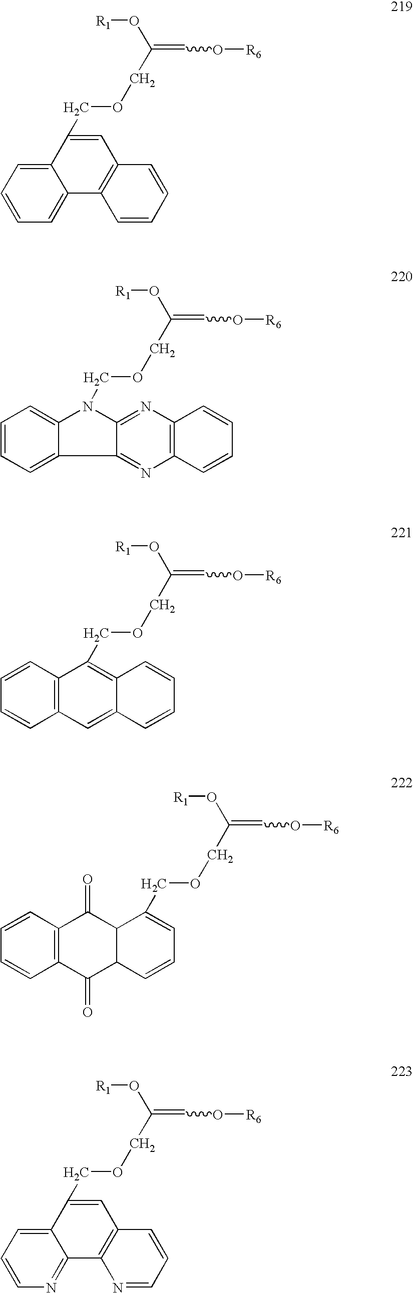

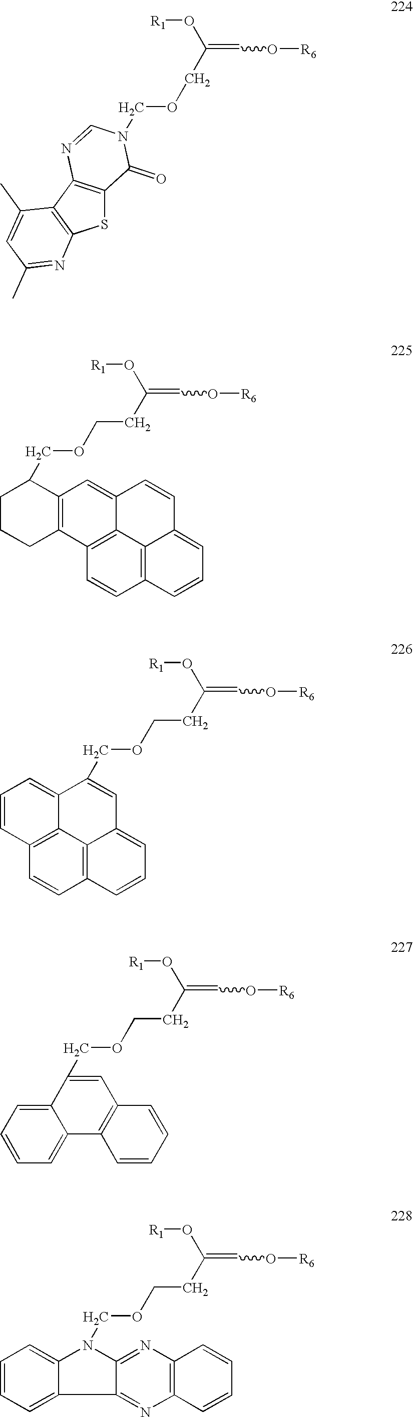

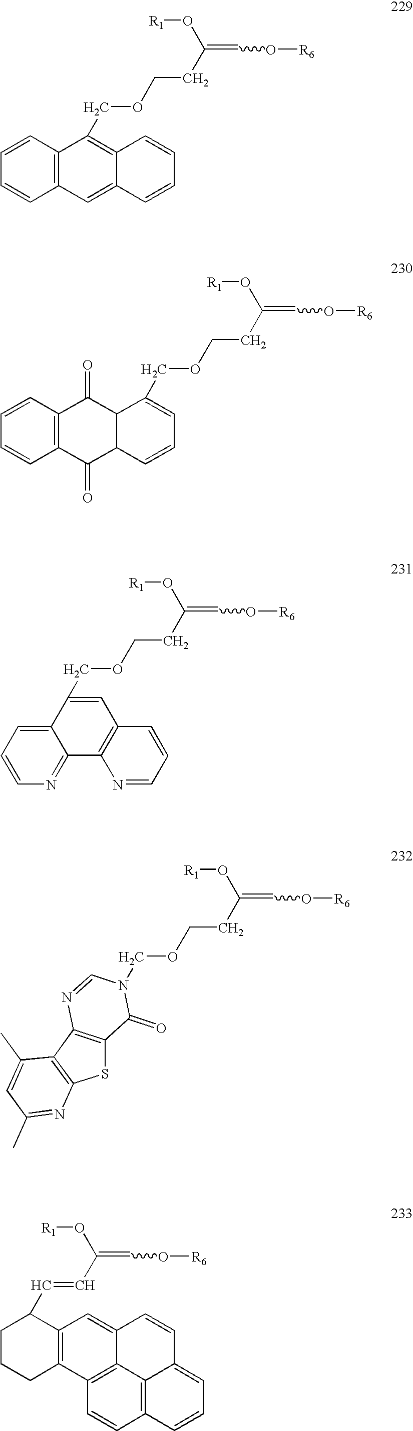

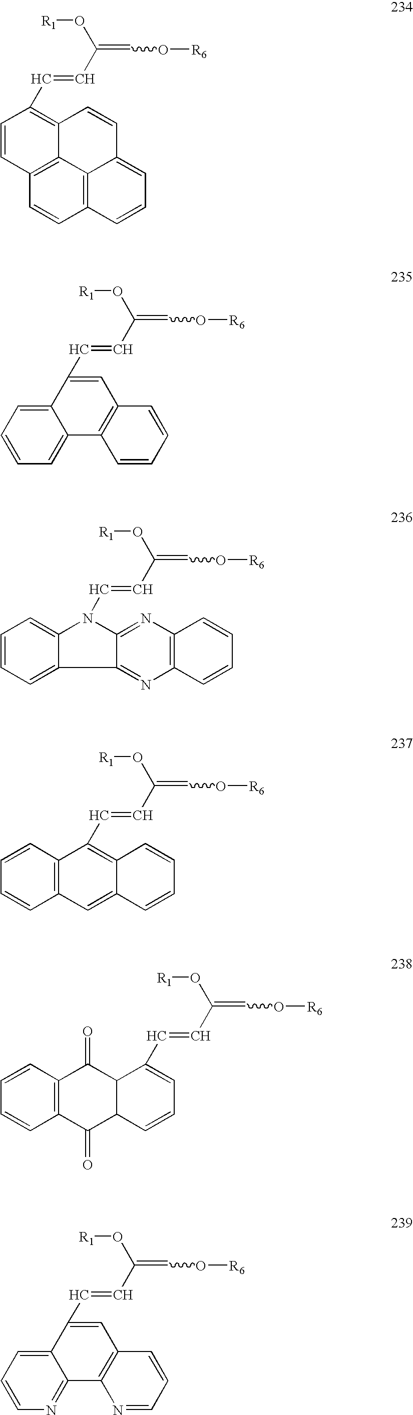

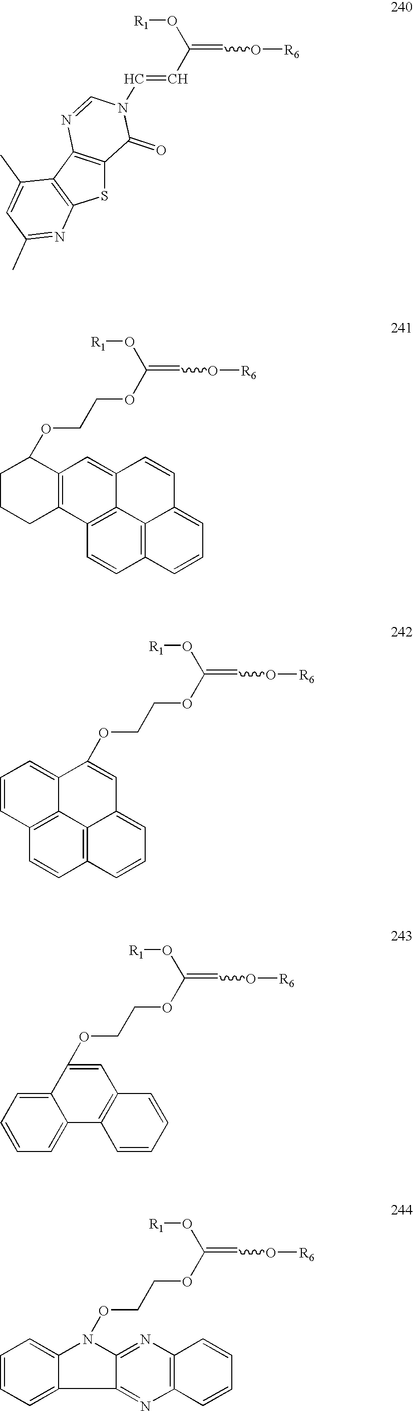

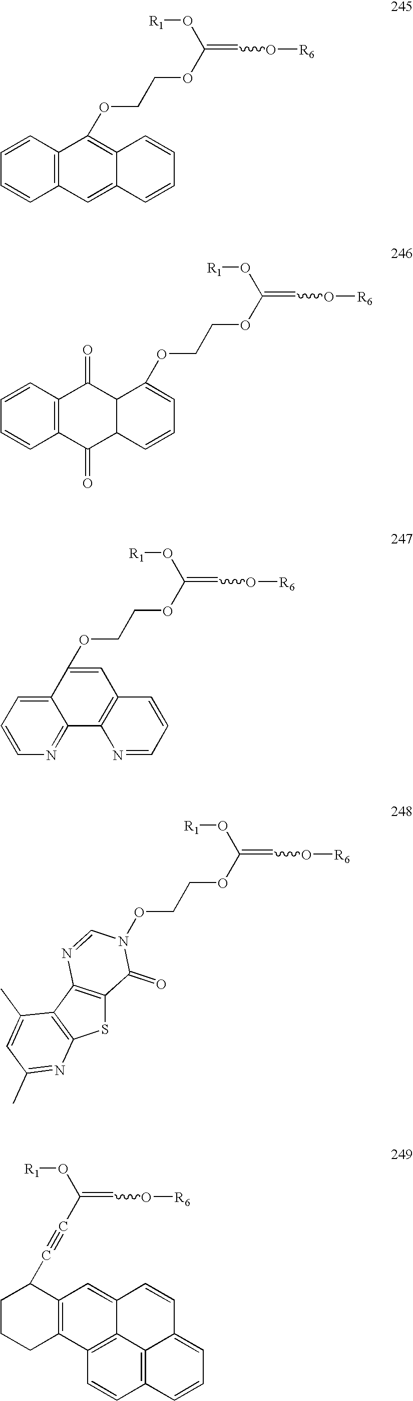

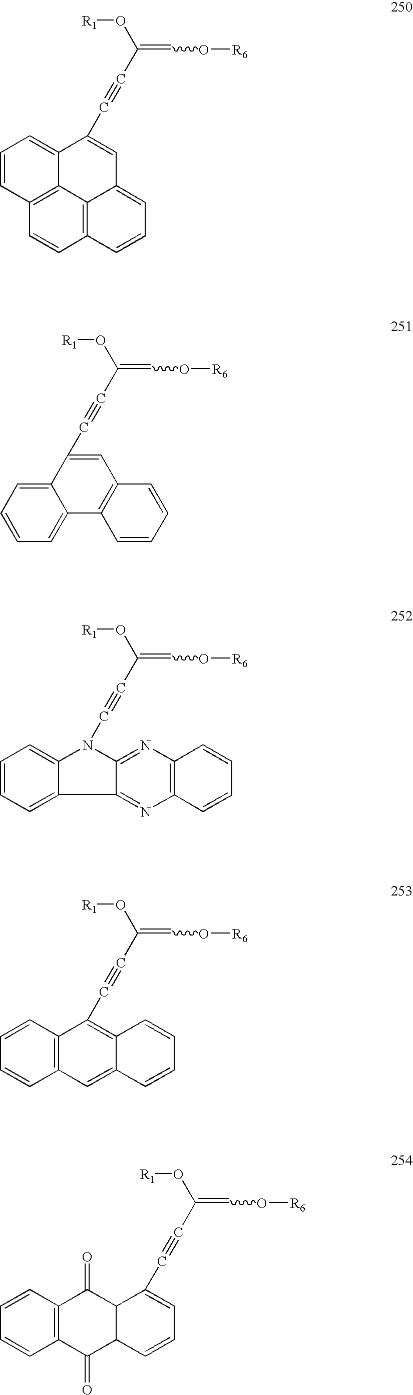

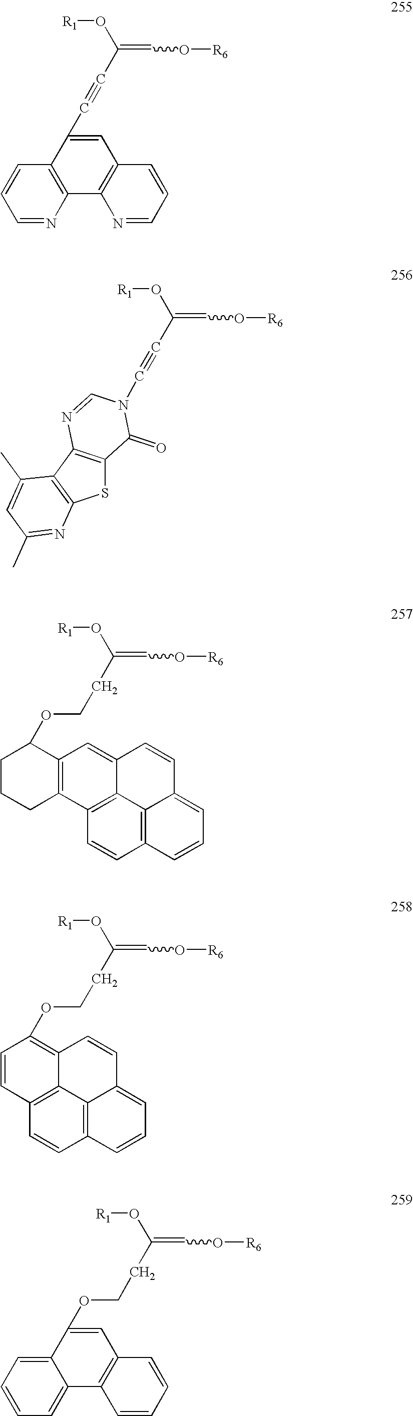

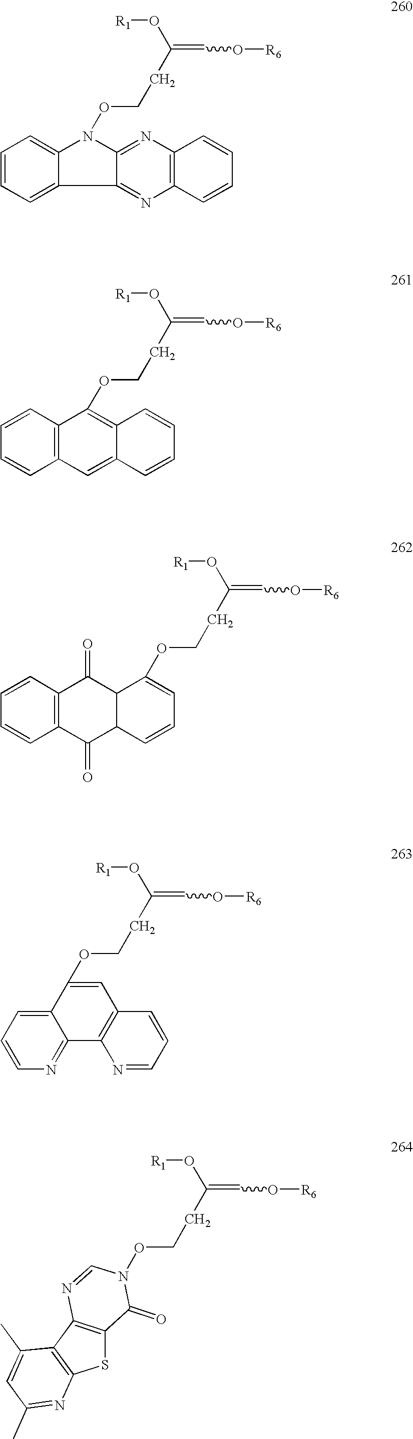

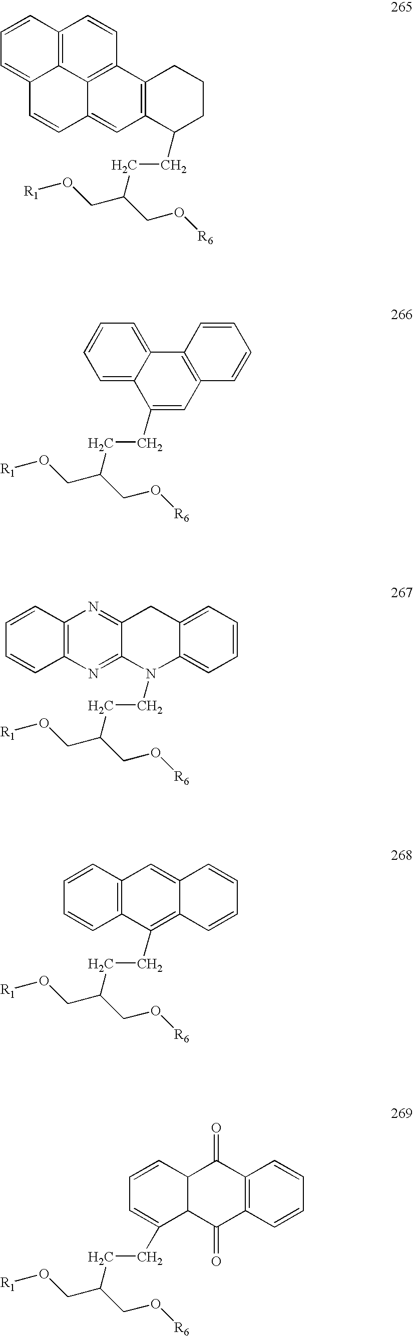

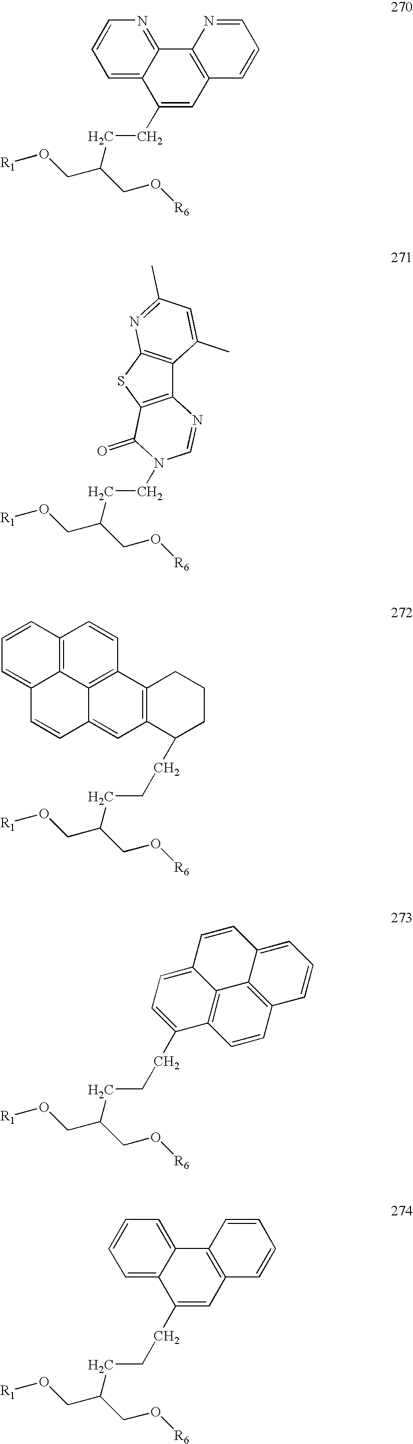

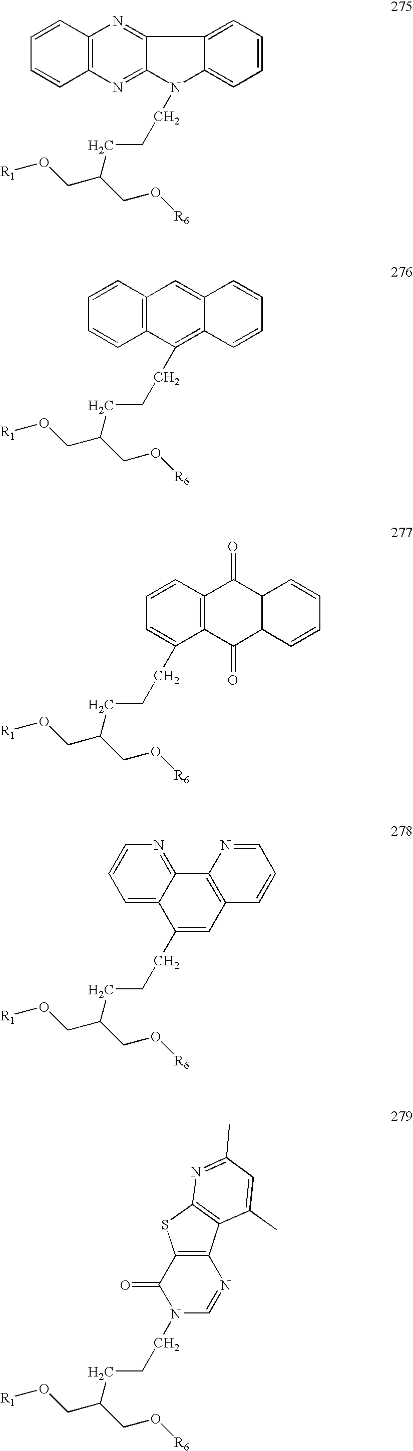

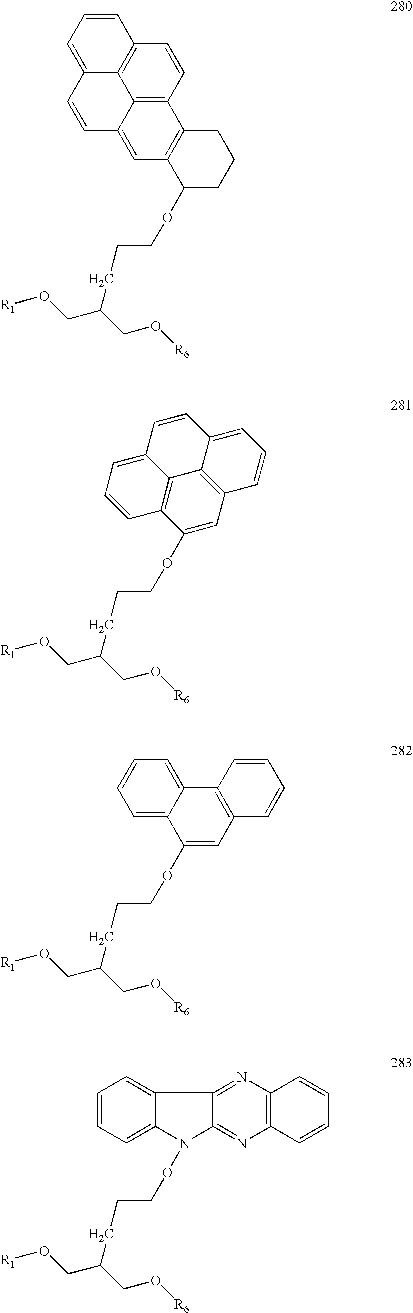

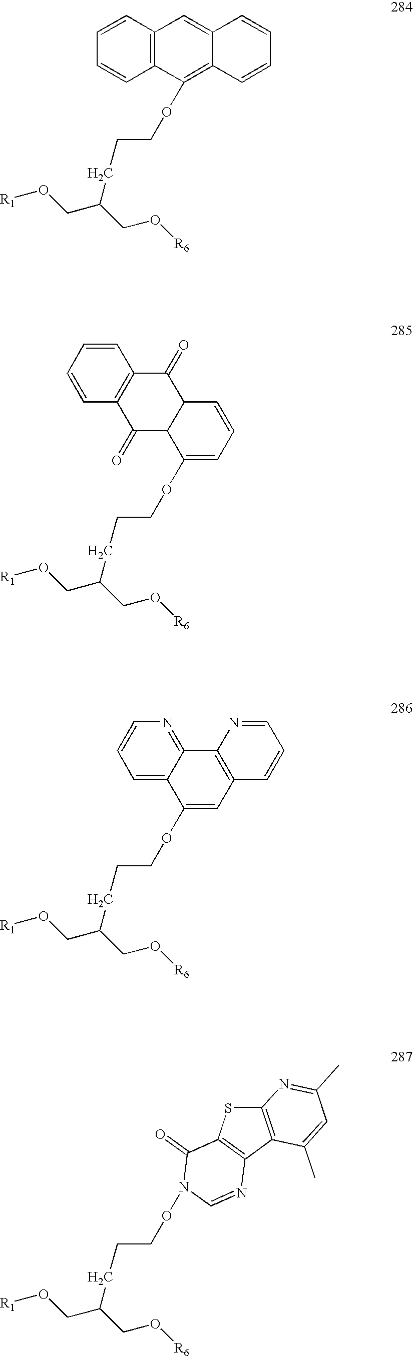

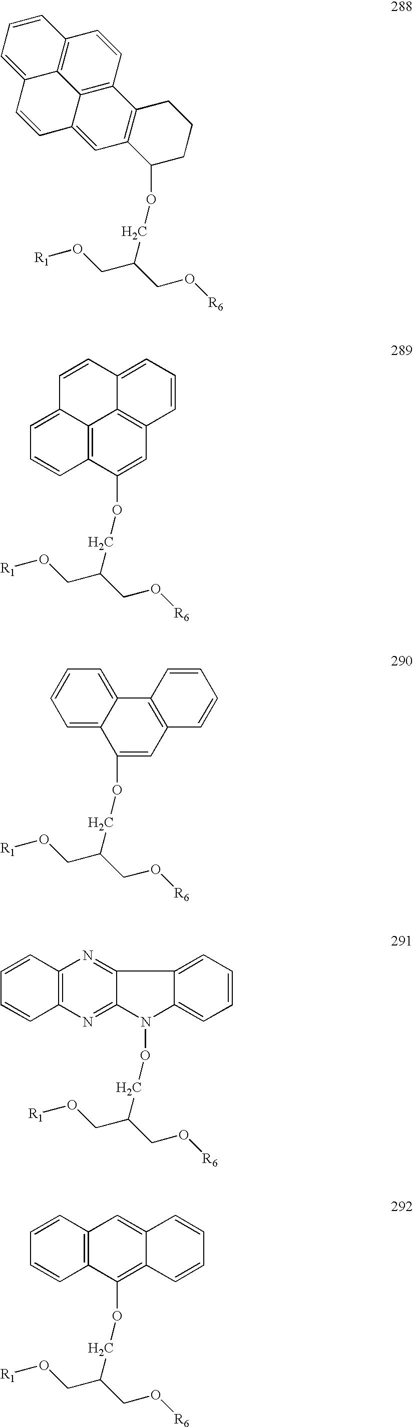

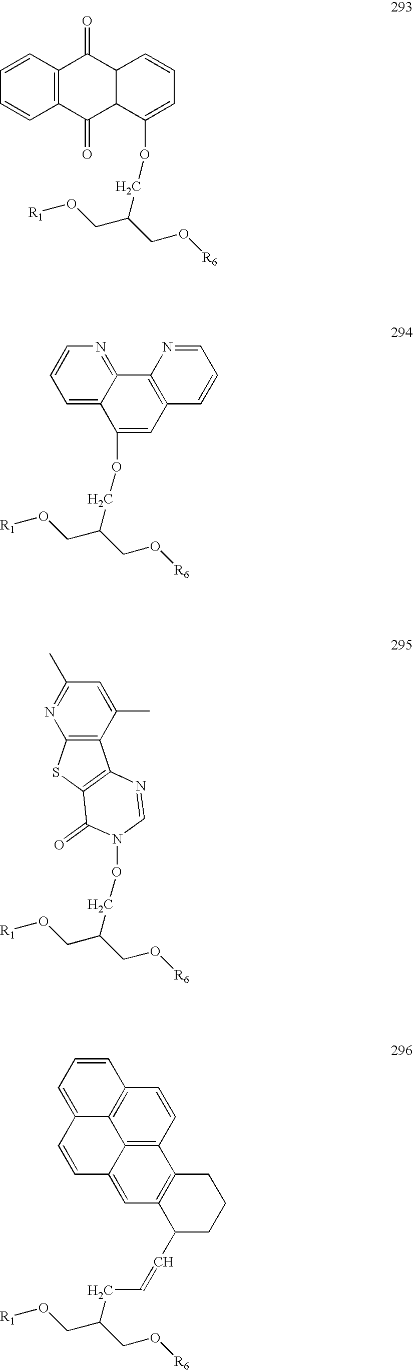

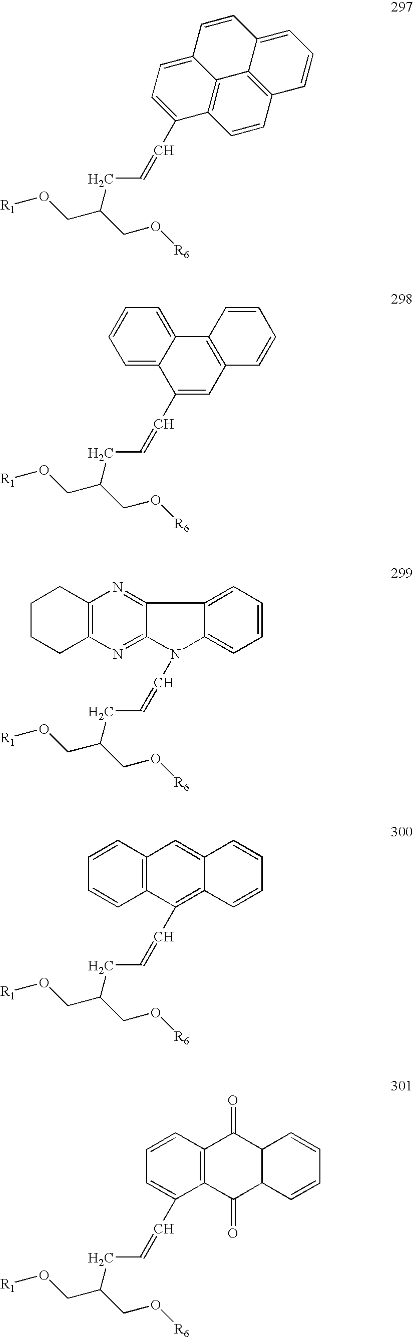

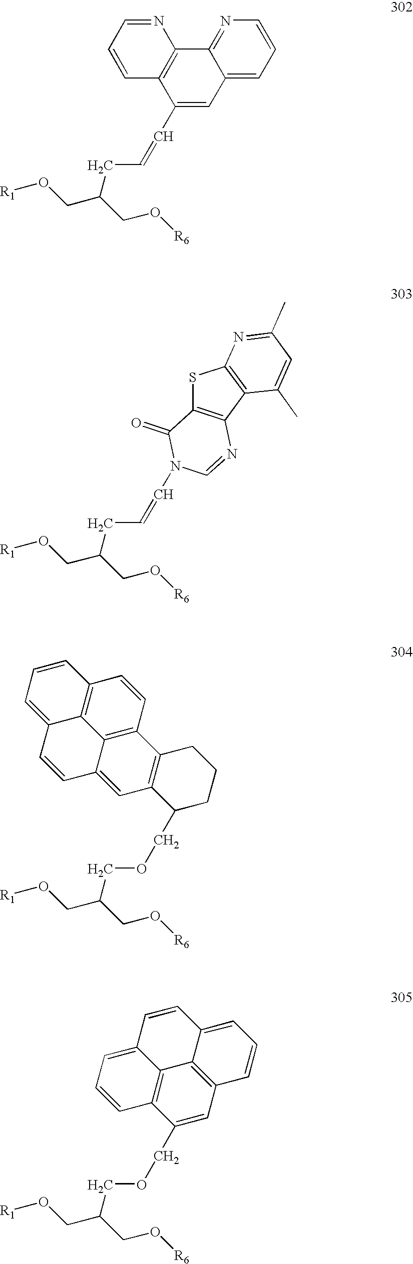

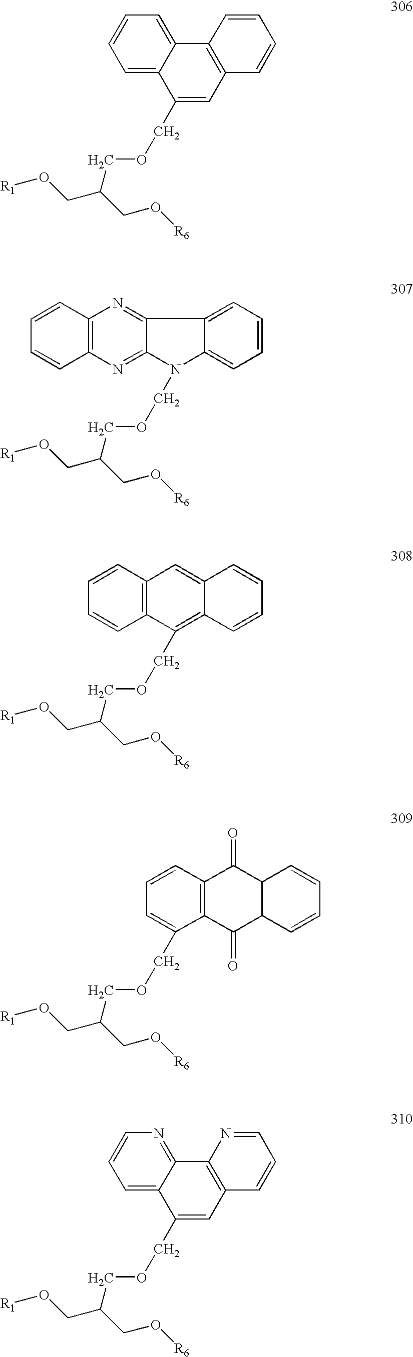

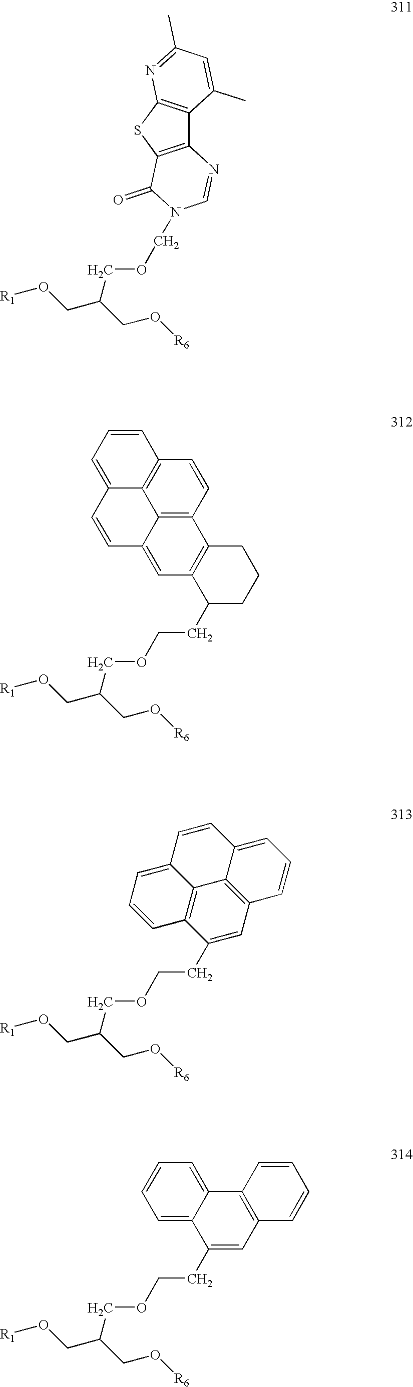

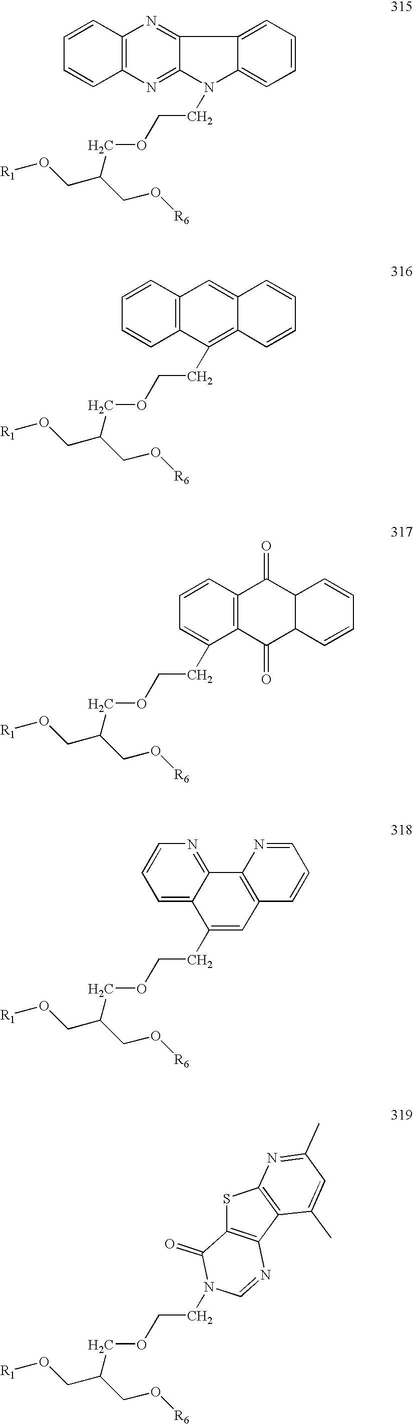

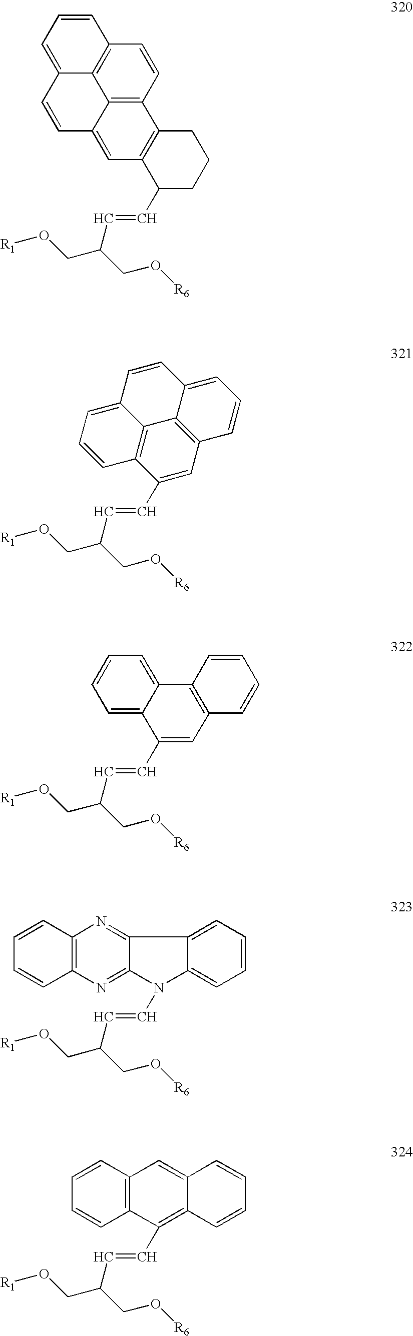

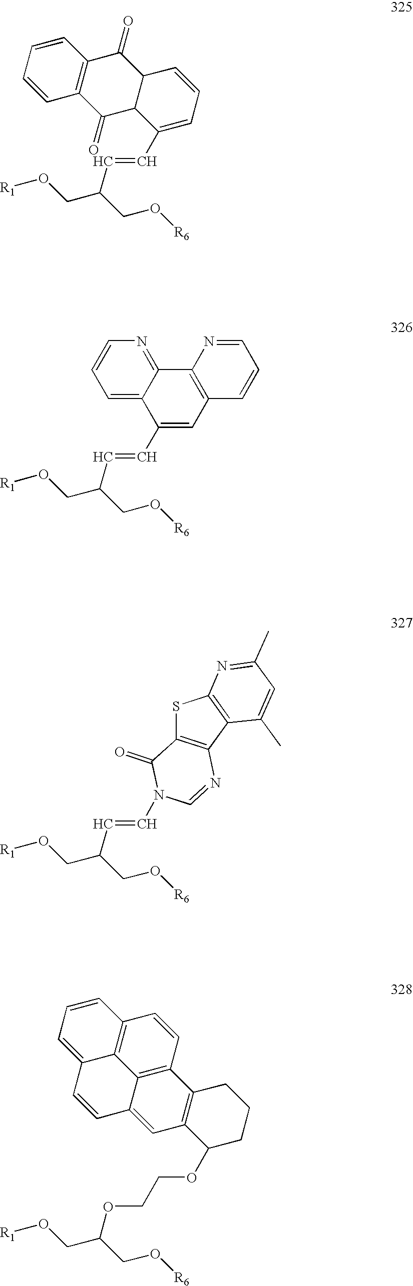

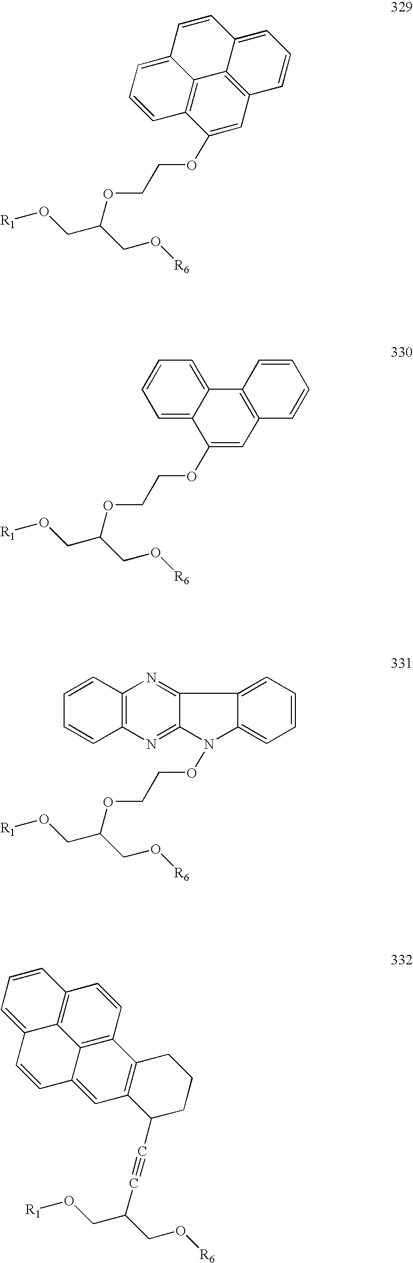

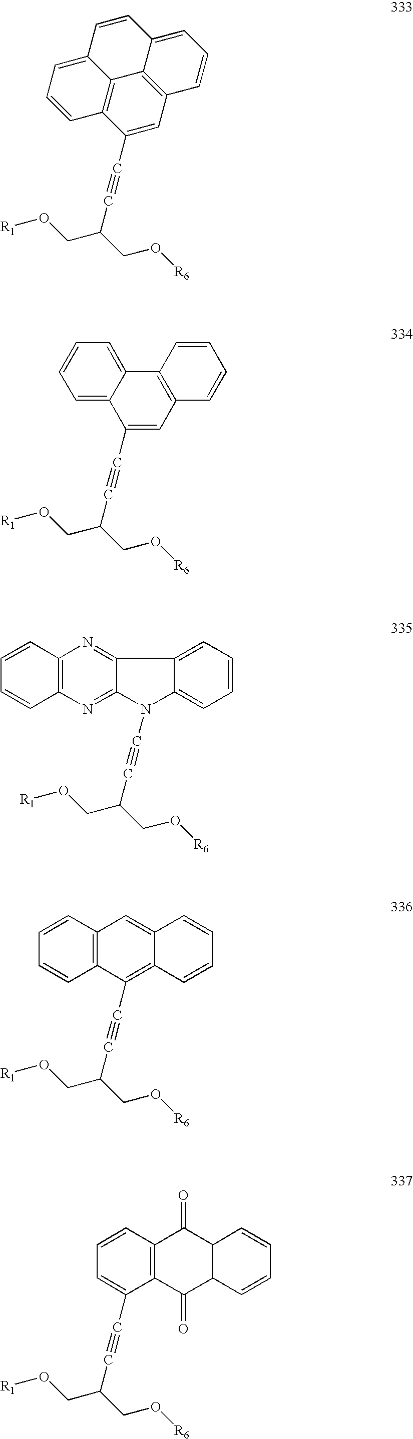

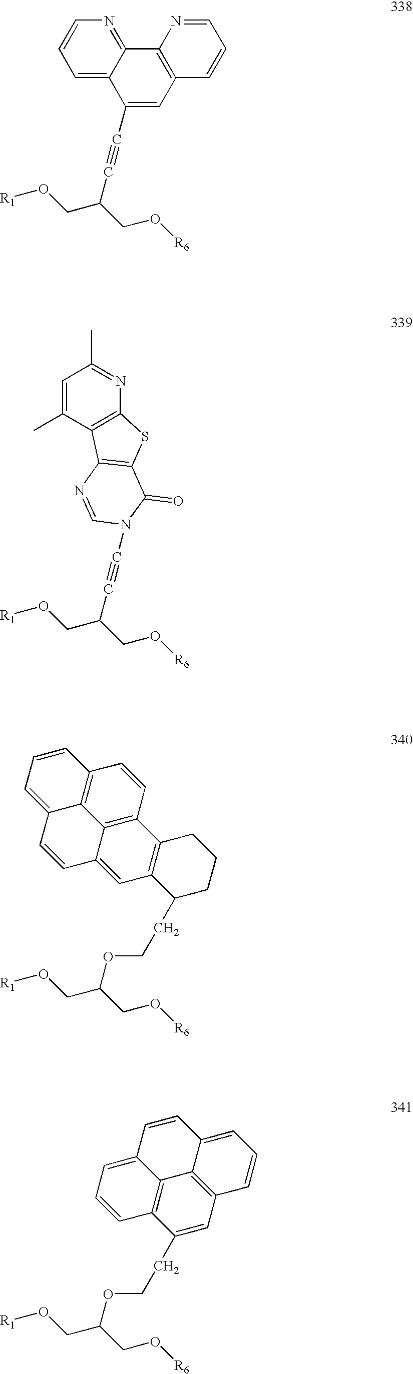

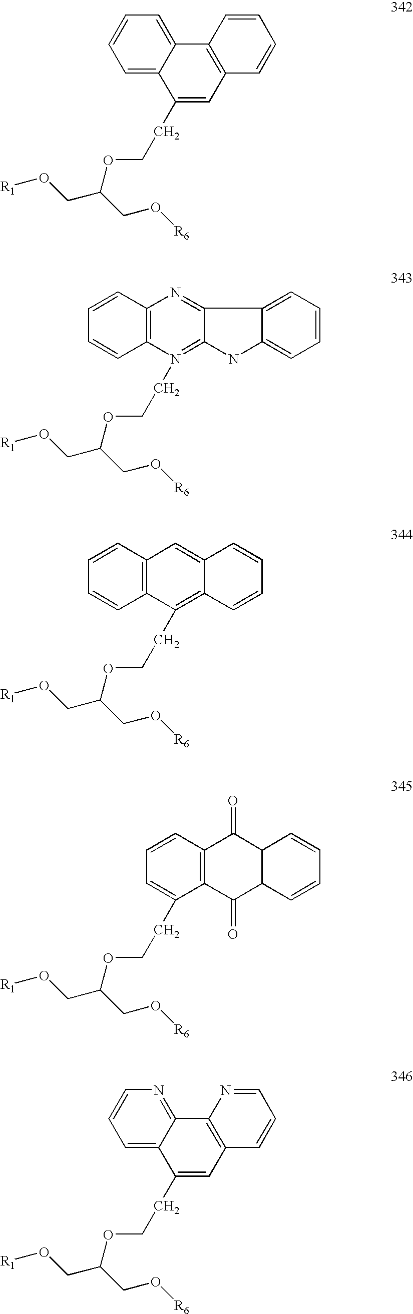

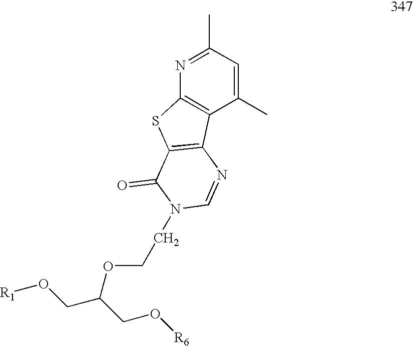

wherein DMT and (CH₂CH₂CN) functions as protecting groups.

In one preferred embodiment of the present invention the intercalator pseudonucleotide is selected from the group consisting of phosphoramidits of 1-(4,4′-dimethoxytriphenylmethyloxy)-3-pyrenemethyloxy-2-propanol. Even more preferably, the intercalator pseudonucleotide is selected from the group consisting of the phosphoramidit of (S)-1-(4,4′-dimethoxytriphenylmethyloxy)-3-pyrenemethyloxy-2-propanol and the phosphoramidit of (R)-1-(4,4′-dimethoxytriphenylmethyloxy)-3-pyrenemethyloxy-2-propanol.

Preparation of Intercalator Pseudonucleotides

The intercalator pseudonucleotides according to the present invention may be synthesised by any suitable method.

However preferably the method may comprise the steps of
a1) providing a compound containing an intercalator comprising at least one essentially flat conjugated system, which is capable of co-stacking with nucleobases of a nucleic acid and optionally a linker part coupled to a reactive group; and
b1) providing a linker precursor molecule comprising at least two reactive groups, said two reactive groups may optionally be individually protected; and
c1) reacting said intercalator with said linker precursor and thereby obtaining an intercalator-linker; and
d1) providing a backbone monomer precursor unit comprising at least two reactive groups, said two reactive groups may optionally be individually protected and/or masked) and optionally comprising a linker part; and
e1) reacting said intercalator-linker with said backbone monomer precursor and obtaining an intercalator-linker-backbone monomer precursor;

or
a2) providing a backbone monomer precursor unit comprising at least two reactive groups, said two reactive groups may optionally be individually protected and/or masked) and optionally comprising a linker part; and
b2) providing a linker precursor molecule comprising at least two reactive groups, said two reactive groups may optionally be individually protected; and
c2) reacting said monomer precursor unit with said linker precursor and thereby obtaining a backbone-linker; and
d2) providing a compound containing an intercalator comprising at least one essentially flat conjugated system, which is capable of co-stacking with nucleobases of a nucleic acid and optionally a linker part coupled to a reactive group; and
e2) reacting said intercalator with said backbone-linker and obtaining an intercalator-linker-backbone monomer precursor;

or
a3) providing a compound containing an intercalator comprising at least one essentially flat conjugated system, which is capable of co-stacking with nucleobases of a nucleic acid and a linker part coupled to a reactive group; and
b3) providing a backbone monomer precursor unit comprising at least two reactive groups, said two reactive groups may optionally be individually protected and/or masked), and a linker part; and
c3) reacting said intercalator-linker part with said backbone monomer precursor-linker and obtaining an intercalator-linker-backbone monomer precursor;

and
j) optionally protecting and/or de-protecting said intercalator-linker-backbone monomer precursor; and
k) providing a phosphor containing compound capable of linking two psedonucleotides, nucleotides and/or nucleotide analogues together; and
l) reacting said phosphorous containing compound with said intercalator-linker-backbone monomer precursor; and
m) obtaining an intercalator pseudonucleotide

Preferably, the intercalator reactive group is selected so that it may react with the linker reactive group. Hence, if the linker reactive group is a nucleophil, then preferably the intercalator reactive group is an electrophile, more preferably an electrophile selected from the group consisting of halo alkyl, mesyloxy alkyl and tosyloxy alkyl. More preferably the intercalator reactive group is chloromethyl. Alternatively, the intercalator reactive group may be a nucleophile group for example a nucleophile group comprising hydroxy, thiol, selam, amine or mixture thereof.

Preferably, the cyclic or non cyclic alkane may be a polysubstituted alkane or alkoxy comprising at least three linker reactive groups. More preferably the polysubstituted alkane may comprise three nucleophilic groups such as, but not limited to, an alkane triole, an aminoalkan diol or mercaptoalkane diol. Preferably the polysubstituted alkane contain one nucleophilic group that is more reactive than the others, alternatively two of the nucleophilic groups may be protected by a protecting group. More preferably the cyclic or non cyclic alkane is 2,2-dimethyl-4-methylhydroxy-1,3-dioxalan, even more preferably the alkane is D-α,β-isopropylidene glycerol.

Preferably, the linker reactive groups should be able to react with the intercalator reactive groups, for example the linker reactivegroups may be a nucleophile group for example selected from the group consisting of hydroxy, thiol, selam and amine, preferably a hyhroxy group. Alternatively the linker reactive group may be an electrophile group, for example selected from the group consisting of halogen, triflates, mesylates and tosylates. In a preferred embodiment at least 2 linker reactive groups may be protected by a protecting group.

The method may furthermore comprise a step of attaching a protecting group to one or more reactive groups of the intercalator-precursor monomer. For example a DMT group may be added by providing a DMT coupled to a halogen, such as Cl, and reacting the DMT-Cl with at least one linker reactive group. Accordingly, preferably at least one linker reactive group will be available and one protected. If this step is done prior to reaction with the phosphor comprising agent, then the phosphor comprising agent may only interact with one linker reactive group.

The phoshphor comprising agent may for example be a phosphoramidit, for example NC(CH₂)₂OP(Nprⁱ₂)₂ or NC(CH₂)₂OP(Nprⁱ₂)Cl. Preferably the phosphor comprising agent may be reacted with the intercalator-precursor in the presence of a base, such as N(et)₃, N(′pr)₂Et and CH₂Cl₂.

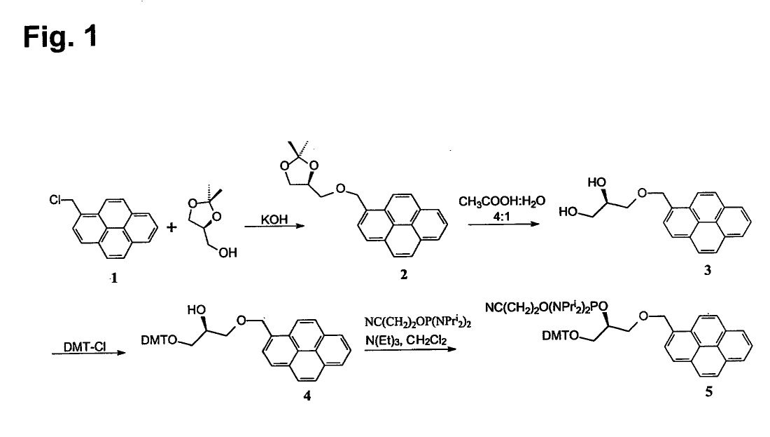

One specific non-limiting example of a method of synthesising an intercalator pseudonucleotide according to the present invention is outlined in example 1 and in FIG. 1.

Once the appropriate sequences of oligonucleotide or oligonucleotide analogue are determined, they are preferably chemically synthesised using commercially available methods and equipment: For example, the solid phase phosphoramidite method can be used to produce short oligonucleotide or oligonucleotide analogue comprising intercalator pseudonucleotides.

For example the oligonucleotides or oligonucleotide analogues may be synthesised by any of the methods described in “Current Protocols in Nucleic acid Chemistry” Volume 1, Beaucage et al., Wiley.

It is one objective of the present invention to provide methods of synthesising oligonucleotides or oligonucleotide analogues comprising at least one intercalator pseudonucleotide, wherein synthesis may comprise the steps of
a) bringing an intercalator pseudonucleotide according to the present invention into contact with a growing chain of a support-bound nucleotide, oligonucleotide, nucleotide analogue or oligonucleotide analogue; and
b) reacting said intercalator pseudonucleotide with said support-bound nucleotide, oligonucleotide, nucleotide analogue or oligonucleotide analogue; and
c) optionally capping unreacted said support-bound oligonucleotide; and
d) optionally further elongating said oligonucleotide analogue by adding one or more nucleotides, nucleotide analogues or intercalator pseudonucleotides to the oligonucleotide analogue in a desired sequence; and
e) cleaving said oligonucleotide analogue from said solid support; and
f) thereby obtaining said oligonucleotide analogue comprising at least one intercalator pseudonucleotide.

In one embodiment of the present invention the synthesis may comprise the steps of
a. bringing an intercalator pseudonucleotide according to the invention comprising a reactive group, which may be protected by an acid labile protection group into contact with a growing chain of a support-bound oligonucleotide or oligonucleotide analogue; and
b. reacting said intercalator pseudonucleotide with said support-bound oligonucleotide or oligonucleotide analogue; and
c. washing away excess reactants from product on the support; and
d. optionally capping unreacted said support-bound oligonucleotide; and
e. oxidizing the phosphite product to phosphate product; and
f. washing away excess reactants from product on support; and
g. optionally capping unreacted said support-bound oligonucleotide; and
h. repeating steps a)-g) until the desired number of intercalator pseudonucleotides are inserted; and
i. optionally elongating said support-bound oligonucleotide containing at least one intercalator pseudonucleotide; and
j. optionally repeating step a-i)
k. cleaving oligonucleotide analogue from solid support and removing base labile protecting groups in basic media; and
l. purifying oligonucleotide analogue containing acid labile protecting group; and
m. removing acid labile protecting group with acidic media; and
n. obtaining a terminus pseudonucleotide modified oligonucleotide analogue containing at least one intercalator pseudonucleotide

In another embodiment of the present invention the synthesis may comprise the steps of
a) bringing an intercalator pseudonucleotide according to the present invention into contact with an universal support; and
b) reacting said intercalator pseudonucleotide with the universal support; followed by step c) to j) as described in the method herein above.

It is also contained within the present invention that the last acid labile protection group may be removed prior to cleavage of the support-bound oligonucleotide analogue. Subsequent purification of the oligonucleotide analogue is optional.

In yet another embodiment of the present invention the method comprises the synthesis an oligonucleotide or oligonucleotide analogue comprising at least one internally positioned intercalator pseudonucleotide, wherein synthesis may comprise the steps of
a) bringing a nucleotide or nucleotide analogue protected with an acid labile protection group into contact with a growing chain of a support-bound nucleotide, oligonucleotide, nucleotide analogue or oligonucleotide analogue; and
b) reacting the protected nucleotide analogue with the growing chain of said support-bound nucleotide, oligonucleotide, nucleotide analogue or oligonucleotide analogue; and
c) washing away excess reactants from product on support; and
d) optionally capping unreacted said support-bound nucleotide; and
e) oxidizing the phosphite product to phosphate product; and
f) washing away excess reactants from product on support; and
g) optionally capping unreacted said support-bound nucleotide; and
h) removing acid labile protecting group; and
i) washing away excess reactants from product on the support; and
j) repeating steps a)-f) to obtain the desired oligonucleotide analogue sequence; and
k) cleaving the oligonucleotide analogue from solid support and removing base labile protecting groups in basic media; and
l) purifying oligonucleotide containing acid labile protecting group; and
m) removing acid labile protecting group; and
n) obtaining an intercalator modified oligonucleotide analogue.

Alternatively the last acid labile protection group may be removed prior to cleavage of the support-bound oligonucleotide analogue. Purification of the oligonucleotide analogue is optional.

Oligonucleotides Comprising Intercalator Pseudonucleotides

One objective of the present invention is to provide oligonucleotides or oligonucleotide analogues comprising at least one intercalator pseudonucleotide as described herein above. For example, the present invention relates to oligonucleotides or oligonucleotide analogues synthesised by any of the methods described herein above or any other method known to the person skilled in the art.

High affinity of synthetic nucleic acids towards target nucleic acids may greatly facilitate detection assays and furthermore synthetic nucleic acids with high affinity towards target nucleic acids may be useful for a number of other purposes, such as gene targeting and purification of nucleic acids. Oligonucleotides or Oligonucleotide analogues comprising intercalators have been shown to increase affinity for homologously complementary nucleic acids.

Accordingly it is an object of the present invention to provide oligonucleotides or oligonucleotide analogues comprising at least one intercalator pseudonucleotide wherein the melting temperature of a hybrid consisting of said oligonucleotides or oligonucleotide analogues and a homologously complementary DNA (DNA hybrid) is significantly higher than the melting temperature of a hybrid between an oligonucleotide or oligonucleotide analogue lacking intercalator pseudonucleotide(s) consisting of the same nucleotide sequence as said oligonucleotide or oligonucleotide analogue and said homologously complementary DNA (corresponding DNA hybrid).

Preferably, the melting temperature of the DNA hybrid is from 1 to 80° C., more preferably at least 2° C., even more preferably at least 5° C., yet more preferably at least 10° C. higher than the melting temperature of the corresponding DNA hybrid.

The present invention may also provide oligonucleotides or oligonucleotide analogues comprising at least one internal intercalator pseudonucleotide. Positioning intercalator units internally allows for greater flexibility in design. Nucleic acid analogues comprising internally positioned intercalator pseudonucleotides may thus have higher affinity for homologously complementary nucleic acids than nucleic acid analogues that does not have internally positioned intercalator pseudonucleotides. Oligonucleotides or Oligonucleotide analogues comprising at least one internal intercalator pseudonucleotide may also be able to discriminate between RNA (including RNA-like nucleic acid analogues) and DNA (including DNA-like nucleic acid analogues). Furthermore internally positioned fluorescent intercalator monomers could find use in diagnostic tools.

For example such oligonucleotide analogues may comprise 1, such as 2, for example 3, such as 4, for example 5, such as from 1 to 5, such as, for example from 5 to 10, such as from 10 to 15, for example from 15 to 20, such as more than 20 intercalatorpseudonucleotides.

In one embodiment the oligonucleotide or oligonucleotide analogue comprises at least 2 intercalator pseudonucleotides.

The intercalator pseudonucleotides may be placed in any desirable position within a given oligonucleotide or oligonucleotide analogue. For example, an intercalator pseudonucleotide may be placed at the end of the oligonucleotide or oligonucleotide analogue or an intercalator pseudonucleotide may be placed in an internal position within the oligonucleotide or oligonucleotide analogue.

When the oligonucleotide or oligonucleotide analogue comprise more than 1 intercalator pseudonucleotide, the intercalator pseudonucleotides may be placed in any position in relation to each other. For example they may be placed next to each other, or they may be positioned so that 1, such as 2, for example 3, such as 4, for example 5, such as more than 5 nucleotides are separating the intercalator pseudonucleotides. In one preferred embodiment two intercalator pseudonucleotides within an oligonucleotide or oligonucleotide analogue are placed as next nearest neighbours, i.e. they can be placed at any position within the oligonucleotide or oligonucleotide analogue and having 1 nucleotide separating said two intercalator pseudonucleotides. In another preferred embodiment two intercalators are placed at or in close proximity to each end respectively of said oligonucleotide or oligonucleotide analogue.

The oligonucleotides or oligonucleotide analogues may comprise any kind of nucleotides and/or nucleotide analogues, such as the nucleotides and/or nucleotide analogues described herein above. For example, the oligonucleotides or oligonucleotide analogues may comprise nucleotides and/or nucleotide analogues comprised within DNA, RNA, LNA, PNA, ANA and HNA. Accordingly, the oligonucleotides or oligonucleotide analogue may comprise one or more selected from the group consisting of subunits of PNA, Homo-DNA, b-D-Altropyranosyl-NA, b-D-Glucopyranosyl-NA, b-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, 2′-OR-RNA, α-L-RNA, α-D-RNA, β-D-RNA, i.e. the oligonucleotide analogue may be selected from the group of PNA, Homo-DNA, b-D-Altropyranosyl-NA, b-D-Glucopyranosyl-NA, b-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, 2′-OR-RNA, α-L-RNA, α-D-RNA, β-D-RNA and mixtures thereof.

One advantage of the oligonucleotides or oligonucleotide analogues according to the present invention is that the melting temperature of a hybrid consisting of an oligonucleotide or oligonucleotide analogue comprising at least one intercalator pseudonucleotide and an essentially complementary DNA (DNA hybrid) is significantly higher than the melting temperature of a duplex consisting of said essentially complementary DNA and a DNA complementary thereto.

Accordingly, oligonucleotides or oligonucleotide analogues according to the present invention may form hybrids with DNA with higher affinity than naturally occurring nucleic acids. The melting temperature is preferably increased with 2 to 30° C., for example from 5 to 20° C., such as from 10° C. to 15° C., for example from 2° C. to 5° C., such as from 5° C. to 10° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C. higher.

In particular, the increase in melting temperature may be achieved due to intercalation of the intercalator, because said intercalation may stabilise a DNA duplex. Accordingly, it is preferred that the intercalator is capable of intercalating between nucleobases of DNA: Preferably, the intercalator pseudonucleotides are placed as a bulge insertions or end insertions in the duplex (see herein below), which in some nucleic acids or nucleic acid analogues may allow for intercalation.

In one particular embodiment of the present invention the melting temperature of an oligonucleotide or oligonucleotide analogue comprising at least one intercalator pseudonucleotide and an essentially complementary RNA (RNA hybrid) or a RNA-like nucleic acid analogue (RNA-like hybrid) is significantly higher than the melting temperature of a duplex consisting of said essentially complementary RNA or RNA-like target and said oligonucleotide analogue comprising no intercalator pseudonucleotides. Preferably most or all of the intercalator pseudonucleotides of said oligonucleotide or oligonucleotide analogue are positioned at either or both ends.

Accordingly, oligonucleotides and/or oligonucleotide analogues according to the present invention may form hybrids with RNA or RNA-like nucleic acid analogues or RNA-like oligonucleotide analogues with higher affinity than naturally occurring nucleic acids. The melting temperature is preferably increased with from 2 to 20° C., for example from 5 to 15° C., such as from 10° C. to 15° C., for example from 2° C. to 5° C., such as from 5° C. to 10° C., such as from 15° C. to 20° C. or higher.

Said embodiment is particular in the sense that intercalator pseudonucleotides will preferably only stabilise towards RNA and RNA-like targets when positioned at the end of said oligonucleotide or oligonucleotide analogue. This does however not exclude the positioning of intercalator pseudonucleotides in oligonucleotides or oligonucleotide analogues to be hybridised with RNA or RNA-like nucleic acid analogues such that said intercalator pseudonucleotides are placed in regions internal to the formed hybrid. This may be done to obtain certain hybrid instabilities or to affect the overall 2D or 3D structure of both intra- and inter-molecular complexes to be formed subsequent to hybridisation.

In another embodiment of the present invention an oligonucleotide and/or oligonucleotide analogue comprising one or more intercalator pseudonucleotides according to the present invention may form a triple stranded structure (triplex-structure) consisting of said oligonucleotide and/or oligonucleotide analogue bound by Hoogstein base pairing to a homologously complementary nucleic acid or nucleic acid analogue or oligonucleotide or oligonucleotide analogue.

In another preferred embodiment of the present invention said oligonucleotide or oligonucleotide analogue may increase the melting temperature of said Hoogstein base pairing in said triplex-structure.

In another even more preferred embodiment of the present invention said oligonucleotide or oligonucleotide analogue may increase the melting temperature of said Hoogstein base pairing in said triplex-structure in a manner not dependent on the presence of specific sequence restraints like purine-rich•pyrimidine-rich nucleic acid or nucleic acid analogue duplex target sequences. Accordingly, said Hoogstein basepairing in said triplex-structure has significantly higher melting temperature than the melting temperature of said Hoogstein basepairing to said duplex target if said oligonucleotide or oligonucleotide analogue had no intercalator pseudonucleotides.

Accordingly, oligonucleotides or oligonucleotide analogues according to the present invention may form triplex-structures with homologously complementary nucleic acid or nucleic acid analogue or oligonucleotide or oligonucleotide analogue with higher affinity than naturally occurring nucleic acids. The melting temperature is preferably increased with from 2-50° C., such as from 2-40° C., such as from 2 to 30° C., for example from 5 to 20° C., such as from 10° C. to 15° C., for example from 2° C. to 5° C., such as from 5° C. to 10° C., for example from 10° C. to 15° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C.

In particular, the increase in melting temperature may be achieved due to intercalation of the intercalator, because said intercalation may stabilise a DNA triplex. Accordingly, it is preferred that the intercalator is capable of intercalating between nucleobases of a triplex-structure. Preferably, the intercalator pseudonucleotide is placed as a bulge insertion in the duplex (see herein below), which in some nucleic acids or nucleic acid analogues may allow for intercalation.

Triplex-formation may or may not proceed in strand invasion, a process where the Hoogstein base-paired third strand invades the target duplex and displaces part or all of the identical strand to form Watson-Crick base pairs with the complementary strand. This can be exploited for several purposes.

The oligonucleotides and oligonucleotides according to the invention are suitably used for if only double stranded nucleic acid or nucleic acid analogue target is present and it is not possible, feasible or wanted to separate said target strands, detection by single strand invasion of the region or double strand invasion of complementary regions, without prior melting of double stranded nucleic acid or nucleic acid analogue target, for triplex-formation and/or strand invasion.

Accordingly, in one embodiment of the present invention an oligonucleotide or oligonucleotide analogue comprising at least one intercalator pseudonucleotide is provided that is able to invade a double stranded region of a nucleic acid or nucleic acid analogue molecule.

In a more preferred embodiment of the present invention an oligonucleotide or oligonucleotide analogue comprising at least one intercalator pseudonucleotide that is able to invade a double stranded nucleic acid or nucleic acid analogue in a sequence specific manner is provided.

In a further embodiment of the present invention, said invading oligonucleotide and/or oligonucleotide analogue comprising at least one intercalator pseudonucleotide will bind to the complementary strand in a sequence specific manner with higher affinity than the strand displaced.

In one embodiment of the present invention the melting temperature of a hybrid consisting of an oligonucleotide analogue comprising at least one intercalator pseudonucleotide and a homologously complementary DNA (DNA hybrid), is significantly higher than the melting temperature of a hybrid consisting of said oligonucleotide or oligonucleotide analogue and a homologously complementary RNA (RNA hybrid) or RNA-like nucleic acid analogue target or RNA-like oligonucleotide analogue target.

Said oligonucleotide may be any of the above described oligonucleotide analogues. For example, the oligonucleotide may be a DNA oligonucleotide (analogue) comprising at least one intercalator pseudonucleotide or a Homo-DNA, b-D-Altropyranosyl-NA, b-D-Glucopyranosyl-NA, b-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, 2′-OR-RNA, α-L-RNA, α-D-RNA, β-D-RNA oligonucleotide or mixtures hereof comprising at least one intercalator pseudonucleotide.

Accordingly, the affinity of said oligonucleotide or oligonucleotide analogue for DNA is significantly higher than the affinity of said oligonucleotide or oligonucleotide analogue for RNA or an RNA-like target. Hence in a mixture comprising a limiting number of said oligonucleotide or oligonucleotide analogue and a homologously complementary DNA and a homologously complementary RNA or homologously complementary RNA-like target, the oligonucleotide or oligonucleotide analogue will preferably hybridise to said homologously complementary DNA.

Preferably, the melting temperature of the DNA hybrid is at least 2° C., such as at least 5° C., for example at least 10° C., such as at least 15° C., for example at least 20° C., such as at least 25° C., for example at least 30° C., such as at least 35° C., for example at least 40° C., such as from 2 to 30° C., for example from 5 to 20° C., such as from 10° C. to 15° C., for example from 2° C. to 5° C., such as from 5° C. to 10° C., for example from 10° C. to 15° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C., for example from 50° C. to 55° C., such as from 55° C. to 60° C. higher than the melting temperature of a homologously complementary RNA or RNA-like hybrid.

In a preferred embodiment of the present invention an oligonucleotide or oligonucleotide analogue containing at least one intercalator pseudonucleotide is hybridized to secondary structures of nucleic acids or nucleic acid analogues. In a more preferred embodiment said oligonucleotide or oligonucleotide analogue is capable of stabilizing such a hybridization to said secondary structure. Said secondary structures could be, but are not limited to stem-loop structures, Faraday junctions, fold-backs, H-knots, and bulges. In a special embodiment the secondary structure is a stem-loop structure of RNA, where an oligonucleotide or oligonucleotide analogue comprising at least one intercalator pseudonucleotide is designed in a way so said intercalator pseudonucleotide is hybridizing at the end of one of the three duplexes formed in the three-way junction between said secondary structure and said oligonucleotide or oligonucleotide analogue.

Position of Intercalator Pseudonecleotide.

In a preferred embodiment of the present invention an oligonucleotide or oligonucleotide analogue is designed in a manner so it may hybridise to a homologously complementary nucleic acid or nucleic acid analogue (target nucleic acid). Preferably, the oligonucleotide or oligonucleotide analogue may be substantially complementary to the target nucleic acid. More preferably, at least one intercalator pseudonucleotide is positioned so that when the oligonucleotide analogue is hybridised with the target nucleic acid, the intercalator pseudonucleotide is positioned as a bulge insertion, i.e. the upstream neighbouring nucleotide of the intercalator pseudonucleotide and the downstream neighbouring nucleotide of the analogue comprising at least one intercalator pseudonucleotide and a second sequence capable of hybridising to said first sequence. In one embodiment said second sequence does not comprise any intercalator pseudonucleotides.

Hence, the present invention relates to a pair of corresponding oligonucleotides or oligonucleotide analogues, wherein one oligonucleotide analogue of the pair is designated first sequence and the other oligonucleotide analogue of the pair is designated second sequence, and wherein said pair of oligonucleotides or oligonucleotide analogues comprises at least one intercalator pseudonucleotide.

Preferably, the pair of oligonucleotide or oligonucleotide analogue sequences (designated first sequence and second sequence) comprises a first sequence capable of hybridising with the second sequence. It is furthermore preferred that both the first sequence and the second sequence comprises at least one intercalator pseudonucleotide.

In one embodiment of the present invention, the first sequence is essentially complementary to the second sequence. Furthermore, in that embodiment it is preferred that the first sequence has essentially the same length as the second sequence.

It is preferred that the melting temperature of a hybrid between first sequence and the second sequence is significantly lower than the melting temperature of a hybrid between the first sequence and a corresponding nucleic acid or nucleic acid analogue selected as defined above. In particular, it is preferred that the melting temperature of a hybrid of the first sequence and the second sequence is significantly lower than the melting temperature of a hybrid between the first sequence and a corresponding DNA.

In particular, if the second sequence is complementary to the first sequence and the first sequence and the second sequence are of equal length, it is preferred that the melting temperature of a hybrid of the first sequence and the second sequence is significantly lower than the melting temperature of a hybrid between the second sequence and a corresponding nucleic acid or nucleic acid analogue as defined above. In particular, it is preferred that the melting temperature of a hybrid of the first intercalator pseudonucleotide are hybridised to neighbouring nucleotides in the target nucleic acid.

In another preferred embodiment an intercalator pseudonucleotide is positioned next to either or both ends of a duplex formed between the oligonucleotide analogue comprising said intercalator pseudonucleotide and its target nucleotide or nucleotide analogue, for example said intercalator pseudonucleotide may be positioned as a dangling, co-stacking end.

Even more preferably, all intercalator pseudonucleotides of an oligonucleotide or oligonucleotide analogue are positioned so that when the oligonucleotide analogue is hybridised with the target nucleic acid, all intercalator pseudonucleotides are positioned as bulge insertions and/or as dangling, co-stacking ends.

In one embodiment the present invention relates to the following oligonucleotides:

N₁—(P)_{q}—N₂,

N₁—(P—N₃)_{q}—N₂,

(P)_{q}—N₂,

N₁—(P)_{q},

(P)_{q}—N₂—(P)ᵣ,

N₁—(P)_{q}—N₂,

N₁—(P—N₃)_{q}—N₂—(P—N₃)ᵣ—N₄,

wherein

N₁, N₂, N₃, N₄ individually denotes a sequence of nucleotides and/or nucleotides analogues of at least one nucleotide,

P denotes an intercalator pseudonucleotide, and

q and r are individually selected from an integer of from 1-10.

A Pair of Oligonucleotide Oligonucleotide and/or Analogue Sequences

The invention also relates to a pair of oligonucleotides or oligonucleotide analogues comprising a first sequence, which is an oligonucleotide and/or oligonucleotide sequence and the second sequence is significantly lower than the melting temperature of a hybrid between the second sequence and an essentially complementary DNA.

However it is possible when the second sequence is complementary to only a part of the first sequence that the melting temperature of the hybrid between the first and second sequence can be higher, equal to or lower than the melting temperature of a hybrid between the second sequence and an essentially complementary DNA.

When the melting temperature of a hybrid of the first sequence and the second sequence is significantly lower than the melting temperature of a hybrid between the first sequence and a corresponding DNA, this results in the advantageous effect that in a mixture comprising the first sequence, the second sequence and a DNA corresponding to the first sequence, the first sequence will preferably hybridize with the corresponding DNA, rather than with the second sequence.

Analogously, if the second sequence is complementary to the first sequence and the first sequence and the second sequence are of equal length, in a mixture comprising the first sequence, the second sequence and a DNA corresponding to the second sequence, the second sequence will preferably hybridize with the corresponding DNA rather than with the first sequence. However, in a mixture comprising the first sequence and the second sequence, but no corresponding nucleic acid or nucleic acid analogue which does not comprise intercalator pseudonucleotides, the first sequence will hybridize to the second sequence. If the second sequence is complementary to only a part of the first sequence, the melting temperature of the hybrid between first and second sequence can be either higher, equal to or lower than the melting temperature of a hybrid between the second sequence and an essentially complementary DNA.

Accordingly, in a mixture comprising the first sequence and the second sequence, if the first sequence and the second sequence are hybridized, this is indicative of the fact that only a limiting amount of corresponding target nucleic acids is available.

Vice versa, in a mixture comprising the first sequence and the second sequence, if the first sequence and the second sequence are not hybridized, this is indicative of the fact that the mixture furthermore comprises corresponding target nucleic acids.

Preferably said corresponding nucleic acid or nucleic acid analogue, which does not comprise said intercalator pseudonucleotides, is DNA.

The melting temperature is dependent on a number of features, for example the melting temperature is dependent on the amount of intercalator pseudonucleotides, on the kind of intercalator pseudonucleotides, on the length of the first sequence and/or second sequence, on the nucleobase composition, on the position of these intercalator pseudonucleotides within the pair of oligonucleotide or oligonucleotide analogue sequences and on the position of intercalator pseudonucleotides in relation to one another.

Preferably, the above-mentioned features are all selected in order to ensure specific binding to corresponding target nucleic acid.

Accordingly, the first nucleotide sequence preferably comprises between 5 and 10, such as between 10 and 15, for example between 15 and 20, such as between 20 and 30, for example between 30 and 50 nucleotides and/or nucleotide analogues and/or intercalator pseudonucleotide. More preferably, the first nucleotide sequence consists of between 5 and 10, such as between 10 and 15, for example between 15 and 20, such as between 20 and 30, for example between 30 and 50 nucleotides and/or nucleotide analogues and/or intercalator pseudonucleotides.

Furthermore, the second nucleotide sequence preferably comprises between 5 and 10, such as between 10 and 15, for example between 15 and 20, such as between 20 and 30, for example between 30 and 50 nucleotides and/or nucleotide analogues and/or intercalator pseudonucleotides. More preferably, the second nucleotide sequence consists of between 5 and 10, such as between 10 and 15, for example between 15 and 20, such as between 20 and 30, for example between 30 and 50 nucleotides and/or nucleotide analogues and/or intercalator pseudonucleotides.

In a preferred embodiment the first nucleotide sequence and the second nucleotide sequence consist of the same number of nucleotides and/or nucleotide analogues and/or intercalator pseudonucleotides.

In addition each of the oligonucleotide analogues of said pair may individually consist of between 5 and 100 nucleotides and/or nucleotide analogues and/or intercalator pseudonucleotides, preferably, the oligonucleotide or oligonucleotide analogue may consist of between 10 and 75 nucleotides and/or nucleotide analogues and/or intercalator pseudonucleotides, more preferably, the oligonucleotide or oligonucleotide analogue may consist of between 15 and 50 nucleotides and/or nucleotide analogues and/or intercalator pseudonucleotides.

The first sequence should comprise at least one intercalator pseudonucleotide, for example 2, such as 3, for example 4, such as 5, for example 6, such as from 6 to 10, for example from 10 to 15, such as from 15 to 20 intercalator pseudonucleotides.

The second sequence may or may not comprise intercalator pseudonucleotide(s). In one preferred embodiment of the invention and in particular when the second sequence is complementary to the first sequence and of equal length therewith, it is preferred that the second sequence comprises at least one intercalator pseudonucleotide, such as 1, for example 2, such as 3, for example 4, such as 5, for example 6, such as from 6 to 10, for example from 10 to 15, such as from 15 to 20 intercalator pseudonucleotides.

The intercalator pseudonucleotides may be placed in any desirable position within the first and/or second sequence. For example intercalator pseudonucleotides may be placed at the end of the first and/or second sequence or intercalator pseudonucleotide(s) may be placed in an internal position within the first and/or second sequence.

Furthermore, if the first sequence comprises more than one intercalator pseudonucleotide, said pseudonucleotides may be placed in relation to each other in any desirable manner. For example, they may be placed so that 1, for example 2, such as 3, for example 4, such as 5, for example from 5 to 10, such as from 10 to 15, for example from 15 to 20, such as more than 20 nucleotides are separating the intercalator pseudonucleotides.

Analogously, if the second sequence comprises more than one intercalator pseudonucleotide, said pseudonucleotides may be placed in relation to each other in any desirable manner. For example, they may be placed so that 1, for example 2, such as 3, for example 4, such as 5, for example from 5 to 10, such as from 10 to 15, for example from 15 to 20, such as more than 20 nucleotides are separating the intercalator pseudonucleotides.

In a preferred embodiment at least two intercalator pseudonucleotides are placed in relation to each other within the first sequence and/or second sequence in order to obtain high selectivity and affinity for corresponding target nucleic acid. Accordingly, preferably the intercalator pseudonucleotides are placed so that the oligonucleotide analogue preferably hybridises with the corresponding target sequence rather than with any other nucleic acid sequence, including single point mutations of said corresponding target nucleic acid. For example, in one embodiment of the present invention, the intercalator pseudonucleotides may be positioned as next nearest neighbours.

The first and/or second sequence may individually comprise more than one intercalator pseudonucleotide, wherein said intercalator pseudonucleotides may be similar or said intercalator pseudonucleotides may be different.

Especially if the second sequence is complementary to the first sequence and of equal length therewith, it is of importance how the intercalator pseudonucleotides within the first sequence are positioned in relation to intercalator pseudonucleotides within the second sequence. Preferably, when the first sequence is hybridized with the second sequence at least one intercalator pseudonucleotide within the first sequence is placed in such a manner that it is positioned opposite of a nucleotide substitute within the second sequence that cannot form Watson-Crick hydrogen bonds. In addition, preferably at least one intercalator pseudonucleotide of the second sequence is placed in such a manner that it is positioned opposite of a nucleotide substitute of the first sequence that cannot form Watson-Crick hydrogen bonds when the first sequence is hybridized with the second sequence.

Said nucleotide substitute may for example be a nucleotide lacking the nucleobase or a nucleotide comprising a nucleobase, which has been modified in a manner so that it can no longer form Watson-Crick hydrogen bonds. However, in a preferred embodiment the nucleotide substitute is another intercalator pseudonucleotide as described herein above.

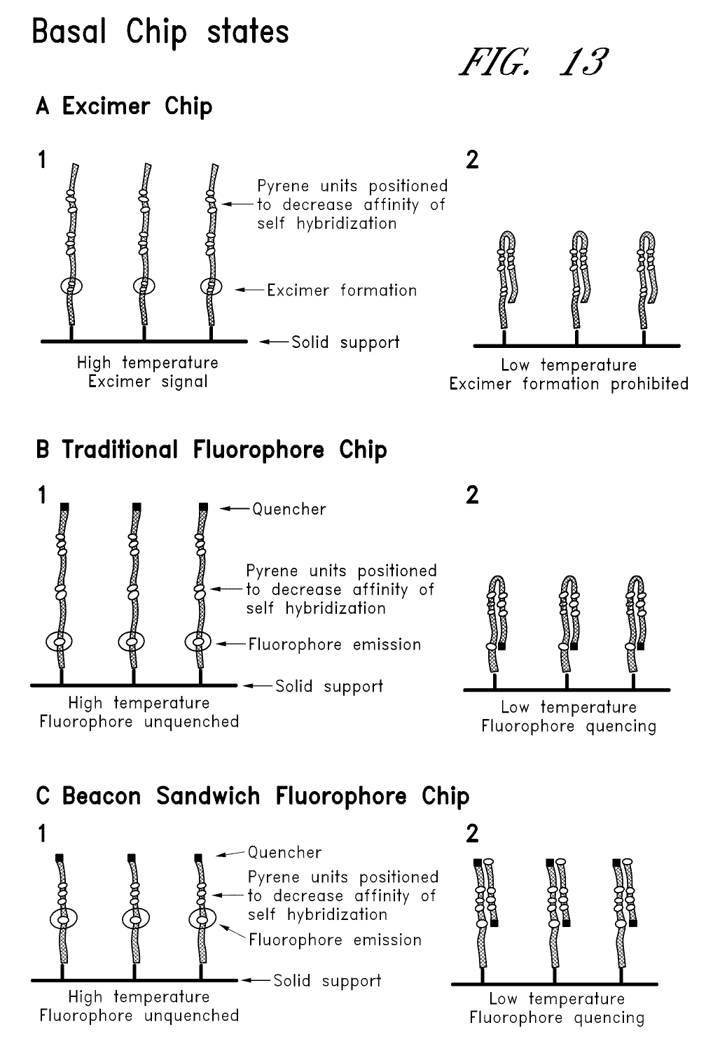

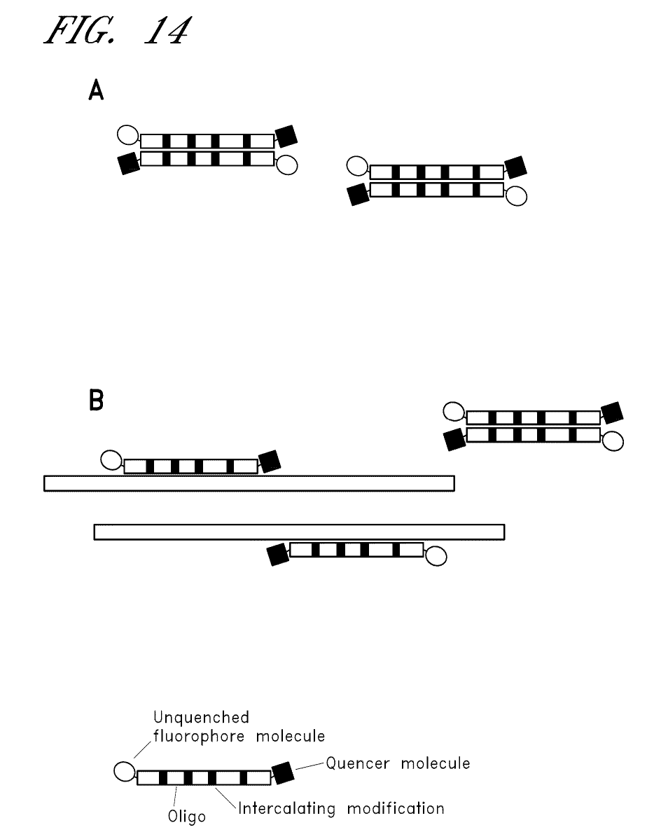

Nucleotides, nucleotide substitutes, nucleotide analogues and intercalator pseudonucleotides from first and second sequences are said to be positioned “opposite” of each other when they are placed in close proximity upon hybridization. Preferably, nucleotides, nucleotide substitutes, nucleotide analogues and intercalator pseudonucleotides are said to be positioned “opposite” when they are directly opposite of each other. However, nucleotides, nucleotide substitutes, nucleotide analogues and intercalator pseudonucleotides are also said to be positioned “opposite” when they are positioned in a small region surrounding the nucleotide/nucleotide substitute/nucleotide analogues/intercalator pseudonucleotide directly opposite of said nucleotide/nucleotide substitute/nucleotide analogues/intercalator pseudonucleotide. One example of the opposite positioned intercalator pseudonucleotides is illustrated in FIG. 14, wherein oligonucleotide analogue pairs comprising opposite positioned intercalator pseudonucleotides are shown.

Accordingly, it is preferred that the at least one intercalator pseudonucleotide of the first sequence is placed in such a manner that it is positioned opposite of the at least one intercalator pseudonucleotide of the second sequence, when the first sequence is hybridized to the second sequence.

It is even more preferred that every intercalator pseudonucleotide of the second sequence is placed in such a manner that they are positioned opposite of an intercalator pseudonucleotide of the first sequence, when the first sequence is hybridized to the second sequence.

It is yet another object of the present invention to provide a system where the first sequence is connected to the second sequence.

The first sequence and the second sequence may be connected to each other directly or indirectly, for example they may be coupled covalently to each other or they may be coupled covalently via a third sequence, or they may only be connected to each other when they are hybridized for example via hydrogen bonds. Accordingly, both sequences may be comprised within one nucleic acid analogue. Alternatively, the first nucleotide sequence may be comprised within a first nucleic acid or nucleic acid analogue and the second nucleotide sequence may be comprised within a second nucleic acid or nucleic acid analogue.

When the first sequence and the second sequence are comprised within one oligonucleotide analogue, then said oligonucleotide analogues are preferably as a minimum as long as the first sequence and the second sequence together; however, the oligonucleotide analogues may be longer than the first sequence and the second sequence together, and accordingly the oligonucleotide analogues may comprise other parts than the first sequence and the second sequence.

For example, the oligonucleotide analogue preferably comprises between 5 and 100, such as between 5 and 10, such as between 10 and 15, for example between 15 and 20, such as between 20 and 30, for example between 30 and 40, such as between 40 and 50, for example between 50 and 60, such as between 60 and 80, for example between 60 and 100 nucleotides and/or nucleotide analogues and/or intercalator pseudonucleotides. More preferably the oligonucleotide analogue consists of in the range from 15 to 50 nucleotides.

In one embodiment, one oligonucleotide analogue may comprise the first sequence and a corresponding second sequence, wherein said first sequence and said second sequence are separated by a third sequence consisting of p nucleotides and/or nucleotide analogues.

p may be any desirable integer, for example p may be an integer between 1 and 5, for example 5 and 10, such as between 10 and 15, for example between 15 and 20, such as between 20 and 30, for example between 30 and 50.

The oligonucleotide analogues according to the present invention may comprise any desirable number of intercalator pseudonucleotides. For example the oligonucleotide analogue may comprise between 2 and 5, such as between 5 and 10, such as between 10 and 15, for example between 15 and 20 intercalator pseudonucleotides.

The intercalator pseudonucleotides may be dispersed at any position in the first, second and/or third sequence of the oligonucleotide analogue.

Oligonucleotides Comprising Fluorescent Groups

In one embodiment, oligonucleotide analogues according to the present invention are labeled with a detectable label. For example intercalator pseudonucleotides comprised within an oligonucleotide analogue may often comprise fluorescent properties, in particular many intercalators are capable of fluorescing.

In some embodiments of the present invention, an oligonucleotide comprising at least one intercalator pseudonucleotide may comprise at least one additional fluorescent group. For example the fluorescent group may be selected from the group consisting of, but not limited to, fluorescein, FITC, rhodamine, lissamine rhodamine, rhodamine 123, Acridine Orange, coumarin, CY-2, CY-3, CY 3.5, CY-5, CY 5.5, ethidium bromide, FAM, GFP, YFP, BFP, YO-YO, HEX, JOE, Nano Orange, Nile Red, OliGreen, Oregon Green,. Pico green, Propidium iodide, Radiant Red, Ribo Green, ROX, R-phycoerythrin, SYBR Gold, SYBR Green I, SYBR Green II, SYPRO Orange, SYPRO Red, SYPRO Ruby, TAMRA, Texas Red and XRITC.

In a preferred embodiment the label is a complex of at least two intercalator pseudonucleotides according to the present invention, capable of forming an intramolecular excimer, exciplex, FRET or charge-transfer complex.

Furthermore, it is also contained within the present invention that an oligonucleotide analogue according to present invention may comprise at least one quencher molecule.

A quencher molecule according to the present invention is any molecule that is capable of quenching the fluorescence of particular fluorescent group(s) in its vicinity. The quencher may function by absorbing energy from the fluorescent group and dissipating the energy as heat or radiative decay. Hence, the signal from the fluorescent group will be reduced or absent. Accordingly, if a fluorescent group and a suitable quencher molecule are placed close to each other, the fluorescence of the fluorescent group will be quenched.

Examples of quencher molecules include, but are not limited to, DABCYL, DABSYL TAMRA, Methyl red, Black Hole-1, Black Hole-2, ElleQuencher and QSY-7. However, the quencher molecule should generally be selected according to the fluorescent group.

A preferred embodiment of the present invention is to provide an oligonucleotide analogue comprising at least one intercalator pseudonucleotide, and which furthermore also comprises one fluorophore and a quencher molecule, which are able to quench the fluorescence from the fluorophore.

Preferred pairs of fluorescent groups-quencher molecules according to the present invention include, but are not limited to:

| Fluorescent group | Quencher |
|---|---|
| FAM | TAMRA |
| TET | TAMRA |
| Rhodamine | TAMRA |
| Coumarin | DABCYL |
| EDANS | DABCYL |
| Fluorescein | DABCYL |
| Lucifer Yellow | DABCYL |
| Eosin | DABCYL |
| TAMRA | DABCYL |

The fluorescent groups and the quencher molecules may individually be placed at any position within the nucleic acids. However, in one preferred embodiment at least one fluorescent group is attached as a dangling end, more preferably all fluorescent groups which are not comprised within an intercalator pseudonucleotide are attached as dangling ends. The fluorescent group may be placed as a dangling end in the 5′ end or in the 3′ end or in both ends. It is also preferred that at least one quencher molecule is attached as a dangling end, more preferably all quencher molecules which are not comprised within an intercalator pseudonucleotide are attached as dangling ends. The quencher molecule may be placed as a dangling end in the 5′ end or in the 3′ end or in both ends.

A preferred embodiment of the present invention is to provide a pair of probes according to the present invention comprising a first sequence and a corresponding second sequence. The first sequence preferably comprises a fluorophore and the second sequence preferably comprises a quencher molecule capable of quenching said fluorophore. Accordingly, fluorescence will be detectable only when the first nucleic acid is not hybridized to the second nucleic acid.

Alternatively, each sequence (first sequence and second sequence) may comprise a fluorophore and a quencher, wherein the fluorophore of first sequence may be quenched of either the quencher comprised in first sequence and/or the quencher comprised in the second sequence; and vice versa the fluorophore of second sequence may be quenched of either the quencher comprised in first strand and/or the quencher comprised in the second strand.

In particular, when the first sequence is comprised within a first nucleic acid and the second sequence is comprised within a second nucleic acid, the first nucleic acid may comprise at least one fluorescent group and/or the second nucleic acid may comprise at least one fluorescent group.

To obtain a stronger signal it is possible to use more than one fluorescent group, for example the first sequence may comprise two fluorescent groups and the second nucleic acid may comprise two quencher molecules. Preferably, the quencher molecules and the fluorescent groups are positioned in a manner so that each quencher group is capable of quenching fluorescence of one fluorescent group when the first sequence and the second sequence are hybridized with each other.

In an even more preferred embodiment, an oligonucleotide analogue comprises a first sequence and a corresponding second sequence that are separated by a third sequence (a hairpin probe), where at least one of the sequences comprises at least one intercalator pseudonucleotide and the oligonucleotide analogue has an additional fluorescent group placed as a dangling end in the 5′ end or in the 3′ end and an additional quencher molecule placed as a dangling end in the end opposite to said fluorophore.

The length, degree of complementarity, number and placement of intercalator pseudonucleotides are some of the parameters that may be varied in order to obtain a desired melting temperature between the first and second sequence.

Accordingly it is a preferred embodiment of the present invention to provide a hairpin probe that will self-hybridize unless subjected to a fully complementary target under hybridization conditions.

When the label is a complex of at least two intercalator pseudonucleotides capable of forming an intramolecular excimer, exciplex, FRET or charge-transfer complex (see herein below), preferably the at least two intercalator pseudonucleotides are separated by at least one nucleotide or nucleotide analogue. In such an embodiment, quenching of signal could be obtained by hybridization of the nucleotides in a region of at least one nucleotide to either side of any intercalator pseudonucleotide in the complex to a complementary sequence.

Hence it is a preferred embodiment of the present invention to provide an oligonucleotide analogue comprising at least two intercalator pseudonucleotides where the spectral properties are changed upon hybridization to a target nucleic acid or as a consequence of amplification of a target nucleic acid. In a preferred embodiment the spectral signal is low when there is no or small amounts of target nucleic acids, and high when there is larger amounts of target nucleic acids present. When used during an amplification reaction of target sequence, e.g. by PCR, it is preferred that the spectral signal increases in correspondence to the increase of said target nucleic acid sequence.

Accordingly, it is preferred to provide an oligonucleotide analogue that comprises a first sequence and a complementary second sequence that are separated by a third sequence (a hairpin probe), where the second or third sequence comprise at least one intercalator pseudonucleotide and where the first sequence comprises an additional complex of intercalator pseudonucleotides according to the present invention, where the spectral signal is low when said first sequence is hybridized to the second sequence and high when they are not hybridized.

Fluorescence resonance energy transfer (FRET) is a distance-dependent interaction between the electronic excited states of two dye molecules in which excitation is transferred from a donor molecule to an acceptor molecule without emission of a photon. FRET is dependent on the inverse 6^{th} power of the intermolecular separation, making it useful over ‘ distances comparable with the dimension of biological macromolecules. Preferably the donor and the acceptor must be in close proximity (typically between 10 to 100 Å) for FRET to occur. Furthermore, the absorption spectrum of the acceptor must overlap with the fluorescence emission spectrum of the donor. It is further preferred that the donor and the acceptor transition dipole orientations must be approximately parallel.

It is also comprised within the present invention that the first sequence may comprise a donor for FRET and the second sequence may comprise an acceptor for FRET. Preferably said donor and said acceptor are positioned so that FRET may occur when the first sequence is hybridised to the second sequence. The fluorescent groups may for example be useful for detecting the pair of oligonucleotide analogue sequences.

FRET donor and acceptor pairs for example include:

| Donor | Acceptor |
|---|---|
| Fluorescein | Tetramethylrhodamine |
| IAEDANS | fluorescein |
| EDANS | DABCYL |
| Fluorescein | Fluorescein |
| BODIPY FL | BODIPY FL |
| Fluorescein | QSY 7 dye |

In a preferred embodiment, at least two intercalator pseudonucleotides are placed as next nearest neighbours, i.e. 1 nucleotide is separating the intercalator pseudonucleotides.

Consequently one object of the present invention is to provide an oligonucleotide analogue comprising at least one intercalator pseudonucleotide, wherein the spectral properties comprised within said oligonucleotide analogue may be used for detection of the presence of target nucleic acid sequence. In a preferred embodiment, this can be done real-time during an amplification reaction of the target nucleic acid sequence, either by taking advantage of changed spectral properties due to hybridization to target nucleic acid sequence of said oligonucleotide analogue or by taking use of the 5’-3′ exo- or endonuclease activity, of DNA polymerases that may enhance the spectral signal from any probes according to the present invention.

Alternatively or additionally fluorescence detection can be carried out after the amplification process, so-called end-point detection.

Method of Detecting Hybridization

In one embodiment the present invention relates to a method of detecting hybridization between a target nucleic acid and a first sequence comprising at least one intercalator pseudonucleotide of the general structure

X—Y-Q

wherein
X is a backbone monomer unit capable of being incorporated into the backbone of a nucleic acid as described herein above; and
Q is an intercalator comprising at least one essentially flat conjugated system, which is capable of co-stacking with nucleobases of a nucleic acid or nucleic acid analogue as described herein above; and
Y is a linker moiety linking said backbone monomer unit and said intercalator as described herein above; comprising the steps of
a) providing the target nucleic acid and optionally a complementary target nucleic acid; and
b) providing at least one oligonucleotide analogue comprising said first sequence, wherein the first sequence is capable of hybridizing with said target nucleic acid; and
c) incubating the target nucleic acid and the oligonucleotide or oligonucleotide analogue under conditions allowing for hybridization; and
d) detecting hybridization.

In one embodiment of the present invention, the first sequence comprises at least two intercalator pseudonucleotides, each comprising an intercalator capable of forming an excimer, an exciplex or a charge transfer complex.

Preferably the intercalators of the at least two intercalator pseudonucleotides are capable of forming an intramolecular excimer, an intramolecular exciplex, intramolecular FRET complex or a charge transfer complex, when the first sequence is unhybridised and said intercalators are not capable of forming an intramolecular excimer, an intramolecular exciplex, intramolecular FRET complex or a charge transfer complex, when said first sequence is hybridised to a corresponding nucleic acid or nucleic acid analogue, more preferably when at least one of the nucleotides separating the intercalator pseudonucleotides is hybridised to complementary nucleotides. Preferably only one nucleotide is separating said intercalator pseudonucleotides.

Accordingly, in said embodiment hybridization of the first sequence to any corresponding sequence may be determined by determining the excimer fluorescence, exciplex fluorescence, intramolecular FRET complex fluorescence or charge-transfer absorption. Said oligonucleotide analogues may preferably be designed so that high excimer fluorescence, exciplex fluorescence, FRET fluorescence or charge-transfer absorption is indicative of no hybridization or vice versa said oligonucleotide analogues may be designed so that high excimer, exciplex or charge-transfer absorption is indicative of hybridization.

An excimer is a dimer of compounds, which is associated in an electronic excited state, and which is dissociative in its ground state. When an isolated compound is excited it may lose its excitation or it may associate with another compound of the same kind (which is not excited), whereby an excimer is formed. An excimer emits fluorescence at a wavelength different from monomer fluorescence emission. When the excimer loses its excitation, the association is no longer favourable and the two species will dissociate. An exciplex is an excimer like dimer, wherein the two compounds are different.

Intramolecular excimers are formed by two moieties comprised within one molecule, for example 2 polyaromatic groups within the same molecule. Similar intramolecular exciplexes are formed by two moieties comprised within one molecule, for example by 2 different polyaromatic groups.

A charge transfer complex in which there is weak coordination involving the transfer of charge between two molecules. An example is phenoquinone, in which the phenol and quinone molecules are not held together by formal chemical bonds but are associated by transfer of charge between the aromatic ring systems of the compounds.

In another embodiment of the present invention, in step b) of the above mentioned method of detecting hybridization, a pair of oligonucleotide analogue sequences as described herein above are provided instead.

Preferably, the target nucleic acid comprises a sequence capable of hybridizing with the first sequence and the complementary target nucleic acid comprises a sequence capable of hybridizing with the second nucleotide sequence of the oligonucleotide analogue pair as described above.

In particular, it is preferred to use a pair of oligonucleotides or oligonucleotide analogues comprising a fluorescent group and/or a quencher molecule as described herein above. For example the first sequence may comprise a fluorescent group and/or a quencher molecule as described herein above and/or the second sequence may comprise a fluorescent group and/or a quencher molecule as described herein above.

Detecting hybridization may thus, be determined by determining the spectral properties of the first sequence and/or determining the spectral properties of the second sequence.

In particular the spectral properties may be fluorescent properties, and preferably said fluorescent properties are the fluorescence of the non-intercalator pseudonucleotides.

In a preferred embodiment according to the present invention, the fluorescent group of the first sequence will be close to a quencher group of the second sequence when the first sequence and the second sequence are hybridised to each other (see herein above). Accordingly, if the first sequence is hybridised to the second sequence, there will be no detectable fluorescent signal; however, if the first sequence is hybridised to a corresponding nucleic acid, i.e. the target nucleic acid, there will be a detectable fluorescent signal.

In said embodiment, a spectral signal above a predetermined limit may thus be indicative of hybridization.

Alternatively, hybridization may be determined by determining the melting temperature. This may be done because the melting temperature of a hybrid between the first sequence and the second sequence is lower than the melting temperature of a hybrid consisting of the first sequence and a corresponding nucleic acid or nucleic acid analogue which does not comprise said intercalator pseudonucleotides.

Accordingly, low melting temperature is indicative of hybridization between first and second sequence, whereas high melting temperature is indicative of hybridization between first sequence and a corresponding nucleic acid and/or second sequence and a corresponding nucleic acid.

Hence a melting temperature above a predetermined limit may be indicative of hybridization.

The method of detecting hybridization may be used for a number of different purposes. For example, the method may be employed for quantification of a polymerase chain reaction.

Alternatively, the method may be employed for detecting hybridization in an assay dependent on specific hybridization, for example Southern blotting, Northern blotting, FISH or other kinds of in situ hybridization.

Alternatively probes labeled with non-quencheable signal molecules can be used, requiring the removal of unspecific bound probe e.g. by wash.

Method for Real-Time Detection of Nucleic Acid Sequences During Amplification Reactions

The present invention also relates to a method for real-time detection of nucleic acid or nucleic acid analogue sequences during amplification reactions, comprising the steps of
a) providing at least one template comprising one or more nucleic acid sequence(s) which is desirable to amplify; and
b) providing at least one oligonucleotide analogue sequence as described herein above, wherein said oligonucleotide analogue(s) is capable of hybridizing with the nucleic acid sequence(s), which is desirable to amplify; and
c) providing at least one set of primers which is capable of hybridizing with a nucleic acid sequence complementary to the template nucleic acid, which is desirable to amplify; and
d) incubating said template nucleic acid(s) with said oligonucleotide analogue(s) and said set of primers under conditions allowing for hybridization; and
e) optional detection; and
f) elongating said primers in the 5′ to 3′ direction in a template dependent manner; and
g) optional detection.

Such a method may furthermore comprise the steps of
a) incubating the mixture comprising nucleic acids and nucleic acid analogues under conditions that do not allow hybridization; and
b) repeating steps d), e), f), g) and optionally step h).

Each step may be performed more than once. In particular, steps d), e), f), g) and h) may be repeated at least once, for example between 2 and 5, such as between 5 and 10, such as between 10 and 15, for example between 15 and 20, such as between 20 and 30, for example between 30 and 50 times.

The primer may be any nucleic acid or nucleic acid analogue sequence which is preferably between 5 and 100 base pairs long.

The amplification of specific nucleic acid sequences may for example be a polymerase chain reaction (PCR).

Generally, PCR temperature cycling involves at least two incubations at different temperatures. One of these incubations is for primer hybridization and a catalysed primer extension reaction. The other incubation is for denaturation, i.e., separation of the double stranded extension products into single strand templates for use in the next hybridization and extension incubation intervals.

The details of the polymerase chain reaction, the temperature cycling and reaction conditions necessary for PCR as well as the various reagents and enzymes necessary to perform the reaction are for example described in U.S. Pat. Nos. 4,683,202, 4,683,195, EPO Publication 258,017 and 4,889,818 (Taq polymerase enzyme patent), which are hereby incorporated by reference.

However, more frequently, the PCR consists of an initial denaturation step which separates the strands of a double stranded target nucleic acid sample, followed by the repetition of: 1. an annealing step, which allows amplification primers to anneal specifically to opposite strands of the target and at positions flanking a target sequence; 2. an extension step which extends the primers 5′ to 3′ in a template directed manner, thereby forming a complementary copy of the target, and; 3. a denaturation step which causes the separation of the copy and the target. Each of the above steps may be conducted at a different temperature, where the temperature changes may be accomplished using a thermocycler apparatus. Repetition of steps 1-3 by simple temperature cycling of the sample results in an exponential phase of replication, typically generating millions of copies or more of the target duplex in 20-40 cycles (Innis et al., PCR Protocols: A Guide to Methods and Applications, (1990) Academic Press, Saiki et al., Science, (1988) 239: 487).

The purpose of a polymerase chain reaction is to manufacture a large volume of DNA, which is identical to or largely resembles an initially supplied small volume of “seed” DNA. The reaction involves copying the strands of the DNA and then using the copies to generate other copies in subsequent cycles. Under ideal conditions, each cycle will double the amount of DNA present thereby resulting in a geometric progression in the volume of copies of the “target” or “seed” DNA strands present in the reaction mixture. However a PCR may also be used to introduce mutations and amplify them.

One example of a typical PCR programme may be as follows: The programme starts at a sample temperature of 94° C. held for 30 seconds to denature the reaction mixture. Then, the temperature of the reaction mixture is lowered to a temperature in the range from 35° C. to 65° C. and held for in the range of 15 seconds to 2 minutes to permit primer hybridization. Next, the temperature of the reaction mixture is raised to a temperature in the range from 50° C. to 72° C. where it is held for in the range of 30 seconds to 5 minutes to promote the synthesis of extension products. This completes one cycle. The next PCR cycle then starts by raising the temperature of the reaction mixture to 94° C. again for strand separation of the extension products formed in the previous cycle (denaturation). Typically, the cycle is repeated 20 to 50 times.

Generally, it is desirable to change the sample temperature to the next temperature in the cycle as rapidly as possible for several reasons. First, the chemical reaction has an optimum temperature for each of its stages. Thus, less time spent at nonoptimum temperatures means a better chemical result is achieved. Another reason is that a minimum time for holding the reaction mixture at each incubation temperature is required after each said incubation temperature is reached. These minimum incubation times establish the “floor” or minimum time it takes to complete a cycle. Any time transitioning between sample incubation temperatures is time which is added to this minimum cycle time. Since the number of cycles is fairly large, this additional time unnecessarily lengthens the total time needed to complete the amplification.

In addition, the above-mentioned amplification may comprise a step of determining the fluorescent properties of the pair of nucleotide sequences. Determining the spectral properties may for example include determining one or more selected from the group consisting of monomer fluorescence, excimer fluorescence, exciplex fluorecence, FRET and charge-transfer absorption. In particular the spectral properties may include monomer fluorescence, excimer fluorescence, exciplex fluorescence, FRET fluorescence or charge transfer absorption of the intercalators of the intercalator pseudonucleotides.

Alternatively, determining the spectral properties may include determining FRET of a donor/acceptor pair coupled to the first and/or second sequence.

One advantage of the method according to the present invention is that it is possible to determine spectral properties simultaneously to performing the amplification reaction. Hence information on spectral properties may be used to determine for example the number of cycles, the length of each step, the temperature of each step during the reaction, and accordingly the reaction may be adjusted to the specific needs.

In a preferred embodiment the oligonucleotide analogue comprising a label is hybridized to the template nucleic acid during the elongation step of the method. Thereby it is possible to use the endo- and/or 5′-3′ exo-nuclease activity of the DNA polymerase to break a bond in the backbone of said oligonucleotide analogue and hence cleave it. In particular it is preferred that the smallest part cleaved off comprises the label of said oligonucleotide analogue, and that this enhances the detection signal from said label.

In another preferred embodiment the oligonucleotide analogue comprising a label is only hybridized to the template nucleic acid at a temperature lower than the elongation temperature. At this temperature it is also preferred that if said oligonucleotide analogue is not hybridized to the target nucleic acid sequence, the signal from the detection label in said oligonucleotide analogue is quenched. Most preferably the detection signal is measured at said low temperature and said oligonucleotide analogue is unhybridized at the temperature used in the elongation step.

Method of Modulating Gene Transcription

The present invention furthermore relates to a method of modulating transcription of one or more specific genes, comprising the steps of
e) providing a transcription system; and
f) providing at least one oligonucleotide and/or oligonucleotide analogue comprising a first sequence comprising at least one intercalator pseudonucleotide of the general structure

X—Y-Q

wherein
X is a backbone monomer unit capable of being incorporated into the phosphate backbone of a nucleic acid; and
Q is an intercalator comprising at least one essentially flat conjugated system, which is capable of co-stacking with nucleobases of a nucleic acid; and
Y is a linker moiety linking said backbone monomer unit and said intercalator;
and wherein said first sequence is capable of hybridizing with said gene and/or regulatory sequences thereof or the complementary strand of said gene and/or regulatory sequences thereof; and
g) introducing said first sequence into the transcription system; and
h) hybridizing the first sequence with said one or more genes and/or regulatory sequences hereof or the complementary strand of the gene and/or regulatory sequences hereof; and
thereby modulating transcription of said gene.

Said oligonucleotide or oligonucleotide analogue may comprise one or more selected from the group consisting of subunits of DNA, RNA, LNA, PNA, ANA, 2′-O-methyl RNA, MNA and HNA.

The transcription of the gene is preferably modulated, because the oligonucleotide is capable of hybridising with the gene, the complementary strand and/or sequences regulating the gene. In one embodiment of the present invention the modulation of transcription is based on the antigene strategy. In the antigene strategy, hybridisation may for example result in sterical interference with the transcription machinery and accordingly in one embodiment of the present invention result in halt in transcription or block of transcription.

The antigene strategy usually involves strand invasion, meaning that the oligonucleotide or oligonucleotide analogue may invade a double stranded DNA molecule, and hybridise with one of the strands. Accordingly, it is preferred that the oligonucleotide or oligonucleotide analogue has higher affinity for DNA than DNA has for DNA.

In a preferred embodiment of the present invention, the antigene strategy involves double strand invasion. For example, the strategy involves using a pair of oligonucleotides or oligonucleotide analogues, wherein each oligonucleotide and/or oligonucleotide analogue may hybridise to the gene and/or regulatory sequences hereof or the complementary strand of the gene and/or regulatory sequences hereof with higher affinity than the gene and/or regulatory sequences hereof to its complementary strand and with higher affinity that the pair of oligonucleotides or oligonucleotide analogues to each other.

The principle behind double strand invasion is described in FIG. 15.

In a preferred embodiment of the present invention, step b) of the method of modulating transcription involves providing a pair of nucleotide and/or nucleotide analogue sequences as described herein above, wherein the first sequence is capable of hybridizing with the gene and/or regulatory sequences thereof and the second sequence is capable of hybridizing with a nucleic acid sequence complementary to the gene/or regulatory sequences thereof.

In this embodiment, said pair of nucleotide and/or nucleotide analogue sequences is then introduced into the transcription system and the first sequence is hybridized with the gene or regulatory sequences hereof and the second sequence is hybridized to the other strand.

There are several advantages of using the pair of nucleotide and/or nucleotide analogue sequences according to the present invention for modulating gene expression. For example, many organisms or cell types comprise defense mechanisms against foreign genetic material, which may destroy foreign genetic material, for example nucleases. However, frequently single stranded oligonucleotides are more susceptible to said defence mechanisms compared to double stranded oligonucleotides or oligonucleotide analogues. Therefore it is an advantage to use a pair of oligonucleotides or oligonucleotide analogues to modulate gene transcription in vivo or ex vivo.

Furthermore, the pair of oligonucleotides and/or oligonucleotide analogues according to the invention may hybridize to both strands of the gene of interest, which may modulate gene activity more rigidly than when there is hybridization to only one strand.

Because the melting temperature of a hybrid between the first sequence and the second sequence is preferably lower than the melting temperature of a hybrid between the first sequence and a corresponding DNA and a hybrid between the second sequence and a corresponding DNA, the pair of oligonucleotide analogues comprising intercalator pseudonucleotides according to the present method preferably hybridize with the gene and/or complementary gene sequence rather than with each other.

It is preferred that the melting temperature of a self-hybrid consisting of said oligonucleotide and/or oligonucleotide analogue, such as the first sequence is significantly lower than the melting temperature of a hybrid consisting of the first sequence and the said gene or regulatory sequences thereof or the complementary strand of said gene or the complementary strand of regulatory sequences thereof.

The transcription system may be any useful transcription system, including both in vitro systems, in vivo systems and ex vivo systems. For example the transcription system may be selected from the group consisting of yeast cells, fungi cells, mammalian cells, plant cells, bacterial cells, archeabacterial cells and vira.

Preferably, the transcription system is an intact cell. For example the cell may be a human cell. The cell may be an isolated cell or the cell may be comprised within a living organism, such as an animal, a human or a plant.

Oligonucleotides and/or Nucleotide Analogues Having Reduced Cross-Hybridisation

For many purposes it is desirable that oligonucleotides and/or oligonucleotide analogues only hybridise with target nucleic acids (usually RNA or DNA) and not with other homologously complementary nucleic acids and nucleic acid analogues including the said oligonucleotide itself. Especially β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA and PNA comprising probes, but also other nucleic acids and nucleic acid analogues, have a tendency to cross-hydridise with high affinity to homologously complementary nucleic acids or nucleic acid analogues of the same type as said probes.

It is often undesirable to use probes that have higher affinity for nucleic acid analogues than for the homologously complementary RNA or DNA targets. However many known oligonucleotide analogues for example, but not limited to, β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, and PNA have a higher affinity for a homologously complementary oligonucleotide analogue of the same type than for an equally homologously complementary unmodified RNA or DNA oligonucleotide probes. Accordingly said oligonucleotide analogues may suffer from a high self-affinity and have difficulties to be employed for simultaneous hybridisation to both complementary strands of the same region of a target duplex DNA nucleic acid. Furthermore it can be a problem to use oligonucleotide analogues that are correspondingly complementary to it self.

Accordingly, it is an object of the present invention to provide oligonucleotides or oligonucleotide analogues comprising at least one intercalator pseudonucleotide, wherein the melting temperature of a duplex consisting of said oligonucleotide or oligonucleotide analogue and a homologously complementary oligonucleotide and/or oligonucleotide analogue comprising at least one intercalator pseudonucleotide is significantly lower than the melting temperature of a hybrid consisting of said oligonucleotide and/or oligonucleotide analogue and a homologously complementary DNA (DNA hybrid).

In one embodiment of the present invention a pair of homologously complementary oligonucleotides and/or oligonucleotide analogues comprises at least 1 intercalator pseudonucleotide in each oligonucleotide and/or oligonucleotide analogues each as described herein above, wherein said at least 2 intercalator pseudonucleotides are positioned in relation to each other, so that they are in close vicinity of each other when the homologously complementary oligonucleotides or oligonucleotide analogues are hybridised. Preferably, the at least 2 intercalator pseudonucleotides are positioned in relation to each other, so that they are opposite each other when the pair of oligonucleotide analogues are hybridised. More preferably the oligonucleotide or oligonucleotide analogues comprise more than one pair of intercalator pseudonucleotides, such as 2, for example 3, such as 4, for example 5, such as more than 5 pairs of intercalator pseudonucleotides, wherein each pair of intercalator pseudonucleotides are positioned in relation to each other, so that at least two intercalator pseudonucleotides are in close vicinity of each other when the oligonucleotide analogues are hybridised. Preferably, each intercalator pseudonucleotides in one strand is positioned in relation to each intercalator pseudonucleotides in the other strand, so that they two and two are in close vicinity of each other when the oligonucleotide or oligonucleotide analogues are hybridized. Most preferably each intercalator pseudonucleotides in one strand is positioned in relation to each intercalator pseudonucleotides in the other strand, so that they two and two are opposite of each other when the oligonucleotide or oligonucleotide analogues are hybridized. The result of such a system will be a pair of homologously complementary nucleic acid analogues consisting of two strands (strand 1 and strand 2) where strand 1 have a higher affinity for a complementary DNA strand than for strand 2, and strand 2 have a higher affinity for a complementary DNA strand than for strand 1. In this way it is possible to make a pair of probes, which have higher affinity for target sequences than for its complementary probe strand and a pair of probes, which can address both strands of a complementary region of duplex DNA. The strands in the probe pair may be directly or indirectly detectable. The result of such a procedure would be a probe system with lower risk for false negatives and false positives, having increased sensitivity giving a better signal to noise ratio of the target DNA.

Oligonucleotides and/or Oligonucleotide Analogues Having Reduced Self-Hybridisation

In another preferred embodiment of the present invention the intercalator pseudonucleotides may inhibit or largely reduce cross hybridisation to self when incorporated into an oligonucleotide and/or oligonucleotide analogue.

Accordingly, it is an objective of the present invention to provide oligonucleotides and/or oligonucleotide analogues comprising at least 2 intercalator pseudonucleotides as described herein above, wherein said 2 intercalator pseudonucleotides are positioned in relation to each other, so that they are close vicinity of each other if the oligonucleotide and/or oligonucleotide analogue self-hybridise.

Preferably, the at least 2 intercalator pseudonucleotides are positioned in relation to each other, so that they are opposite to each other if the oligonucleotide and/or oligonucleotide analogue is self-hybridised. More preferably the oligonucleotide and/or oligonucleotide analogue comprises more than one pair of intercalator pseudonucleotide, such as 2, for example 3, such as 4, for example 5, such as more than 5 pairs of intercalator pseudonucleotides, wherein each pair of intercalator pseudonucleotide are positioned in relation to each other, so that they two and two are in close vicinity of each other when the oligonucleotide and/or oligonucleotide analogue is self hybridised. Preferably, each pair of intercalator pseudonucleotides are positioned in relation to each other, so that they are opposite to each other when the oligonucleotide and/or oligonucleotide analogue self hybridise. In a preferred embodiment the fluorescence features of at least one of said intercalator pseudonucleotide pair may be used as a signal molecule for detection.

The melting temperature of a duplex between two homologously complementary oligonucleotide analogue sequences comprising intercalator pseudonucleotide(s), will be dependent on the number of intercalator pseudonucleotides inserted and where said intercalator pseudonucleotides are inserted. The melting temperature may be decreased compared to a duplex of the same oligonucleotide (analogue) not comprising any intercalator pseudonucleotides. Preferably, the melting temperature is decreased with at least 2° C., such as at least 5° C., for example at least 10° C., such as at least 15° C., such as from 2 to 50° C., such as from 2 to 40° C., such as from 2 to 30° C., for example from 5 to 20° C., such as from 10° C. to 15° C., for example from 2° C. to 5° C., such as from 5° C. to 10° C., for example from 10° C. to 15° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C.

In one embodiment of the present invention the invention relates to inhibiting or largely reducing self- and/or cross-hybridisation of probes comprising one or more nucleotides selected from, but not limited to: β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α- α-D-RNA, β-D-RNA or RNA.

In a preferred embodiment of the present invention the invention relates to inhibiting or largely reducing self- and/or cross-hybridisation of probes comprising one or more nucleotides selected from LNA, α-D-Ribo-LNA, [3.2.1]-LNA, 2′-R-RNA, 2′-OR-RNA, α-L-RNA, α-D-RNA, β-D-RNA or RNA.

Accordingly, it is an object of the present invention to provide oligonucleotide analogues comprising at least one intercalator pseudonucleotide as described herein above, wherein the melting temperature of a self-hybrid consisting of said oligonucleotide analogue is significantly lower than the melting temperature of a hybrid consisting of said oligonucleotide analogue and the oligonucleotide analogue when comprising no intercalator pseudonucleotides or a homologously complementary DNA (DNA hybrid).

Preferably said oligonucleotide analogue is selected from the group consisting of DNA, β-D-Homo-DNA, f3-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or PNA and mixtures thereof, more preferably the oligonucleotide analogue may be selected from the groups comprising β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1J-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or PNA and mixtures comprising a significant amount of β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or PNA.

The melting temperature of the hybrid between said oligonucleotide and/or oligonucleotide analogue comprising at least one intercalating pseudonucleotide and DNA is at least 2° C., such as at least 5° C., for example at least 10° C., such as at least 15° C., such as from 2 to 50° C., such as from 2 to 40° C., such as from 2 to 30° C., for example from 5 to 20° C., such as from 10° C. to 15° C., for example from 2° C. to 5° C., such as from 5° C. to 10° C., for example from 10° C. to 15° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C., such as from 55° C. to 60° C., for example from 60° C. to 65° C. higher than the melting temperature of the self-hybrid of said oligonucleotide analogue.

In an embodiment of the present invention the oligonucleotide analogue comprises at least one intercalator pseudonucleotide as described herein above, wherein said intercalator pseudonucleotide is positioned in relation to non-DNA nucleotides such as, but not limited to β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or

PNA, or other RNA-like nucleotides, so that they are in close vicinity of each other when the oligonucleotide analogue is hybridised or self-hybridised. Preferably, the at least one intercalator pseudonucleotide is positioned in relation to the non-DNA nucleotides such as, but not limited to β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or PNA, or other RNA-like nucleotides, so that the intercalator pseudonucleotide(s) are opposite and between two non-DNA-like nucleotides when the oligonucleotide analogue is hybridised or self-hybridised.

Furthermore the present invention relates to methods for designing and producing sequences with reduced melting temperature of a self-hybrid compared to the melting temperature of the homologously complementary non-intercalator pseudonucleotide modified nucleotides or nucleotide analogues. Preferably the self-hybrid comprises an oligonucleotide or oligonucleotide analogue selected from the group consisting of RNA, β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA or PNA that tends to self-hybridise under low, medium or even high stringency conditions and mixtures thereof, comprising the steps of
a) synthesizing an oligonucleotide analogue sequencing with at least one intercalator pseudonucleotide, placed in the oligonucleotide in a manner so it is positioned in close proximity to at least one nucleotide selected from the group of: β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or PNA that is part of the self-hybrid said intercalator pseudonucleotide having the general structure

X—Y-Q

wherein
X is a backbone monomer unit capable of being incorporated into the phosphate backbone of a nucleic acid; and
Q is an intercalator comprising at least one essentially flat conjugated system, which is capable of co-stacking with nucleobases of a DNA-like nucleic acid ; and
Y is a linker moiety linking said backbone monomer unit and said intercalator;
preferably, an intercalator pseudonucleotide as described herein above; and
obtaining an oligonucleotide analogue with a lower melting temperature of said self-hybrid than when compared to the oligonucleotide or oligonucleotide analogue not comprising any intercalator pseudonucleotides.

The melting temperature may be decreased according to the nature and number of intercalator pseudonucleotides inserted and according to where said intercalator pseudonucleotides are inserted and between which nucleotides and/or nucleotide analogues that said intercalating pseudonucleotides are positioned in the case of a self hybrid. Preferably, the melting temperature is decreased with at least 2° C., such as at least 5° C., for example at least 10° C., such as at least 15° C., such as from 2 to 50° C., such as from 2 to 40° C., such as 2 to 30° C., for example from 5 to 20° C., such as from 10° C. to 15° C., for example from 2° C. to 5° C., such as from 5° C. to 10° C., for example from 10° C. to 15° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C.

In the design of an oligonucleotide and/or oligonucleotide analogue preferably at least 2 intercalator pseudonucleotides are introduced into said oligonucleotide or oligonucleotide analogue. More preferably, they are positioned as described above with respect to reducing self-hybridisation.

More preferably, the at least 2 intercalator pseudonucleotides are introduced into said oligonucleotide or oligonucleotide analogue so that they are positioned in relation to least one nucleotide or nucleotide analogue from the group comprising, but not limited to β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or PNA, in a manner so that they are close vicinity of each other when the oligonucleotide analogue is hybridised to itself. More preferably, the at least 2 intercalator pseudonucleotide are introduced into said oligonucleotide or oligonucleotide analogue so that they are positioned in relation to two nucleotides comprised in the following group; β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, β-D-RNA, RNA or PNA, so that they each are between said nucleotide analogues when the oligonucleotide is hybridised to itself.

Method for Avoiding Unspecific Hybridisation

It is an object of the present invention to provide methods of decreasing unspecific hybridisation between oligonucleotides and/or oligonucleotide analogues and non-target nucleic acids and/or nucleic acid analogues and/or oligonucleotides and/or oligonucleotide analogues. This is achieved by providing an oligonucleotide and/or oligonucleotide analogue comprising at least one intercalator pseudonucleotide.

In one embodiment of the present invention an oligonucleotide and/or oligonucleotide analogue comprising at least one intercalator pseudonucleotide has higher hybridisation specificity towards a complementary nucleic acid and/or nucleic acid analogue target than for homologously but not fully complementary nucleic acid targets.

In a preferred embodiment of the present invention said oligonucleotide and/or oligonucleotide analogue comprising at least one intercalator pseudonucleotide has higher hybridisation specificity towards a complementary nucleic acid and/or nucleic acid analogue than for homologously but not fully complementary nucleic acid and/or nucleic acid analogue targets compared to a homologously complementary oligonucleotide or oligonucleotide analogue not comprising any intercalator pseudonucleotide(s).

Accordingly, at identical hybridisation conditions, said oligonucleotide analogue comprising at least one intercalator pseudonucleotide will bind a proportion of fully complementary versus not fully complementary nucleic acid and/or nucleic acid analogue that is significantly larger than the proportion of fully complementary versus not fully complementary nucleic acid and/or nucleic acid analogue bound by said same oligonucleotide or oligonucleotide analogue not comprising any intercalator pseudonucleotide(s).

Preferentially said oligonucleotide analogue comprising at least one intercalator pseudonucleotide will bind a significantly larger proportion of nucleic acid and/or nucleic acid analogue target, versus non-target nucleic acid and/or nucleic acid analogue.

Detection of Single Nucleotide Polymorphism (SNP)

In one embodiment the present invention relates to detection of SNP, i.e. detection of a nucleic acid target differing with one nucleobase from another nucleic nucleic acid sequence. The detection is conducted using a probe consisting of an oligonucleotide and/or oligonucleotide analogue comprising at least one intercalator pseudonucleotide. The detection is based on a difference in the melting temperature between a duplex of the probe to a complementary target sequence and a duplex of the probe to a target sequence comprising at lease one SNP. Preferably said oligonucleotide and/or oligonucleotide analogue have a higher melting temperature difference than an oligonucleotide or oligonucleotide analogue not comprising said intercalator pseudonucleotides when comparing the melting temperatures of said oligonucleotides or oligonucleotide analogues to their complementary target and said oligonucleotides or oligonucleotide analogues to the SNP sequences. In a preferred embodiment said oligonucleotide analogue comprises at least one intercalator pseudonucleotide. In yet another preferred embodiment said intercalator pseudonucleotides is placed in close vicinity of the non-complementary nucleobases of the duplex consisting of said probe and said SNP target nucleic acid and/or nucleic acid analogue and/or oligonucleotide and/or oligonucleotide analogue.

In another preferred embodiment of the present invention said oligonucleotide and/or oligonucleotide analogue comprises at least two intercalator pseudonucleotides positioned in a way so that at least one intercalator pseudonucleotide is positioned upstream and at least one intercalator pseudonucleotide is positioned downstream from the at least one nucleobase mismatch when said probe is hybridized to the SNP comprising sequence.

In an even more preferred embodiment of the present invention, said intercalator pseudonucleotides are placed within 4 nucleobases to each side of said non-complementary nucleobases of said hybrid formed due to hybridization between said probe and said SNP comprising complementary sequence.

Most preferably said intercalator pseudonucleotides are placed within 2 nucleobases to each side of said non-complementary nucleobases of said hybrid formed due to hybridization between said probe and said SNP comprising complementary sequence.

Method for Leveling Melting Temperature Differences in Multiplex Assays

It is an object of the present invention to provide methods of leveling the difference in melting temperature encountered in multiplex hybridisation assays, when conducted in the same reaction vessel or at a common surface and/or to enable the standardisation of experimental conditions under which hybridisation experiments are performed, particularly with regard to temperature and/or buffer conditions, to increase the validity of cross-experiment comparisons. These objects are important to most nucleic acid based screens since the methodology of this type of screens is almost based exclusively on nucleic acid hybridisation. Furthermore such screens are now to an increasing extent being done in multiple ways. The practical restraints on choosing comparable nucleotide sequences with regard to hybridisation in such assays naturally impose a great deal of variability on the melting temperatures of different sequences.

Accordingly, it is an aspect of the present invention to provide a system for multiple hybridisation assays as described above. Said system may be in any suitable design, such as an array, a chip, an electronic chip, a reaction vessel such as a microtiter plate well, a glass capillary tube, a chamber or capillary in a flow cytometer or any other flow controlled device, wherein at least one oligonucleotide and/or oligonucleotide analogue is arranged.

Providing oligonucleotides and/or oligonucleotide analogues that hybridise with strong affinity towards AT-rich targets will compensate for the low contribution per nucleobase to the melting temperature of nucleic acids and nucleic acid analogues provided by A- and T-nucleobases due to the fact that the A-T basepair only possess two hydrogen bonds where G-C basepairs have three hydrogen bonds.

It is therefore an aspect of the present invention to provide a system having oligonucleotides and/or oligonucleotide analogue comprising at least one intercalator pseudonucleotide according to the present invention where said intercalator pseudonucleotide will stabilize the hybridization between said oligonucleotide and/or oligonucleotide analogue and its homologously complementary nucleic acid or nucleic acid analogue due to intercalation next to at least one AT basepair.

It is a another aspect of the present invention to provide oligonucleotide analogues with approximately same number of nucleobases comprising at least one intercalator pseudonucleotide according to the present invention where the hybridization affinity towards homologously complementary AT-rich nucleic acids and/or nucleic acid analogues are comparable to the hybridization affinity towards GC-rich targets.

It is also an object of the present invention to provide methods of leveling the melting temperature in multiplex hybridisation assays between different oligonucleotide and/or oligonucleotide analogue sequences comprising at least one intercalator pseudonucleotide and their homologously complementary nucleic acid target and/or nucleic acid analogue target, wherein the melting temperatures of said oligonucleotide analogue sequences and said nucleic acids and/or nucleic acid analogues are significantly more homogeneous than the melting temperatures of said oligonucleotide analogue sequences comprising no intercalator pseudonucleotides and said homologously complementary nucleic acids target or nucleic acid analogues target.

In one embodiment of the present invention an oligonucleotide analogue comprising at least one intercalator pseudonucleotide has one or more intercalator pseudonucleotides placed to specifically intercalate in A- and/or T-rich regions of a homologously complementary nucleic acid or nucleic acid analogue upon hybridization.

In another embodiment of the present invention said oligonucleotide analogue comprising at least one intercalator pseudonucleotide placed to intercalate in A- and/or T-rich regions of said homologously complementary nucleic acid or nucleic acid analogue upon hybridisation, said one or more intercalator pseudonucleotides is placed to specifically increase binding affinity towards A- and/or T-rich targets.

If more than one different homologously complementary nucleic acids or nucleic acid analogues are provided whereto hybridisation of oligonucleotides or oligonucleotide analogues needs to be detected simultaneously, the hybrid between said oligonucleotides and/or oligonucleotide analogues and said homologously complementary nucleic acids and/or nucleic acid analogues typically needs to be carried out under identical conditions. Accordingly, it is an advantage to have approximately equal melting temperatures of said hybrids.

In a preferred embodiment of the present invention at least two oligonucleotides or oligonucleotide analogues of which at least one comprises at least one intercalator pseudonucleotide according to present invention is provided for multiplex detection of at least two different homologously complementary nucleic acids or nucleic acid analogues. In another preferred embodiment the melting temperature of at least two of the hybrids between said oligonucleotides or oligonucleotide analogues and said homologously complementary nucleic acids or nucleic acid analogues are small. More preferred all of the said hybrids have melting temperatures that are comparable, such as essentially identical, i.e. the difference between the melting temperature being less than 5 degrees Celcius, more preferably less than 3 degrees Celcius.

By placing intercalator pseudonucleotides preferentially in A- and/or T-rich regions of said oligonucleotides or oligonucleotide analogues, the small contribution to hybridisation strength normally provided by A- and/or T-rich regions of hybridized nucleic acid and/or nucleic acid analogue duplex structures are consequently leveled and accordingly the hybridization temperature of the said nucleic acid or nucleic acid analogue targets toward said essentially complementary oligonucleotide analogues are more similar than without intercalator pseudonucleotides.

In one embodiment of the present invention the multiple hybridisation system comprises at least 1, such as from 2 to 10⁶, such as from 2 to 10⁵, such as from 2 to 10⁴, such as from 2 to 10³, such as from between 2 to 5, for example from 5 to 10, such as from 10 to 50, for example from 50 to 100, such as from 100 to 1000, for example from 1000 to 5000, such as from 5000 to 10000, for example from 10000 to 50000, such as from 50000 to 100000, for example from 100000 to 1000000 different sequences of oligonucleotides and/or oligonucleotide analogues according to the present invention may be provided, wherein at least one oligonucleotide and/or oligonucleotide analogue comprises at least one intercalator pseudonucleotide, preferably all oligonucleotides and/or oligonucleotide analogues comprise at least one intercalator pseudonucleotide.

In another embodiment of the present invention at least 1, such as between 2 and 5, for example between 5 and 10, such as between 10 and 50, for example between 50 and 100, such as between 100 and 1000, for example between 1000 and 5000, such as between 5000 and 10000, for example between 10000 and 50000 different sequences of oligonucleotide analogues according to the present invention may be provided affixed to a solid support.

Method for Providing Nuclease-Stable Oligonucleotides and/or Oligonucleotide Analogues

In one aspect the present invention relates to an oligonucleotide and/or oligonucleotide analogue that is nuclease stable. This is achieved by providing an oligonucleotide and/or oligonucleotide analogue comprising at least one intercalating pseudonucleotide according to the invention.

The oligonucleotide and/or nucleotide analogue comprising at least one pseudonucleotide is of use in most assays where nuclease-mediated breakdown of oligonucleotides and/or oligonucleotide analogues can cause non-optimal results, e.g. in probe assays performed in a living cell or in DNA amplification processes where DNA polymerases possessing exonuclease activity is used.

Separating Sequence Specific DNA(s)

The present invention provides methods of separating a sequence specific DNA(s) from a mixture comprising nucleic acids comprising the steps of
a) providing a mixture comprising nucleic acids; and
b) providing one or more different oligonucleotides or oligonucleotide analogues, wherein the melting temperature of a hybrid consisting of said oligonucleotide or oligonucleotide analogue and a homologously complementary DNA (DNA hybrid), is significantly higher than the melting temperature of a hybrid consisting of said oligonucleotide or oligonucleotide analogue and a homologously complementary RNA (RNA hybrid), and wherein said oligonucleotides or oligonucleotide analogues are capable of hybridising with said sequence specific DNA (target DNA); and
c) incubating said mixture with said oligonucleotide or oligonucleotide analogue under conditions that allow for hybridisation with a homologously complementary oligonucleotide or oligonucleotide analogue; and
d) separating the oligonucleotides or oligonucleotide analogues together with nucleic acids hybridised to said oligonucleotide or oligonucleotide analogues from the mixture; and
thereby obtaining separated sequence specific DNA(s) and a separated remaining mixture comprising nucleic acids.

The mixture comprising nucleic acids may comprise any nucleic acids or nucleic acid analogues, for example it may comprise, but not limited to DNA, β-D-Homo-DNA, β-D-Altropyranosyl-NA, β-D-Glucopyranosyl-NA, β-D-Allopyranusyl-NA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, α-L-Lyxopyranosyl-NA, 2′-R-RNA, 2′-OR-RNA, α-L-RNA, α-D-RNA, 6-D-RNA, RNA or PNA. Preferably however the nucleic acids are RNA and DNA.

The mixture comprising nucleic acids may for example be an intact cell comprising nucleic acids or the mixture may be a cellular extract comprising nucleic acids. Furthermore the mixture may be purified nucleic acids, the mixture may be a synthetically prepared mixture of nucleic acids or the mixture may be a chemically or enzymatically modified mixture of nucleic acids or nucleic acid analogues, for example bisulphate converted DNA or partially restriction enzyme digest of DNA.

Preferred oligonucleotides or oligonucleotide analogues to be employed with the methods of separating a sequence specific DNA(s) from a mixture comprising nucleic acids, are oligonucleotide or oligonucleotide analogues selected from the group consisting of oligonucleotides or oligonucleotide analogues comprising at least one intercalator pseudonucleotide as described herein above.

Such oligonucleotides or oligonucleotide analogues may for example consists of from 5-100, such as from 5-50, such as from 5-30, such as from 5 to 10, such as from 10 to 15, for example from 15 to 20, such as from 20 to 30, for example from 30 to 100 nucleotides and/or nucleotide analogues and/or intercalator pseudonucleotides. Preferably said oligonucleotides or oligonucleotide analogues consists of from 10 to 50 nucleotides and/or nucleotide analogues and/or intercalator pseudonucleotides.

It is possible to separate more than one kind of sequence specific DNA(s) from said mixture. To do so it is necessary to provide different oligonucleotides or oligonucleotide analogues, wherein at least one of the provided oligonucleotides or oligonucleotide analogues are capable of hybridising with each of the sequence specific DNA(s). In this embodiment a multiple hybridisation system may be used, said system preferably comprises at least 1, such as from 2 to 10⁶, such as from 2 to 10⁵, such as from 2 to 10⁴, such as from 2 to 10³, such as from between 2 to 5, for example from 5 to 10, such as from 10 to 50, for example from 50 to 100, such as from 100 to 1000, for example from 1000 to 5000, such as from 5000 to 10000, for example from 10000 to 50000, such as from 50000 to 100000, for example from 100000 to 1000000 different sequences of oligonucleotides and/or oligonucleotide analogues according to the present invention may be provided.

Such a method may be performed to achieve one of several goals. For example, the goal may be to remove sequence specific DNA from a mixture comprising RNA and DNA with the same or essentially the same sequence. The same sequence is be understood so that DNA contains the nucleotide T is the place of the nucleotide U in RNA, but that the sequence otherwise is identical. A mixture of RNA and DNA from which sequence specific DNA has been removed may for example serve as a template for Reverse transcription polymerase chain reaction (RT-PCR) and has the advantage of eliminating false positives, which may arise due to DNA contamination. It is frequently a problem to obtain an RNA sample free of DNA. For example the company Ambion describes in its 2001 catalogue, p. 6, how a number of commonly used RNA purification methods results in an RNA preparation which is not free of DNA. In the prior art, DNA contamination has been eliminated by DNase treatment, which is expensive and time consuming, because it is usually necessary to remove the DNase after treatment.

The present invention provides a method to remove sequence specific DNA from an RNA sample. RNA may have been isolated by any method known to the person skilled in the art in particular the method may be useful when RNA has been isolated from complex biological samples.

Total RNA may for example be routinely isolated by several methods including guanidium thiocyanate/acid phenol:chloroform based procedures, filter binding based procedures and centrifugation through CsCl gradients. Regardless of the method, contaminating chromosomal DNA are usually present in isolated RNA samples (Ambion TechNotes 7, 1, 2000), which may give rise to problems during the procedure(s) for which the RNA may be used. For an introduction to the problems related to DNA contamination of isolated RNA, see for example Critical factors for PCR, Chap 18, Qiagen, www.qiagen.com. Due to the selective nature of the oligonucleotide or oligonucleotide analogue comprising intercalator pseudonucleotides according to the present invention, it is possible to remove DNA contaminations fast and easy and without excessive loss of RNA material.

Accordingly it is preferred that the remaining mixture comprising nucleic acids is essentially free of the sequence specific DNA(s). Hence, preferably sufficient oligonucleotides or oligonucleotide analogues should be provided, so that every sequence specific DNA molecule may hybridise with one oligonucleotide or oligonucleotide analogue.

In another embodiment of the present invention, the method relates to removal of DNA contamination from a sample of poly(A) containing RNA (mRNA). Poly(A) containing RNA is routinely purified based on its specific association with poly(dT) (DNA oligonucleotide consisting only of thymidine) or poly(U) (RNA oligonucleotide consisting only of uridine). For example columns with oligo(dT) attached to cellulose or oligo(U) attached to sephadex, are typically used in mRNA purification procedures (Current Protocols in Molecular Biology, 1995, John Wiley & Sons Inc., USA, Chap. 4.5). However, purified poly(A) RNA frequently contains DNA contamination, in particular it often contains DNA species capable of associating with poly dT and/or oligo(U). To obtain mRNA free of DNA contamination, it is thus necessary to conduct two rounds of poly(dT) selection. This is often too labour intensive for routine analysis. Furthermore it may also change the relative amounts of individual transcripts, probably due to differential polyadenylation between tissues or in response to biological stimuli of (Ambion Technical bulletin 176, www.ambion.com). The present invention relates to a method of removing contaminating DNA using the method outlined herein above, wherein the oligonucleotide or oligonucleotide analogue is consisting essentially of intercalator pseudonucleotides and thymidine nucleotides or nucleotide analogues.

In yet another embodiment, practically all genomic DNA in a mixture of nucleic acids can be removed by sequence specific hybridisation to a restricted set of oligonucleotides or oligonucleotide analogues. Throughout mammalian genomes are dispersed different types of 100 to 500 base pair repeated sequences of which the most abundant are the SINES (short interspersed repeats) and LINES (long interspersed repeats). In particular the Alu LINES recur around one million times in the human genome, corresponding to an average of one repeat per 3000 bases.

It is an objective of the present invention to provide a selection of RNA/DNA discriminating RNA-selective oligonucleotide or oligonucleotide analogue sequences preferentially comprising intercalator pseudonucleotides according to the present invention, where said sequences cover a range of known repeated elements of eukaryotic and preferentially mammalian genomes. Accordingly, when providing a mixture of nucleic acids from eukaryotic or mammalian origin, preferentially treated by enzymatic restriction digestion of DNA, sonication or any other method of partially fragmenting nucleic acid known to the person skilled in the art, said selection of RNA-selective sequences will under appropriate stringency conditions hybridise to said repeated elements in genomic DNA of said eukaryotic or mammalian nucleic acid mixture. If providing said oligonucleotide or oligonucleotide analogue sequences bound to a solid support and separating said solid support with oligonucleotide or oligonucleotide analogue bound DNA from said nucleic acid mixture, remaining RNA will be separated from said genomic DNA and thus said RNA will be purified.

Yet another way to obtain an RNA mixture essentially free of contaminating DNA is to provide a selection of RNA/DNA discriminating RNA-selective oligonucleotide or oligonucleotide analogue sequences preferentially comprising intercalator pseudonucleotides according to the present invention, where said sequences are of a certain length and randomised to cover all possible sequences of this length. Thus said selection of oligonucleotide or oligonucleotide analogue sequences will be able to hybridise with all homologously complementary DNA sequences represented by said randomised pool of sequences. If providing said oligonucleotide or oligonucleotide analogue sequences bound to a solid support and separating said solid support with oligonucleotide or oligonucleotide analogue bound DNA from said nucleic acid mixture, remaining RNA will be separated from said genomic DNA and thus said RNA will be purified.

In still another object of the present invention, the two above described objectives of providing a selection of RNA/DNA discriminating RNA-selective oligonucleotide or oligonucleotide analogue sequences preferentially comprising intercalator pseudonucleotides according to the present invention that is either representing a set of repeated elements or randomised sequences is combined. The combination of the two objectives may compromise for individual shortcomings of the two methods.

Another goal of such a method may be to obtain sequence specific DNA from a mixture of DNA and RNA in a one step procedure, which does not involve further removal of sequence specific RNA by for example treatment with RNase.

For example the methods described herein above allows one to separate pure DNA samples from complex biological samples or specimens. Accordingly the method may for example be employed for isolating DNA released from a lysed complex biological mixture containing nucleic acids, such as a cell, or DNA may be purified from a reasonably pure sample. Such methods may for example include lysing the cells in a hybridisation medium comprising a strong chaotropic agent, contacting the lysate under hybridisation conditions with the oligonucleotide or oligonucleotide analogue, and isolating the sequence for further use.

The isolated DNA may for example be employed for cloning, as template for implication reactions, for hybridisation assays, for diagnosis or any other method known to the person skilled in the art.

Incubation of the mixture of nucleic acids with said oligonucleotide or oligonucleotide analogue under conditions that allow for hybridisation may be done in any manner known to the person skilled in the art.

In one embodiment of the present invention the nucleic acid mixture will comprise a chaotropic agent, a target nucleic acid, and the oligonucleotide or oligonucleotide analogue substantially complementary to the target nucleic acid. Preferably, the nucleic acid mixture will be heated to disrupt protein/nucleic acid interactions prior to or simultaneous to hybridisation in order to maximise hybridisation between the oligonucleotide or oligonucleotide analogue and its target.

For example, the nucleic acid mixture may be heated to disrupt protein/nucleic acid interactions and subsequently cooled until hybridisation between the oligonucleotide or oligonucleotide analogue and the target DNA has occurred.

When high affinity oligonucleotides or oligonucleotide analogues are employed, hybridisation may take place at the increased temperature, preferably the temperature needed to fully disrupt DNA:DNA and DNA:RNA interactions. Preferably the temperature is low enough to allow hybridisation between the oligonucleotide or oligonucleotide analogue and the target DNA (DNA hybrid).

Because the melting temperature of the DNA hybrid, preferably is higher than the melting temperature of homologously complementary DNA:DNA duplex and the RNA hybrid, the temperature may be selected in order to allow hybridisation of the DNA hybrid but not the RNA hybrid or DNA:DNA duplex.

Other features than the melting temperature may also be selected to optimise specific hybridisation of the DNA hybrid, for example salt concentration and/or pH of the buffer.

Different methods may be employed to separate the oligonucleotides or oligonucleotide analogues together with nucleic acids, preferably DNA hybridised to said oligonucleotides from the mixture. For example the separation may be done by gel electrophoresis, by gel filtration or any other method known to the person skilled in the art.

In one preferred embodiment of the present invention the oligonucleotides or oligonucleotide analogues are coupled to a solid support. The separation of oligonucleotides or oligonucleotide analogues together with nucleic acids hybridised to said oligonucleotides from the mixture may then be performed by separating said solid support from the mixture.

Many different kinds of solid supports are suitable for the method, depending of the desired outcome.

In one embodiment the solid support is an activated surface. An activated surface facilitates coupling of oligonucleotides or oligonucleotide analogues to the solid support.

The solid support may for example be selected from the group consisting of magnetic beads, metal beads, aluminium beads, agarose beads, sepharose beads, coded beads of any kind, e.g. barcoded beads, glass, plastic surfaces, heavy metals and chip surfaces.

Magnetic beads include beads comprising a magnetic material that allows the beads to be separated from a suspension using a magnet.

Agarose beads and sepharose beads may for example be separated from a suspension by centrifugation or filtration.

Plastic surfaces include for example microtiter plates or other plastic devices that may be suitable for example for diagnosis. Chips surfaces may be made of any suitable materials, for instance, a glass plate, a resin plate, a metal plate, a glass plate covered with polymer coat, a glass plate covered with metal coat, and a resin plate covered with metal coat. Also employable is a SPR (surface plasmon resonance) sensor plate, which is described in Japanese Patent Provisional Publication No. 11-332595. CCD is also employable as described in Nucleic Acids Research, 1994, Vol. 22, No. 11, 2124-2125.

Chip surfaces include small polyacrylamide gels on a glass plate whereto oligonucleotides or oligonucleotide analogues may be fixed by making a covalent bond between the polyacrylamide and the oligonucleotide (Yershov, G., et al., Proc. Natl. Acad. Sci. USA, 94, 4913(1996)).

Chips surfaces may also be silica chips as described by Sosnowski, R. G., et al., Proc. Natl. Acad. Sci. USA, 94, 1119-1123 (1997). Such chips are prepared by a process comprising the steps of placing an array of microelectrodes on a silica chip, forming on the microelectrode a streptavidin-comprising agarose layer, and attaching biotin-modified DNA fragments to the agarose layer by positively charging the agarose layer.

Furthermore, chips surfaces may be prepared as described by Schena, M., et al., Proc. Natl. Acad. Sci. USA, 93, 10614-10619 (1996) wherein a process comprising the steps of preparing a suspension of an amino group-modified PCR product in SSC (i.e., standard sodium chloride-citric acid buffer solution), spotting the suspension onto a slide glass, incubating the spotted glass slide, treating the incubated slide glass with sodium borohydride, and heating thus treated slide glass.

The methods described herein are additionally advantageous because they allow for minimal handling of the samples and assay reagents. Hence it is possible to provide a ready-to-use reagent solution, for example, such a ready-to-use reagent solution may contain a chaotropic agent, other appropriate components such as buffers or detergents, an oligonucleotide or oligonucleotide analogue which may be bound to a solid support capable of hybridising with a target nucleic acid.

Conveniently, a sample, for example a complex biological sample may be directly combined with the pre-prepared reagent for hybridisation, thus allowing the hybridisation to occur in one step. The combined solution may be heated to the desired temperature as described herein and then cooled until hybridization has occurred. The resulting hybridization complex is then simply washed to remove unhybridized material, and the extent of hybridization is determined.

Method of Detecting a Sequence Specific DNA

In one aspect the present invention relates to a method of detecting a sequence specific DNA (target DNA) in a mixture comprising nucleic acids and/or nucleic acid analogues comprising the steps of
a) providing a mixture of nucleic acids; and
b) providing one or more different oligonucleotides or oligonucleotide analogues, wherein the melting temperature of a hybrid consisting of said oligonucleotide or oligonucleotide analogue and a homologously complementary DNA (DNA hybrid), is significantly higher than the melting temperature of a hybrid consisting of said oligonucleotide or oligonucleotide analogue and a homologously complementary RNA (RNA hybrid), and wherein said oligonucleotides or oligonucleotide analogues are substantially complementary to said sequence specific DNA (target DNA); and
c) incubating said mixture with said oligonucleotide or oligonucleotide analogue under conditions that allow for hybridisation; and
d) detecting the oligonucleotide or oligonucleotide analogue hybridised to sequence specific DNA.

The advantage of said method over methods known in the prior art is that this method allows for sequence specific detection of DNA in a mixture comprising said sequence specific DNA as well as RNA with a similar sequence. Accordingly, background signal from RNA is reduced significantly without treatment with RNase.

Preferably the mixture comprises DNA and RNA. More preferably the mixture does not comprise other nucleic acids or nucleic acids analogues than DNA and RNA.

Preferred oligonucleotides or oligonucleotide analogues to be employed with the methods of detecting sequence specific DNA in a mixture comprising nucleic acids and/or nucleic acid analogues, are oligonucleotide analogues selected from the group consisting of oligonucleotide analogues comprising at least one intercalator pseudonucleotide as described herein above.

Such oligonucleotide analogues may for example consists of 3 to 10, such as 10 to 15, for example 15 to 20, such as 20 to 30, for example 30 to 100 nucleotides and/or nucleotide analogues and/or intercalator pseudonucleotides. Preferably said oligonucleotide analogues consist of between 3 and 50 nucleotides and/or nucleotide analogues and/or intercalator pseudonucleotides.

Moreover, the oligonucleotide analogue may be any of the oligonucleotide analogues comprising intercalator pseudonucleotides described herein above.

The mixture may be comprised within a cell, for example within an intact cell. The cell may for example be a prokaryotic cell or a eukaryotic cell, such as a plant cell or a mammalian cell. In such an embodiment the method may be employed for in situ hybridisation.

The method may involve a separation step prior to detection, wherein hybridised oligonucleotide or oligonucleotide analogue is separated from unhybridised oligonucleotide or oligonucleotide analogue, which may facilitate specific detection of only hybridised oligonucleotide or oligonucleotide analogue. For example, the mixture of nucleic acids may be immobilised on a solid support prior to hybridisation with the oligonucleotide or oligonucleotide analogue. After hybridisation, unhybridised oligonucleotide or oligonucleotide analogue may be washed away and hybridised oligonucleotide or oligonucleotide analogue may be detected.

Alternatively, the method may involve the method of separation of sequence specific DNA(s) from a mixture as outlined herein above, prior to detection. For example, the oligonucleotide or oligonucleotide analogue may be bound to a solid support and after hybridisation unbound nucleic acids, may be washed away and bound nucleic acids may be detected.

The target DNA may for example be a particular gene, a gene segment, a microsatellite or any other DNA sequence. Of particular interest is the detection of particular DNAs, which may be of eukaryotic, prokaryotic, Archae or viral origin. Importantly, the invention may assist in the diagnosis of various infectious diseases by assaying for particular sequences known to be associated with a particular microorganism. The target DNA may be provided in a complex biological mixture of nucleic acid (RNA, DNA and/or rRNA) and non-nucleic acid, for example an intact cell or a crude cell extract.

If target DNA is double stranded or otherwise have significant secondary and tertiary structure, they may need to be heated prior to hybridisation. In this case, heating may occur prior to or after the introduction of the nucleic acids into the hybridisation medium containing the oligonucleotide or oligonucleotide analogue. It may also be desirable in some cases to extract the nucleic acids from the complex biological samples prior to the hybridisation assay to reduce background interference by any methods known in the art. Double stranded target DNA may also be detected by triplex formation and/or strand invasion as discussed herein.

The hybridisation and extraction methods of the present invention may be applied to a complex biological mixture of nucleic acid (DNA and/or RNA) and non-nucleic acid. Such a complex biological mixture includes a wide range of eukaryotic and prokaryotic cells, including protoplasts; or other biological materials that may harbor target deoxyribonucleic acids. The methods are thus applicable to tissue culture animal and human cells, animal and human cells from e.g., blood, serum, plasma, reticulocytes, lymphocytes, urine, bone marrow tissue, cerebrospinal fluid or any product prepared from blood or lymph or any type of tissue biopsy (e.g. a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, an uterus biopsy, a testicular biopsy, an eye biopsy or a brain biopsy, homogenized in lysis buffer), plant cells or other cells sensitive to osmotic shock and cells of bacteria, yeasts, viruses, mycoplasmas, protozoa, rickettshia, fungi and other small microbial cells and the like. Said mixture comprising nucleic acic may also be used for samples derived from or extracts of food, beverages, pharmaceutical products, personal care products, dairy products or environmental mental samples. The assay and isolation procedures of the present invention are useful, for instance, for detecting non-pathogenic or pathogenic microorganisms of interest. By detecting specific hybridisation between oligonucleotide or oligonucleotide analogue comprising intercalator pseudonucleotide and nucleic acids resident in the biological sample, the presence of the microorganisms may be established.

Solutions containing high concentrations of guanidine, guanine thiocyanate or certain other chaotropic agents and detergents are capable of effectively lysing prokaryotic and eukaryotic cells while simultaneously allowing specific hybridisation of the oligonucleotide analogues according to the invention to the released endogenous DNA. The solutions need not contain any other component other than common buffers and detergents to promote lysis and solubilisation of cells and nucleic acid hybridisation.

If extraction procedures are employed prior to hybridisation, organic solvents such as phenol and chloroform may be used in techniques employed to isolate nucleic acid. Traditionally, organic solvents, such as phenol or a phenol-chloroform combination is used to extract nucleic acid, using a phase separation (Ausubel et. al in Current Protocols in Molecular Biology, pub. John Wiley & Sons (1998). These methods may be used effectively with the lysis solutions of the present invention; however, an advantage of the methods of the present invention is that tedious extraction methods are not necessary, thus improving the performance of high throughput assays. Preferably, the lysis buffer/hybridisation medium will contain standard buffers and detergents to promote lysis of cells while still allowing effective hybridization of oligonucleotide analogues comprising intercalator pseudonucleotides. A buffer such as sodium citrate, Tris-HGI, PIPES or HEPES, preferably Tris-HGI at a concentration of about 0.05 to 0.1M can be used. The hybridisation medium will preferably also contain about 0.05 to 0.5% of an ionic or non-ionic detergent, such as sodium dodecylsulphate (SDS) or Sarkosyl (Sigma Chemical Go., St. Louis, Mo.) and between 1 and 10 mM EDTA. Other additives may also be included, such as volume exclusion agents which include a variety of polar water-soluble or swellable agents, such as anionic polyacrylate or polymethacrylate, and charged saccharidic polymers, such as dextran sulphate and the like. Specificity or the stringency of hybridisation may be controlled, for instance, by varying the concentration and type of chaotropic agent and the NaCl concentration which is typically between 0 and 1 M NaCl, such as 0.1, 0.2, 0.3, 0.4, 0.5, 0.6, 0.7, 0.8, 0.9 or 1.0 M NaCl.

Chaotropic agents which disturb the secondary and tertiary structure of proteins, for example, guanidine salts such as guanidine hydrochloride (GnHCl) and thiocyanate (GnSCN), or urea, lithium chloride and other thiocyanates may be used in combination with detergents and reducing agents such as beta-mercaptoethanol or DTT to dissociate natural occurring nucleic acids and inhibit nucleases. The use of chaotropic agents in the extraction and hybridization of nucleic acids is described in EP Publication No.0127 327, which is incorporated by reference herein.

An oligonucleotide analogue comprising intercalator pseudonucleotides substantially complementary to the target DNA will be provided in the hybridisation process.

In order to detect oligonucleotide analogues comprising intercalator pseudonucleotides they may be linked to a group (e.g. biotin, fluorescein, magnetic micro-particle etc.). Alternatively, they may be permanently bound to a solid phase or particle in advance e.g. by anthraquinone photochemistry (WO 96/31557).

An attractive possibility of the invention is the use of different oligonucleotide analogues directed against different sequences in the genome, which are spotted in an array format and permanently affixed to the surface (Nature Genetics, suppl. vol. 21, January 1999, 1-60 and WO 96/31557). Such an array can subsequently, be incubated with the mixture of the lysis buffer/hybridisation medium containing dissolved cells and a number of suitable detection oligonucleotides or oligonucleotide analogues. The lysis and hybridisation would then be allowed to occur, and finally the array would be washed and appropriately developed. The result of such a procedure would be a semi-quantitative assessment of a large number of different target DNAs.

As for DNA or RNA the degree of complementarity required for formation of a stable hybridisation complex (duplex) with an oligonucleotide or oligonucleotide analogue varies with the stringency of the hybridisation medium and/or wash medium. The complementary nucleic acid may be present in a pre-prepared hybridisation medium or introduced at some later point prior to hybridisation.

The hybridisation medium is combined with the biological sample to facilitate lysis of the cells and nucleic acid pairing. Preferably, the volume of biological sample to the volume of the hybridisation medium should not be to large, e.g. the ratio of the volume of biological sample to the volume of the hybridisation medium could be about 1:10.

In one embodiment of the present invention the hybridisation methods of the present invention may preferably be carried out in one step on complex biological samples. However, minor mechanical or other treatments may be considered under certain circumstances. For example, it may be desirable to clarify the lysate before hybridisation such as by slow speed centrifugation or filtration or to extract the nucleic acids before hybridisation as described above.

The hybridisation assay of the present invention can be performed by any method known to those skilled in the art or analogous to immunoassay methodology given the guidelines presented herein. Preferred methods of assay are the sandwich assays and variations thereof and the competition or displacement assay. Hybridization techniques are generally described in “Nucleic Acid Hybridization, A Practical Approach,” Ed. Hames, B. D. and Higgins, S. J., IRL Press, 1985; Gall and Pardue (1969), Proc. Natl. Acad. Sci., U.S.A., 63:378-383; and John, Burnsteil and. Jones (1969) Nature, 223:582-587. Further improvements in hybridisation techniques will be well known to the person of skill in the art and can readily be applied.

In one embodiment of the present invention the oligonucleotide or oligonucleotide analogue comprising intercalator pseudonucleotides serves as capturing probe in an assay. Preferably said capturing probe is attached to a solid surface e.g. the surface of a microtiter tray well, a chip surface or a microbead. Therefore a convenient and very efficient washing procedure can be performed thus opening the possibility for various enzymatically based reactions that may add to the performance of the invention. Most noteworthy is the possibility that the sensitivity of the hybridisation assays may be enhanced through use of a nucleic acid amplification system which multiplies the target DNA being detected. Examples of such systems include the polymerase chain reaction (PCR) system, the isothermal amplification and the ligase chain reaction (LCR) system. Other methods known to the person of skill in the art such as the nucleic acid sequence based amplification (NASBA™, Cangene, Mississauga, Ontario) and Q Beta Replicase systems. PCR is a template dependent DNA polymerase primer extension method of replicating selected sequences of DNA. The method relies upon the use of an excess of specific primers to initiate DNA polymerase replication of specific sub-sequences of a DNA polynucleotide followed by repeated denaturation and polymerase extension steps. The PCR system is well known in the art (see U.S. Pat. No. 4,683,195 and U.S. Pat. No. 4,683,202). For additional information regarding PCR methods, see also PCR Applications Manual 2^{nd} ed. Roche Diagnostics or PCR Protocols: A Guide to Methods and Applications, ed. Innis, Gelland, Shinsky and White, Academic Press, Inc. (1990).

LCR, like PCR, uses multiple cycles of alternating temperature to amplify the numbers of a targeted sequence of DNA. LCR, however, does not use individual nucleotides for template extension. Instead, LCR relies upon an excess of oligonucleotides that are complementary to both strands of the target region. Following the denaturation of a double stranded template DNA, the LCR procedure begins with the ligation of two oligonucleotide primers complementary to adjacent regions on one of the target strands. Oligonucleotides complementary to either strand can be joined. After ligation and a second denaturation step, the original template strands and the two newly joined products serve as templates for additional ligation to provide an exponential amplification of the targeted sequences. This method has been detailed in Genomics, 4:560-569 (1989), which is incorporated herein by reference. As other amplification systems are developed, they may also find use in this invention.

The hybridisation medium and processes of the present invention are uniquely suited to a one-step assay. The medium may be pre-prepared, either commercially or in the laboratory to contain all the necessary components for hybridization. For instance, in a sandwich assay the medium could comprise a chaotropic agent (e.g. guanidine thiocyanate) desired buffers and detergents, a capturing probe comprising intercalator pseudonucleotides bound to a solid support such as a microbead, and a detecting nucleic acid which could also comprise intercalator pseudonucleotides, however it must not necessarily comprise intercalator pseudonucleotides. This medium then only needs to be combined with the sample containing the target nucleic acid at the time the assay is to be performed. Once hybridization occurs the hybridization complex attached to the solid support may be washed and the extent of hybridization determined.

Sandwich assays are commercially useful hybridisation assays for detecting or isolating nucleic acid sequences. Such assays utilise a “capturing” nucleic acid covalently immobilised on a solid support and labeled “signal” nucleic acid in solution. The sample will provide the target nucleic acid. The “capturing” nucleic acid and “signal” nucleic acid probe hybridise with the target nucleic acid to form a “sandwich” hybridisation complex. To be effective, the signal nucleic acid is designed so that it cannot hybridise with the capturing nucleic acid, but will hybridise with the target nucleic acid in a different position than the capturing probe. This can be ensured using intercalator pseudonucleotides as described herein reducing the affinity for nucleic acids or nucleic acid analogues either with properly positioning of intercalator pseudonucleotides in relation to each other, or with properly positioned intercalator pseudonucleotidesin relation to RNA or RNA-like molecules, while at the same time enhancing affinity for some nucleic acids or nucleic acid analogues as described herein above.

The oligonucleotide analogues according to the present invention comprise an intercalator and hence already comprises a signal system (fluorophore(s)). Accordingly, when oligonucleotide analogues according to the present invention are employed as capturing probe, the intercalator may be used for detecting the extent of hybridisation. The capturing probe may also be used in combinations with a labeled “signal” nucleic acid, wherein the signal nucleic acid may or may not be an oligonucleotide analogue according to the present invention and in this way the specificity and/or the sensitivity of the assay may be enhanced.

Virtually any solid surface may be used as a support for hybridisation assays, including membranes, glass, metals and plastics. Two types of solid surfaces are generally available, namely:

a) Membranes, polystyrene beads, nylon, Teflon, polystyrene/latex beads, latex beads or any solid support possessing an activated silane, carboxylate, sulfonate, phosphate or similar activate-able group are suitable for use as solid surface substratum to which nucleic acids or oligonucleotides can be immobilized.

b) Porous membranes possessing pre-activated surfaces which may be obtained commercially (e.g., Pall Immunodyne Immunoaffinity Membrane, Pall BioSupport Division, East Hills, N.Y., or Immobilon Affinity membranes from Millipore, Bedford, Mass.) and which may be used to immobilize capturing oligonucleotides. Microbeads, including magnetic beads, aluminia beads, beads of polystyrene, teflon, nylon, silica or latex may also be used.

The capturing probe comprising intercalator pseudonucleotide(s) may be attached to surfaces of containers that are compatible with commonly employed PCR amplification techniques.

Sequences suitable for capturing or signal nucleic acids for use in hybridization assays can be obtained from the entire sequence, or portions thereof, of an organism's genome, from messenger RNA, or from cDNA obtained by reverse transcription of messenger RNA. Methods for obtaining the nucleotide sequence from such obtained sequences are well known in the art (see Ausubel et. at in Current Protocols in Molecular Biology, pub. John Wiley & Sons (1998), and Sambrook et al. in Molecular Cloning, A Laboratory Manual, Cold Spring Harbor Laboratory Press, 1989).

Furthermore, a number of both public and commercial sequence databases are accessible and can be approached to obtain the relevant sequences.

The determination of the extent of hybridisation may be carried out by any of the methods well known in the art. If there is no detectable hybridisation, the extent of hybridisation is said to be 0. The oligonucleotide analogues according to the present invention comprises intercalators, which may be used to detect hybridisation directly. In addition the oligonucleotides or oligonucleotide analogues according to the present invention may be coupled to one or more detectable labels. Complementary nucleic acids or signal nucleic acids may be labeled by anyone of several methods typically used to detect the presence of hybridised polynucleotides. The most common method of detection is the use of ligands that bind to labeled antibodies, fluorophores or chemiluminescent agents. However, probes may also be labeled with ³H, 125I, ³⁵S, ¹⁴C, ³³P or ³² P and subsequently detected by autoradiography. The choice of radioactive isotope depends on research preferences due to ease of synthesis, varying stability, and half-lives of the selected isotopes. Other labels include antibodies, which can serve as specific binding pair members for a labeled ligand. The choice of using the oligonucleotide analogues according to the present invention with or without one or more additional labeled nucleotides depends on sensitivity required, the specificity as well as personal preferences. The choice label depends on the sensitivity, ease of conjugation with the probe, stability requirements, and available instrumentation.

Situations can be envisioned in which the detection probes are DNA or RNA. Such probes can be labeled in various ways depending on the choice of label. Radioactive probes are typically made by using commercially available nucleotides containing the desired radioactive isotope. The radioactive nucleotides may for example be incorporated into probes by several means such as by nick translation of double-stranded probes; by copying single-stranded M 13 plasmids having specific inserts with the Klenow fragment of DNA polymerase in the presence of radioactive dNTP; by transcribing cDNA from RNA templates using reverse transcriptase in the presence of radioactive dNTP; by transcribing RNA from vectors containing SP6 promoters or T7 promoters using SP6 or T7 RNA polymerase in the presence of radioactive rNTP; normal PCR including hot dNTPs; by tailing the 3′ ends of probes with radioactive nucleotides using terminal transferase; or by phosphorylation of the 5′ ends of probes using [³²P]-A TP and polynucleotide kinase.

Non-radioactive probes are often labeled by indirect means. Generally, one or more ligand molecule(s) is/are covalently bound to the probe. The ligand(s) then binds to an anti-ligand molecule, which is either inherently detectable or covalently bound to a signal system, such as a detectable enzyme, a fluorescent compound, or a chemiluminescent compound. Ligands and anti-ligands may be varied widely. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

As mentioned the oligonucleotide analogues according to the present invention may in some embodiments also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, AMPPD ([3-(2′-spiroamantane)-4-methoxy-4-(3′-phosphoryloxy)-phenyl-1,2-dioxetane]) and 2,3-dihydrophthalazinediones, e.g., luminol.

The amount of labeled probe that is present in the hybridisation medium or extraction solution may vary widely. Generally, substantial excesses of probe over the stoichiometric amount of the target nucleic acid will be employed to enhance the rate of binding of the probe to the target DNA. Treatment with ultrasound by immersion of the reaction vessel into commercially available sonication baths can often accelerate the hybridisation rates.

After hybridisation at a temperature and time period appropriate for the particular hybridisation solution used, the support to which the capturing probe:target DNA hybridisation complex is attached is introduced into a wash solution typically containing similar reagents (e.g., sodium chloride, buffers, organic solvents and detergent), as provided in the hybridisation solution. These reagents may be at similar concentrations as the hybridisation medium, but often they are at lower concentrations when more stringent washing conditions are desired. The time period for which the support is maintained in the wash solutions may vary from minutes to several hours or more. Either the hybridisation or the wash medium can be stringent. After appropriate stringent washing, the correct hybridisation complex may now be detected in accordance with the nature of the label.

The probe may be conjugated directly with the label. For example where the label is fluorescent, the probe with associated hybridisation complex substrate is detected by first irradiating with light of a particular wavelength. The sample absorbs this light and then emits light of a different wavelength, which is picked up by a detector (Physical Biochemistry, Freifelder, D., W. H. Freeman & Co. (1982), pp. 537-542). Where the label is radioactive, the sample is exposed to X-ray film or a phosphorimagescreen etc. Where the label is an enzyme, the sample is detected by incubation on an appropriate substrate for the enzyme. The signal generated may be a colored precipitate, a colored or fluorescent soluble material, or photons generated by bioluminescence or chemiluminescence.

When the label is an enzyme preferably an assay generating a colored precipitate to indicate a positive reading may be employed, e.g. the enzyme may be selected from the group consisting of horseradish peroxidase, alkaline phosphatase, calf intestine alkaline phosphatase, glucose oxidase and beta-galactosidase. For example, alkaline phosphatase will dephosphorylate indoxyl phosphate, which will then participate in a reduction reaction to convert tetrazolium salts to highly coloured and insoluble formazans.

Detection of a hybridisation complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies, or in some cases, by attachment to a radioactive label. (Tijssen, P. “Practice and Theory of Enzyme Immunoassays,” Laboratory Techniques in Biochemistry and Molecular Biology, Burdon, R. H., van Knippenberg, P. H., Eds., Elsevier (1985), pp. 9-20.)

In the present context, the term “label” thus means a group that is detectable either by itself or as a part of a detection series. Examples of functional parts of reporter groups are biotin, digoxigenin, fluorescent groups (groups that are able to absorb electromagnetic radiation, e.g. light or X-rays, of a certain wavelength, and which subsequently reemits the energy absorbed as radiation of longer wavelength; illustrative examples are dansyl (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetra-methylpiperidine), dinitrophenyl, acridines, coumarins, Cy3 and Cy5 (trademarks for Biological Detection Systems, Inc.), erytrosine, coumaric acid, umbelliferone, Texas Red, rhodamine, tetramethyl rhodamine, Rox, 7-nitrobenzo-2-oxa-1-diazole (NBD), pyrene, fluorescein, Europium, Ruthenium, Samarium, and other rare earth metals), radioisotopic labels, chemiluminescence labels (labels that are detectable via the emission of light during a chemical reaction), spin labels (a free radical (e.g. substituted organic nitroxides) or other paramagnetic probes (e.g. Cu²+, Mg²+) bound to a biological molecule being detectable by the use of electron spin resonance spectroscopy), enzymes (such as peroxidases, alkaline phosphatases, β-galactosidases, and glycose oxidases), antigens, antibodies, haptens (groups which are able to combine with an antibody, but which cannot initiate an immune response by themselves, such as peptides and steroid hormones), carrier systems for cell membrane penetration such as: fatty acid residues, steroid moieties cholesteryl), vitamin A, vitamin D, vitamin E, folic acid peptides for specific receptors, groups for mediating endocytose, epidermal growth factor (EGF), bradykinin, and platelet derived growth factor (PDGF). Especially interesting examples are pyrene, anthracene, anthraquinone, biotin, fluorescein, Texas Red, rhodamine, dinitrophenyl, digoxigenin, Ruthenium, Europium, Cy5, Cy3, etc.

Method of Detecting a Sequence Specific RNA

The present invention also relates to methods of detecting a sequence specific RNA in a mixture comprising nucleic acids and/or nucleic acid analogues comprising the steps of
a) providing a mixture of nucleic acids; and
b) providing one or more different oligonucleotides or oligonucleotide analogues, wherein the melting temperature of a hybrid consisting of said oligonucleotide or oligonucleotide analogue and a homologously complementary DNA (DNA hybrid), is significantly higher than the melting temperature of a hybrid consisting of said oligonucleotide or oligonucleotide analogue and a homologously complementary RNA (RNA hybrid), and wherein said oligonucleotides or oligonucleotide analogues are substantially complementary to said sequence specific RNA; and
c) providing a probe comprising a detectable label and a nucleic acid sequence capable of hybridising with said sequence specific RNA; and
d) incubating said mixture with said oligonucleotide or oligonucleotide analogue under conditions that allow for hybridisation, thereby blocking any sequence specific DNA; and
e) incubating said mixture with said probe under conditions that allow for hybridisation; and
f) detecting said detectable label; and
thereby detecting said sequence specific RNA.

One advantage of said method is that it allows for sequence specific detection of RNA in a mixture comprising RNA as well as DNA with a similar sequence. Accordingly, background signal from DNA is reduced significantly. This is of particular importance when a high ratio of DNA with a sequence identical to or similar to the RNA target is present (except that DNA comprise dT in place of U in RNA).

In one embodiment of the present invention RNA may be detected by in situ hybridisation targeting mRNA transcribed from genes harboured by high-copy plasmid containing cells (e.g. bacteria or yeast) or rRNA may be detected.

Preferably the mixture comprises DNA and RNA. More preferably the mixture does not comprise other nucleic acids or nucleic acids analogues than DNA and RNA.

Preferred oligonucleotides or oligonucleotide analogues to be employed with the methods of detecting sequence specific RNA in a mixture comprising nucleic acids and/or nucleic acid analogues, are oligonucleotide or oligonucleotide analogues selected from the group consisting of oligonucleotides or oligonucleotide analogues comprising at least one intercalator pseudonucleotide as described herein above.

Such oligonucleotides or oligonucleotide analogues may for example consists of 3 to 100, such as 3 to 30, such as 3 to 20, such as 3 to 10, such as 10 to 15, for example 15 to 20, such as 20 to 30, for example 30 to 100 nucleotides and/or nucleotide analogues and/or intercalator pseudonucleotides. Preferably said oligonucleotides or oligonucleotide analogues consist of between 10 and 50 nucleotides and/or nucleotide analogues and/or intercalator pseudonucleotides.

The mixture may be comprised within a cell, for example within an intact cell. The cell may for example be a prokaryotic cell or a eukaryotic cell, such as a plant cell or a mammalian cell. In such an embodiment the method may be employed for in situ hybridisation.

The mixture however may also be a cellular extract, which may be crude or may have been subjected to a purification procedure. For example the mixture may be a cellular extract, which has been subjected to gel electrophoresis and blotting to a membrane.

The detectable label may be detectable either directly or indirectly. For example the detectable label may be an enzyme, a fluorescent group, a chromophore, a radioactive isotope or a heavy metal or any of the labels described herein above. Furthermore the label may be an epitope specifically recognised by an antibody comprising a label or a chemical group specifically recognised by a binding partner comprising a label.

Hence, the method of detecting RNA comprises a step of blocking specific association with DNA, by hybridising DNA to the oligonucleotides or oligonucleotide analogues according to the present invention. Subsequently a labeled probe may not interact with DNA, but only with homologously complementary RNA. Such a method may be performed in a number of different ways. For example any of the methods described herein above for detection of DNA may be adapted to detection of RNA by adding a step of blocking specific association with DNA.

Method of Inhibiting RNases and DNases

Since oligonucleotide analogues comprising intercalator pseudonucleotides according to the present invention are significantly more stable towards nuclease action than normal nucleic acids, one or more particular oligonucleotide analogue sequences comprising intercalator pseudonucleotides may be suited for binding and inhibiting nucleases.

Accordingly, it is an object of the present invention to provide oligonucleotide analogues comprising RNA or RNA-like nucleobases and intercalator pseudonucleotides according to the present invention where said oligonucleotide analogues are capable of binding one or more of the different kinds of RNases and inhibiting the action of said RNases for a significantly longer time, at identical conditions, than the same amount of an identical oligonucleotide analogue differing only by the lack of said intercalator pseudonucleotides.

Likewise, it is an object of the present invention to provide oligonucleotide analogues comprising DNA or DNA-like nucleobases and intercalator pseudonucleotides according to the present invention where said oligonucleotide analogues are capable of binding one or more of the different kinds of DNases and inhibiting the action of said DNases for a significantly longer time, at identical conditions, than the same amount of an identical oligonucleotide analogue differing only by the lack of said intercalator pseudonucleotides.

Hence the present invention relates in one aspect to a method for inhibiting the activity of RNAses and/or DNAses comprising adding at least one oligonucleotide and/or oligonucleotide comprising at least one intercalator pseudonucleotide as defined herein to the RNAses and/or DNAses, allowing the oligonucleotide and/or oligonucleotide analogue to bind to the RNAse or DNAse and thereby inhibit the activity of said RNAse or DNAse.

It is also an object of the present invention to provide oligonucleotide analogues comprising any type nucleobase or nucleobase analogue and intercalator pseudonucleotides according to the present invention where said oligonucleotide analogues are capable of binding one or more of the different kinds of nucleases and inhibiting the action of said nucleases for a significantly longer time, at identical conditions, than the same amount of an identical oligonucleotide analogue differing only by the lack of said intercalator pseudonucleotides.

Applications

The oligonucleotides and/or oligonucleotide analogues comprising at least one intercalator pseudonucleotide as defined herein may be used in any application wherein oligonucleotides are conventionally used.

In particular the oligonucleotides and/or oligonucleotide analogues comprising at least one intercalator pseudonucleotide may be used in the following applications and/or as the following products:

Linear oligonucleotides and/or oligonucleotide analogues comprising intercalating pseudonuclotide(s)
Probes for hybridisation in the broadest sense
Probes for Watson-Crick base-pairing
Probes for triplex formation (Hoogstein base-pairing)
Probes for strand invasion
Probes for double strand invasion
Probes for capture/purification of several sequences of random DNA
Probes for capture/purification of sequence specific DNA
Probes for purification of RNA by removing sequence specific DNA
Probes for purification of RNA by removing genomic DNA via repeated DNA elements (sequence specific)
Probes for purification of RNA by removing genomic DNA via random sequences
Probes for blocking of DNA background hybridisation sites when doing RNA hybridisation
Probes for sequence specific detection of nucleic acids by IPN fluorescence
Probes for sequence specific detection of nucleic acids by conventional methods
Probes for sequence specific detection of nucleic acids by IPN fluorescence in combination with conventional methods
Primers for normal PCR (high affinity, high sensitivity, reduction of unspecific hybridisation, reduction of primer dimers, reduction of false negatives, reduction of false positives)
Primers for single-base extension
Primers for non-PCR dependent amplification (isothermal amplification, rolling circle amplification)
Primers with detection by fluorescence (w./wo. separation of amplified sequences from primers)
Primers with detection by conventional methods
Primers with detection by fluorescence in combination with conventional methods
Primers with detection mode used for amplification and subsequent array hybridisation and detection
SNP detection probes by fluorescence
Two adjacent probes for SNP detection probes by fluorescence
Single SNP detection probes by melting temperature
Two adjacent probes for SNP detection probes by melting temperature
Arrays of oligonucleotide analogues
Gene blockage (Transcription blockage)
Oligonucleotides or Oligonucleotide analogues comprising one intercalator pseudonucleotide for inhibiting RNases and DNases
Intercalator pseudonucleotide modified oligonucleotides or oligonucleotide analogues forming liposome-like formulations

A pair of oligonucleotide analoguescomprising intercalating pseudonuclotide(s), preferably both with intercalating pseudonuclotide(s)
Sequence specific detection of nucleic acids using conventional fluorophore/quencher detection
Sequence specific detection of nucleic acids using fluorescence detection
Nuclease-stable oligonucleotides or oligonucleotide analogues
Nuclease-stable oligonucleotides or oligonucleotide analogues and high affinity-binding duplex structures for Decoy targets
Duplex delivery for double strand invasion
Gene blockage
Intercalator pseudonucleotide modified oligonucleotides or oligonucleotide analogues duplexes forming liposome-like formulations

A hairpin nucleotide analogue with IPNs
Hairpin probes for hybridisation in the broadest sense
Hairpin probes for strand invasion
Hairpin probes for double strand invasion
Hairpin probes for sequence specific detection of nucleic acids using fluorescence detection
Hairpin probes for sequence specific detection of nucleic acids using conventional methods
Hairpin probes for sequence specific detection of nucleic acids using fluorescence in combination with conventional methods
Hairpin primers for normal PCR (high affinity, high sensitivity, reduction of unspecific hybridisation, reduction of primer dimers, reduction of false negatives, reduction of false positives)
Hairpin primers w. detection by fluorescence (w./wo. separation of amplified sequences from primers)
Hairpin primers with detection by conventional methods
Hairpin primers w. detection mode used for amplification and subsequent array hybridisation and detection
Single SNP detection hairpin probes by fluorescence
Double SNP detection hairpin probes by fluorescence
Arrays of oligonucleotides or oligonucleotide analogues comprising one intercalator pseudonucleotide for the different purposes mentioned above
Liposome-like formulations

A pair of oligonucleotides and/or oligonucleotide analogues
Duplex delivery
Inhibition of RNA-binding molecules and gene-blockage
Antisense (vs. RNA)
Antisense in combination with gene-blockage

and the combination of these areas.

### Examples

The following examples illustrate selected embodiments of the invention and should not be regarded as limiting for the invention.

In the examples the following abbreviations are used:

ODN: Oligodeoxynucleotide

INA: Intercalating nucleic acid corresponding to intercalator pseudonucleotide

### Example 1

### Preparation of an Intercalator Pseudonucleotide 1-Pyrenemethanol is commercially available, but it is also easily prepared from pyrene by Vilsmeier-Haack formulation followed by reduction with sodium borohydride and subsequent conversion of the alcohol with thionyl chloride affords 1-(chloromethyl)pyrene in 98% yield.

The acyclic amidite 5 (FIG. 1) was prepared from (S)-(+)-2,2-dimethyl-1,3-dioxalane-4-methanol and 1-(chloromethyl)pyrene in 52% overall yield. The synthesis of 5 (FIG. 1) is accomplished using KOH for the alkylation reaction, and using 80% aqueous acetic acid to give the diol 3 (FIG. 1), which is protected with dimethoxytritylchloride (DMT-Cl) and finally reaction with 2-cyanoethyl N,N,N′,N′-tetraisopropylphosphorodiamidite affords target compound 5 (FIG. 1) in 72% yield. The yield in the latter reaction step was decreased from 72% to 14% when 2-cyanoethyl N,N-diisopropylchlorophosphor amidite was used as the phosphitylating reagent. The synthesis of the acyclic amidite 5 is shown schematically in FIG. 1.

### 1-Pyrenylcarbaldehyde

A mixture of N-formyl-N-methylaniline (68.0 g; 41.4 mL; 503 mmol) and o-dichlorobenzene (75 mL) is cooled on an ice bath and added phosphoroxychloride (68g; 440 mmol) over 2 hours so that the temperature do not exceed 25° C. Pulverized Pyrene (50 g; 247 mmol) is added in small portions over 30 min. and the reaction mixture is equipped with a condenser and heated at 90-95° C. for 2 hours. After cooling to room temperature the dark red compound is filtered off and washed with benzene (50 mL.). Then it is transferred to water (250 mL) and stirred over night. The yellow aldehyde is filtered of and washed with water (3×50 mL). Recrystallized from 75% ethanol 3 times. Yield: 30.0 g (52.7%).

### 1-Pyrenylmethanol

1-Pyrenylcarbaldehyde (10.0 g; 43.4 mmol) is dissolved in dry THF (50 mL) under inert atmosphere and NaBH₄ (0.82 g; 22 mmol) is added in small portions over 10 min. The reaction mixture is stirred over night at r.t. and crystallizing the product by pouring into stirring water (350 mL). The product is filtered off, washed with water (4×25 mL) and dried under reduced pressure. Recrystallized from ethyl acetate. Yield: 8.54 g (84.7%).

### 1-(Chloromethyl)-pyrene

1-Pyrenylmethanol (6.40 g; 27.6 mmol) is dissolved in a mixture of pyridine (3.3 mL; 41.3 mmol) and CH₂Cl₂ (100 mL) and the mixture is cooled to 0° C. SOCl₂ (3.0 mL; 41.3 mmol) is added slowly over 15 min. and the temperature is allowed to rise slowly to r.t. Stir over night. The mixture is poured into stirring water (200 mL) and added CH₂Cl₂ (100 mL). The mixture is stirred for 30 min. The organic phase is washed with 5% NaH₂CO₃ (2×75 mL) and brine (2×75 mL) respectively, dried with sodium sulfate and concentrated under reduced pressure. Recrystallized from toluene/petroleum ether. Yield 6.75 g (97.8%).

### (S)-(+)-2,2-dimethyl-1,3-dioxalane-4-methanol

Pulverized KOH (25 g) and 1-(Chloromethyl)-pyrene (6.0 g; 23.9 mmol) is added to a solution of (S)-(+)-2,2-dimethyl-1,3-dioxalane-4-methanol (2.6 g; 19.7 mmol) in dry toluene (250 mL). The mixture is refluxed under Dean-Stark conditions in 16 h, then cooled to r.t. and added water (150 mL). The organic phase is washed with water (3×100 mL) dried with a combination of magnesium sulfate and sodium sulfate and concentrated under reduced pressure to a thick oil. Silica gel chromatography (CH₂Cl₂) afforded the pure compound in 6.1 g (90%).

### (R)-3-(1-Pyrenemethoxy)-propane-1,2-diol

(S)-(+)-2,2-dimethyl-1,3-dioxalane-4-methanol (6.1 g; 17.6 mmol) is added to a mixture of acetic acid and water (100 mL; 4:1) and is stirred at r.t. for 19 h. Concentrated under reduced pressure. Giving an oil in quantitatively yield.

### (S)-1-(4,4′-dimethoxytriphenylmetyloxy)-3-pyrenemethyloxy-2-propanol

(R)-3-(1-Pyrenemethoxy)-propane-1,2-diol (760 mg; 2.48 mmol) is dissolved in dry pyridine (20 mL) and added dimethoxytrityl chloride (920 mg; 2.72 mmol). The reaction mixture is stirred in 24 h and concentrated under reduced pressure. Purified by silica gel chromatography (ethyl acetate/cyclohexane/triethylamine 49:49:2) to give a white foam. Yield 1.20 g (79.5%).

### Phosphoramidite of (S)-1-(4,4′-dimethoxytriphenylmethyloxy)-3-pyrenemethyloxy-2-propanol

(S)-1-(4,4′-Dimethoxytriphenylmetyloxy)-3-pyrenemethyloxy-2-propanol (458 mg; 753 μmol), 2-cyanoethyl-N,N,N′,N′-tetraisopropylphosphan (453 mg; 429 μL; 1.51 mmol) and diisopropylammonium tetrazolide (193 mg; 1.13 mmol) is mixed in dry CH₂Cl₂ (7 ml) and stirred under nitrogen atmosphere for 6 days. Purified by silica gel chromatography (ethyl acetate/cyclohexane/triethylamine 49:49:2) and dried under reduced pressure. Yield 438 mg (72%) as a white foam.

### Example 2

Alternative Synthesis Procedure for 3-(1-Pyrenylmethoxy)-propane-1,2-diol

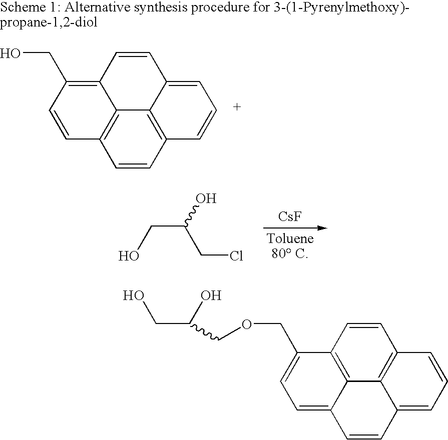

1-Pyrenylmethanol (232 mg; 1.0 mmol) is dissolved in hot toluene (2 mL over Na). CsF (7 mg; 0,046 mmol) is added and stirred for approx. 1 h at room temperature when 3-chloro-1,2-propandiol (170 mg; 1.53 mmol) is added. The mixture is stirred at 80° C. for 2h, cooled off to room temperature and the precipitated product is separated from the mixture by filtration. Washed with cold toluene (2×1 mL). Yield 220 mg (72%).

### Example 3

### Synthesis of the 2-O phosphoramidite of 1-O-4,4′-dimetoxytrityl-4-O-(9-antracenylmethyl)-1,2,4-butanetriol

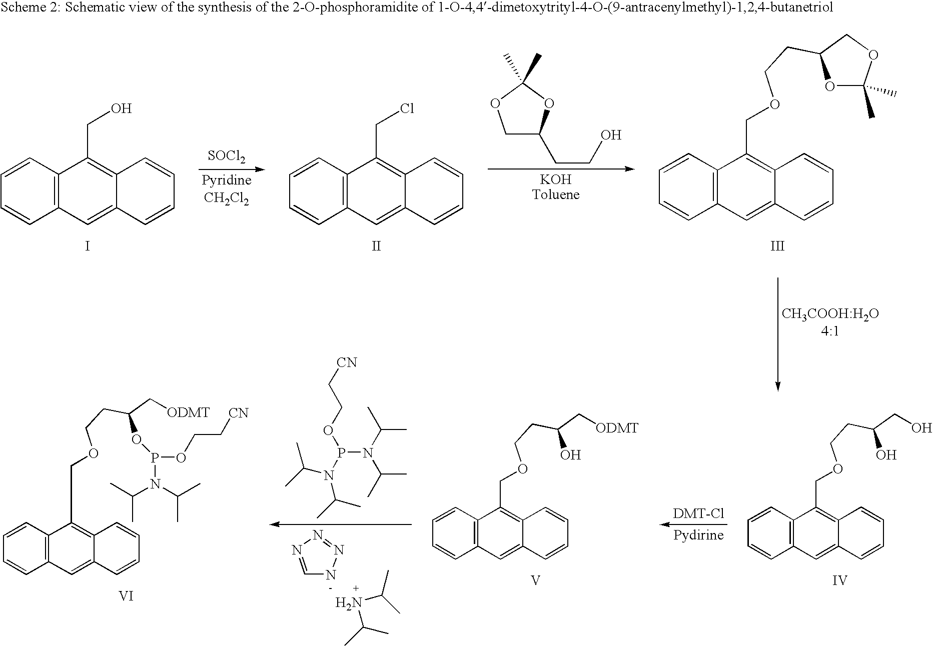

### 9-anthracenemethylchlorid (II)

9-anthracenemethanol (0.81 g; 3.89 mmol; I) was dissolved in dry pyridine (467 μL; 5.83 mmol) and dry CH₂Cl₂. Under stirring and at 0° C. SOCl₂ (423 μL; 5.83 mmol) was added dropwise, and the mixture was stirred for 24 h during which the temperature is allowed to rise to r.t. within 2h. The reaction is poured onto stirring H₂O (60 mL) and was added additional CH₂Cl₂ (40 mL). The organic phase was washed with a 5% NaHCO₃ (100 mL) solution, brine (100 mL) and water (100 mL) respectively. Dried over Na₂SO₄ and concentrated in vacuo. Yield 665 mg (75%).

### 1,2-D-□□-isopropylidene-4-(9-anthracenylmethyl)-1,2,4-butanetriol (III)

9-anthracenemethylchlorid (628 mg; 277 mmol) was dissolved in dry toluene (25 mL over Na) and 2-[(S)-2′,2′-dimethyl-1′,3′-dioxalan-4′-yl]-ethanol (506 mg; 3.5 mmol) and 3 small spoons of KOH was added. The mixture was connected to a Dean-Stark apparatus and stirred under reflux conditions over night. The reaction mixture was slowly cooled to r.t. and washed with H₂O (4×25 mL). Dried over Na₂SO₄ and concentrated in vacuo.

### 4-O-(9-anthracenylmethyl)-1,2,4-butanetriol (IV)

To the dried compound was added 80% AcOH (50 mL) and the reaction mixture was stirred in 24 h at r.t. The mixture was concentrated in vacuo over night and purified by short, fast silica gel chromatography (impurities was first eluated with CH₂Cl₂, and product was then eluated with 5% MeOH in CH₂Cl₂). Yield 56.3% over 2 steps.

### 1-O-(4,4′-dimethoxytrityl)-4-O-(9-anthracenylmethyl)-1,2,4-butanetriol (V)

The diol (425 mg; 1.40 mmol) and DMT-Cl is mixed in dry pyridine (5 mL) and stirred at r.t. for 36 h. The reaction mixture was concentrated in vacuo and purified by silica gel chromatography (EtOAc:^{c}C₆H₁₂:N(Et)₃ 63:35:2). Co-evaporated with ether (5 mL over Na) after which a yellowish foam was isolated. Yield 630 mg (74%).

### The phosphoramidite of 1-O-(4,4′-dimethoxytrityl)-4-O-(9-anthracenylmethyl)-1,2,4-butanetriol (VI)

The DMT protected anthracene compound was dissolved in dry CH₂Cl₂ (7 mL) and diisopropylammonium tetrazolide (252 mg; 1.5 mmol) and 2-Cyanoethyl N,N,N′,N′-tetraisopropyl Phosphane was added. The reaction mixture was stirred for 20 h at r.t. Concentrated in vacuo and purified by silica gel chromatography (EtOAc:^{c}C₆H₁₂:N(Et)₃ 24:74:2). Co-evaporated with ether (5 mL over Na) to give a yellowish foam (67%).

### Example 4

### Synthesis of the Phosphoramidite of (S)-1-(4,4′-dimethoxytriphenylmethyloxy)-4-(7,9-dimethyl-pyrido[3′,2%4,5]thieno[3,2-d]pyrimidin-4(1H)-one)-2-butanol (V)

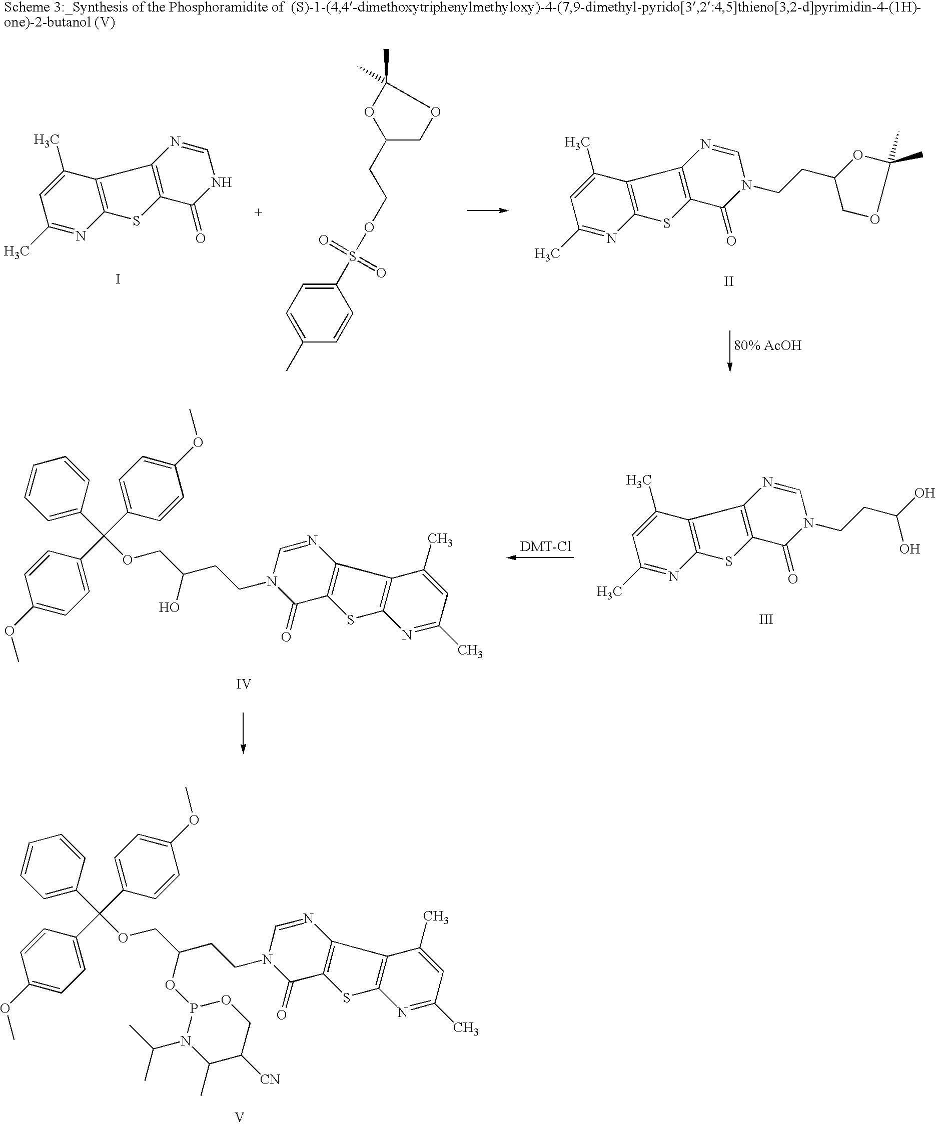

### 7,9-dimethyl-pyrido[3′,2′:4,5]thieno[3,2-d]pyrimidin-4(1H)-one (I)

7,9-dimethyl-pyrido[3′,2′:4,5]thieno[3,2-d]pyrimidin-4(1 H)-one was prepared according to literature procedures.^{1,2,3} ¹Schimdt, U. & Kubitzek, H., Chem. Ber., 93, 1559 (1960)² Hassan, K. M. et al. Phosporous, Sulfur, Silicon Relat. Elem., 47, 181 (1990)³ Gewald, K. & Jänsch, H. J., Prakt. Chemie 313-320 (1976)

### 3-N—((S)-2″,2″-dimethyl-1″,3″-dioxalane-4″-ethanyl)-7,9-dimethyl-pyrido[3′,2′:4,5]thieno[3,2-d]pyrimidin-4(1H)-one (II)

7,9-dimethyl-pyrido[3′,2′:4,5]thieno[3,2-d]pyrimidin-4(1H)-one (1.16 g; 5.0 mmol) was suspended in anhydrous DMF (20 mL) and NaH (0.2 g; 5.0 mmol, 60% dispersion in mineral oil) was added. The mixture was stirred for 2 h until all H₂ evolving ceased. Then (S)-2,2-dimethyl-1,3-dioxalane-4-ethanol-O-para-toluenesulfonate (0.78 g; 5.1 mmol) was added in one portion and the mixture was stirred for 24 h at 80° C. The mixture was evaporated to dryness in vacuo, co evaporated with dry toluene (3×10 mL) in vacuo and the residue was purified silica gel chromatography (5% EtOAc in CHCl₃) to get a colorless product. Yield 0.81 g; 45%.

### (S)-4-(7,9-dimethyl-pyrido[3′,2′:4,5]thieno[3,2-d]pyrimidin-4(1H)-one)-butan-1,2-diol (III)

3-N—((S)-2″,2″-dimethyl-1″,3″-dioxalane-4″-ethanyl)-7,9-dimethyl-pyrido[3′,2′:4,5]thieno[3,2-d]pyrimidin-4(1H)-one (0.75 g; 2.1 mmol) was stirred at r.t. in 80% AcOH (20 mL) for 24 h. The product was obtained by concentration in vacuo and co-evaporation with EtOH. Purified by silica gel chromatography (5% MeOH in CHCl₃) to get the colorless product. Yield 0.5 g (75%).

### (S)-1-(4,4′-dimethoxytriphenylmethyloxy)-4-(7,9-dimethyl-pyrido[3′,2′:4,5]thieno[2,3-d]pyrimidin-4(1 H)-one)-2-butanol (IV)

(S)-4-(7,9-dimethyl-pyrido[3′,2′:4,5]thieno[3,2-d]pyrimidin-4(1H)-one)-butan-1,2-diol (0.6 g; 1.9 mmol) was dissolved in dry pyridine (5 mL) and DMT-Cl (0.71 g; 2.1 mmol) was added. Stirred at r.t. over night. Concentrated in vacuo and co evaporated using dry toluene (3×10 mL). The residue was purified by silica gel chromatography (EtOAc:^{c}C₆H₁₂:N(Et)₃ 49:49:2) to yield a white foam. Yield 0.77 g (65%)

### Phosphoramidite of (S)-1-(4,4′-dimethoxytriphenylmethyloxy)-4-(7,9-dimethylpyrido[3′,2′:4,5]thieno[3,2-d]pyrimidin-4(1H)-one)-2-butanol (V)

(S)-1-(4,4′-dimethoxytriphenylmethyloxy)-4-(7,9-dimethyl-pyrido[3′,2′:4,5]thieno[3,2-d]pyrimidin-4(1H)-one)-2-butanol (310 mg; 0.5 mmol) was dissolved under nitrogen in anhydrous dichloromethane (10 mL). Diisopropylammoniumtetrazolide (0.11 g; 0.67 mmol)was added followed by dropwise addition of 2-Cyanoethyl-N,N,N′,N′-tetraisopropylphophorodiamidite (0.3 g; 1.0 mmol) the reaction was stirred over night under nitrogen atmosphere, concentrated in vacuo and purified by silica gel chromatography (EtOAc:^{c}C₆H₁₂:N(Et)₃ 49:45:12) to give a white foam. Yield 345 mg (84%)

### Example 5

### Duplexes with Dangling Ends

To investigate the stacking ability of the nucleoside analogue 5 (FIG. 1) it was incorporated into the 5′ end of two different self-complementary strands (5′-XCGCGCG and 5′-XTCGCGCGA).

The ODN synthesis is carried out on a Pharmacia LKB Gene Assembler Special using Gene Assembler Special software version 1.53. The pyrene amidite is dissolved in dry acetonitrile, making a 0.1M solution and inserted in the growing oligonucleotides chain using same conditions as for normal nucleotide couplings (2 min. coupling). The coupling efficiency of the modified nucleotides is greater than 99%. The ODNs are synthesized with DMT on and purified on a Waters Delta Prep 3000 HPLC with a Waters 600E controller and a Waters 484 detector on a Hamilton PRP-1 column. Buffer A: 950 ml. 0.1 M NH₄⁺HCO₃⁻+50 ml MeCN pH=9.0; buffer B: 250 ml. 0.1 M NR₄⁺HCO₃⁻+750 ml MeCN pH=9.0. Gradients: 5 min. 100% A, linear gradient to 100% B in 40 min., 5 min. with 100% B, linear gradient to 100% A in 1 min. and the 100% A in 29 min (product peak≈37 min.). The ODNs were DMT deprotected in 925 μl H₂O+75 μl CH₃COOH and purified by HPLC again using the same column, buffer system and gradients (product peak≈26 min.). To get rid of the liable salts, the ODNs were re-dissolved in 1 ml of water and concentrated in vacuo three times.

All oligonucleotides were confirmed by MALDI-TOF analysis made on a Voyager Elite Biospectrometry Research Station from PerSeptive Biosystems. The transition state analyses were carried out on a Perkin Elmer UV/VIS spectrometer Lambda 2 with a PTP-6 temperature programmer using PETEMP rev. 5.1 software and PECSS software package ver. 4.3. Melting temperature measurements of the self-complementary sequences are made in 1 M NaCl, 10 mM Na.Phosphate pH 7.0, 1.5 μM of each DNA strand. All other ODNs are measured in a 150 mM NaCl, 10 mM, Na.Phosphate, 1 mM EDTA pH 7.0, 1.5 μM of each strand. All melting temperatures giving are with an uncertainty on ±0.5° C.

The Amber forcefield calculations were done in MacroModel 6.0 and 7.0 with water as solvent and minimization is done by Conjugant Gradient method. The starting oligonucleotide sequences for calculation with the inserted pyrenes is taken from Brookhavens Protein Databank, and modified in MacroModel before minimazation is started. Lam and Au-Yeung solved a structure of a self-complementary sequence, equal to the one used in this work, by NMR. Their structure is prolonged with the pyrene amidite at the 5′-end of each strand and used for the structural calculations. The other sequence is a 13-mer highly conserved HIV-1 long terminal repeat region. G-7 is replaced by the pyrene amidite and calculations are made with and without an across lying C-nucleotide. The pyrene is placed in the interior of the duplex from the beginning. All bonds are free to move and to rotate.

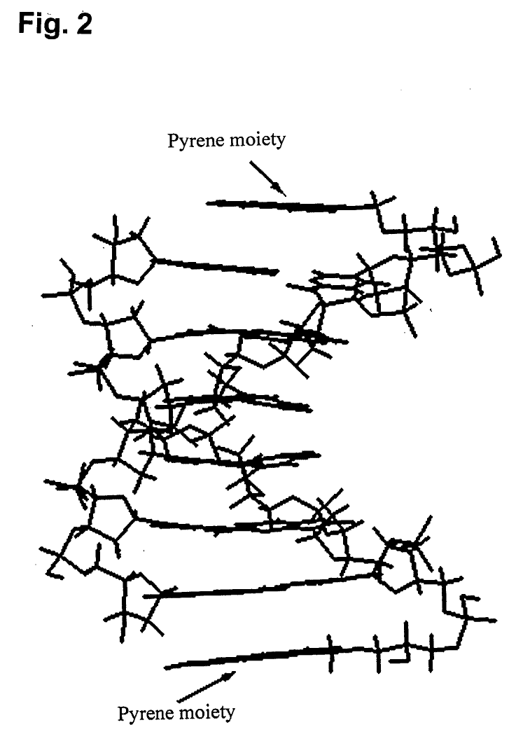

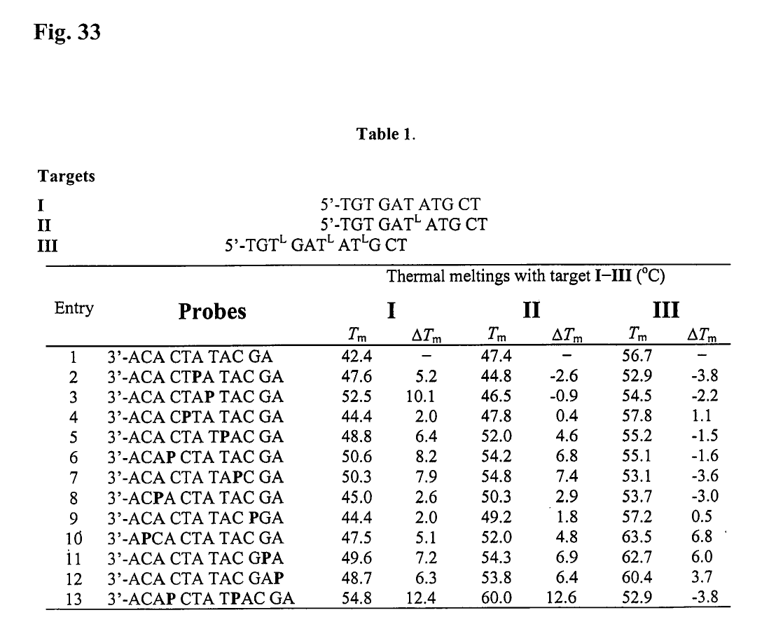

The melting temperature of modified and unmodified, self-complementary DNA are shown in FIG. 33. Incorporation of the pyrene amidite in the 5′ end as a dangling end stabilises the DNA duplex with 19.2° C.-21.8° C. (8.6° C.-10.9° C. per modification) depending on the underlying base pair. The stabilizations of the duplexes due to incorporation of 5 at the 5′ termini of the nucleic acid strands are similar to those found by Guckian et al. who inserted a pyrene nucleoside at the 5′ termini of self complementary ODNs (oligo deoxynucleic acids). The stabilisation can be explained by calculations using “MacroModel” which predict a structure were the pyrene moiety interacts with both nucleosides in the underlying basepair (FIG. 2).

TABLE 1
Melting temperatures of self-complementary
sequences with 5′ modification.

| | Tm(° C.) | ΔT(° C.) |
|---|---|---|
| 5′ C-G-C-G-C-G | 41.0 | |
| 3′ G-C-G-C-G-C | | |
| 5′ T-C-G-C-G-C-G-A | 46.9 | |
| 3′ A-G-C-G-C-G-C-T | | |
| 5′ X-C-G-C-G-C-G | 62.8 | 21.8 |
| 3′ G-C-G-C-G-C-X | | |
| 5′ X-T-C-G-C-G-C-G-A | 64.1 | 17.2 |
| 3′ A-G-C-G-C-G-C-T-X | | |

### Example 6

### End-Positioned Intercalating Pseudonucleotides—Stabilisation Dependent on Intercalator-Linker Length

Introduction

In this example is shown the dependence on linker length for the increase of affinity by the addition of intercalating pseudonucleotide to the 5′-end of an oligonucleotide. There is further more shown two examples of intercalating pseudonucleotides of comparable stabilisation effect.

Material and Methods

Oligonucleotides:

Probe I:
3′-CGA ACT CX
Probe II:
3′-CGA ACT CD
Probe III:
3′-CGA ACT CY
Ref:
3′-CGA ACT C
Target:
5′-GCT TGAG

Below is shown the amidites that were used in the preparation of the above mentioned oligonucleotides:

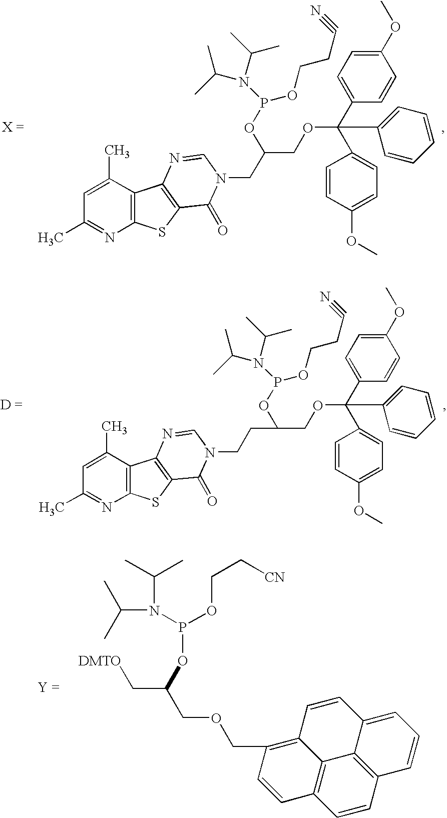

All hybridisation experiments were carried out with 1.5 μM of both target and probe strands in 2 mL of a buffer solution containing:

140 mM NaCl

10 mM Na₂HPO₄.2 H₂O

1 mM EDTA

pH=7.0

The target strands and probes were annealed by mixing them in the above mentioned buffer at 95° C. for 3 min. after which they are slowly cooled to room temperature. The melting temperatures of the hybridised probe-target hybrids were found by slowly heating the solution in a quartz cuvette, while simultaneously determining the absorbance. All melting temperatures presented in this example are with an uncertainty of ±1.0° C. as determined by repetitive experiments.

Results and Discussion

The results of the melting experiments is shown in Table 5:

| | | |
|---|---|---|
| | Melting temperature | ΔTm |
| Name | (° C.) | (° C.) |

Hybridisation to Target

| | | |
|---|---|---|
| Ref | 22.8 | — |
| Probe I | 28.4 | 5.6 |
| Probe II | 34.4 | 11.6 |
| Probe III | 33.8 | 11.0 |

The difference in melting temperature between probe I and II is due to the short linker of probe I. Hence it is important that the combined length of linker and intercalator is optimal, to obtain a large increase in affinity between intercalating pseudonucleotide modified oligonucleotides and their target DNA sequences. Probes II and III have nearly the same affinity for their target sequences, even though the intercalating moieties in the two probes are very different. This shows that the intercalating pseudonucleotides are a class' of compounds that, dependent on the wanted feature it should introduce into an oligonucleotide or oligonucleotide analogue, it should be designed by more or less strict rules.

### Example 7

### Oligonucleotides Synthesized on Universal Supports—Obtaining 3′ Intercalator Pseudonucleotide Modified Oligonucleotides

In a preferred embodiment of the present invention, oligonucleotides or oligonucleotide analogues comprise intercalating pseudonucleotides at either or both ends. In this example it is shown that oligonucleotides or oligonucleotide analogues with intercalating pseudonucleotides in the 3′-end can be synthesised using Universal supports. It is furthermore shown that selfcomplementary oligonucleotides comprising intercalating pseudonucleotides positioned in the 3′-end form very thermal stable hybrids.

Material and Methods

The following two types of self-complementary probes were synthesised. Design A was synthesized using a universal support while B was synthesized using standard nucleotide coupled columns and procedures:

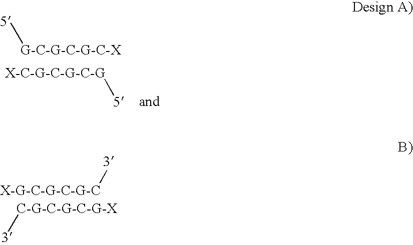

Two different intercalating pseudonucleotides were used for design A (I and II), and II was used for design B as well. One reference sequence without any intercalating pseudonucleotides (III) was synthesised. Hence X represents either:

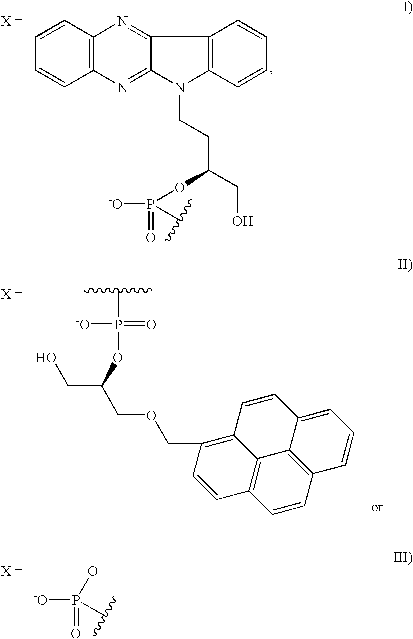

After synthesis the oligo nucleotide analogues were treated with 2% LiCl in a 32% NH₄OH solution in order to remove protection groups from the heterocyclic amines and to cleave the oligonucleotide from the universal support. Oligonucleotides comprising intercalating pseudonucleotides were tested on MALDI-TOF and found at the expected values.

All hybridisation experiments were carried out with 1.5 μM of both target and probe strands in 1 mL of a buffer solution containing:

140 mM NaCl

10 mM Na₂HPO₄.2 H₂O

1 mM EDTA

The target strands and probes were annealed by mixing them in the above mentioned buffer at 95° C. for 3 min. after which they are slowly cooled to room temperature. The melting temperatures of the hybridised probe-target hybrids were found by slowly heating the solution in a quartz cuvette, while simultaneously determining the absorbance. All melting temperatures presented in this example are with an uncertainty of ±1.0° C. as determined by repetitive experiments.

Results and Discussion

| X\Design | A | B |
|---|---|---|
| I | 57.3° C. | — |
| II | 59.2° C. | 62.8° C. |
| III | 41.0° C. | 41.0° C. |

From the above table it can be seen that the insertion of intercalating pseudonucleotides in either end of an oligonucleotide increases the affinity for a complementary target nucleic acid. It is also shown that intercalating pseudonucleotides can be inserted into the 3′ end of an oligonucleotide or oligonucleotide analogue by using standard universal base chemistry.

### Example 8

### Substituting a nucleotide with a 1-O-(pyrenylmethyl)glycerol nucleotide

The ODN synthesis is carried out as described in example 5. Phosphoramidite 5 (FIG. 1) is prepared as described in example 1.

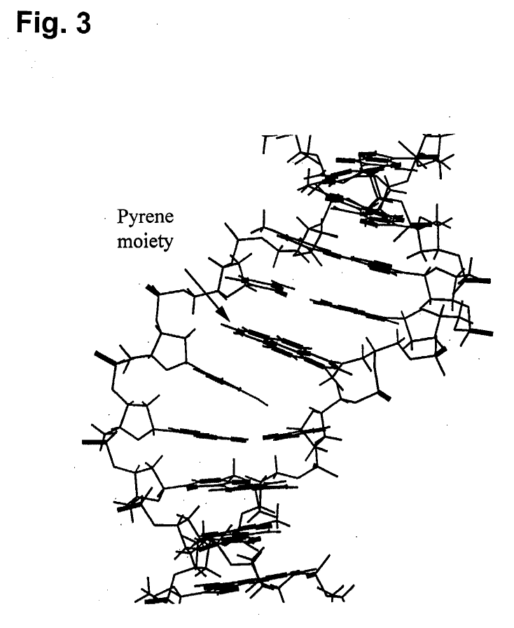

UV melting temperature measurements (Table 2) where a G nucleotide is replaced by the flexible, abasic linkers ethylene glycol and 1,3-propandiol shows, not surprisingly, a decrease in duplex stability compared to the unmodified, fully complementary sequence. The required DMT protected cyanoethyl N,N-diisopropylphosphoramidites of ethylene glycol and 1,3-propandiol were synthesized by standard methods. Having the 1-O-(pyrenylmethyl)glycerol nucleotide in the same position instead of the abasic diols increases the melting temperature by 16.4° C.-18.0° C. for the DNA/DNA duplex, indicating that the pyrene is co-axial stacking with both sides of the duplex, as the stabilization per modification exceeds the effect of placing the pyrene module at one end of a duplex (Table 1). Calculations from “MacroModel” shows that the pyrene module only makes a minor distortion of the double helix when intercalated into the duplex, having interaction with nucleobases both to the 5′ side and to the 3′ side of the intercalation site (FIG. 3). The stabilization of the duplex by co-axial stacking of the pyrene moiety is not large enough to compensate for the loss in binding affinity due to the reduced number of hydrogen bonds by substitution of G with the pyrene moiety, the modified duplex being less stable than the unmodified fully complementary by 8.6° C. The same trend is found for DNA/RNA duplexes although these have lower melting temperatures in general than the corresponding DNA/DNA duplexes. The stabilization of the pyrene moiety is only 8.2° C. for the DNA/RNA duplex when compared with ethylene glycol whereas the stabilization is 16.4 for the DNA/DNA duplex. The pyrene insertion results in an improved discrimination between ssDNA and ssRNA with 9.0° C. difference in the melting temperatures of their corresponding duplexes.

TABLE 2
DNA/DNA and DNA/RNA duplexes where one nucleotides is either an
abasic, flexible linker, the pyrene module (5), a deletion or a complementary G.

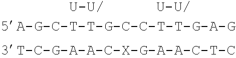

| | | DNA | RNA | Discrimination |
|---|---|---|---|---|
| Entry | | Tₘ (° C.) | Tₘ (° C.) | ΔT_{m, DNA-RNA} (° C.) |
| 1 | 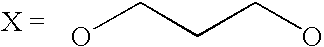 | 26.0 | 25.8 | 0.2 |
| 2 | 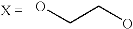 | 27.6 | 26.8 | 0.8 |
| 3 | X = 5 | 44.0 | 35.0 | 9.0 |
| 4 | X = -(12-mer) | 35.2 | 29.6 | 4.6 |
| 5 | X = G(13-mer) | 52.6 | 47.2 | 5.4 |

### Example 9

### 1-O-(pyrenylmethyl)glycerol as a Bulge

Normally the introduction of a bulge into the double helix decreases the melting temperature. This is also observed here (Table 3), but if the pyrene module is built in as the bulge, the melting temperature of the DNA duplex goes up by 3° C. This is in accordance with the observations made by Ossipov et al., finding it necessary to introduce a bulge to prevent a large destabilization of the duplex when introducing a non-Watson-Crick binding intercalator. One pyrene moiety stabilises the duplex by 11.2° C. compared to the flexible ethylene glycol linker indicating that the pyrene moiety is intercalated into the duplex.

The difference in the melting temperature between the pyrene modified DNA/DNA duplex and the pyrene modified DNA/RNA duplex is increased to 12.6° C. when inserting one pyrene modification as a bulge. This difference is 7.4° C. larger than in the unmodified duplexes and much larger than the differences between the duplexes containing natural nucleoside or flexible ethylene glycol bulges. This means that the pyrene moiety is selective and only able to stabilize DNA/DNA duplexes and not the DNA/RNA duplex. For the latter duplex it occurs that the duplex have the same melting temperature with the glycerol linker than with the pyrene moiety, indicating that the pyrene does not intercalate into the strands.

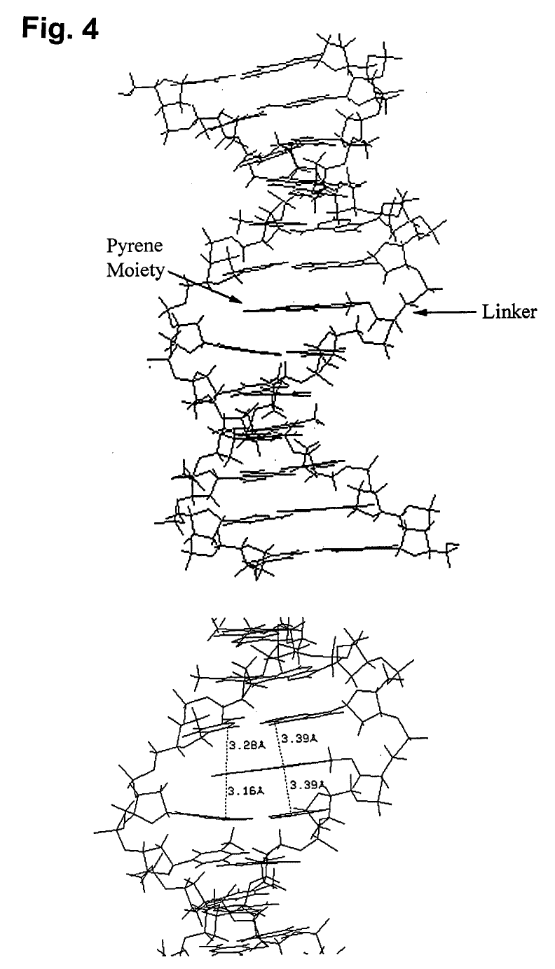

Structural calculation of the pyrene modified DNA/DNA structure (FIG. 4) shows that the pyrene module only makes minor distortion in the duplex, and that the linker introduces enough flexibility in the backbone to have a distance of 3.4 Å between the pyrene moiety and the nucleobases of the same strand. The nucleobases of the opposite strand has a little shorter spacing between the pyrene moiety and the nucleobases than the optimum 3.4 Å.

To investigate the discrimination and stabilization phenomena further, some ODNs with two insertions of the pyrene amidite were prepared, to see if the effects of the pyrene module is additive, and indeed it is. The results (Table 3) show that, depending on the distance between two insertions and their neighboring base pairs, it is possible to stabilize the double inserted pyrene DNA/DNA duplex up to 13.4° C. (6.7° C. per modification) compared to the natural DNA duplex. The destabilization of the pyrene modified DNA/RNA duplex is also somewhat additive, so that the difference in melting temperature between the pyrene modified DNA/DNA duplex and the pyrene modified DNA/RNA duplex is up to 25.8° C. when two pyrene modifications is inserted. For both stabilization and discrimination the best results are obtained, when the insertions are separated by four base pairs. When two insertions of the pyrene amidites are placed next to each other in the ODN there is a decrease in melting temperature of 5.2° C. compared to the unmodified DNA/DNA duplex and a decrease of 8.2° C. compared to the mono modified duplex. It is noteworthy that one basepair between two insertions in the DNA/DNA duplex is sufficient to improve

DNA/DNA stabilization and DNA/RNA discrimination, when compared with the duplex with only one insertion.

Table 3 Melting temperatures of oligonucleotides with different substitutions hybridized to either DNA or RNA.

### Example 10

### Five Different Intercalating Pseudonucleotides as Bulge Insertion with Increased Affinity for the Complementary DNA Target

Below is shown an overview of five different intercalating pseudonucleotides inserted in the middle of a DNA oligonucleotide. When hybridised to target said intercalating pseudonucleotides act as bulge insertions. All intercalating pseudonucleotide modified oligonucleotides shown here have an increased affinity for the complememtary DNA target compared to the unmodified oligonucleotide:

| | | T_{m, DNA} |
|---|---|---|
| Target DNA strand | 5′ A-G-C-T-T-G-C-T-T-G-A-G | |
| | 3′ T-C-G-A-A-C-G-A-A-C-T-C | 47.4° C. |
| 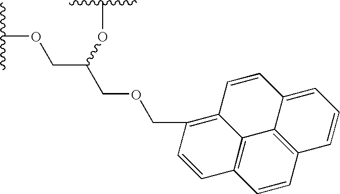 | 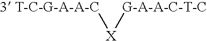 | 50.4° C. |
| 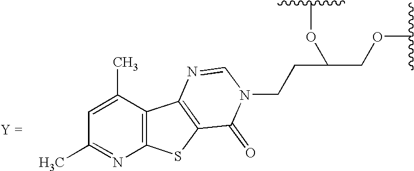 | 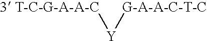 | 50.6° C. |
| 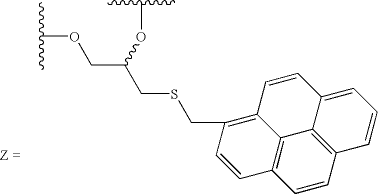 | 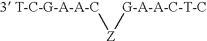 | 49.4° C. |
| 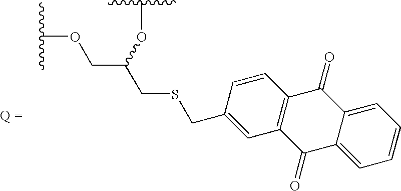 | 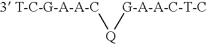 | 49.2° C. |
| 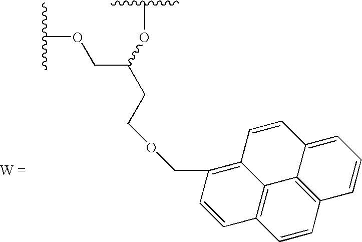 | 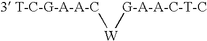 | 48.8° C. |

Below is shown the combined length of the linker and intercalator, it is clear that all of the shown examples have nearly the same combined length of intercalator and linker (9.9±1.3 Å).

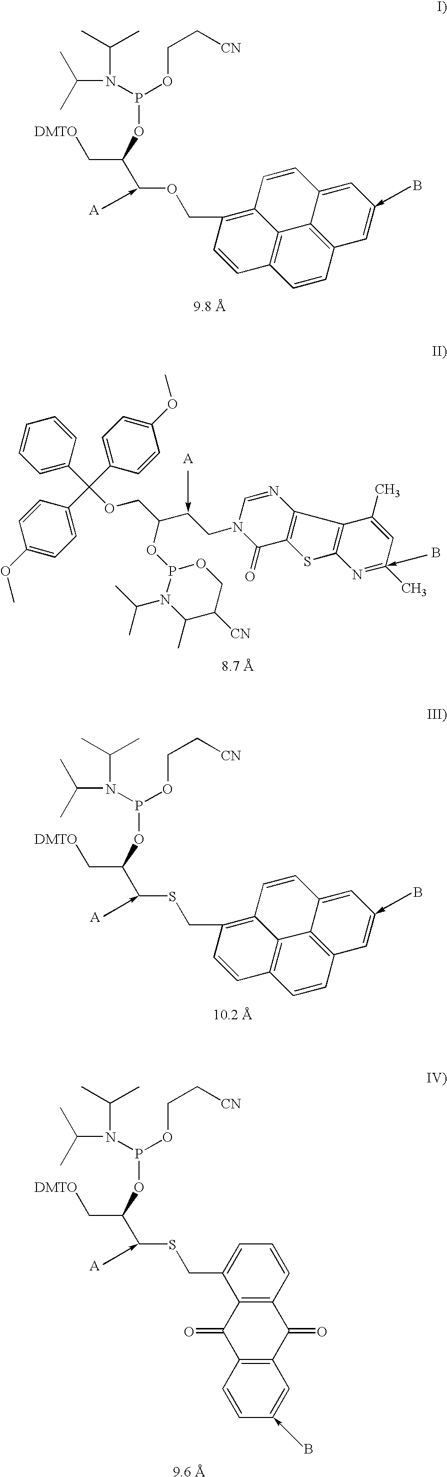

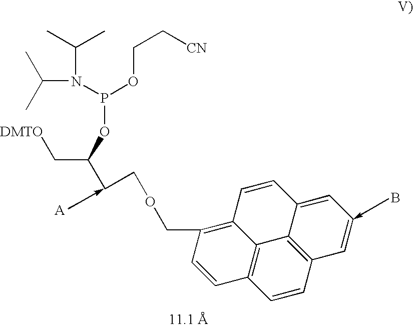

We can from this conclude that intercalating pseudonucleotides are a broad group of compounds that obeys some simple rules regarding the combined length of the intercalator and linker.

### Example 11

### Higher Affinity for DNA—Lower Affinity for RNA

In this example it is shown that when the target nucleic acid sequence for a probe is DNA, the melting temperature of the hybrid increases by introduction of intercalating pseudonucleotides into the probe—regardless if the probe is DNA or RNA (see Table 4 below). Additional the affinity for a RNA target is reduced regardless if the probe is DNA or RNA. Hence, intercalator pseudonucleotides can be introduced to oligonucleotides or oligonucleotide analogues giving the oligonucleotide or oligonucleotide analogue increased affinity for DNA and reduced affinity for RNA and RNA-like compounds like LNA, 2′-O-METHYL RNA.

TABLE 4
Three different situations.

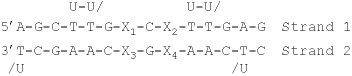

| Strand 1 | | | Strand 2 | | | |
|---|---|---|---|---|---|---|
| Type | X₁ | X₂ | Type | X₃ | X₄ | Tₘ (° C.) |

DNA duplexes and hybrids

| | | | | | | |
|---|---|---|---|---|---|---|
| DNA | — | — | DNA | — | — | 48.4 |
| DNA | 5 | 5 | DNA | — | — | 51.4 |
| DNA | — | — | DNA | 5 | 5 | 48.4 |

RNA duplexes and hybrids

| | | | | | | |
|---|---|---|---|---|---|---|
| RNA | — | — | RNA | — | — | 57.8 |
| RNA | 5 | 5 | RNA | — | — | 46.8 |
| RNA | — | — | RNA | 5 | 5 | 47.2 |

DNA/RNA hybrids

| | | | | | | |
|---|---|---|---|---|---|---|
| DNA | — | — | RNA | — | — | 42.2 |
| DNA | 5 | 5 | RNA | — | — | 34.2 |
| DNA | — | — | RNA | 5 | 5 | 45.2 |

At the top: DNA duplex affinity is increased or unaltered by the presence of intercalator pseudonucleotides in the hybrid compared to the duplex where none of the strands comprise intercalator pseudonucleotides.
In the middle: The RNA duplex is destabilized by the presence of intercalator pseudonucleotides in the hybrid compared to the duplex where none of the strands comprise intercalator pseudonucleotides.
At the bottom: Here it is shown how the hybrid between a DNA and a RNA strand is stabilized by intercalator pseudonucleotides if these are comprised by the RNA strand. Furthermore it is shown than when incorporated into the DNA strand the affinity for RNA is decreased.
5 = amidite 5 from example 1 incorporated into the strand according to the procedure described herein above.

By different relative positioning of the intercalator pseudonucleotides it is possible to gain higher affinity for DNA than shown in the Table above. Examples of this are given in the table in example 9.

### Example 12

### Reduced Cross-Hybridization

Cross-hybridization between two corresponding oligonucleotides comprising at least one intercalator pseudonucleotide with reduced affinity depending of the relative positioning of the intercalator pseudonucleotides. In the table below it is shown how the melting temperature is decreased if intercalator pseudonucleotides are positioned right opposite each other.

TABLE 5
Three different situations.

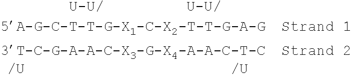

| | | |
|---|---|---|
| Strand 1 | Strand 2 | Tₘ (° C.) |

DNA duplexes and hybrids

| | | | | | | |
|---|---|---|---|---|---|---|
| DNA | — | — | DNA | — | — | 48.4 |
| DNA | 5 | 5 | DNA | — | — | 51.4 |
| DNA | 5 | 5 | DNA | 5 | 5 | 42.6 |

RNA duplexes and hybrids

| | | | | | | |
|---|---|---|---|---|---|---|
| RNA | — | — | RNA | — | — | 57.8 |
| RNA | 5 | 5 | RNA | — | — | 46.8 |
| RNA | 5 | 5 | RNA | 5 | 5 | 45.4 |

RNA and DNA hybrids

| | | | | | | |
|---|---|---|---|---|---|---|
| DNA | — | — | RNA | — | — | 42.2 |
| DNA | — | — | RNA | 5 | 5 | 45.2 |
| DNA | 5 | 5 | RNA | 5 | 5 | 37.8 |

At the top: DNA duplex is stabilized by the presence of intercalator pseudonucleotides in the hybrid compared to the duplex where none of the strands comprise intercalator pseudonucleotides. If intercalator pseudonucleotides are positioned in relation to each other, so that they are in close vicinity of each other when the oligonucleotides or oligonucleotide analogues are hybridized the melting temperature is decreased compared to when only one strand comprises intercalator pseudonucleotides.
In the middle: RNA duplex is destabilized by the presence of intercalator pseudonucleotides in the hybrid compared to the duplex where none of the strand comprise the intercalator pseudonucleotides.
At the bottom: Here it is shown how the hybrid between a DNA and a RNA strand is stabilized by intercalator pseudonucleotides if these are comprised in the RNA strand. Further it is shown that if intercalator pseudonucleotides are positioned in relation to each other, so that they are in close vicinity of each other when the oligonucleotides or oligonucleotide analogues are hybridized the melting temperature is decreased compared to when only the RNA strand comprises intercalator pseudonucleotides.
5 = amidite 5 from example 1 incorporated into the strand according to the procedure described herein above.

### Example 13

### Fluorescence

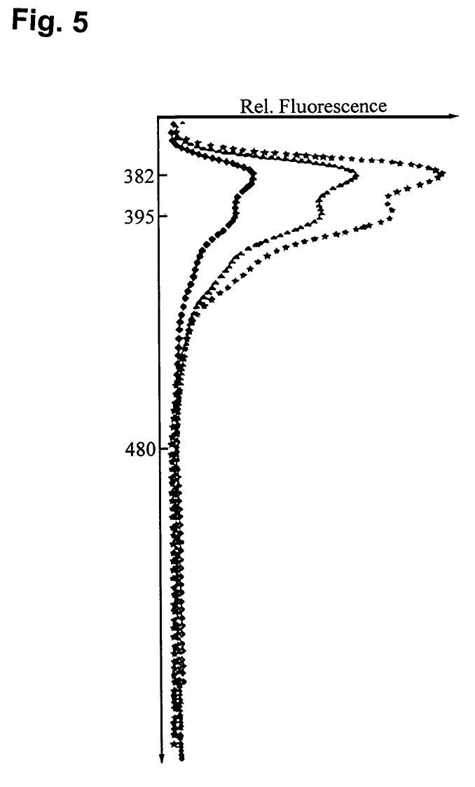

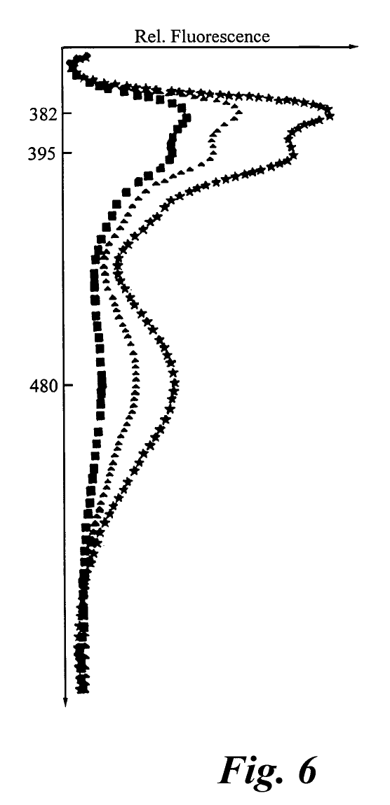

A decreased fluorescence of the mono pyrene modified DNA strands upon binding to the complementary strands, indicates that the pyrene intercalates into the double helix. Double pyrene inserted oligonucleotides gives the same result for all of the different ODNs. This effect is more pronounced when the modified DNA is hybridized with ssDNA than when hybridized with ssRNA (FIGS. 5 and 6), indicating less intercalation of pyrene into the DNA/RNA Duplexes. This supports the conclusion from the thermal melting experiments about lacking of pyrene intercalation into the bulged DNA/RNA duplexes as deduced from the nearly identical melting temperatures with glycerol and pyrene bulges in the DNA/RNA duplexes (Table 3).

Two pyrene moieties separated by only one nucleotide generates a third peak at 480 nm, due to excimer formation of the pyrene residues. However this band is almost extinguished, when this type of DNA with two insertions with pyrene hybridizes to a complementary DNA strand. This indicates intercalation around an intact basepair preventing the two pyrene moieties to get into the physical distance of approximately 3.4 nm needed for excimer formation. When a double inserted DNA hybridizes to a complementary RNA the two pyrene moieties are still able to interact since a substantial excimer band is found.

### Example 14

### 3-Exonuclease Stability of Oligonucleotides or Oligonucleotide Analogues Comprising Intercalating Pseudonucleotides

Materials and Methods:

Time course of Snake Venom phosphordiesterase digestion of the DNA reference I, the INA oligo II and a mixture of both .l and II. A 1.5 μM solution of all the strands was use (1.5 μM of each strand in the mixed assay) in 2 mL of buffer (0.1 M Tris-HCl; pH=8.6; 0.1 M NaCl; 14 mM MgCl₂) was digested with 1.2 U Snake Venom phosphordiesterase (30 μl of the following buffer solution: 5 mM Tris-HCl; pH=7.5; 50% glycerol (v/v)] at room temperature.

DNA oligo:
3′-TGT CGA GGG CGT CGA
INA oligo:
5′-YAC AGC YTC CCY GCA GCY T

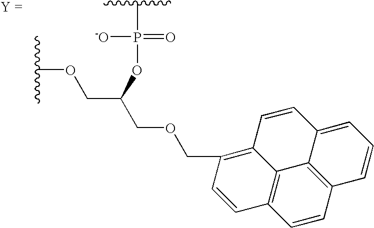

Results

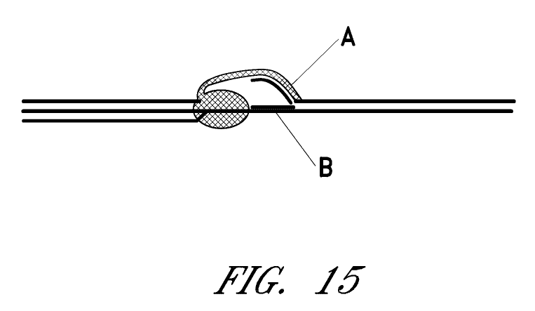

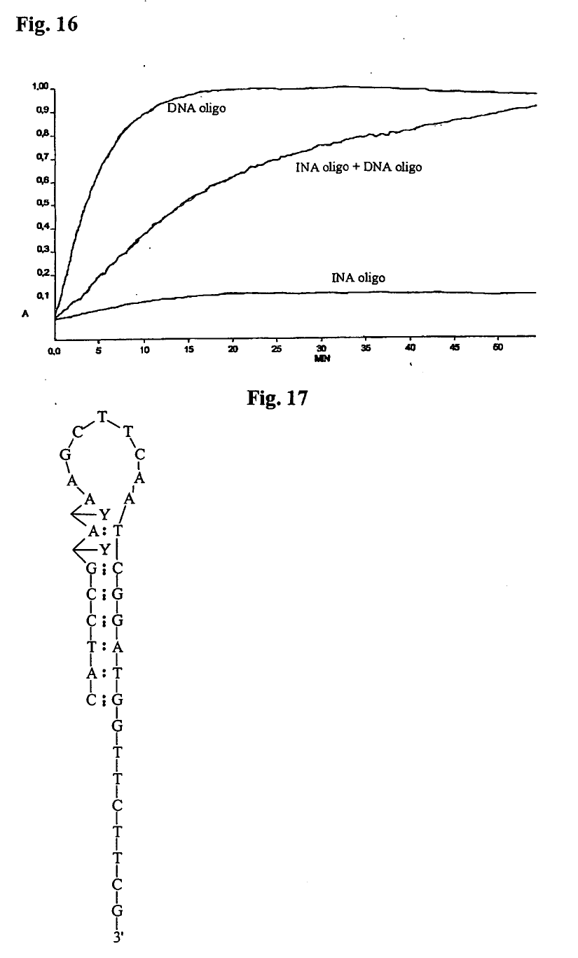

The stability of an INA (Intercalating Pseudonucleotide comprised Nucleic Acid) oligonucleotide toward 3′-exonucleolytic degradation in vitro was evaluated and compared to normal DNA's stability using the Snake Venom phosphordiesterase (SVPDE), FIG. 16. It is shown that the reference DNA oligonucleotide, I, is totally digested by SVPDE within 15 min. In contrast the INA oligonucleotide only shows a small hyperchromicity within the first 15 min. and thereafter no significant hyperchromicity is observed. These experiments indicates that the DNA nucleotide in the 3′-end of the INA oligonucleotide is digested by SVPDE, but when the enzyme meets the first intercalating pseudonucleotide it is stalled and unable to digest further.

The experiment where strand I and II are mixed in the SVPDE assay, giving a hybrid. A slow digestion compared to the reference DNA strand alone is observed. Almost a full degradation of the DNA strand is observed after 60 min. This result indicates that the hybrid, is degraded slower than the single stranded DNA.

### Example 15

### Hairpin Shape Oligonucletides Comprising Intercalating Pseudonucleotides for the Detection of Nucleic Acid

Introduction

In this example it is shown how hairpin shaped oligonucleotides comprising intercalating pseudonucleotides (probe I) can be used for the detection of nucleic acids. It is further more shown that using this principle it is possible to detect as low as a 5 nM solution (1 pmol in 200 μL) of target nucleic acid. It is also shown that the addition of Hexadecyl trimethyl ammoniumbromide (HTMAB) can enhance the signal sensitivity in a concentration dependent matter.

Materials and Methods

Below is shown the sequence of the detection probe comprising intercalating pseudonucleotides. The nucleotides which is involved in the hairpin formation is underlined and the nucleotides that are involved in the binding to target is in shown in bold letters:

Probe I:
5′- CAT CCG YAY AAG CTT CAA TCG GAT GGT TCT TCG

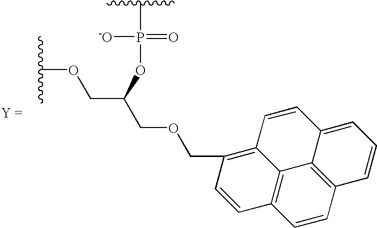

In figure FIG. 17 is shown the secondary structure of the hairpin. The hydrogen bonds of the basepairs in the stem is shown as dots.

Below is shown the sequence of the target used in these experiments. The nucleotides participating in the binding of the detection probe is shown in bold letters:

Target:
3′- ATA GTA TTT ATT CGA AGT TAG CCT ACC AAG AAG
CCT TTT TTG

All hybridisation experiments were carried out in a buffer solution containing:

140 mM NaCl

10 mM Na₂HPO₄.2 H₂O

1 mM EDTA

pH=7.0

The surfactant used in the experiments was HTMAB:

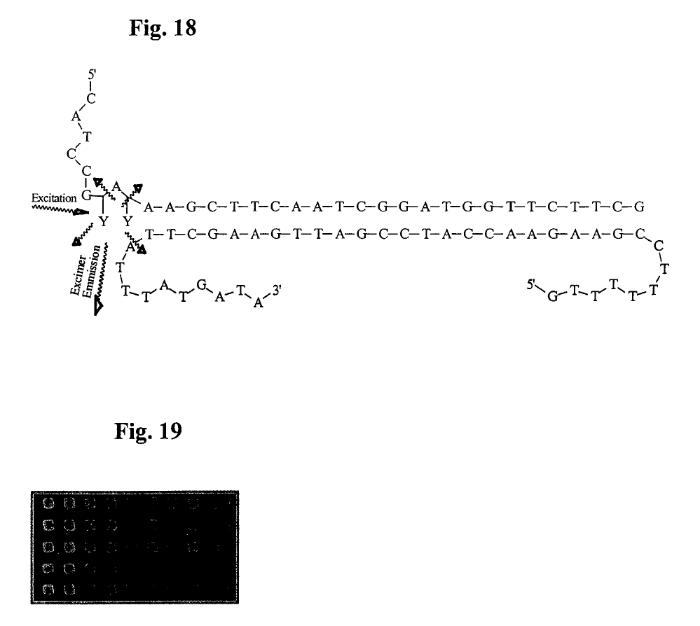

In Figure FIG. 18 is shown a figure that illustrates when the probe binds to its target sequence. It is shown that when the probe is hybridised to the Target, the two pyrene moieties from the intercalating pseudonucleotides are no longer separated by an intact base pair. This makes it possible for them to interact more freely, giving rise to higher excimer fluorescence:

In the table below is shown the designed of the experiment:

| Wells | 1 | 2 | 3 | 4 | 5 | 6 | 7 | 8 |
|---|---|---|---|---|---|---|---|---|
| a | H₂O | buffer | Probe I | Probe I | Probe I | Probe I | Probe I | Probe I |
| | | | 100 pmol | 10 pmol | 1 pmol | 100 pmol + | 10 pmol + | 1 pmol + |
| | | | | | | Target | Target | Target |
| | | | | | | 100 pmol | 10 pmol | 1 pmol |
| b | H₂O + | buffer + | Probe I | Probe I | Probe I | Probe I | Probe I | Probe I |
| | surfactant | sufactant | 100 pmol + | 10 pmol + | 1 pmol + | 100 pmol + | 10 pmol + | 1 pmol + |
| | 10⁻⁶ | 10⁻⁶ | surfactant | surfactant | surfactant | Target | Target | Target |
| | | | 10⁻⁶ | 10⁻⁶ | 10⁻⁶ | 100 pmol + | 10 pmol + | 1 pmol + |
| | | | | | | surfactant | surfactant | surfactant |
| | | | | | | 10⁻⁶ | 10⁻⁶ | 10⁻⁶ |
| c | H₂O + | buffer + | Probe I | Probe I | Probe I | Probe I | Probe I | Probe I |
| | surfactant | sufactant | 100 pmol + | 10 pmol + | 1 pmol + | 100 pmol + | 10 pmol + | 1 pmol + |
| | 10⁻⁵ | 10⁻⁵ | surfactant | surfactant | surfactant | Target | Target | Target 1 pmol + |
| | | | 10⁻⁵ | 10⁻⁵ | 10⁻⁵ | 100 pmol + | 10 pmol + | surfactant |
| | | | | | | surfactant | surfactant | 10⁻⁵ |
| | | | | | | 10⁻⁵ | 10⁻⁵ | |
| d | H₂O + | buffer + | Probe I | Probe I | Probe I | Probe I | Probe I | Probe I |
| | surfactant | sufactant | 100 pmol + | 10 pmol + | 1 pmol + | 100 pmol + | 10 pmol + | 1 pmol + |
| | 10⁻⁴ | 10⁻⁴ | surfactant | surfactant | surfactant | Target | Target | Target 1 pmol + |
| | | | 10⁻⁴ | 10⁻⁴ | 10⁻⁴ | 100 pmol + | 10 pmol + | surfactant |
| | | | | | | surfactant | surfactant | 10⁻⁴ |
| | | | | | | 10⁻⁴ | 10⁻⁴ | |

All the probes and targets were annealed in 200 μL buffer separately in Eppendorf tubes at 95° C. for 2.5 min. and then slowly cooled to room temperature and transferred to a 96-well black plate from NUNC. The fluorescence was measured on a Wallac Victor², 1420 Multilabel counter, with the following specifications:

Emission filter: F340

Excitation filter: 500-10F

Measurement time: 0.1 s, 4.0 mm from the bottom of the plate.

CW-lamp energy: 50054, Constant Voltage control.

Results and Discussion

Below is shown the results of the measurement:

| Wells | 1 | 2 | 3 | 4 | 5 | 6 | 7 | 8 |
|---|---|---|---|---|---|---|---|---|
| a | 4299 | 3471 | 4927 | 3204 | 2639 | 20643 | 4988 | 3841 |
| b | 5709 | 3619 | 9684 | 4563 | 3644 | 18611 | 3971 | 4308 |
| c | 4021 | 3119 | 8429 | 1879 | 3456 | 13833 | 5337 | 2202 |
| d | 2236 | 3194 | 120959 | 12749 | 1898 | 223956 | 21684 | 4783 |

As can be seen from comparing a3 with a6, there is a large increase in fluorescence on hybridisation of probe I to a target strand showing proofing the principle in using hairpin-shaped oligonucleotides for detection of nucleic acids sequences. If the background level (a2) is deducted from the measurement, nearly a 12 times increase in fluorescence of probe I upon hybridisation to its target sequence is observed.

By comparing a4 with a7 and a5 with a8 it can be seen that it is possible to detect the presence of as low as 10 down to 1 pmol of target nucleic acid.

The addition of surfactants on the fluorescence level is also shown. The addition of the HTMAB surfactant increases the fluorescence in some cases more than 100 times (column 6), and hence increases the sensitivity of the detection up to a 100 times.

These results compared with the fact that probe I can be used as a primer in template directed extension reactions makes oligonucleotides or oligonucleotide analogues a very useful tool in e.g. the detection of nucleic acids, for labelling nucleic acids, for the use in extension reactions like ligation and PCR and in real-time quantitative PCR.

### Example 16

### Control of Oligobinding & INA-signal on SAL-chips.

Method

Chip Production
Oligos, 50 μM, are spotted dissolved in 400 mM Sodium carbonate buffer, pH 9.
The chip is instantly placed in humidity—chamber, 37° C. for one hour.
Oligo binding is effectuated on the surface of the chip by washing with 1% NH₄OH, 5 min.
Unheated deionised water is used to wash for 2×2 min.
The chips are centrifuged, 600 rpm for 5 min, to remove excess water from the surface.
The chip is scanned, or stored refrigerated at 4° C.

SYBR Green II control staining of oligo-binding.
Deionised water, approximately 90° C. is used to wash for 2×2 min. Centrifugation, 600 rpm for 5 min follows.
10 000× dilution of SYBR Green II is added to the chip, apply cover and incubate at ambient temperature for 2-3 min.
Wash for 1 min, using unheated water and centrifuge the chip to dryness, 600 rpm for 5 min.
The chip is now ready to be scanned, use Alexa 488 filterset.

Results

Section of Chip from OUH, HUMAC, with Amino-Linker Oligos Stained with SYBR Green II.

Spot size: 100 μm. Center-center distance: 175 μm

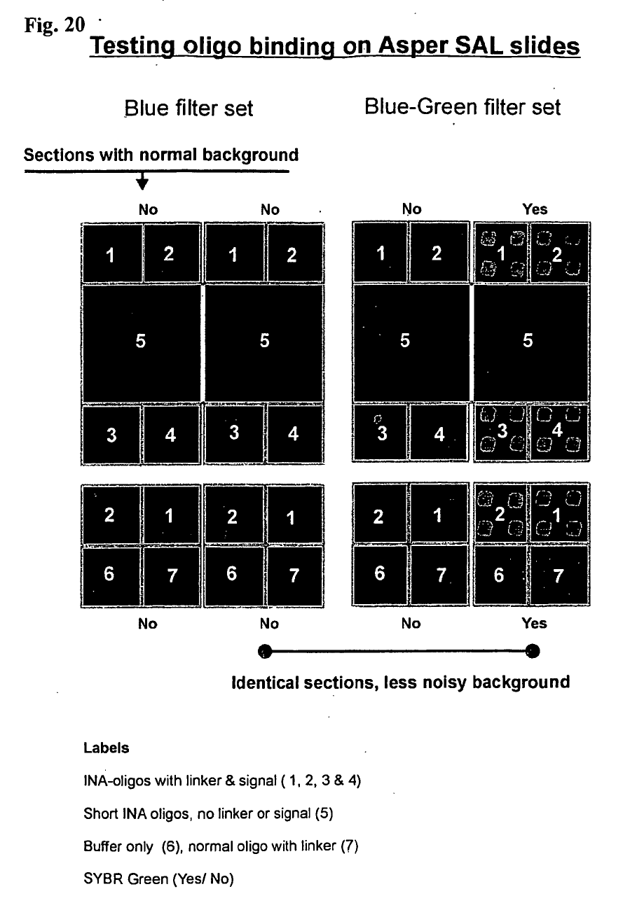

Evaluation of Oligo- and INA-Binding on Asper SAL-chips (see Figure FIG. 20).
INA oligos with signal-modification and aminolinker, presumably bind to SAL chips like normal aminolinker oligos. (see SYBR Green Yes: 1,2,3,4 and 7).
All signal-INAs fluoresce, when blue or blue-green filtersets are utilized on our ArrayWorx scanner (SYBR Green, No: 1, 2, 3, 4).
The position of the signal-modification compared to the oligo-5′-end doesn't seem to be significant. No evident difference is observed between the strength of the signal depending whether the modification is furthest away from the 5′-end (1) or closest to the 5′-end. (4)
The short 10-mere INAs, without linkers, apparently don't bind to the chip—they lack the linker (see 5). The signal is of the same strength as when clean buffer without oligos is spotted. (See SYBR Green Yes, 5 & 6)
The variable background that is shown with different sections of the same chip can be caused by inadequate wash, calibration of scanner, or variation in SAL coating.
Observed tendency: Generally the quality of spots, that is shape and signal-homogeneity, seem to be better, when the oligos contain INA modifications (compare 1 and 7, bottom right.)

### Example 17

### Hybridisation Properties of DNA-DNA, INA-DNA and INA-INA Hybrids at Different pH Values

Introduction

In this example it is shown the hybridisation affinities for DNA-DNA, INA-DNA and INA-INA hybrids at different pH values.

Materials and Methods

Hybrids:

Hybrid I
5′-CTC AAC CAA GCT
3′-GAG TTG GTT CGA
Hybrid II
5′-CTC AAC YCA AGC T
3′-GAG TTG GTT CGA
Hybrid III
5′-CTC AAC CAA GCT
3′-GAG TTG YGT TCG A
Hybrid IV
5′-CTC AAC YCA AGC T
3′-GAG TTG YGT TCG A

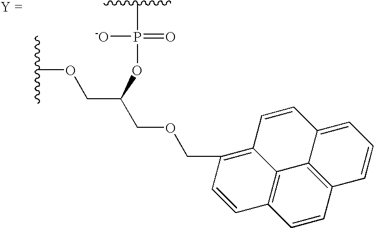

All hybridisation experiments were carried out with 1.5 μM of both target and probe strands in 1 mL of a buffer solution containing:

140 mM NaCl 10 mM Na₂HPO₄.2 H₂O 1 mM EDTA

The target strands and probes were annealed by mixing them in the above mentioned buffer at 95° C. for 3 min. after which they are slowly cooled to room temperature. The melting temperatures of the hybridised probe-target hybrids were found by slowly heating the solution in a quartz cuvette, while simultaneously determining the absorbance. All melting temperatures presented in this example are with an uncertainty of ±1.0° C. as determined by repetitive experiments.

pH was adjusted with a solution of 25% NH₄OH and glacial acetic acid.

Results and Discussion

| | pH | | | | | | |
|---|---|---|---|---|---|---|---|
| Hybrid # | 4.2 | 5.0 | 6.1 | 7.0 | 8.0 | 9.0 | 10.0 |
| I | 30.6 | 43.6 | 47.8 | 47.2 | 49.6 | 49.6 | 43.3 |
| II | — | 49.7 | 54.7 | 54.5 | 55.7 | 54.1 | 51.1 |
| III | — | 45.4 | 51.9 | 52.5 | 54.7 | 52.1 | 46.4 |
| IV | — | 36.1 | 46.0 | 46.5 | 48.5 | 46.5 | 40.9 |

In the table above is shown the results of the melting temperature experiments of hybrid I-IV at different pH values. As can be seen from the table hybrid II and III have higher melting temperatures over the pH range from pH=5 to 10 than the homologous DNA duplex (hybrid I) and hybrid IV. This shows that it is possible to reduce the cross hybridisation between complementary sequences, when both sequences comprise at least one intercalating pseudonucleotide that are positioned opposite each other when said sequences hybridise. It can also be seen that the melting temperatures of all the hybrids are highest at around pH=8, and hence in some preferred embodiments it is preferred to hybridise at pH=8 ±2. The largest difference in melting temperature between the hybrids II and III comprising one intercalating pseudonucleotide and the hybrid IV comprising two opposite positioned intercalating pseudonucleotides is at pH=5.0, namely 13.6° C. and 9.3° C. respectively. Hence in a preferred embodiment hybridisations between an oligonucleotide or oligonucleotide analogue comprising at least one intercalating pseudonucleotide and a nucleic acid or nucleic acid analogue is carried out at pH=5±1.

### Example 18

### Preparing a Sample for RT-PCR

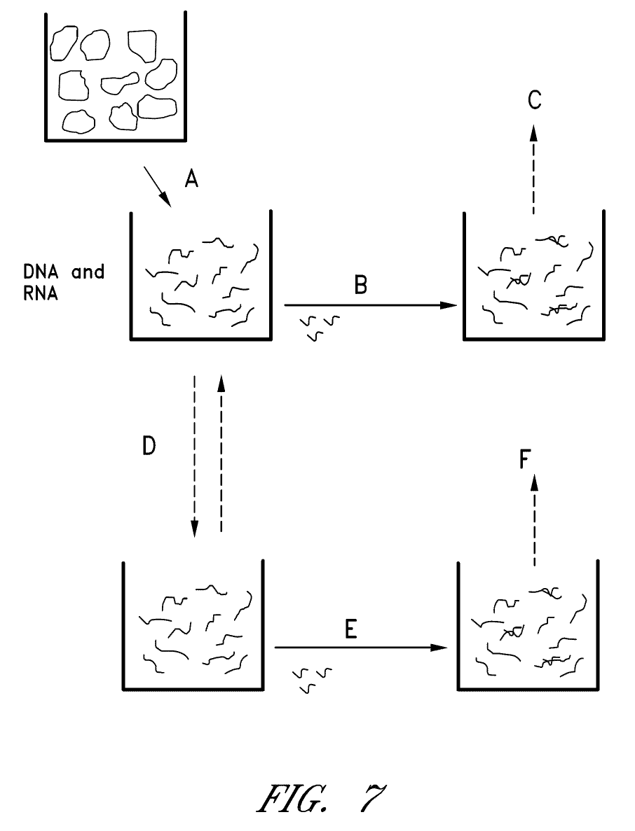

The method of preparing a sample for RT-PCR of a target sequence is depicted in FIG. 7. The method has the advantage that false positive signals from DNA are largely reduced.

A cell sample is provided and the cell walls of the cell are destroyed, thereby releasing DNA and RNA from the cells (FIG. 7A). Subsequently, an oligonucleotide comprising an intercalator pseudonucleotide, which can hybridise to the target sequence is incubated with the DNA/RNA sample under conditions allowing hybridisation between the oligo and DNA (FIG. 7B). The sample is then ready to be upscaled by any standard RT-PCR procedure (FIG. 7C). Because target DNA present in the sample is blocked by hybridisation to the oligonucleotide, then only RNA may be amplified.

Alternatively, after destroying the cell walls, RNA may be purified by any standard method for example by extraction and precipitation (FIG. 7D). Usually, the purified RNA will comprise small amounts of DNA contamination. Hence, an oligonucleotide comprising an intercalator pseudonucleotide, which can hybridise to the target sequence is incubated with the RNA sample under conditions allowing hybridisation between the oligo and DNA (FIG. 7E). The sample is then ready to be upscaled by any standard RT-PCR procedure (FIG. 7F). Because target DNA contamination present in the sample is blocked by hybridisation to the oligonucleotide, then only RNA may be amplified.

### Example 19

### Preparing a Sample for RT-PCR

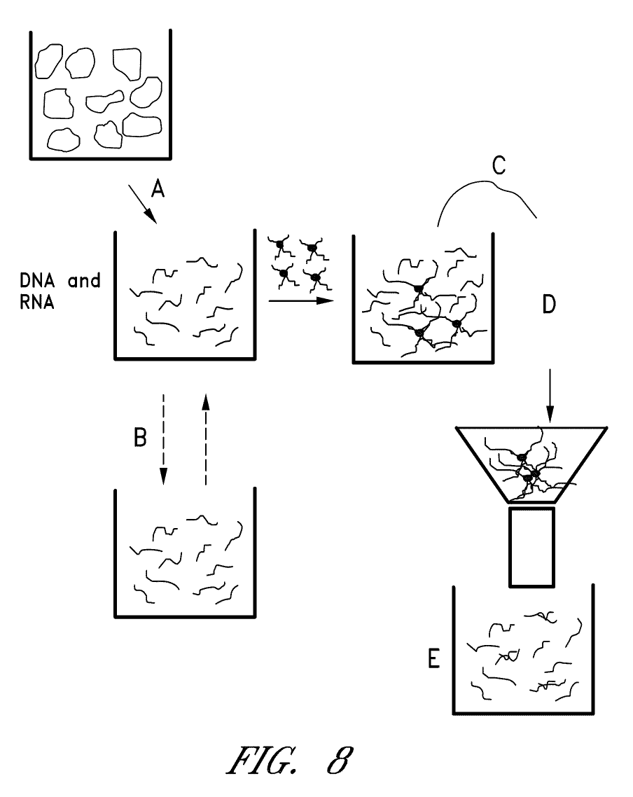

The method of preparing a sample for RT-PCR of a target sequence is depicted in FIG. 8. The method has the advantage that false positive signals from DNA are largely reduced.

A cell sample is provided and the cell walls are destroyed, thereby DNA and RNA is released. RNA may be purified by any standard method from the sample (FIG. 8B), however it is also possible to perform the subsequent steps on the DNA/RNA sample.

The sample is incubated with beads linked to an oligonucleotide comprising an intercalator pseudonucleotide (FIG. 8C), which can hybridise to the target sequence under conditions allowing hybridisation between the oligo and DNA. After hybridisation the sample is filtered to remove the beads together with bound target DNA from the sample (FIG. 8D).

The sample is ready for RT-PCR (FIG. 8E). Because the sequence specific target

DNA has been removed from the sample, the risk of false positives of the RT-PCR due to DNA contamination is largely reduced.

Alternatively, after sample preparation, the sample is incubated with a solid support linked to an oligonucleotide comprising an intercalator pseudonucleotide (FIG. 9B), which can hybridise to the target sequence under conditions allowing hybridisation between the oligo and DNA. After hybridisation the solid support is removed from the sample together with bound target DNA. The sample may once again be incubated with a solid support linked to an oligonucleotide comprising an intercalator pseudonucleotide to remove traces of sequence specific DNA still left in the sample. The solid support is removed from the sample after hybridisation to sequence specific DNA (FIG. 9C).

The sample is then ready for RT-PCR.

### Example 20

### Purification of Sequence Specific DNA

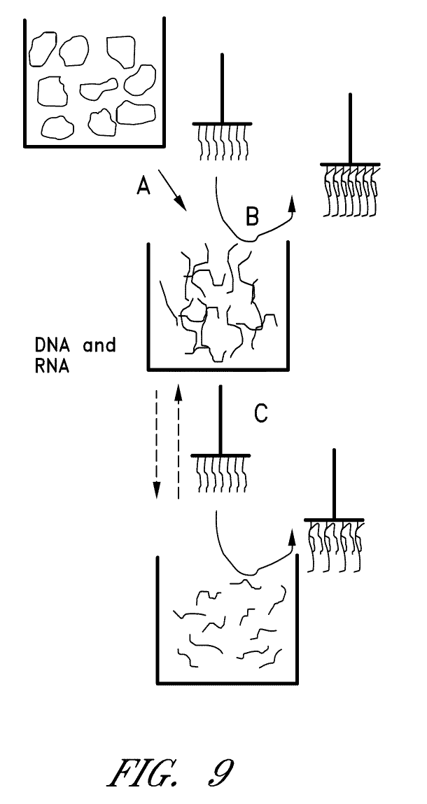

The purification of sequence specific DNA is illustrated in FIGS. 9 and 10.

A cell sample is treated with GnSCN thereby releasing nucleic acids. The sample is incubated with beads linked to an oligonucleotide comprising an intercalator pseudonucleotide (FIG. 10A), which can hybridise to the target sequence under conditions allowing hybridisation between the oligo and DNA. The sample is filtrated and washed to remove non-bound nucleic acids (FIG. 10B). The beads are subjected to heating and filtration, releasing pure, sequence specific DNA largely free of sequence specific RNA (FIG. 10C).

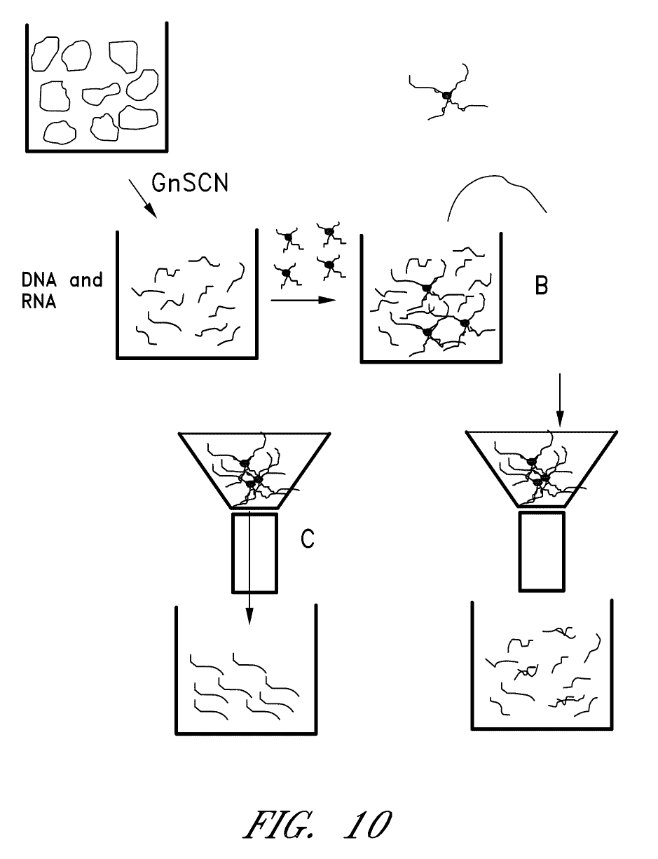

Alternatively, the nucleic acid sample is incubated with a solid support linked to an oligonucleotide comprising an intercalator pseudonucleotide (FIG. 11B), which can hybridise to the target sequence under conditions allowing hybridisation between the oligo and DNA. The solid support is separated from the rest of the sample and subjected to heating, which releases the sequence specific DNA (FIG. 11C). The sequence specific DNA will be largely free of sequence specific RNA and is ready for diagnosis, PCR or other purposes.

### Example 21

### Detection of Target DNA

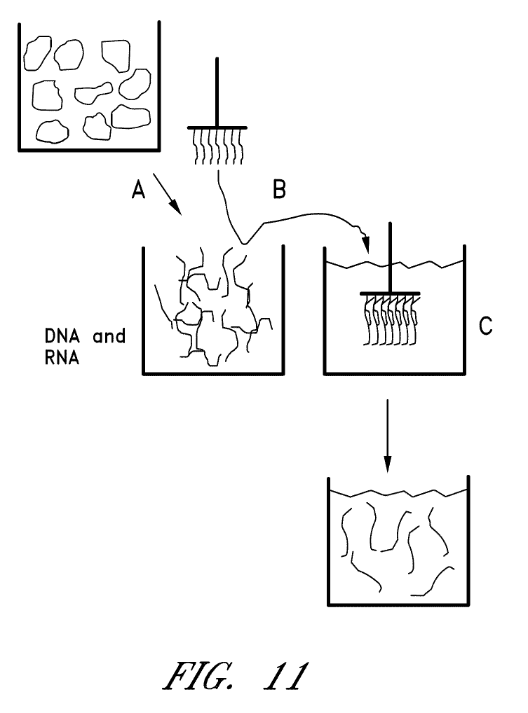

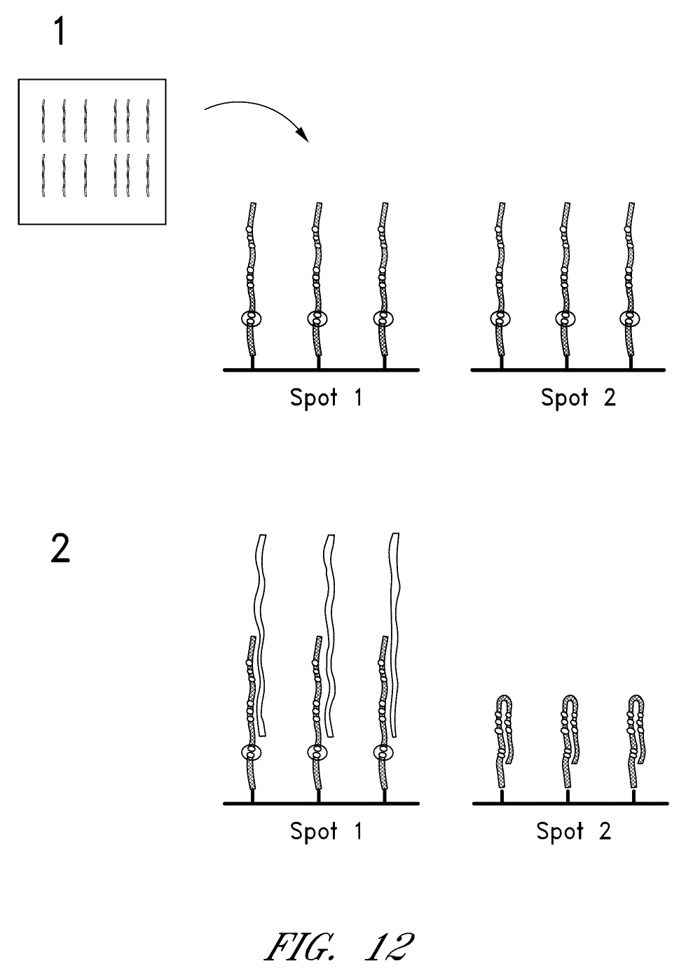

Oligonucleotides comprising pyrene pseudonucleotides are linked to a chip. The oligonucleotides are designed so that a part of it may hybridise to a specific target DNA and so that the oligonucleotide may also self-hybridise. When the oligonucleotide is hybridised to itself, 3 pairs of pyrene pseudonucleotides are facing each other, and accordingly the melting temperature of a DNA/oligo hybrid is higher than the melting temperature of the selfhybrid. Furthermore, the oligonucleotide comprises two pyrenes capable of forming an excimer, only when the probe is not hybridised to itself (FIG. 12 and FIG. 13A).

Different oligonucleotides recognising different target DNAs may be added to various defined regions of the chip. In the present example 2 different oligonucleotides are linked to spot 1 and spot 2, respectively.

A crude mixture of DNA fragments containing the target DNA is added to the chip at a temperature where the oligonucleotide can not selfanneal.

After an annealing step and a washing step, the temperature is lowered to allow self hybridisation of probes. Excimer formation is used to detect the presence of target DNA as well as the amount of target DNA hybridised. The procedure is outlined in FIG. 12.

Alternatively, the oligonucleotide may be designed so that it comprises a fluorophore and a quencher, wherein the fluorophore signal may only be quenched by the quencher when the oligonucleotide is self-hybridised (FIG. 13B).

It is also possible to use two oligonucleotides which each comprises 3 pyrenes pseudonucleotides that are facing each other when the oligonucleotides are hybridised. The oligonucleotides also contains a fluorophore and a quencher each, positioned so that the fluorophore signals may only be quenched by the quencher when the oligonucleotides are hybridised (FIG. 13C).

### Example 22

### Exciplex Fluorescence from Intercalating Pseudonucleotides

Introduction

In this example is shown some exciplex fluorescence emission between two intercalating pseudonucleotides.

Materials, Methods and Results

Three oligonucleotides comprising two different intercalating pseudonucleotides were synthesized using standard procedures:

Sequence 1:
5′-CTCAAYGDCAAGCT
Sequence 2:
5′-CTCAAGYDCAAGCT
Sequence 3:
5′-CTCAAGYXCAAGCT

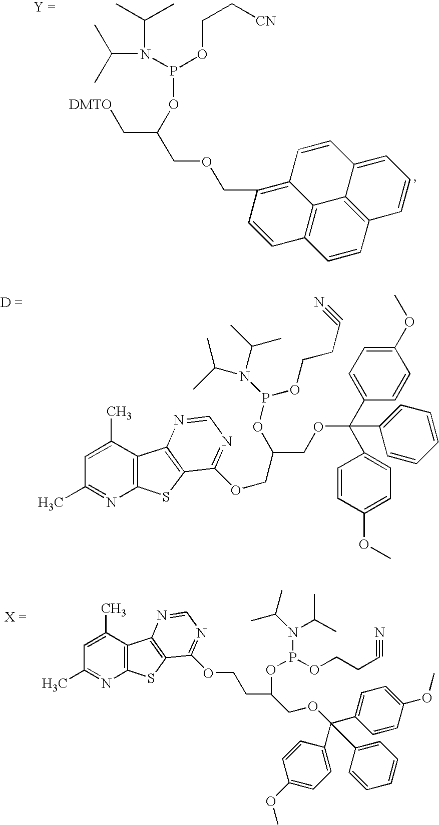

After purification by HPLC the oligonucleotides comprising intercalating pseudonucleotides was dissolved in a buffer solution containing:

140 mM NaCl

10 mM Na₂HPO₄.2 H₂O

1 mM EDTA

pH=7.0

and all fluorescence experiments were carried out in this buffer. Excitation was done at 343 nm on a Perkin Elmer MPF-3 spectrophotometer with at xenon 150 power supply.

As seen from Figures FIG. 21 and FIG. 22, the exciplex to monomer fluorescence ratio was higher when the to intercalating pseudonucleotides were positioned as neighbours (Figure FIG. 21) than when placed as next-nearest neighbours (Figure FIG. 22)—the exciplex transition was however clearly observed in both cases. Similar result was obtained with the amidite X.

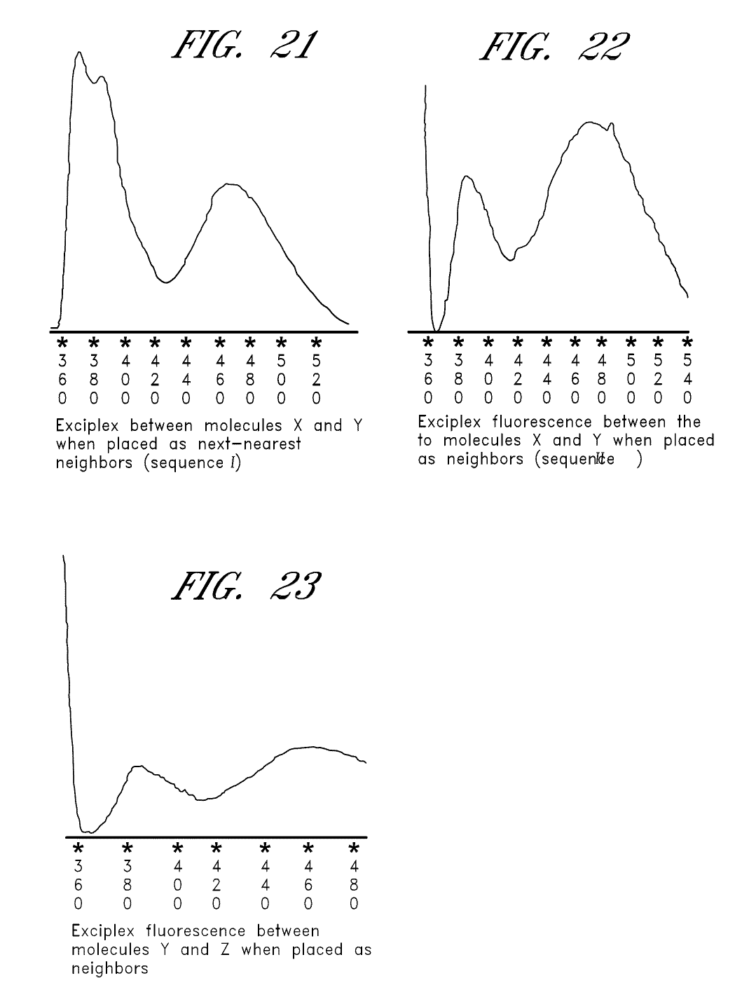

As a conclusion an exciplex between two intercalating pseudonucleotides can be observed for both neighbouring and next-nearest neighbouring intercalating pseudonucleotides, when said pseudonucleotides are positioned internally in an oligonucleotide (See Figure FIG. 23).

### Example 23

### PCR with Oligonucleotide Primers Comprising Intercalator Pseudionucleotides

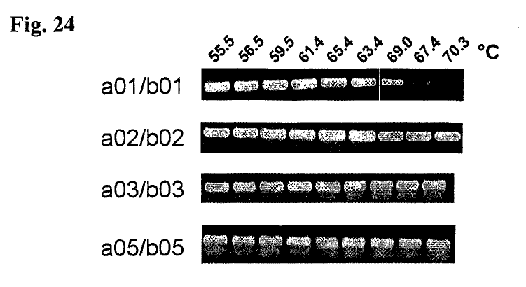

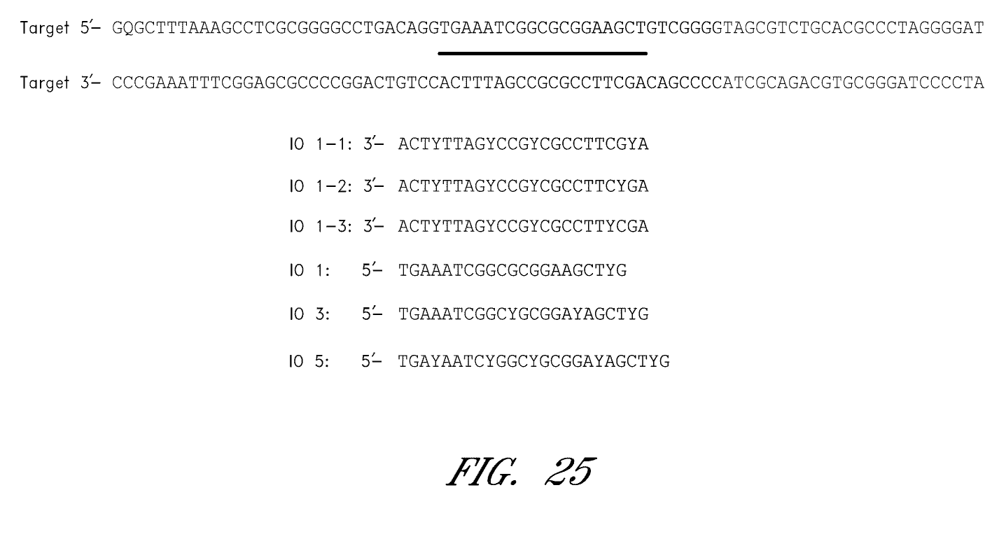

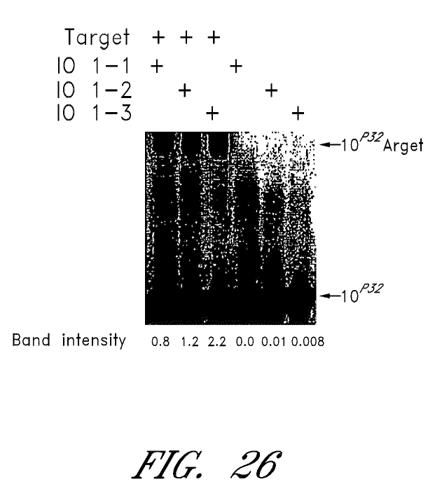

35 cycles of gradient PCR (94° C., 30 sec; gradient annealing temp, 45 sec; 72° C., 60 sec.) were performed with diluted plasmid template in a standard PCR-buffer (1.5 mM MgCl₂; 50 mM KCl; 10 mM Tris-HCl; 0.1% Triton X-100, 200 □M of each dNTP, 5 pmol of each primer) in a final of volume of 25 □I. PCR products were separeated in a 0.7% agorose gel in 1× TBE buffer and visualized by EtBr staining. Temperatures on the FIG. 24 denote the annealing temperature in each well.

Primer designs (upstream and downstream, respectively)

| | |
|---|---|
| a01 | 5′- AAGCTTCAATCGGATGGTTCTTCG |
| a02 | 5′- YAAGCTTCAATCGGATGGTTCTTCG |
| a03 | 5′- YCYATCCGAAAGCTTCAATCGGATGGTTCTTCG |
| a05 | 5′- CYAYTCCGAAAGCTTCAATCGGATGGTTCTTCG |
| b01 | 5′- CACAAGAGCTGACCCAATGGTTGC |
| b02 | 5′- YCACAAGAGCTGACCCAATGGTTGC |
| b03 | 5′- YTYGGGTCACACAAGAGCTGACCCAATGGTTGC |
| b05 | 5′- TYGYGGTCACACAAGAGCTGACCCAATGGTTGC |

Primers 03 and 05 are able to form hairpin loops when not hybridized with target as exemplified below by the a05 primer:

| | |
|---|---|
| Primer alone | TCAA |
| | T TCGGA T GGTTCTTCG-3′ |
| | C AGCCTYAY |
| | GAA C-5′ |
| Primer | 5′- CYAYTCCGA |
| | AAGCTTCAATCGGATGGTTCTTCG |
| | TTCGAAGTTAGCCTACCAAGAAGC |
| Target: | 5′- TA |
| | CT |

Conclusion

As can be observed from the picture of the gel, the addition of one single end-positioned intercalator pseudonucleotide in the linear primers a02/b02 compared to the DNA control primers raises the effective melting temperature significantly.

From the amplification products of bands for the 03 and 05 primers it primers with beacon design primes PCR in a highly efficient manner.

### Example 24

### INA-Oligo Binding to DNA Target

Results and Discussion

Binding Requires Target-Specificity and Occurs Spontaneously.

A series of INA-oligonucleotides were designed and tested for their abilities to spontaneously bind an 80 by complementary target DNA sequence (FIG. 25).

Reactions were carried out by incubation of the double-stranded target DNA with an excess of P32-labelled INA-oligoes (IOs) in a sodium-phosphate buffer containing 120 mM Sodium chloride at 37° C. for 1-3 hours. Results were then evaluated by electrophoretic mobility shift analysis and phospor-imaging of the labelled IOs.

FIG. 26 shows that all three IOs tested (IO-1-1, IO 1-2, IO 1-3) bound the target DNA. The relative amounts of bound IOs were determined by volume analysis of the retarded bands using the ImageQuant software. As the numbers at the bottom of the figure indicates the IOs showed different affinities for the target. The IO 1-3 clearly had an advantage in binding the target and was therefore chosen for further analysis.

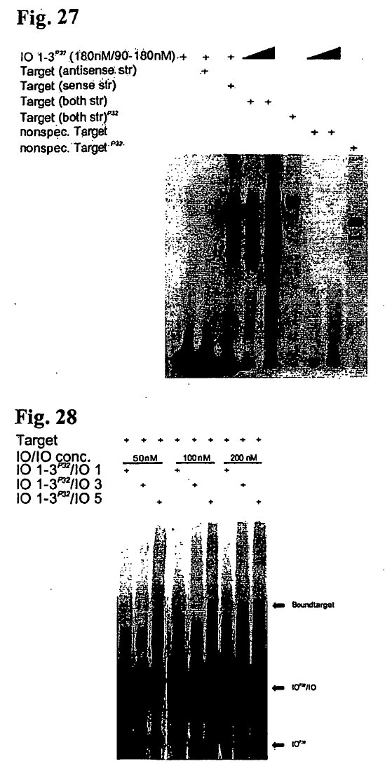

Evidence for the specificity of the observed binding was next ascertained. First P32 labelled IO 1-3 was incubated with either strand of the DNA target alone. As expected based on sequence complimentarity IO 1-3 specifically bound the sense strand of the DNA target (FIG. 27, lane 1-3). Binding to the double stranded DNA target was then assayed at increasing concentration of P32 labelled IO 1-3 and compared to binding to an unrelated 60 by target DNA sequence (compare lanes 4-5 with 7-8). Clearly, target binding by the IO required target complimentarity as no binding was observed with the sequence-unrelated target DNA. To verify the position of the observed retardation a fraction of the target DNAs were P32 labelled and 5. their retardation assayed in parallel (lanes 6 and 9).

IO Pairing does not Inhibit Spontaneous Binding and Gives Variable Target-Affinities by Differential Positioning of the Intercalating Units.

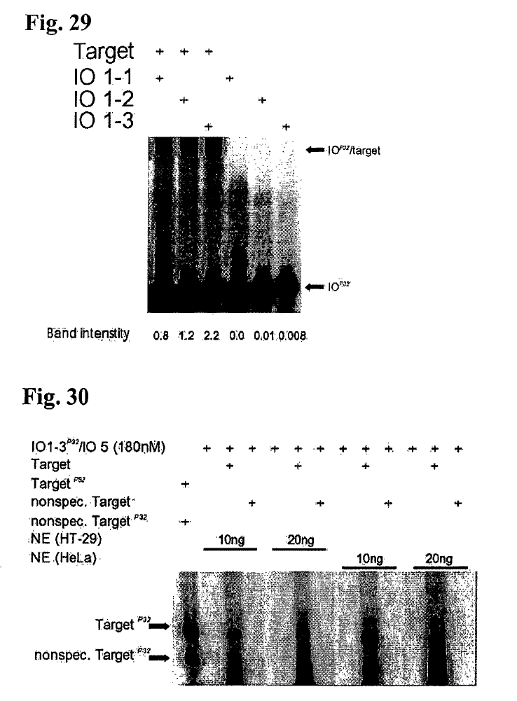

The observed target strand specificity of the IOs (ie. IO 1-3 specifically binding the sense strand of the target DNA, see above) suggested that the antisense strand of the target DNA may be free to be simultaneously attacked by a different IO. To explore this possibility IO 1-3 was annealed to three different, complementary IOs. As shown in FIG. 28 the pairing of the IOs still rendered the P32 labelled IO 1-3 capable of spontaneously binding the target DNA, albeit with different affinity depending on the positioning of the intercalating units in the pairing IO. As the IO 1-3/IO 5 pair gave the best target-binding this pair was chosen for further testing. To investigate whether pairing affects the efficiency of spontaneous target binding, binding of IO 1-3 was assayed with and without previous pairing to IO 5. FIG. 29 shows that pairing did not affect the spontaneous binding of IO 1-3 to the target DNA.

Nuclear Factors aid IO Target Binding and Favours Paired IOs

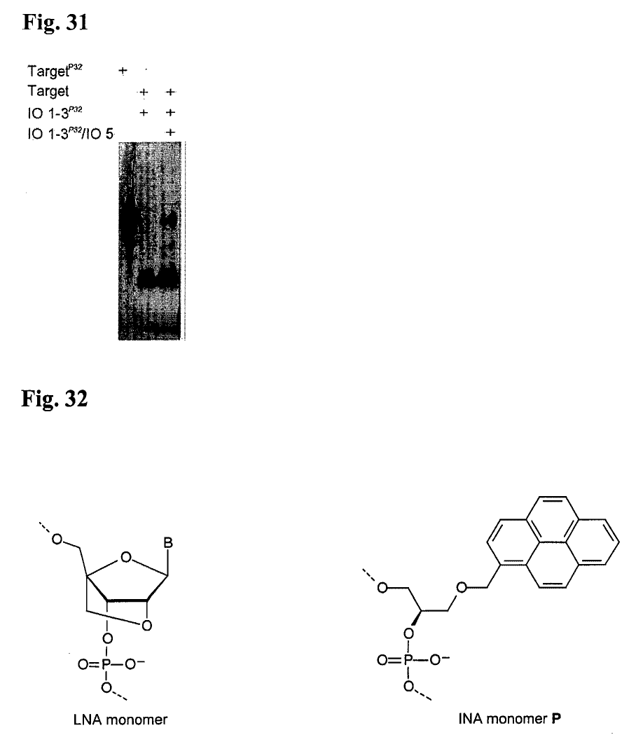

RecA/Rad51 assisted joint molecule formation between DNA targets and small RNA-DNA oligonucleotides have previously been reported (Gamper 2000, Yoon 2002). The IO readily bound the target unassisted and thus hold promise as bona fide agents for DNA targeting for therapeutic purposes. It therefore was of great importance to clarify how this binding would proceed in a nuclear environment. To address this subject we employed nuclear extracts prepared from human cell culture. As shown in FIG. 30, when reactions were carried out in the presence of nuclear extract retardation of IO 1-3 was at least 3-5 fold increased (calculations not shown), and only occurred in the presence of the specific target. Moreover, the degree of binding was dependent on the amount of nuclear protein added, and as such increased until a certain amount of protein was added. It then decreased as would be expected based on similar analysis with addition of protein involved in DNA repair processes. It is generally assumed that this process involves a D-loop formation of the targeted DNA, leaving both strands open for attack by matching oligo-nucleotides. It was therefore interesting to observe that upon addition of nuclear extract pairing IO's did indeed enhance the binding of the P32-labelled IO 1-3 to the DNA target (FIG. 31).

Materials and Methods

Oligo-synthesis: all oligos were prepared by standard procedures.

Radioactive labelling of oligos: oligos were endlabelled by incubation with polynucleotide kinase and γ-P³² ATP. Labelled oligoes were purified by the Mermaid kit procedure.

Nuclear extracts: HT-29 extracts were prepared from pre-confluent HT-29 colon-cancer cells by the NUN extraction procedure. HeLa nuclear extracts were obtained from (a company in Belgium).

Electrophoretic mobility assay: Reactions were carried out in 20 mM sodium-phosphate buffer pH 8.0 containing 120 mM NaCl, 1 mM DTE. Upon incubation at 37° C. reactions were snap-frozen in liquid N2 and stored at −80° C. or applied directly to electrophoresis on 7 or 10% polyacrylamide gel cast in ½× TBE, at 300 V for 2-4 h, at 4° C.

Reactions containing nuclear extracts additionally contained 0.22M Urea, a total of 200 mM NaCl, 0.22% NP-40, 5.52 HEPES, 5 mM MgCl₂ and 2 mM ATP. These reactions were incubated at 37° C. for 10 min. upon which 1.175 μl 10% SDS and 37.5 μg Proteinase K was added and incubation reassumed for another 60 min.

For comparative purposes equal CPM of individual IOs were added to reactions.

Visualisation: EMSA results were evaluated using a STORM phosporimager and the ImageQuant software.

### Example 25

### LNA +INA: Making Locked Nucleic Acid Hairpins Accessible to Targeting by Insertion of Intercalating Nucleic Acid Monomers

Introduction

Hairpin structures are a common feature of single-stranded DNA and RNA sequences. This type of secondary structures can make target sequences inaccessible to intermolecular Watson-Crick base pairing (ⁱ). There is a need to find new techniques to alleviate this problem. The ones previously reported are using fragmentation of the nucleic acid sequence close to the target (ⁱⁱ) or replacing natural 2′-deoxycytidine with N⁴-ethyl-2′-deoxycytidine which forms base pairs with 2′-deoxyguanosine having reduced stability as compared with natural base pairs (ⁱⁱⁱ). A variety of modified of oligonucleotides have been developed during the last two decades in order to develop potential gene inhibitors which possess an enhanced stability towards cellular nucleases, the ability to penetrate the cell membrane and an efficient hybridisation to the target RNA/DNA. If the modified oligonucleotides have efficient hybridisation properties, they are also expected to form secondary structures which can make a large number of sequences inaccessible, but to our knowledge no attempts have been done on modified oligonucleotides to overcome this problem.

LNA oligonucleotides are oligonucleotides containing a conformationally restricted monomer with a 2′-O, 4′C-methylene bridge (FIG. 1) and they have shown helical thermal stability when hybridised to either complimentary DNA or RNA when compared with unmodified duplexes. Due to their hybridisation efficiencies they are also expected to form extremely stable hairpin structures. It is challenging also to make LNA hairpin structures accessible to targeting as LNA seems to be the most promising antisense candidate among modified oligonucleotides.

INAs (Intercalating Nucleic Acids, FIG. 1) composed by insertions of intercalating pseudonucleotides into DNA are strongly discriminating between DNA over RNA when hybridising with them (^{iv}). Properly designed INAs gives more stable

INA/DNA duplexes than its DNA/DNA counterparts whereas the opposite is found when INAs are hybridising to RNA which results in less stable duplexes than the corresponding RNA/RNA duplexes. In this paper it is shown that this property can be used to make LNA hairpins more accessible to targeting of DNA by inserting INA monomers into the stem of the hairpin.

Materials and Methods

Synthesis of DMT Protected LNA and INA Phosphoramidites

The phosphoramidite of LNA and INA, respectively, were prepared as previously described (4a,8a).

ODN, LNA and INA Synthesis, Purification and Measurement of Melting Temperatures.

The ODN, LNA and INA synthesis was carried out on an Expedite™ 8909 Nucleic Acid Synthesis System from Applied Biosystems. The LNA and INA amidite was dissolved in a 1:1 mixture of dry acetonitrile and dry dichloromethane, as a 0.1 M solution, and inserted into the growing oligonucleotides chain using same conditions as for normal nucleotide couplings (2 min coupling). The coupling efficiency of the modified nucleotide was >99%. The ODNs, LNAs and INAs were synthesised with DMT on and purified on a Waters Delta Prep 3000 HPLC with a Waters 600E controller and a Waters 484 detector on a Hamilton PRP-1 column. Buffer A: 950 ml of 0.1 M NH₄HCO₃ and 50 ml MeCN, pH 9.0; buffer B: 250 ml of 0.1 M NH₄HCO₃ and 750 ml MeCN, pH 9.0. Gradients: 5 min 100% A, linear gradient to 100% B in 40 min, 5 min with 100% B, linear gradient to 100% A in 1 min and then 100% A in 29 min (product peak at ˜37 min). The ODNs, LNAs and INAs were DMT deprotected in 925 μl of H₂O and 75 μl CH₃COOH and purified by HPLC, again using the same column, buffer system and gradients (product peak at ˜26 min). To get rid of the salts, the ODNs, LNAs and INAs were redissolved in 1 ml of water and concentrated in vacuo three times.

All ODNs, LNAs and INAs were confirmed by MALDI-TOF analysis on a Voyager Elite Biospectrometry Research Station from PerSeptive Biosystems. The transition state analyses were carried out on a Perkin Elmer UV/VIS spectrometer Lambda 2 with a PTP-6 temperature programmer using PETEMP rev. 5.1 software and PECSS software package v. 4.3. All ODNs were measured in a 120 mM NaCl, 10 mM, sodium phosphate, 1 mM EDTA, pH 7.0, 3.0 μM each strand. All melting temperatures are with an uncertainty ±0.5° C. as determined by repetitive experiments.

Results and Discussion

Duplexes

NMR has been used to determine the structure of DNA/LNA duplexes and it was found that only one LNA monomer in the duplex was sufficient to induce a change in the sugar conformation of the flanking nucleotides from a north conformation typically found in B-type DNA/DNA duplexes to a south conformation, the latter being the one typically found in the sugar parts of A-type RNA/RNA duplexes (^{v}). It was therefore found interesting to investigate systematic insertions of the INA monomer P (FIG. 32) at all possible sites of this duplex (FIG. 33). The oligo I without any LNA monomers was used as a reference target for the hybridisations with the probes 2-12 (FIG. 33) having insertions of P at all possible positions, except at the 3′-end. The oligo I had in all cases increased duplex stabilities when compared with the unmodified probe 1. In fact, probe 3 gave a remarkably stable duplex with an increase of 10.1° C. in the thermal melting temperature. This oligo has the INA monomer P inserted in an AT region and when compared with the other probes, this seems generally preferable.

For the oligo II it was observed that insertions of the INA monomer P into regions of its duplexes away from the LNA monomer increased the duplex stability when compared with the unmodified probe 1. In fact the stabilisations were nearly identical with those observed for the oligo I and this confirms that regions away from the LNA monomer have still a B-type structure. Only when the insertion of P was done into the complementary oligo close to the LNA monomer (FIG. 33, entries 2-5), differences could be observed in hybridisation efficiencies between the two oligos upon hybridisation to their complementary sequences. This could be ascribed to a conformational change of the sugar part of the neighbouring nucleotides and this was reflected by a decrease in thermal melting temperatures. The major differences were found for the duplexes with neighbouring insertions to the LNA monomer (FIG. 33, entries 2 and 3) whereas minor differences were found for duplexes with next neighbouring insertions (FIG. 33, entries 4 and 5).

Oligo III with three evenly placed LNA monomers is supposed to induce A type duplex structure in most parts of the duplex formed on its hybridisation with its complementary sequence. This is deduced by comparing ΔTₘs for the oligo III with those for the oligo I. The conclusion is in agreement with NMR structure determination on a similar duplex with three LNA monomers (^{v}). Comparable stabilisations were only found for these two oligos when the INA monomer P was inserted close to the ends of their respective duplexes and thus confirming B-type duplex at the ends (FIG. 33, entries 10 and 11).

The study on the oligos I-111 demonstrates INA monomer P insertions as a versatile tool of distinguishing A and B type duplex regions when a modified nucleotide induces an A type duplex structure into a region of a B type duplex.

Hairpins

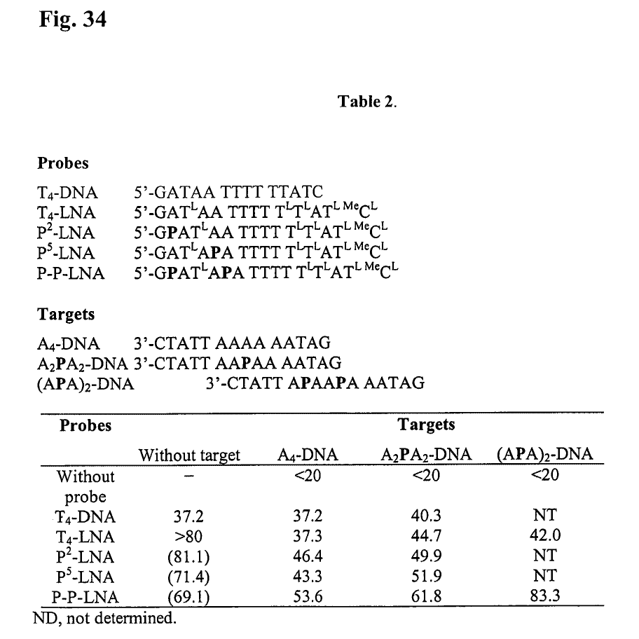

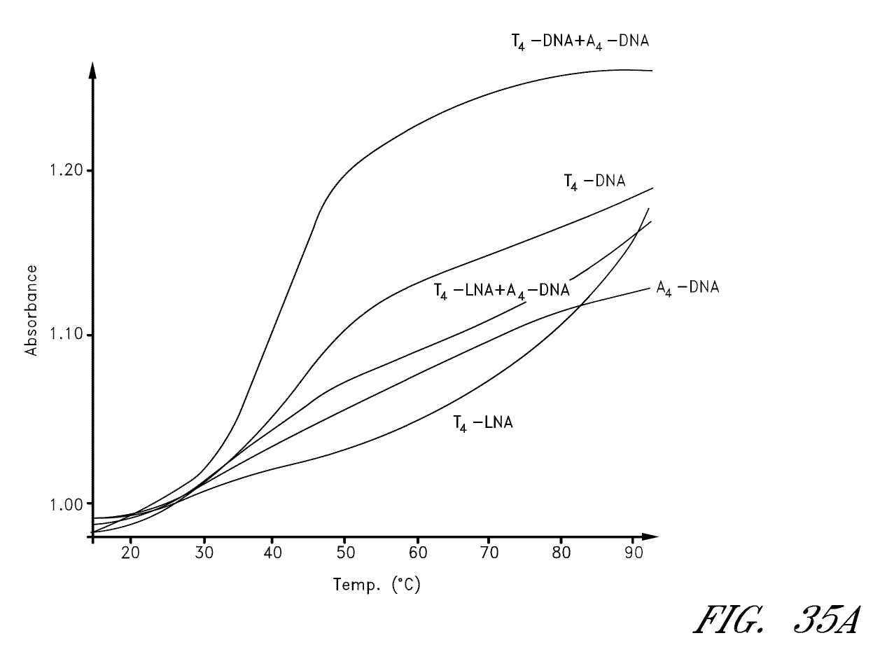

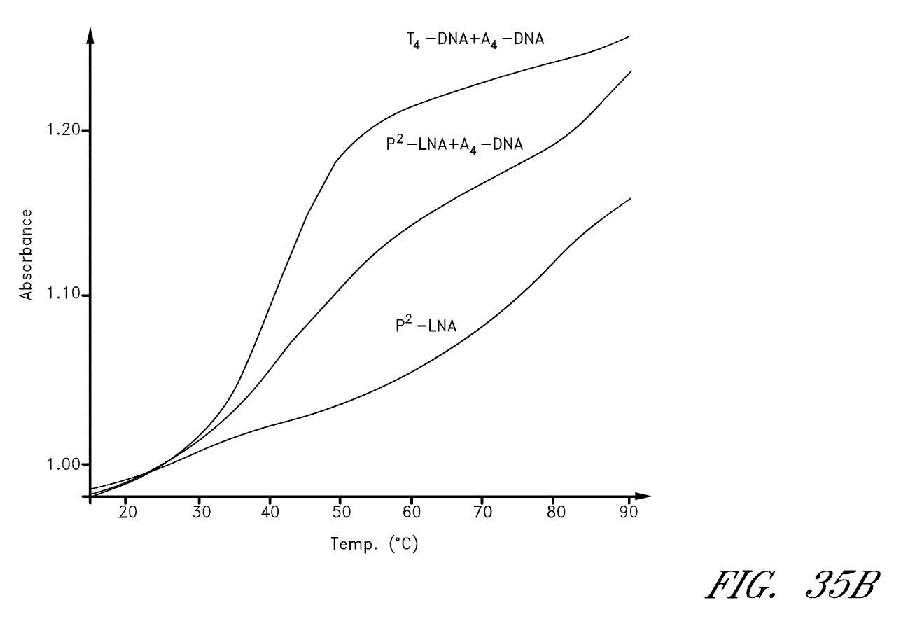

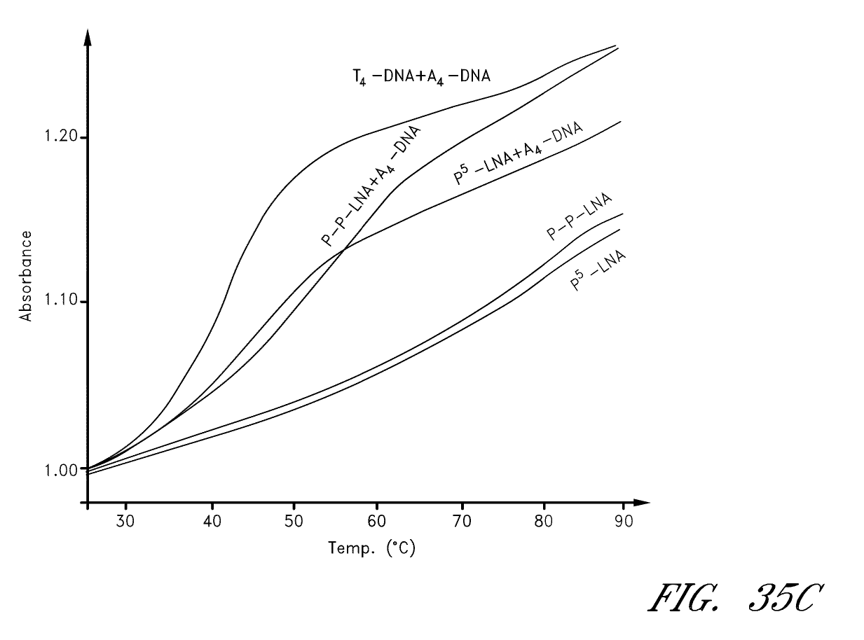

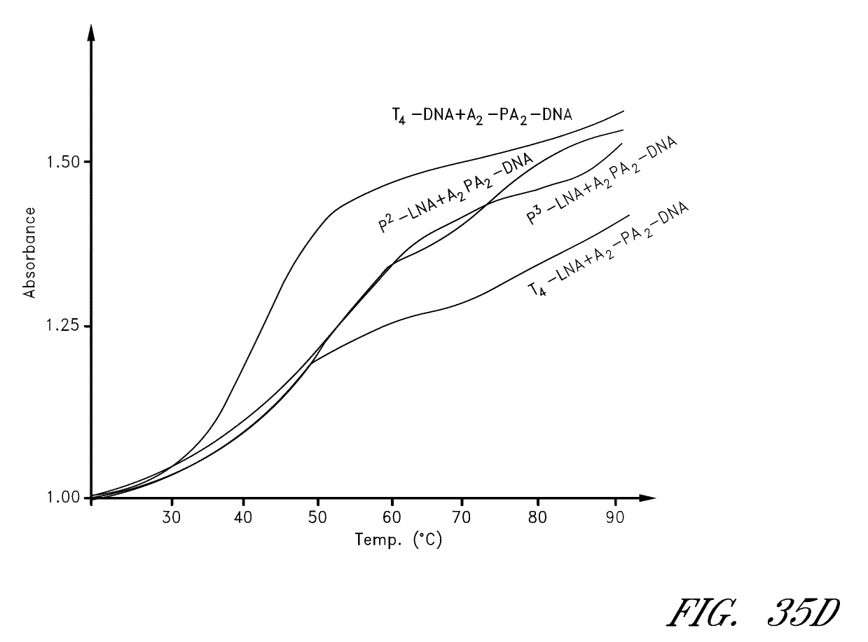

On heating the hairpin forming oligo T₄-DNA (see FIG. 34 for sequence key) alone in a thermal melting experiment a clear transition is observed at 37.2° C. (FIG. 2A and FIG. 34) which is ascribed to opening of the hairpin to an ssDNA. In a similar experiment with T₄-LNA (FIG. 34) a higher transition temperature is to be expected for opening of the corresponding hairpin due to higher stability of the stem which is deduced from the reported higher stabilities of LNA/DNA duplexes. To test this hypothesis T₄-LNA analogous to T₄-DNA was synthesised with five nucleotides in the stem being replaced with the corresponding LNA monomers (T^{L} and ^{Me}C^{L}, respectively). For this modified hairpin only an incipient transition from hairpin to ssLNA could be observed above 80° C. (FIG. 35A). An equimolar mixture of T₄-DNA and A₄-DNA gives a transition at the same temperature as the one observed for T₄-DNA alone, but the increase of optical density (hyperchromacia) is much stronger for the transition of the A₄-DNA/T₄-DNA mixture. The increase in hyperchromacia of the mixture is not due to an additional transition of an A₄-DNA hairpin because this oligo alone has no transition above 20° C. which is in agreement with earlier reports that adenine compared to thymine in the loop destabilises a hairpin (^{vi}). The increased hyperchromacia of the mixture is therefore best explained by the melting of an A₄-DNA/T₄-DNA duplex although it is impossible to estimate the ratio of distribution of T₄-DNA between its hairpin structure and its duplex structure with A₄-DNA.

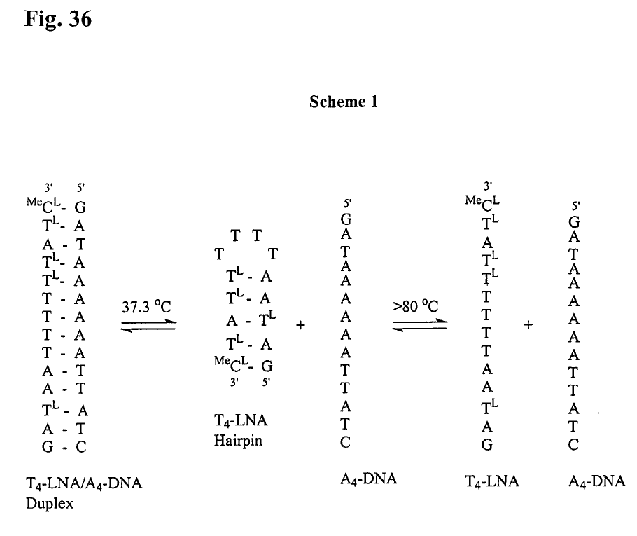

Due to the stability of the T₄-LNA hairpin and the instability of the A₄-DNA hairpin no transitions were expected to be found in the temperature range 20-80° C. for a mixture of these two oligos. It was therefore puzzling to find a transition at 37.3° C. with a rather low hyperchromacia. The extra transition for the A₄-DNA/T₄-LNA mixture is best understood by comparison with the properties of palindromic sequences which have been extensively studied by NMR. For example, it was shown that the self complementary sequence 5′-CGCGTTAACGCG formed a duplex at lower temperatures with a transition to a hairpin at 33° C. at 0.3 mM and again a transition to random coil at 48° C. (^{vii}). As depicted in FIG. 36, Scheme 1 our system is nearly the same, except that the hairpin forming oligo (T₄-LNA) does not have a self complimentary sequence in the loop region and needs another oligo to form the duplex. In our case the required oligo (A₄-DNA) does not form a hairpin at ambient temperatures and is therefore not introducing any complications for the interpretation of the melting of the A₄-DNA/T₄-LNA mixture. By comparison with the nature of the palindromic sequences the melting at 37.3° C. of the A₄-DNA and T₄-LNA mixture is best explained by a transition from DNA/LNA duplex to a mixture of A₄-DNA and T₄-LNA hairpin. For palindromic oligos it has been suggested that the transition from duplex to hairpin takes place through formation of a cruciform structure formed after creation of an initial bulge in the center of the duplex upon melting (vⁱⁱⁱ). Once the cruciform is formed little energy is needed to propagate the mobile junction formed and to complete the separation of the two hairpins. We can argue for a similar mechanism in our case and also for the same type of mechanism operating in the opposite direction, because identical melting curves for up and down temperature modes were obtained. This could implicate that we have found an example of strand invasion into an extremely stable LNA hairpin.

For the 5′-CGCGTTAACGCG sequence, hairpin structures were always observed by NMR at lower temperatures and complete conversion from hairpin to duplex was never observed (^{vii}) which may indicate quenching of the equilibrium at temperatures lower than the transition temperature. Also in our case a rather low hyperchromacia for the transition seems to indicate that the conversion from a mixture of A₄-DNA and T₄-LNA hairpin to DNA/LNA duplex is incomplete. This implies a more complete transition from hairpin to duplex if the melting temperature is higher and closer to the melting of the LNA hairpin. This is indeed what we found when a pyrene pseudonucleotide is inserted in the middle of the A₄ region in the A₄-DNA (FIG. 34). It has previously been found that a single INA insertion in an A/T region of a duplex causes a significant increase in the melting temperature. This is also observed here with a melting of 44.7° C., and furthermore, a significant increase in the hyperchromacia is observed when compared with the DNA/LNA duplex from A₄-DNA (FIGS. 2A and 2D). The higher hyperchromacia in this case indicates that the A₂PA₂-DNA/T₄-LNA duplex has a better ability to be formed in the transition from the LNA hairpin.

From the finding above that pyrene insertions opposite to the LNA monomer lower the melting temperatures of LNA containing duplexes, we tested that proper insertions in the stem of a T₄-LNA hairpin could reduce its stability and make it prone to targeting to A₄-DNA. As the transition temperature of T₄-LNA is too high to be determined, it was promising to observe a thermal transition for the mono pyrene inserted oligos P²-LNA and P⁵-LNA at 81.1° C. and 71.4° C., respectively, and for the double pyrene inserted oligo P-P-LNA at 69.1° C. though it has to be admitted that the hyperchromacia was extremely low for the three transitions. This means that one shall be very cautious about the interpretation and this is symbolised by using parentheses for these transitions in FIG. 34. Irrespectively whether the transitions are due to opening of hairpins or to undefined duplex meltings, we took it as evidence for assuming that these pyrene inserted oligos could be more accessible for duplex formation with their complementary ssDNA targets.

When the oligo P²-LNA with the INA monomer P was inserted after the first nucleotide in the stem, a considerably stronger hyperchromacia was observed on melting of the duplex with A₄-DNA (FIG. 35B) than for T₄-LNA with A₄-DNA without any insertion of P (FIG. 35A). The hyperchromacia is approximately half of the one observed for the T₄-DNA/A₄-DNA duplex which is shown in FIGS. 2A-C as a reference. An increase in the transition temperature is also observed as should be expected because of the stabilising effect of P on hybridisation to a DNA. For both the transition temperature and hyperchromacia a similar result is found for the oligo P⁵-LNA with a P insertion close to the loop of its corresponding hairpin form (FIG. 35C). With two P insertions in the stem region the resulting oligo P-P-LNA shows an even higher transition temperature for its corresponding duplex with A₄-DNA, but more strikingly, the hyperchromacia is nearly the same as the one for the unmodified duplex. This is a clear demonstration that P insertions into LNA with secondary structures can make this special type of LNA more accessible to targeting and at the same time increase the duplex stability with the target, the latter being deduced from higher transition temperatures for the LNA probes with P insertions. It was attempted further to stabilise the duplexes with the LNA probes by inserting P into the target in the region corresponding to the loop in the probes. As seen from FIG. 34, the oligo A₂PA₂ showed even higher transition temperatures and again the highest one was found for two P insertions in the LNA probe (P-P-LNA). Stabilising the duplexes by extra insertions in the target also improved the hyperchromacia as it is seen for the A₂PA₂ oligo (FIG. 35D). The highest melting temperature was found for (APA)₂-DNA when forming a duplex with P-P-LNA. In this case the melting temperature is considerably higher (83.3° C.) than the transition temperature (69.1° C.) measured for what is most likely the P-P-LNA hairpin.

(a) Williams, J. C., Casa-Green, S. C., Mir, K. U. and Southern, E. M. (1994) Studies of oligonucleotide interactions by hybridisation to arrays: the influence of dangling ends on duplex yield. Nucleic Acids Res., 22, 1365-1367. (b) Milner, N., Mir, K. U. and Southern, E. M. (1997) Selecting effective antisense reagents on combinatorial oligonucleotide arrays. Nature Biotechnol., 15, 537-541. (c) Southern, E., Mir, K. and Shchepinov, M. (1999) Molecular interactions on microarrays. Nature Genet., 21, 5-9.

ii (a) Lockhart, D. J., Dong, H. B., Michael, C., Follettie, M. T. Gallo, M. V., Chee, M. S., Mittmann, M. Wang, C., Kobayashi, M., Horton, H. and Brown, E. L. (1996) Expression monitoring by hybridization to high-density oligonucleotide arrays. Nature Biotechnol., 14, 1675-1680. (b) Cronin, M. T., Fucini, R. V., Kim, S. M., Masino, R. S., Wespsi, R. M. and Miyada, C. G. (1996) Cystic fibrosis mutation detection by hybridization to light-generated DNA probe arrays. Hum. Mutat., 7, 244-255.

iii Nguyen, H.-K. and Southern, E. M. (2000) Minimising the secondary structure of DNA targets by incorporation of a modified deoxynucleoside: implications for nucleic acid analysis by hybridisation. Nucleic Acids Res., 28, 3904-3909.

iv (a) Christensen, U. B. and Pedersen, E. B. (2002) Intercalating nucleic acids containing insertions of 1-O-(1-pyrenylmethyl)glycerol: stabilisation of dsDNA and discrimination of DNA over RNA. Nucl. Acids Res., 30, 4918-4925. (b) Filichev, V. V. and Pedersen, E. B. (2003) Intercalating nucleic acids (INAs) with insertion of N-(pyren-1-ylmethyl)-(3R,4R)-4-(hydroxymethyl)pyrrolidin-3-ol. DNA(RNA) duplex and DNA three-way junction stabilities. Org. Biomol., Chem. In press.

v. Petersen, M., Nielsen, C. B., Nielsen, K. E., Jensen, G. A., Bondensgaard, K., Singh, S. K., Rajwanshi, V. K., Koshkin, A. A., Dahl, B. M., Wengel, J. and Jacobsen, J. P. (2000) The conformation of locked nucleic acids (LNA). J. Mol. Recogn., 13, 44-53.

vi Vallone, P. M., Paner, T. M., Hilario, J., Lane M. J., Faldasz, B. D. and Benight, A. S. (1999) Melting studies of short DNA hairpins: influence of loop sequence and adjoining base pair identity on hairpin thermodynamic stability. Biopolymer., 50, 425-442.

vii Hald, M., Pedersen, J. B., Stein, P. C., Kirpekar, F. Jacobsen, J. P. (1995) A comparison of the hairpin stability of the palindromic d(CGCG(A/T)4CGCG) oligonucleotides. Nucleic Acids Res., 23, 4576-4582.

viii Wemmer, D. E., Chou, S. H., Hare, D. R. and Reid, B. R. (1985) Duplex-hairpin transitions in DNA: NMR studies on CGCGTATACGCG. Nucleic Acids Res., 13, 3755-3772.

### Example 26

### Preparation of an Intercalator Pseudo Nucleotide

The example describes preparation and use of N-(pyrene-1-ylmethyl)-(3R,4R)-4-(hydroxymethyl)pyrrolidin-3-ol (4) in the synthesis of several INAs and investigated the hybridisation affinity of INA/DNA, INA/RNA duplexes and DNA TWJ region.

When the N-(pyren-1-ylmethyl)azasugar was inserted as a-bulge good discrimination between stabilities of INA/DNA and INA/RNA duplexes and the increased stability of a DNA three-way junction were observed.

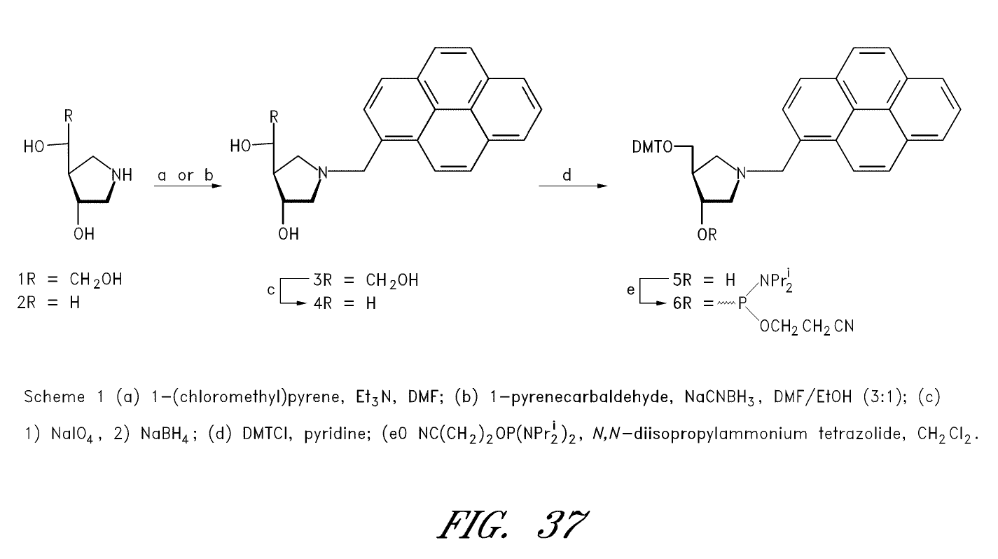

The synthesis of 1′-aza pyrenemethyl pseudonucleoside 4 started from enantiomerically pure 1-aza analogues of 2-deoxy-D-hexofuranose 1 or 2-deoxy-D-ribofuranose 2.¹³ Pyrene substrates having chloromethyl and carbaldehyde functionalities that could be coupled with the secondary amines 1 and 2 were used (FIG. 37).

The DMT protected phosphoroamidite 6 is required for the oligonucleotide synthesis. The primary alcohol 4 was treated with an excess of DMTCl in pyridine with further purification on a silica gel column to give compound 5 in 61% yield. The synthesis of the final phosphoroamidite by treatment with 2-cyanoethyl-N,N-isopropylchlorophosphoramidite in the presence of the excess of Hunig's base¹² failed. To obtain the required phosphoramidite 6 we used an alternative method with 2-cyanoethyl-N,N,N′,N′-tetraisopropylphosphane and N,N-diisopropylammonium tetrazolide.¹⁵ The compound 6 was obtained in 57% yield.

The phosphoramidite 6 was incorporated into different oligonucleotide sequences to give INAs on an automated solid phase DNA synthesizer using an increased coupling time (24 min) and repeating the cycle twice. The coupling efficiencies for the pyrene azasugar derivative 6 were approximately 80-85% compared to approximately 99% for commercial phosphoramidites (2 min coupling).

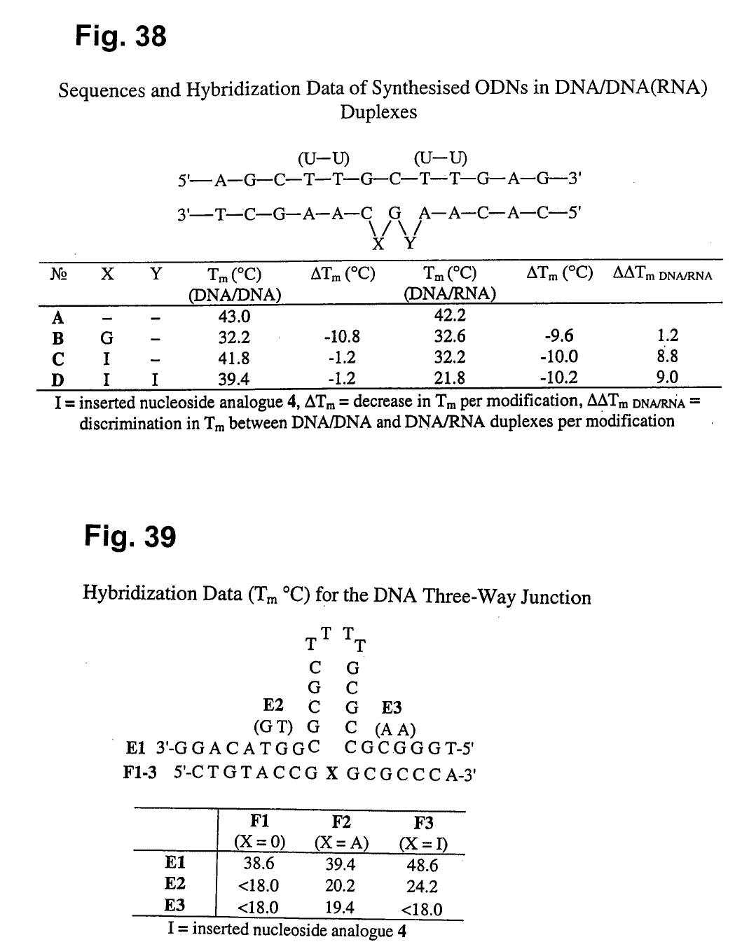

The synthesised INAs were used in the hybridisation studies of INA/DNA and INA/RNA duplexes (FIG. 38) and INA/DNA three way junction (TWJ) (FIG. 39).

INA with incorporation of the N-(pyren-1-ylmethyl)azasugar as the bulge resulted in lowering of the melting temperature with 1.2° C. per modification towards ssDNA (FIG. 15). The corresponding reference duplex in entry B containing a bulging deoxynucleotide (dG) had a considerably lower Tₘ=32.2° C. (ΔTₘ=−10.8° C.). For the INA/RNA duplexes the pyrene containing sequence C and the reference B decreased the stability of the INA/RNA duplex with 10° C. and 9.6° C., respectively, compared to the perfectly matched duplex (entry A). Consequently, INA with pyrene azasugar incorporated as the bulge has better hybridisation affinity towards the complementary ssDNA than towards ssRNA. The differences in melting temperatures for ssDNA and ssRNA seems to be additive with respect to the number of pyrene moieties in the targeting ODN. These results are also in agreement with other investigations where 1-O-(1-pyrenylmethyl)glycerol was inserted twice as bulges. A larger discrimination up to 25.8° C. between INA/DNA and INA/RNA was then observed.⁸ In that case the INA/DNA structure is stabilised compared to the wild type duplex in contrast to our case where a slight decrease is observed for Tₘ. The flexibility of the bulge may be an important factor to obtain both duplex stabilization and discrimination. The synthesis of different linkers and planar aromatic moieties is also in progress. The RNA/DNA discrimination displayed may be applied for purification or detection of DNA targets in a mixture with the very same sequences of RNA.

DNA three way junction (TWJ) composed of two arms linked to a stem (FIG. 39), was observed to lead to a considerable stabilisation when the pyrene azasugar intercalator was inserted in the INA (F3) compared to the ODN having dA at the same position (F2) or without an insertion in the ODN (entry F1). To be sure that hybridization in the arms is important for the stability of the complex; we prepared ODNs with mismatches in either arm of TWJ (entry E2 and E3). In both cases it resulted in a large lowering of the hybridisation affinity.

Experimental

General

NMR spectra were recorded on a Bruker AC-300 FT NMR spectrometer at 300 MHz for ¹H NMR and at 75.5 MHz for ¹³C NMR. Internal standards used in ¹H NMR spectra were TMS (δ: 0.00) for CDCl₃, CD₃OD; in ¹³C NMR were CDCl₃ (δ: 77.0), CD₃OD (δ: 49.0). Accurate ion mass determination was performed on a Kraton MS-50-RF equipped with FAB source. The [M+H]⁺ ions were peakmatched using ions derived from the glycerol matrix. Thin layer chromatography (TLC) analyses were carried out with use of TLC plates 60 F₂₅₄ purchased from Merck and were visualized in an UV light (254 and/or 343 nm) and/or with a ninhydrin spray reagent (0.3 g ninhydrin in 100 cm³ butan-1-ol and 3 cm³ HOAc) for azasugars and its derivatives. The silica gel (0.063-0.200) used for column chromatography was purchased from Merck. ODNs were synthesised on an Assembler Gene Special DNA-Synthesizer (Pharmacia Biotech). Purification of 5′-O-DMT-on and 5′-O-DMT-off ODNs were accomplished using a Waters Delta Prep 4000 Preparative Chromatography System. The modified ODNs were confirmed by MALDI-TOF analysis on a Voyager Elite Elite Biospectrometry Research Station from PerSeptive Biosystems. All solvents were distilled before use. The reagents used were purchased from Aldrich, Sigma or Fluka. The reagents for Gene Assembler were purchased from Cruachem (UK).

### N-(Pyren-1-ylmethyl)-(3R,4S)-4-[(1S)-1,2-dihydroxyethyl]pyrrolidin-3-ol (3)

Method A. Azasugar 1 (50 mg, 0.34 mmol) was dissolved in DMF (5 cm³), 1-(chloromethyl)pyrene (103 mg, 0.41 mmol) and Et₃N (0.057 cm³, 0.41 mmol) were added. The reaction mixture was stirred at room temp. under nitrogen overnight. The solvent was evaporated under reduced pressure and co-evaporated with toluene (2×5 cm³). The residue was chromatographed on a silica gel column with CH₂Cl₂/MeOH (0-20%, v/v) as eluent affording the pure product 3 (70 mg, 57%): R_{f} 0.20 (10% MeOH/CH₂Cl₂); δ_{H}(CD₃OD) 2.36 (1H, m, H-4), 2.95 (1H, m, 5), 3.08 (1H, dd, J 2.8 and 10.5, H-2), 3.22 (1H, dd, J 5.4 and 12.0, H-5), 3.34 (1H, m, H-2), 3.50-3.65 (3H, m, CH[OH]CH₂OH), 4.42 (1H, m, H-3), 4.65 (2H, s, CH₂pyren-1-yl), 4.88 (3H, br. s, 3×OH), 7.90-8.40 (9H, m, Hₐᵣₒₘ); δ_{C}(CD₃OD) 50.7 (C-4), 56.7 (C-5), 57.2 (C-2), 62.7 (CH₂pyren-1-yl), 65.7 (CH₂OH), 71.8 (CH[OH]), 72.7 (C-3), 123.8, 125.5, 125.8, 125.9, 126.6, 126.8, 127.3, 128.0, 128.2, 129.1, 129.4, 129.9, 131.2, 131.9, 132.5, 133.2 (pyren-1-yl); m/z (FAB) 362.1748 [M+H]⁺, C₂₃H₂₄NO₃ requires 362.1756.

Method B. Azasugar 1 (70 mg, 0.48 mmol) was dissolved in DMF/EtOH (3:1, 10 cm³) and 1-pyrenecarbaldehyde (270 mg, 1.18 mmol) and NaCNBH₃ (74 mg, 1.18 mmol) were added. The reaction mixture was stirred at room temp. under nitrogen overnight. Concentrated HCl was added until pH<2. Solvent was evaporated under reduced pressure, co-evaporated with toluene (2×5 cm³). The residue was purified using silica gel column chromatography with CH₂Cl₂/MeOH (0-20%, v/v) affording the compound 3 (110 mg, 63%).

### N-(Pyren-1-ylmethyl)-(3R,4R)-4-(hydroxymethyl)pyrrolidin-3-ol (4)

Method A. Azasugar 2 (100 mg, 0.86 mmol) was dissolved in DMF (10 cm³) and 1-(chloromethyl)pyrene (257 mg, 1.03 mmol) and Et₃N (0.140 cm³, 1.03 mmol) were added. The reaction mixture was stirred at room temp. under nitrogen overnight. The solvent was evaporated under reduced pressure and co-evaporated with toluene (2×5 cm³). The residue was dissolved in H₂O/CH₂Cl₂ (1:1, 40 cm³) and the water layer was extracted with CH₂Cl₂. The combined organic fractions were dried (Na₂SO₄), evaporated in vacuo and chromatographed on a silica gel column with CH₂Cl₂/MeOH (0-20%, v/v) affording the title compound 4 (130 mg, 46%): R_{f} 0.17 (10% MeOH/CH₂Cl₂); δ_{H}(CDCl₃) 2.08 (1H, m, H-4), 2.29 (1H, m, H-5), 2.57 (1H, dd, J 2.8 and 10.2, H-2), 2.85 (2H, m, H-2 and H-5), 3.43 (2H, s, CH₂OH), 3.47 (2H, br. s, 2×OH), 4.08 (1H, m, H-3), 4.19 (2H, s, CH₂pyren-1-yl), 7.90-8.40 (9H, m, Hₐᵣₒₘ); δ_{C}(CDCl₃) 49.8 (C-4), 55.9 (C-5), 57.5 (C-2), 62.3 (CH₂pyren-1-yl), 64.2 (CH₂OH), 73.9 (C-3), 123.3, 124.4, 124.6, 124.8, 125.1, 125.9, 127.3, 127.6, 127.8, 129.5, 130.7, 130.9, 131.1 (pyren-1-yl); m/z (FAB) 332.1631 [M+H]⁺, C₂₃H₂₄NO₃ requires 332.1651.

Method B. Azasugar 2 (1.18 g, 10.1 mmol) was dissolved in DMF/EtOH (3:1, 150 cm³) and 1-pyrene-carbaldehyde (3.47 g, 15.1 mmol) and NaCNBH₃ (950 mg, 15.1 mmol) were added. The reaction mixture was stirred at room temp. under nitrogen overnight. Concentrated HCl was added until pH<2. The solvent was evaporated under reduced pressure and co-evaporated with toluene (2×50 cm³). The residue was dissolved in H₂O/CH₂Cl₂ (1:1, v/v, 150 cm³) and the water layer was extracted with CH₂Cl₂ (3×75 cm³). The combined organic fractions were dried (Na₂SO₄), evaporated under diminished pressure. The residue was purified using silica gel column chromatography with CH₂Cl₂/MeOH (0-20%, v/v) affording the compound 4 as an oil which crystallised on standing (1.9 g, 57%), mp 104-105° C.

Method C. A cooled solution of compound 3 (110 mg, 0.304 mmol) in EtOH (4 cm³) was added to a solution of NaIO₄ (71.6 mg, 0.335 mmol) in H₂O (1.5 cm³) under stirring. After 30 min NaBH₄ (12.3 mg, 0.335 mmol) was added. After 30 min the resulting solution was acidified with 2M HCl until pH 2 under vigorous stirring. The solvent was removed in vacuo. The residue was dissolved in H₂O/CH₂Cl₂ (1:1, v/v, 20 cm³) and extracted with CH₂Cl₂ (4×15 cm³). The combined organic layers were dried (Na₂SO₄), evaporated under diminished pressure to dryness affording compound 4 (40 mg, 40%).

### N-(Pyren-1-ylmethyl)-(3R,4R)-4-[(4,4′-dimethoxytriphenylmethoxy)methyl]pyrrolidin-3-ol (5)

Compound 4 (139 mg, 0.42 mmol) was dissolved in anhydrous pyridine (10 cm³) and DMTCl (178 mg, 0.53 mmol) was added. The mixture was stirred for 24 h under nitrogen at room temp. MeOH (1 cm³) was added to quench the reaction and the solvents were evaporated under reduced pressure and co-evaporated with toluene (2×5 cm³). The residue was re-dissolved in H₂O/CH₂Cl₂ (1:1, v/v, 20 cm³), and the mixture was washed with saturated aqueous NaHCO₃. The organic layer was dried (Na₂SO₄), and concentrated under reduced pressure. Purification using silica gel column chromatography (5-40% EtOAc/cyclohexane, v/v) gave the title compound 5 as a foam (160 mg, 61%) which was used in the next step without further purification: R_{f} 0.45 (49% EtOAc/49% cyclohexane/2% Et₃N, v/v/v); δ_{H}(CDCl₃) 2.20 (1H, m, H-4), 2.34 (1H, m, H-5), 2.53 (1H, br.s, OH), 2.62 (1H, dd, J 5.6 and 9.9, H-2), 2.72 (1H, dd, J 2.5 and 9.8, H-2), 3.06 (3H, m, CH₂ODMT and H-5), 3.71 (6H, s, OCH₃), 4.01 (1H, m, H-3), 4.21 (2H, s, CH₂pyren-1-yl), 6.78 (4H, m, DMT), 7.10-7.40 (9H, m, DMT), 7.90-8.40 (9H, m, Hₐᵣₒₘ); δ_{C}(CDCl₃) 48.8 (C-4), 55.2 (OCH₃), 56.1 (C-5), 58.0 (C-2), 61.9 (CH₂pyren-1-yl), 64.5 (CH₂OH), 74.9 (C-3), 85.9 (C—Ar₃), 113.0, 123.8-132.3 (DMT and pyren-1-yl), 144.9, 158.4 (DMT); m/z (FAB) 634.2740 [M+H]⁺, C₄₄H₄₂NO₅ requires 634.2722.

### N-(Pyren-1-ylmethyl)-(3R,4R)-3-O-(2-cyanoethoxy(diisopropylamino)-phophiino]-4-[(4,4′-dimethoxytriphenylmethoxy)methyl]pyrrolidine (6)

Compound 5 (140 mg, 0.22 mmol) was dissolved under nitrogen in anhydrous CH₂Cl₂ (5 cm³). N,N-Diisopropylammonium tetrazolide (61 mg, 0.42 mmol) was added followed by dropwise addition of 2-cyanoethyl-N,N,N′,N′-etraisopropylphosphane (0.140 cm³, 0.44 mmol). After 2.0 h analytical TLC showed no more starting material and the reaction was quenched with H₂O (1 cm³) followed by addition of CH₂Cl₂ (10 cm³). The mixture was washed with saturated aqueous NaHCO₃ (2×10 cm³). The organic phase was dried (Na₂SO₄) and the solvents were removed under reduced pressure. The residue was purified using silica gel column chromatography with cyclohexane/EtOAc (0-20%, v/v). Combined UV-active fractions were evaporated in vacuo affording 6 (158 mg, 57%) as foam that was co-evaporated with dry acetonitrile (3×30 cm³) before using it in ODN synthesis. R_{f} 0.85 (49% EtOAc/49% cyclohexane/2% Et₃N, v/v/v); δ_{H}(CDCl₃) 0.93 (6H, m, CH₃ [Prⁱ]), 1.04 (6H, m, CH₃ [Prⁱ]), 2.30 (2H, m, H-4 and H-5), 2.48 (2H, m, CH₂CN), 2.64 (1H, m, H-2), 2.78 (1H, m, H-2), 2.98 (2H, m, OCH₂CH₂CN), 3.08 (1H, m, H-5), 3.50 (4H, m, CH [Prⁱ] and CH₂ODMT), 3.65 (6H, s, OCH₃), 4.01 (1H, m, H-3), 4.20 (2H, m, CH₂pyren-1-yl), 6.68 (4H, m, DMT), 7.05-7.40 (9H, m, DMT), 7.85-8.40 (9H, m, Hₐᵣₒₘ); δ_{P}(CDCl₃) 148.2 (s), 149.0 (s) in the ratio 2:1.

### Synthesis and Purification of Modified and Unmodified Oligodeoxynucleotides

The oligodeoxynucleotides were synthesised on a Pharmacia Gene Assembler® Special synthesizer in 0.2 μmol-scale (7.5 μmol embedded per cycle, Pharmacia primer support™) using commercially available 2-cyanoethylphosphoramidites and 6. The synthesis followed the regular protocol for the DNA synthesizer. The coupling time for 6 was increased from 2 to 24 min and the cycle was repeated twice. The 5′-O-DMT-on ODNs were removed from the solid support and deprotected with 32% aqueous NH₃ (1 cm³) at 55° C. for 24 h and then purified on preparative HPLC using a Hamilton PRP-1 column. The solvent systems were buffer A [950 cm³ 0.1 M NH₄HCO₃ and 50 cm³ CH₃CN (pH=9.0)] and buffer B [250 cm³ 0.1 M NH₄HCO₃ and 750 cm³ CH₃CN (pH=9.0)] which were used in the following order: 5 min A, 30 min liner gradient of 0-70% B in A, 5 min liner gradient of 70-100% B in A. Flow rate was 1 cm³ min⁻¹. The purified 5′-O-DMT-on ODNs eluted as one peak after approximately 30 min [UV control 254 nm and 343 nm (for pyrene containing ODNs)]. The fractions were concentrated in vacuo followed by treatment with 10% aqueous HOAc (1 cm³) for 20 min and further purification on HPLC under the same conditions to afford detritylated ODNs which eluted at 23-28 min. The purity of oligos synthesised was 99-100% according to the preparative HPLC. The resulted solutions were evaporated in vacuo and co-evaporated twice with water to remove volatile salts to afford ODNs, which were used in melting temperature measurements. All oligonucleotides containing pyrenylmethylazasugar derivative 6 were confirmed by MALDI-TOF analysis (entry C: found 4005.65, calcd. 4005.76; entry D: 4398.02, calcd. 4398.87; entry F3: found 4903.05, calcd. 4904.89).

Melting Experiments

Melting temperature measurements were performed on a Perkin-Elmer UV/VIS spectrometer fitted with a PTP-6 Peltier temperature-programming element. The absorbance at 260 nm was measured from 18° C. to 85° C. in 1 cm cells. The melting temperature was determined as the maximum of the derivative plots of the melting curve. The oligodeoxynucleotides were dissolved in a medium salt buffer (pH=7.0, 1 mM EDTA, 10 mM Na₂HPO_{4×2}H₂O, 140 mM NaCl) to a concentration of 1.0 μM for each strand.

### Example 27

### Fluorescence when Hybridized to Mismatched Targets

Quenching in fluorescence is a sign of strong interaction of the fluorophore with the duplex. Structural minimization calculations have supported that the pyrene moiety is intercalated into the duplex. It was therefore anticipated that the introduction of mismatches near the site of intercalation results in increased flexibility to the pyrene and hence increased fluorescence. This was also what was found irrespective to which side of the intercalator a mismatch is introduced (Table 6).

TABLE 6
Fluorescent data of mono modified ODN hybridised to either the
complementary sequence or to one of six different neighbouring
single point mutants.

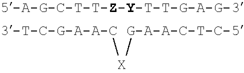

| | | | 382 nm | 395 nm | 480 nm. |
|---|---|---|---|---|---|
| | | | Rel. | Rel. | Rel. |
| Name | Z | Y | Intensity | Intensity | Intensity |
| Probe | — | — | 48 | 40 | 1 |
| alone | | | | | |
| Wt | G | C | 15 | 12 | 1 |
| Mut. 1 | C | C | 59 | 50 | 2 |
| Mut. 2 | A | C | 75 | 63 | 2 |
| Mut. 3 | T | C | 50 | 42 | 2 |
| Mut. 4 | G | T | 34 | 29 | 2 |
| Mut. 6 | G | A | 63 | 53 | 2 |

To test the hypothesis that probe III could be used for detection of single point mutants, it was hybridised to target with all four variants of Y, and the intensity of the excimer band at 480 nm was significantly increased (Table 7) when a mismatch was introduced (Y=G, A, T). Again it was expected that the fluorescence of the bands at 382 and 395 nm would increase upon introduction of a mismatch, which was also observed. Surprisingly the fluorescence at 480 nm also increased with introduction of a mismatch at the 3′ side of both intercalators (Z=C, A, T), indicating that the two pyrene moieties are able to interact with each other (Table 7). This would only be expected if a loop, large enough to let the pyrene moieties interact, is created. It is noteworthy that the fluorescence at 382 and 395 is quenched when hybridised to a complementary sequence, but increased when hybridizing to a sequence with one mismatch. It should therefore be possible to use all three wavelengths (382, 295 and 480) to differentiate between a fully complementary sequence and a complementary sequence with one mismatch.

TABLE 7
Fluorescent data of a double modified ODN hybridised to either
the complementary sequence or to one of six different single
point mutants.

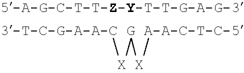

| | | | 382 nm | 395 nm | 480 nm |
|---|---|---|---|---|---|
| | | | Rel. | Rel. | Rel. |
| Name | Z | Y | Intensity | Intensity | Intensity |
| SsDNA | — | — | 44 | 38 | 17 |
| Wt | G | C | 19 | 17 | 4 |
| Mut. 1 | C | C | 84 | 73 | 14 |
| Mut. 2 | A | C | 74 | 64 | 10 |
| Mut. 3 | T | C | 84 | 74 | 12.5 |
| Mut. 4 | G | T | 62 | 54 | 8 |
| Mut. 5 | G | G | 84 | 74 | 17 |
| Mut. 6 | G | A | 70 | 60 | 12 |

### Example 28

### Thermal Denaturation Studies

Insertions of intercalators into DNA have been accompanied by decrease in specificity for hybridization to fully complementary sequences when compared to sequences with mismatches in the basepairs surrounding the intercalator. Experiments were aimed to test if this is also the case for duplexes with pyrene moiety insertions, placing mismatches to either side of the intercalator, next to and between two intercalators

The specificity is measured by the difference in the melting temperature between the fully complementary duplex and the duplex where one mismatch has been introduced. Melting temperature data are presented in Table 8.

TABLE 8
Melting temperature data for ODNs with different insertion patterns hybridised to
either the complementary strand or one of the three possible point mutations at
nucleotide #6 and #7. X = 1.

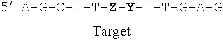

| | |
|---|---|
| | I |
| 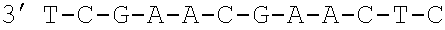 | |

| | |
|---|---|
| | II |
| 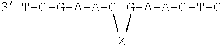 | |
| | III |
| 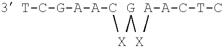 | |
| | IV |
| 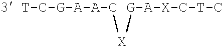 | |
| | V |
| 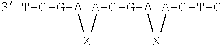 | |

| | Tar- | | Probe | | | | | | | | | |
|---|---|---|---|---|---|---|---|---|---|---|---|---|
| | get | | I | | II | | III | | IV | | V | |
| | Z | Y | [° C.] | | [° C.] | | [° C.] | | [° C.] | | [° C.] | |
| Wt | G | C | 47.4 | ΔT | 50.4 | ΔT | 51.4 | ΔT | 45.4 | ΔT | 60.8 | ΔT |
| Mut. 1 | C | C | 23.4 | −24.0 | 34.0 | −16.0 | 38.0 | −13.4 | 23.4 | −22.0 | 33.8 | −27.0 |
| Mut. 2 | A | C | 30.8 | −16.6 | 34.2 | −16.2 | 36.6 | −14.8 | | | | |
| Mut. 3 | T | C | 27.6 | −18.8 | 33.6 | −16.8 | 35.2 | −16.2 | 25.4 | −20.0 | 37.4 | −23.4 |
| Mut. 4 | G | T | 36.2 | −11.2 | 42.2 | −8.2 | 45.2 | −6.2 | 36.6 | −8.8 | 45.8 | −15.0 |
| Mut. 5 | G | G | 40.0 | −7.4 | 42.4 | −8.0 | 38.6 | −12.8 | 39.4 | −6.0 | 53.2 | −7.6 |
| Mut. 6 | G | A | 39.8 | −7.6 | 39.0 | −11.4 | 39.0 | −12.4 | 39.2 | −6.2 | 49.0 | −11.8 |

As seen from Table 8 the specificity against mismatches of the modified probe is in the range of that of the unmodified probe, though there seems to be a drop in selectivity for C-C mismatches to the 5′ end of the intercalation site (Table 8; Mut. 1 with Probe II and III). The only other consistent trend is that the double modified probe where the two intercalators are separated by four nucleotides (probe V) is more specific against mismatches two nucleotides away from any of the intercalation sites than the unmodified probe (probe I). The rest of the melting temperature differences are close to the values for the unmodified duplexes. Most important in respect to the search for single point mutants is that probe III is selective for its target having the right base in between the pyrene moieties, which is the case and in two out of the three possible mismatches it is even more specific, being less specific in the last instance (Table 8).

### Example 29

### Beacon-Primers

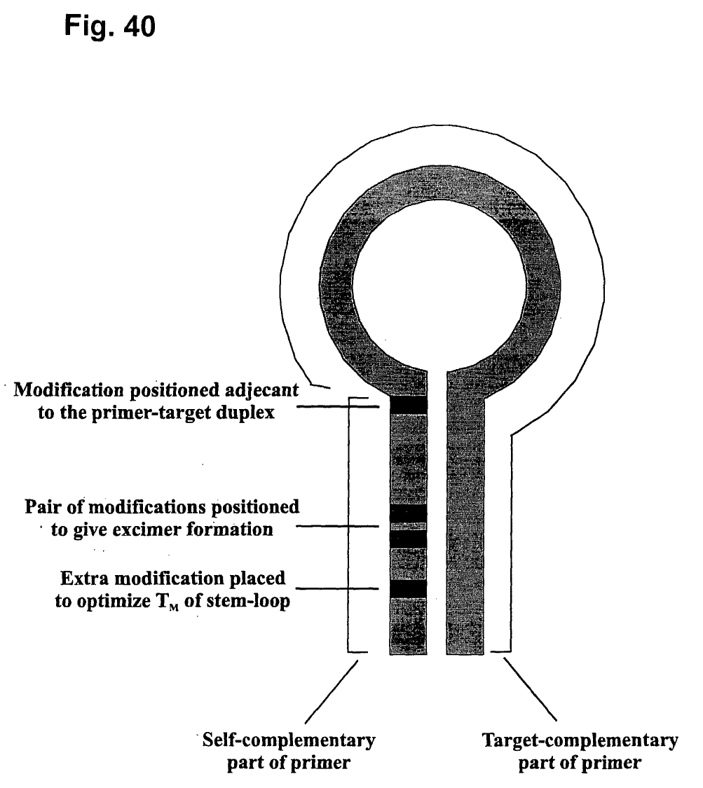

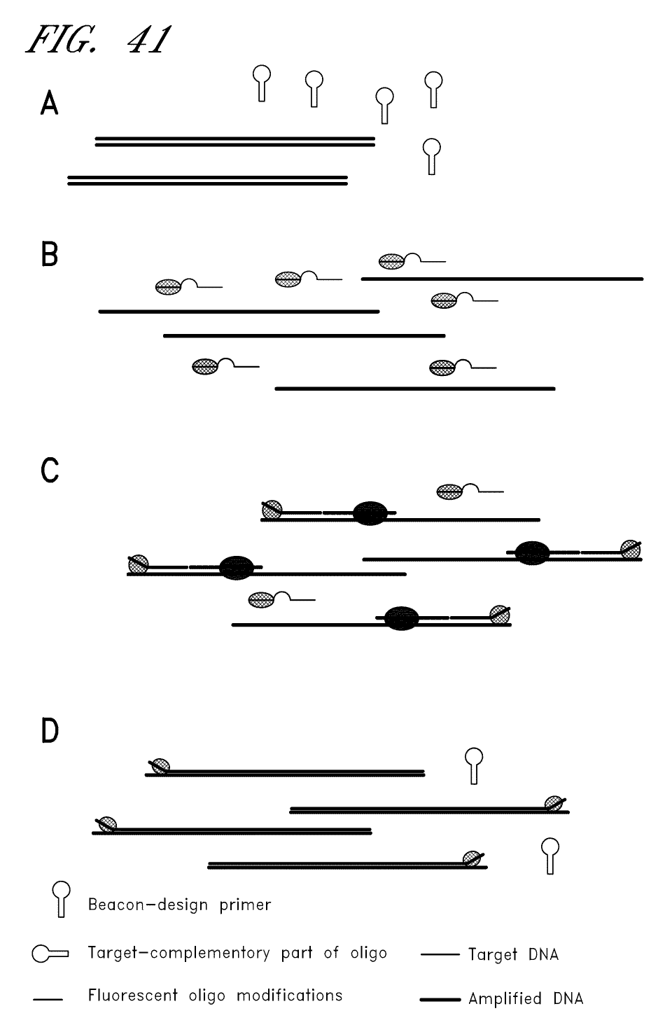

An example of a Beacon-design primer is given in FIG. 40. The primer consists of 39 nucleotides, which are designed so that they can form a stem-loop structure.

The primer has a target-complementary region, that is complementary to the target DNA, which is 24 nucleotides long. Furthermore, the primer has a self-complementary region, that is capable of hybridising to the other end of the primer. The self-complementary region is 15 nucleotides long and comprises furthermore 4 intercalator pseudonucleotides. Two of the intercalator pseudonucleotides are positioned so they are capable of forming an intramolecular excimer.

The melting temperature of the primer/target hybrid is around 67° C., whereas the melting temperature of the selfhybrid is around 46° C.

The beacon primer can be used for PCR and allows quantification of the PCR.

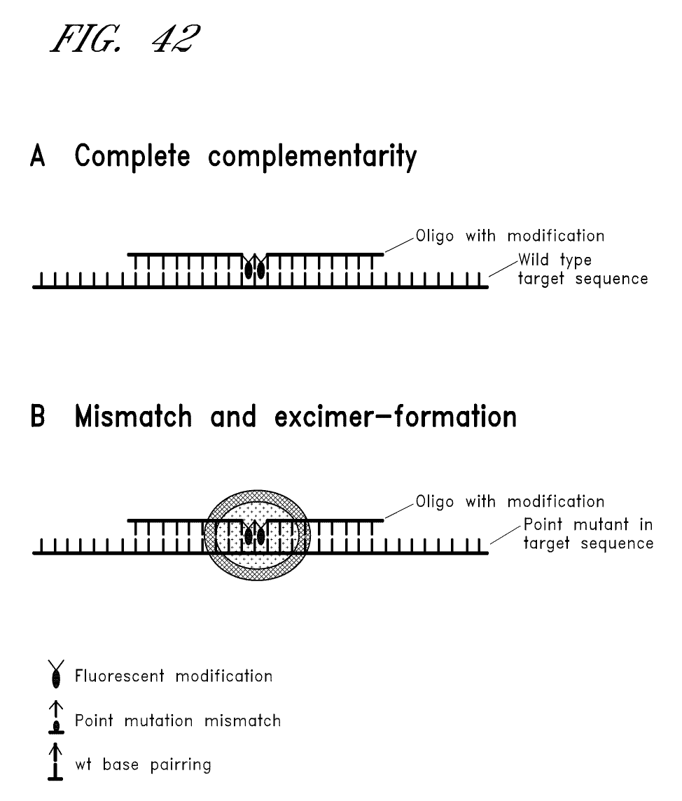

Target specific beacon primers and template DNA is provided (FIG. 41A). The beacon primers and the template DNA is stored at 0-4° C.

Double stranded DNA is denatured at 94° C. (FIG. 41B). The beacon primers are annealed to the target DNA at around 65° C. (FIG. 41C) and the primers are elongated by Taq polymerase.

Subsequently, the temperature is lowered to around 45° C. and unhybridised beacon primers are hybridised to itself (FIG. 41 D). Excimer fluorescence is determined and correlated to the amount of elongated beacon primer.

### REFERENCES

Van Aerschot, A et al., Angew. Chem. Int. Ed. Engl., 1995, 34:1338-1339

Allan, B. et al., Chem. Eur. J., 1999, 5:2424-2431,

Hossain, N et al., J. Org. Chem., 1998, 63: 1574-1582

Korshun V A, Balakin K V, Proskurina T S, Mikhalev I I, Malakhov A F D and Berlin Y A, 1999, A pyrene SECO-pseudonucleotide in constructing interaction-sensitive fluorescent DNA probes; Nucleosides & Nucleotides, 18:2661-2676

Koshkin, A A et al., Tetrahedron, 1998, 54:3607-3630

Nielsen, P E et al., Science, 1991, 254:1497

Obika, S et al., Tetrahedron lett., 1997, 38: 8735-8738

Singh, S K et al., Chem. Commun., 1998, 455-456

Strässler C, Davis N E and Kool E T, 1999, Novel nucleoside analogues with fluorophores replacing the DNA base; Helvetica Chimica Acta, 82:2160-2171

Yamana K, Takei M and Nakano H, 1997, Synthesis of oligonucleotide derivatives containing pyrene labeled glycerol linkers: enhanced fluorescence on binding to a complementary DNA sequence; Tetrahedron Letters, 38:6051-6054.

Yamana K, Iwase R, Furutani S, Tsuchida H, Zako H, Yamaoka T and Murakami A, 1999, 2′-pyrene modified oligonucleotide provides a highly sensitive fluorescent probed of RNA; Nucleic acids research, 27:2387-2392.Claims

(a) E. Uhlmann, Curr. Opin. Drug Discovery Dev., 2000, 3, 203-; (b) A. De Mesmaeker, R. Häner, P. Martin, H. E. Moser, Acc. Chem. Res., 1995, 28, 366-374; (c) M. Egli, Angew. Chem. Int. Ed. Engl., 1996, 35, 1894-1910; (d) S. Verma, F. Eckstein, Annual Rev. Biochemistry, 1998, 67, 99-134; (e) D. Praseuth, A. L. Guieysse, C. Helene, Biochim. Biophis. Acta, 1999, 1489, 181-206; (f) J. Micklefield, Curr. Med. Chem., 2001, 8, 1157-1179; (g) E. T. Kool, Chem. Rev., 1997, 97, 1473-1487.

J. Wengel, Acc. Chem. Res., 1999, 32, 301-310.

(a) A. Van Aerschot, I. Verheggen, C. Hendrix, P. Herdewijn, Angew. Chem. Int. Ed. Engl. 1995, 34, 1338-1339. (b) C. Hendrix, H. Rosemeyer, I. Verheggen, F. Seela, A. Van Aerschot, P. Herdewijn, Chem. Eur. J. 1997, 3, 110-120.

C. J. Leumann, Bioorg. Med. Chem., 2002, 10, 841-854.

(a) I. Luyten, P. Herdewijn, Eur. J. Med. Chem., 1998, 33, 515-576; (b) P. Herdewijn, Antisense & Nucl. Acid Drug Development, 2000, 10, 293-310; (c) K. M. Guckian, B. A. Schweitzer, R. X.-F. Ren, C. J. Sheils, D. C. Tahmassebi, E. T. Kool, J. Am. Chem. Soc. 2000, 122, 2213-2222.

M. J. Davies, A. Shah, I. J. Bruce, Chem. Soc. Rev., 2000, 29, 97-107. C. F. Bleczinski, C. Richert, J. Am. Chem. Soc. 1999, 121, 10889-10894.

U. B. Christensen, E. B. Pedersen, Nucl. Acids Res., in the press.

(a) R. T. Batey, R. P. Rambo, J. A. Doudna, Angew. Chem. Int. Ed. Engl., 1999, 38, 2326-2343; (b) F. Belmont, J.-F. Constant, M. Demeunyck, Chem. Soc. Rev., 2001, 30, 70-81.

(a) S. T. Cload, A. Schepartz, J. Amer. Chem. Soc., 1994, 116, 437-442; (b) M. Zhong, M. S. Rashes, N. B. Leontis, N. R. Kallenbach, Biochemistry, 1994, 33, 3660-3667; (c) Q. Guo, M. Lu, M. E. A. Churchill, T. P. Tullius, N. R. Kallenbach, Biochemistry, 1990, 29, 10927-10934; (d) F. Stuhneier, J. B. Welch, A. I. H. Murchie, D. M. J. Lilley, R. M. Clegg, Biochemistry, 1997, 36, 13530-13538.

(a) M. A. Rosen, D. J. Patel, Biochemistry, 1993, 32, 6563-6575; (b) M. A. Rosen, D. J. Patel, Biochemistry, 1993, 32, 6576-6587.

(a) O. M. Ali, T. Franch, K. Gerdes, E. B. Pedersen, Nucleic Acids Res., 1998, 26, 4919-4924; (b) C. S. Poulsen, E. B. Pedersen, C. Nielsen, Acta Chem. Scand., 1999, 53, 425-431; (c) A. A.-H. Abdel-Rahman, O. M. Ali, E. B. Pedersen, Tetrahedron, 1996, 52, 15311-15324; (d) A. F. Khattab, E. B. Pedersen, Nucleosides, Nucleotides, 1998, 17, 2351-2365; (e) S. A. El-Kafrawy, M. A. Zahran, E. B. Pedersen, C. Nielsen, Helv. Chim. Acta, 2000, 83, 1408-1416.

V. V. Filichev, M. Brandt, E. B. Pedersen, Carbohydr. Res., 2001, 333, 115-122.

V. V. Filichev, E. B. Pedersen, Tetrahedron, 2001, 57, 9163-9168.

J. Burmeister, A. Azzawi, G. Von Kiedrowski, Tetrahedron lett 1995, 36, 3667-3668.

What is claimed is:

1. An intercalator pseudonucleotide capable of being chemically inserted between two nucleotides and having the general structure:
X—Y-Q
wherein
X is a backbone monomer unit capable of being incorporated into the backbone of a nucleic acid or nucleic acid analogue, the backbone monomer unit having the general formula: 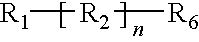
wherein n=3 or 4, R₁ is a trivalent or pentavalent substituted phosphoratom, R₂ is individually selected from an atom capable of forming at least two bonds, R₂ optionally being individually substituted, and R₆ is a protecting group;
Q is an intercalator comprising at least one essentially flat conjugated system containing pyrene which is capable of co-stacking with nucleobases of DNA or RNA;
Y is a linker moiety linking any of R₂ of the backbone monomer unit and the intercalator, wherein Y comprises a chain of m atoms selected from the group consisting of C, O, S, N, P, Se, Si, Ge, Sn, and Pb, wherein one end of the chain is connected to the intercalator and the other end of the chain is connected to the backbone monomer unit, wherein the m atoms are optionally individually substituted and m is 3 or 4;
wherein the total length of Q and Y is in the range from 9 Å to 13 Å;
wherein the intercalator pseudonucleotide does not contain a nucleotide; and
wherein, when the intercalator pseudonucleotide is incorporated into an oligonucleotide or oligonucleotide analog to form an intercalating nucleic acid (INA), the INA is substantially unable to carry out intermolecular hybridization to a corresponding complementary INA, or is substantially unable to carry out intramolecular hybridization

2. The intercalator pseudonucleotide according to claim 1, wherein when said intercalator pseudonucleotide is incorporated into an oligonucleotide or oligonucleotide analog to form an intercalating nucleic acid (INA), the INA further has one or more of the following properties:
capable of discriminating between complementary DNA or RNA;
increased specificity to a complementary oligonucleotide or oligonucleotide analogue as compared with the corresponding oligonucleotide or oligonucleotide analogue without an intercalator pseudonucleotide;
increased nuclease stability;
autofluorescence properties; or
stably hybridizes to a complementary oligonucleotide or oligonucleotide analogue.

3. The intercalator pseudonucleotide according to claim 1, wherein the backbone monomer unit is capable of being incorporated into the phosphate backbone of a nucleic acid or nucleic acid analogue in a manner so that at most 4 atoms separate the two phosphoratoms of the backbone that are closest to the intercalator.

4. The intercalator pseudonucleotide according to claim 1, wherein the linkage from at least one phosphor atom to at least one atom capable of forming a linkage to a neighboring nucleotide is at most 4 atoms long.

5. The intercalator pseudonucleotide according to claim 1, wherein the backbone monomer unit comprises an acyclic backbone monomer unit.

6. The intercalator pseudonucleotide according to claim 5, wherein the acyclic backbone monomer unit is capable of stabilising a bulge insertion.

7. The intercalator pseudonucleotide according to claim 6, wherein the backbone monomer unit comprises a phosphoramidit.

8. The intercalator pseudonucleotide according to claim 7, wherein the backbone monomer unit comprises a trivalent phosphoramidit.

9. The intercalator pseudonucleotide according to claim 1, wherein the backbone monomer unit comprises a removable protecting group, wherein removal of the protecting group allows for a chemical reaction between the intercalator pseudonucleotide and a nucleotide, a nucleotide analogue or another intercalator pseudonucleotide.

10. The intercalator pseudonucleotide according to claim 9, wherein the protecting group is removable by acid treatment.

11. The intercalator pseudonucleotide according to claim 10, wherein the protecting group is 4,4′-dimethoxytriphenylmethyloxy.

12. The intercalator pseudonucleotide according to claim 1 selected from the group consisting of (S)-1-(4,4′-dimethoxytriphenylmethyloxy)-3-pyrenemethyloxy-2-propanol and (R)-1-(4,4′-dimethoxytriphenylmethyloxy)-3-pyrenemethyloxy-2-propanol.

13. The intercalator pseudonucleotide according to claim 1 having fluorescence properties

14. An intercalating nucleic acid (INA) comprising an oligonucleotide or oligonucleotide analogue containing at least one intercalator pseudonucleotide according to claim 1.

15. The intercalating nucleic acid (INA) according to claim 14, containing two or more intercalator pseudonucleotides.

16. The intercalating nucleic acid (INA) according to claim 15, containing three or more intercalator pseudonucleotides.

17. The intercalating nucleic acid (INA) according to claim 14, wherein the oligonucleotide or oligonucleotide analogue is a subunit of DNA.

18. The intercalating nucleic acid (INA) according to claim 14, wherein fluorescence properties of the intercalator pseudonucleotide are altered upon hybridisation of the oligonucleotide or oligonucleotide analogue to a corresponding nucleic acid or nucleic acid analogue under a predetermined stringency.

19. The intercalating nucleic acid (INA) according to claim 14, wherein the melting temperature of a hybrid consisting of the INA and a homologously complementary DNA is higher than the melting temperature of a hybrid between a corresponding oligonucleotide or oligonucleotide analogue lacking an intercalator pseudonucleotide and the homologously complementary DNA.

20. The intercalating nucleic acid (INA) according to claim 19, wherein the melting temperature of the INA hybrid is at least 3° C. higher than the melting temperature of the corresponding DNA hybrid.

21. The intercalating nucleic acid (INA) according to claim 14, wherein the melting temperature of a hybrid consisting of the INA and a homologously complementary DNA is higher than the melting temperature of a hybrid consisting of the INA and a homologously complementary RNA.

22. The intercalating nucleic acid (INA) according to claim 21, wherein the melting temperature of the DNA hybrid is at least 5° C. higher than the melting temperature of the RNA hybrid.

23. The intercalating nucleic acid (INA) according to claim 22, wherein the melting temperature of the DNA hybrid is at least 10° C. higher than the melting temperature of the RNA hybrid.

24. The intercalator pseudonucleotide according to claim 1, wherein m is 3.

25. The intercalator pseudonucleotide (INA) according to claim 19, wherein the melting temperature of the INA hybrid is at least 5° C. higher than the melting temperature of the corresponding DNA hybrid.

26. The intercalator pseudonucleotide according to claim 1, wherein said intercalator pseudonucleotide is 1-(4,4′-dimethoxytriphenylmethyloxy)-3-pyrenemethyloxy-2-propanol.


SEQUENCE LISTING (21 sequences)

SEQ ID NO: 1 - DNA, 13 bp, Artificial Sequence
  Oligonucleotide
agcttgccttgag

SEQ ID NO: 2 - DNA, 13 bp, Artificial Sequence
  Oligonucleotide
agcttgccttgag

SEQ ID NO: 3 - DNA, 12 bp, Artificial Sequence
  Oligonucleotide
agcttgcttgag

SEQ ID NO: 4 - DNA, 12 bp, Artificial Sequence
  Oligonucleotide
tcgaacgaactc

SEQ ID NO: 5 - DNA, 15 bp, Artificial Sequence
  Oligonucleotide
tgtcgagggcgtcga

SEQ ID NO: 6 - DNA, 24 bp, Artificial Sequence
  Oligonucleotide
aagcttcaatcggatggttcttcg

SEQ ID NO: 7 - DNA, 42 bp, Artificial Sequence
  Oligonucleotide
atagtatttattcgaagttagcctaccaagaagccttttttg

SEQ ID NO: 8 - DNA, 12 bp, Artificial Sequence
  Oligonucleotide
ctcaaccaagct

SEQ ID NO: 9 - DNA, 12 bp, Artificial Sequence
  Oligonucleotide
gagttggttcga

SEQ ID NO: 10 - DNA, 24 bp, Artificial Sequence
  Oligonucleotide
aagcttcaatcggatggttcttcg

SEQ ID NO: 11 - DNA, 24 bp, Artificial Sequence
  Oligonucleotide
aagcttcaatcggatggttcttcg

SEQ ID NO: 12 - DNA, 30 bp, Artificial Sequence
  Oligonucleotide
atccgaaagcttcaatcggatggttcttcg

SEQ ID NO: 13 - DNA, 29 bp, Artificial Sequence
  Oligonucleotide
tccgaaagcttcaatcggatggttcttcg

SEQ ID NO: 14 - DNA, 24 bp, Artificial Sequence
  Oligonucleotide
cacaagagctgacccaatggttgc

SEQ ID NO: 15 - DNA, 24 bp, Artificial Sequence
  Oligonucleotide
cacaagagctgacccaatggttgc

SEQ ID NO: 16 - DNA, 30 bp, Artificial Sequence
  Oligonucleotide
gggtcacacaagagctgacccaatggttgc

SEQ ID NO: 17 - DNA, 29 bp, Artificial Sequence
  Oligonucleotide
ggtcacacaagagctgacccaatggttgc

SEQ ID NO: 18 - DNA, 12 bp, Artificial Sequence
  Oligonucleotide
cgcgttaacgcg

SEQ ID NO: 19 - DNA, 12 bp, Artificial Sequence
  Oligonucleotide
tcgaacgaactc

SEQ ID NO: 20 - RNA, 13 nt, Artificial Sequence
  Oligonucleotide
agcuugccuugag

SEQ ID NO: 21 - DNA, 12 bp, Artificial Sequence
  Oligonucleotide
agcttgcttgag